SEQUENCE LISTING (127 sequences)

SEQ ID NO: 1 - DNA, 100 bp, Artificial Sequence
  Target sequence of ATF2
ccaggcaatacaaggacctgtggaatatgagtgatgacaaaccctttctatgtactgcgcctggatgtggccagcgttttaccaacgaggatcatttggc

SEQ ID NO: 2 - DNA, 100 bp, Artificial Sequence
  Target sequence of AGER
gaaaggagaccaagtccaactaccgagtccgtgtctaccagattcctgggaagccagaaattgtagattctgcctctgaactcacggctggtgttcccaa

SEQ ID NO: 3 - DNA, 100 bp, Artificial Sequence
  Target sequence of AREG
acaagacggaaagtgaaaatacttcagataaacccaaaagaaagaaaaagggaggcaaaaatggaaaaaatagaagaaacagaaagaagaaaaatccatg

SEQ ID NO: 4 - DNA, 100 bp, Artificial Sequence
  Target sequence of C1QA
ggtgaccaggtctgggttgaaaaagaccccaaaaagggtcacatttaccagggctctgaggccgacagcgtcttcagcggcttcctcatcttcccatctg

SEQ ID NO: 5 - DNA, 100 bp, Artificial Sequence
  Target sequence of C1S
actgcactgattggggagattgcaagtcccaattatcccaaaccatatccagagaactcaaggtgtgaataccagatccggttggagaaagggttccaag

SEQ ID NO: 6 - DNA, 100 bp, Artificial Sequence
  Target sequence of C2
atctcatgatgaacaaccaaatgcgactcctcggcatggaaacgatggcctggcaggaaatccgacatgccatcatccttctgacagatggaaagtccaa

SEQ ID NO: 7 - DNA, 100 bp, Artificial Sequence
  Target sequence of C3AR1
cagtgtcttcctgcttactgccattagcctggatcgctgtcttgtggtattcaagccaatctggtgtcagaatcatcgcaatgtagggatggcctgctct

SEQ ID NO: 8 - DNA, 100 bp, Artificial Sequence
  Target sequence of CCL4
ttctgcagcctcacctctgagaaaacctctttgccaccaataccatgaagctctgcgtgactgtcctgtctctcctcatgctagtagctgccttctgctc

SEQ ID NO: 9 - DNA, 100 bp, Artificial Sequence
  Target sequence of CCL17
gcctggagtacttcaagggagccattccccttagaaagctgaagacgtggtaccagacatctgaggactgctccagggatgccatcgtttttgtaactgt

SEQ ID NO: 10 - DNA, 100 bp, Artificial Sequence
  Target sequence of CCL20
atctgttctttgagctaaaaaccatgtgctgtaccaagagtttgctcctggctgctttgatgtcagtgctgctactccacctctgcggcgaatcagaagc

SEQ ID NO: 11 - DNA, 100 bp, Artificial Sequence
  Target sequence of CCL24
atagtaaccagccttctgttccttggtgtctgtgcccaccacatcatccctacgggctctgtggtcatcccctctccctgctgcatgttctttgtttcca

SEQ ID NO: 12 - DNA, 100 bp, Artificial Sequence
  Target sequence of CCR2
tctgatctgctttttcttattactctcccattgtgggctcactctgctgcaaatgagtgggtctttgggaatgcaatgtgcaaattattcacagggctgt

SEQ ID NO: 13 - DNA, 100 bp, Artificial Sequence
  Target sequence of CD4
agacatcgtggtgctagctttccagaaggcctccagcatagtctataagaaagagggggaacaggtggagttctccttcccactcgcctttacagttgaa

SEQ ID NO: 14 - DNA, 100 bp, Artificial Sequence
  Target sequence of CD40
cccaggaagccatatacacagatgcccattgcagcattgtttgtgatagtgaacaactggaagctgcttaactgtccatcagcaggagactggctaaata

SEQ ID NO: 15 - DNA, 100 bp, Artificial Sequence
  Target sequence of CD163
catctgtgattcggacttctctctggaagctgccagcgttctatgcagggaattacagtgtggcacagttgtctctatcctggggggagctcactttgga

SEQ ID NO: 16 - DNA, 100 bp, Artificial Sequence
  Target sequence of CFD
ctggttggtctttattgagcacctactatatgcagaaggggaggccgaggtgggaggatcattggatctcaggagttcgagatcagcatgggccacgtag

SEQ ID NO: 17 - DNA, 100 bp, Artificial Sequence
  Target sequence of CSF1
tttctatgagacacctctccagttgctggagaaggtcaagaatgtctttaatgaaacaaagaatctccttgacaaggactggaatattttcagcaagaac

SEQ ID NO: 18 - DNA, 100 bp, Artificial Sequence
  Target sequence of CXCL9
caccatctcccatgaagaaagggaacggtgaagtactaagcgctagaggaagcagccaagtcggttagtggaagcatgattggtgcccagttagcctctg

SEQ ID NO: 19 - DNA, 100 bp, Artificial Sequence
  Target sequence of CXCR4
attgatgtgtgtctaggcaggacctgtggccaagttcttagttgctgtatgtctcgtggtaggactgtagaaaagggaactgaacattccagagcgtgta

SEQ ID NO: 20 - DNA, 100 bp, Artificial Sequence
  Target sequence of FOS
actcaagtccttacctcttccggagatgtagcaaaacgcatggagtgtgtattgttcccagtgacacttcagagagctggtagttagtagcatgttgagc

SEQ ID NO: 21 - DNA, 100 bp, Artificial Sequence
  Target sequence of GNGT1
aagaagtaagagattacgttgaagaacgatctggcgaggatccactggtaaagggcatcccagaggacaaaaatcccttcaaggagctcaaaggaggctg

SEQ ID NO: 22 - DNA, 100 bp, Artificial Sequence
  Target sequence of HDAC4
tctgtcagctcactccagcttcacaaatgtgctgagagcattactgtgtagccttttctttgaagacacactcggctcttctccacagcaagcgtccagg

SEQ ID NO: 23 - DNA, 100 bp, Artificial Sequence
  Target sequence of HMGB2
ctgtcaacatgggtaaaggagaccccaacaagccgcggggcaaaatgtcctcgtacgccttcttcgtgcagacctgccgggaagagcacaagaagaaaca

SEQ ID NO: 24 - DNA, 100 bp, Artificial Sequence
  Target sequence of HSPB2
agctgctctctcccatgatggcatcttaaacctggaagcacctcggggtggccgacatttggacacagaggtcaatgaggtctacatctccctgctccct

SEQ ID NO: 25 - DNA, 100 bp, Artificial Sequence
  Target sequence of IFIT2
tgcatcccatagaggttagtcctgcatagccagtaatgtgctaagttcatccaaaagctggcggaccaaagtctaaatagggctcagtatcccccatcgc

SEQ ID NO: 26 - DNA, 100 bp, Artificial Sequence
  Target sequence of IFNG
atactatccagttactgccggtttgaaaatatgcctgcaatctgagccagtgctttaatggcatgtcagacagaacttgaatgtgtcaggtgaccctgat

SEQ ID NO: 27 - DNA, 100 bp, Artificial Sequence
  Target sequence of IL4
gacactcgctgcctgggtgcgactgcacagcagttccacaggcacaagcagctgatccgattcctgaaacggctcgacaggaacctctggggcctggcgg

SEQ ID NO: 28 - DNA, 100 bp, Artificial Sequence
  Target sequence of IL6
tgacaaacaaattcggtacatcctcgacggcatctcagccctgagaaaggagacatgtaacaagagtaacatgtgtgaaagcagcaaagaggcactggca

SEQ ID NO: 29 - DNA, 100 bp, Artificial Sequence
  Target sequence of IL6R
ctttctacatagtgtccatgtgcgtcgccagtagtgtcgggagcaagttcagcaaaactcaaacctttcagggttgtggaatcttgcagcctgatccgcc

SEQ ID NO: 30 - DNA, 100 bp, Artificial Sequence
  Target sequence of IL8
acagcagagcacacaagcttctaggacaagagccaggaagaaaccaccggaaggaaccatctcactgtgtgtaaacatgacttccaagctggccgtggct

SEQ ID NO: 31 - DNA, 100 bp, Artificial Sequence
  Target sequence of IL10RB
ttctaccagattatggatggactgatctgaaaatcgacctcaactcaagggtggtcagctcaatgctacacagagcacggacttttggattctttgcagt

SEQ ID NO: 32 - DNA, 100 bp, Artificial Sequence
  Target sequence of IL18RAP
gcttgatggacaatggagtgggattgagactgtggtttagagcctttgatttcctggactggactgacggcgagtgaattctctagaccttgggtacttt

SEQ ID NO: 33 - DNA, 100 bp, Artificial Sequence
  Target sequence of IRF1
ctgtgcgagtgtaccggatgcttccacctctcaccaagaaccagagaaaagaaagaaagtcgaagtccagccgagatgctaagagcaaggccaagaggaa

SEQ ID NO: 34 - DNA, 100 bp, Artificial Sequence
  Target sequence of IRF7
cgcagcgtgagggtgtgtcttccctggatagcagcagcctcagcctctgcctgtccagcgccaacagcctctatgacgacatcgagtgcttccttatgga

SEQ ID NO: 35 - DNA, 100 bp, Artificial Sequence
  Target sequence of JUN
acacagccagccagccaggtcggcagtatagtccgaactgcaaatcttattttcttttcaccttctctctaactgcccagagctagcgcctgtggctccc

SEQ ID NO: 36 - DNA, 100 bp, Artificial Sequence
  Target sequence of LTB4R
gtagtgccctggaaaacgaacatgagcctgtgcttcccgcggtaccccagcgaagggcaccgggccttccatctaatcttcgaggctgtcacgggcttcc

SEQ ID NO: 37 - DNA, 100 bp, Artificial Sequence
  Target sequence of MAP2K6
agccaggaacagaaacggctactgatggatttggatatttccatgaggacggtggactgtccattcactgtcaccttttatggcgcactgtttcgggagg

SEQ ID NO: 38 - DNA, 100 bp, Artificial Sequence
  Target sequence of MAP3K5
tttaggaaaaggcacttatgggatagtctacgcaggtcgggacttgagcaaccaagtcagaattgctattaaggaaatcccagagagagacagcagatac

SEQ ID NO: 39 - DNA, 100 bp, Artificial Sequence
  Target sequence of MEF2C
acatttgccaaaagcggcagatctttactgaaagagagagcagctgttatgcaacatatagaaaaatgtatagatgcttggacagacccggtaatgggtg

SEQ ID NO: 40 - DNA, 100 bp, Artificial Sequence
  Target sequence of MRC1
ctatggaaccacagacaatctgtgctccagaggttatgaagccatgtatacgctactaggcaatgccaatggagcaacctgtgcattcccgttcaagttt

SEQ ID NO: 41 - DNA, 100 bp, Artificial Sequence
  Target sequence of MX1
gcctttaatcaggacatcactgctctcatgcaaggagaggaaactgtaggggaggaagacattcggctgtttaccagactccgacacgagttccacaaat

SEQ ID NO: 42 - DNA, 100 bp, Artificial Sequence
  Target sequence of MYC
tcggacaccgaggagaatgtcaagaggcgaacacacaacgtcttggagcgccagaggaggaacgagctaaaacggagcttttttgccctgcgtgaccaga

SEQ ID NO: 43 - DNA, 100 bp, Artificial Sequence
  Target sequence of MYD88
acgtttttctaggtacagctcccaggaacagctaggtgggaaagtcccatcactgagggagcctaaccatgtccctgaacaaaaattgggcactcatcta

SEQ ID NO: 44 - DNA, 100 bp, Artificial Sequence
  Target sequence of NLRP3
agtggggttcagataatgcacgtgtttcgaatcccactgtgatatgccaggaagacagcattgaagaggagtggatgggtttactggagtacctttcgag

SEQ ID NO: 45 - DNA, 100 bp, Artificial Sequence
  Target sequence of NOD1
agaggatgctttcctgttcatggggtttttgccctggagcctcagcagcaaatgccactctgggcagtcttttgtgtcagtgtcttaaaggggcctgcgc

SEQ ID NO: 46 - DNA, 100 bp, Artificial Sequence
  Target sequence of OASL
ggcgtttctgagctgtttccacagcttccaggaggcagccaagcatcacaaagatgttctgaggctgatatggaaaaccatgtggcaaagccaggacctg

SEQ ID NO: 47 - DNA, 100 bp, Artificial Sequence
  Target sequence of PIK3C2G
ctggaatatctcccacagctagttcaggctgtcaagtttgaatggaaccttgagagtcctttagtgcaacttctactccaccgctccttgcagagcatcc

SEQ ID NO: 48 - DNA, 100 bp, Artificial Sequence
  Target sequence of PLCB1
tcaaaacaaggagacagagctactggatctcagccttgtcaaagatgccagatgtgggagacacgccaaagctcccaaggaccccaaattacgtgaactt

SEQ ID NO: 49 - DNA, 100 bp, Artificial Sequence
  Target sequence of PRKCB
gcatttggagtcctgctgtatgaaatgttggctgggcaggcaccctttgaaggggaggatgaagatgaactcttccaatccatcatggaacacaacgtag

SEQ ID NO: 50 - DNA, 100 bp, Artificial Sequence
  Target sequence of PTGER2
gtcagaaggagctacaaaacctaccctcagtgagcatggtacttggcctttggaggaacaatcggctgcattgaagatccagctgcctattgatttaagc

SEQ ID NO: 51 - DNA, 100 bp, Artificial Sequence
  Target sequence of PTGFR
aacagccttgccatcgccattctcatgaaggcatatcagagatttagacagaagtccaaggcatcgtttctgcttttggccagtggcctggtaatcactg

SEQ ID NO: 52 - DNA, 100 bp, Artificial Sequence
  Target sequence of STAT1
tttgctgtatgccatcctcgagagctgtctaggttaacgttcgcactctgtgtatataacctcgacagtcttggcacctaacgtgctgtgcgtagctgct

SEQ ID NO: 53 - DNA, 100 bp, Artificial Sequence
  Target sequence of STAT2
ccgtacacgaaggaggtgctgcagtcactcccgctgactgaaatcatccgccattaccagttgctcactgaggagaatatacctgaaaacccactgcgct

SEQ ID NO: 54 - DNA, 100 bp, Artificial Sequence
  Target sequence of TCF4
tggaatcattggaccttctcataatggagccatgggtggtctgggctcagggtatggaaccggccttctttcagccaacagacattcactcatggtgggg

SEQ ID NO: 55 - DNA, 100 bp, Artificial Sequence
  Target sequence of TGFB1
tatatgttcttcaacacatcagagctccgagaagcggtacctgaacccgtgttgctctcccgggcagagctgcgtctgctgaggctcaagttaaaagtgg

SEQ ID NO: 56 - DNA, 100 bp, Artificial Sequence
  Target sequence of TGFBR1
ggggaaatacgacttagtgaggcatagacatccctggtccatcctttctgtctccagctgtttcttggaacctgctctcctgcttgctggtccctgacgc

SEQ ID NO: 57 - DNA, 100 bp, Artificial Sequence
  Target sequence of TLR4
actcagaaaagccctgctggatggtaaatcatggaatccagaaggaacagtgggtacaggatgcaattggcaggaagcaacatctatctgaagaggaaaa

SEQ ID NO: 58 - DNA, 100 bp, Artificial Sequence
  Target sequence of TLR5
gttcctggcactactacaagattcatactcctgatgctactgacaacgtggcttctccacagtcaccaaaccagggatgctatactggacttccctactc

SEQ ID NO: 59 - DNA, 100 bp, Artificial Sequence
  Target sequence of TLR8
tttaactgatagcctatctgactttacatcttcccttcggacactgctgctgagtcataacaggatttcccacctaccctctggctttctttctgaagtc

SEQ ID NO: 60 - DNA, 100 bp, Artificial Sequence
  Target sequence of TNF
agcaacaagaccaccacttcgaaacctgggattcaggaatgtgtggcctgcacagtgaagtgctggcaaccactaagaattcaaactggggcctccagaa

SEQ ID NO: 61 - DNA, 100 bp, Artificial Sequence
  Target sequence of TREM2
ttcccacccacttccatccttctcctcctggcctgcatctttctcatcaagattctagcagccagcgccctctgggctgcagcctggcatggacagaagc

SEQ ID NO: 62 - DNA, 100 bp, Artificial Sequence
  Target sequence of CCL13
ccagaattatatgaaacacctgggccggaaagctcacaccctgaagacttgaactctgctacccctactgaaatcaagctggagtacgtgaaatgacttt

SEQ ID NO: 63 - DNA, 100 bp, Artificial Sequence
  Target sequence of C4A
ggtccgtgagatgtcaggctccccagcttctggcattcctgtcaaagtttctgccacggtgtcttctcctgggtctgttcctgaagtccaggacattcag

SEQ ID NO: 64 - DNA, 100 bp, Artificial Sequence
  Target sequence of CFB
taagctgaaatatggccagactatcaggcccatttgtctcccctgcaccgagggaacaactcgagctttgaggcttcctccaactaccacttgccagcaa

SEQ ID NO: 65 - DNA, 100 bp, Artificial Sequence
  Target sequence of CXCL5
agagagctgcgttgcgtttgtttacagaccacgcaaggagttcatcccaaaatgatcagtaatctgcaagtgttcgccataggcccacagtgctccaagg

SEQ ID NO: 66 - DNA, 100 bp, Artificial Sequence
  Target sequence of CXCL6
agtaacaaaaaagaccatgcatcataaaattgcccagtcttcagcggagcagttttctggagatccctggacccagtaagaataagaaggaagggttggt

SEQ ID NO: 67 - DNA, 100 bp, Artificial Sequence; identical to SEQ ID NO: 18
  Target sequence of CXCL9

SEQ ID NO: 68 - DNA, 100 bp, Artificial Sequence
  Target sequence of CXCR2
aggagaaactggaactctcgagcgttgctgggggggattgtaaaatggtgtgaccactgcagaagacagtatggcagctttcctcaaaacttcagacata

SEQ ID NO: 69 - DNA, 100 bp, Artificial Sequence
  Target sequence of HLA-DRA
ggccaacatagctgtggacaaagccaacctggaaatcatgacaaagcgctccaactatactccgatcaccaatgtacctccagaggtaactgtgctcacg

SEQ ID NO: 70 - DNA, 100 bp, Artificial Sequence
  Target sequence of HSH2D
gtgtcagcaccaggatttgaagccaggtgaatccgagcccttttcccatatcatctgtttgttctgttgtctaaaagcacactgcaagccgggctcagtg

SEQ ID NO: 71 - DNA, 100 bp, Artificial Sequence
  Target sequence of IFI44
tttccaagggcatgtaacgcatcaggctttggtgggcactaatacaactgggatatctgagaagtataggacatactctattagagacgggaaagatggc

SEQ ID NO: 72 - DNA, 100 bp, Artificial Sequence
  Target sequence of IL7
aataacccagcttgcgtcctgcacacttgtggcttccgtgcacacattaacaactcatggttctagctcccagtcgccaagcgttgccaaggcgttgaga

SEQ ID NO: 73 - DNA, 100 bp, Artificial Sequence
  Target sequence of IRF3
tcatggccccaggaccagccgtggaccaagaggctcgtgatggtcaaggttgtgcccacgtgcctcagggccttggtagaaatggcccgggtagggggtg

SEQ ID NO: 74 - DNA, 100 bp, Artificial Sequence
  Target sequence of ITGB2
catcgacctgtactatctgatggacctctcctactccatgcttgatgacctcaggaatgtcaagaagctaggtggcgacctgctccgggccctcaacgag

SEQ ID NO: 75 - DNA, 100 bp, Artificial Sequence
  Target sequence of LTB4R2
cagaggtcagtgttctgggacatttggggacccttctttgactagagtttggatctggctgggtaggattactatacacttggggcaggcccaggctcct

SEQ ID NO: 76 - DNA, 100 bp, Artificial Sequence
  Target sequence of MAFF
aggcctgaggtcacatttcacttagtggttgtgttgggaccaaaacctgggtgtcctcactgctgccctgagtccagccatggttttcagggggacagtg

SEQ ID NO: 77 - DNA, 100 bp, Artificial Sequence
  Target sequence of MX2
aatttaccggctcactcaggcgcgacacgcactctgtcaattctccagcaaagagatccactgaagggcggcgatgcctgtggttgttttcttgtgcgta

SEQ ID NO: 78 - DNA, 100 bp, Artificial Sequence
  Target sequence of NOX1
tcattttgcagccgcacactgagaaagcaattggatcacaacctcaccttccacaagctggtggcctatatgatctgcctacatacagctattcacatca

SEQ ID NO: 79 - DNA, 100 bp, Artificial Sequence
  Target sequence of OAS2
tgaaaaacaatttcgagatccagaagtcccttgatgggttcaccatccaggtgttcacaaaaaatcagagaatctctttcgaggtgctggccgccttcaa

SEQ ID NO: 80 - DNA, 100 bp, Artificial Sequence; identical to SEQ ID NO: 46
  Target sequence of OASL

SEQ ID NO: 81 - DNA, 100 bp, Artificial Sequence
  Target sequence of PTGER4
cgcgggcgccgagatccagatggtcatcttactcattgccacctccctggtggtgctcatctgctccatcccgctcgtggtgcgagtattcgtcaaccag

SEQ ID NO: 82 - DNA, 100 bp, Artificial Sequence
  Target sequence of RELB
cactctcgctcgccgtttccaggagcacagatgaattggagatcatcgacgagtacatcaaggagaacggcttcggcctggacgggggacagccgggccc

SEQ ID NO: 83 - DNA, 100 bp, Artificial Sequence
  Target sequence of TNFAIP3
caaagccctcatcgacagaaacatccaggccaccctggaaagccagaagaaactcaactggtgtcgagaagtccggaagcttgtggcgctgaaaacgaac

SEQ ID NO: 84 - DNA, 100 bp, Artificial Sequence
  Target sequence of CCL22
ctcgcccaagcagctggtaattccatttcatgtattagatgtcccctggccctctgtcccctcttaataaccctagtcacagtctccgcagattcttggg

SEQ ID NO: 85 - DNA, 100 bp, Artificial Sequence
  Target sequence of CD40LG
gcatttgatttatcagtgaagatgcagaagggaaatggggagcctcagctcacattcagttatggttgactctgggttcctatggccttgttggaggggg

SEQ ID NO: 86 - DNA, 100 bp, Artificial Sequence; identical to SEQ ID NO: 64
  Target sequence of CFB

SEQ ID NO: 87 - DNA, 100 bp, Artificial Sequence
  Target sequence of CXCL10
gcagaggaacctccagtctcagcaccatgaatcaaactgcgattctgatttgctgccttatctttctgactctaagtggcattcaaggagtacctctctc

SEQ ID NO: 88 - DNA, 100 bp, Artificial Sequence
  Target sequence of ELK1
ttttcaataggggagagggagtcatctcttcctatatttggtggggtgggtgggaaggaagggatttgggggggaatcttcctgccgcctcccccactcc

SEQ ID NO: 89 - DNA, 100 bp, Artificial Sequence
  Target sequence of IFIT1
gagaaaggcattagatctggaaagcttgagcctccttgggttcgtctacaaattggaaggaaatatgaatgaagccctggagtactatgagcgggccctg

SEQ ID NO: 90 - DNA, 100 bp, Artificial Sequence
  Target sequence of IFIT3
cgcctgctaagggatgccccttcaggcataggcagtattttcctgtcagcatctgagcttgaggatggtagtgaggaaatgggccagggcgcagtcagct

SEQ ID NO: 91 - DNA, 100 bp, Artificial Sequence
  Target sequence of IL1R1
ccagagagtgggggtgatgatgaccaagaattacaagtagaatggcagctggaatttaaggagggacaagaatcaatggataagcgtgggtggaggaaga

SEQ ID NO: 92 - DNA, 100 bp, Artificial Sequence
  Target sequence of IL22
ctatctgatgaagcaggtgctgaacttcacccttgaagaagtgctgttccctcaatctgataggttccagccttatatgcaggaggtggtgcccttcctg

SEQ ID NO: 93 - DNA, 100 bp, Artificial Sequence
  Target sequence of IL23A
cagggacaacagtcagttctgcttgcaaaggatccaccagggtctgattttttatgagaagctgctaggatcggatattttcacaggggagccttctctg

SEQ ID NO: 94 - DNA, 100 bp, Artificial Sequence
  Target sequence of LY96
agaagttatttgccgaggatctgatgacgattactctttttgcagagctctgaagggagagactgtgaatacaacaatatcattctccttcaagggaata

SEQ ID NO: 95 - DNA, 100 bp, Artificial Sequence
  Target sequence of MAFK
tgggtccctacagagcaaaggcctgcgtgttcccaaacagccgttgcccccgtggccgtggtaggtaatccatattggatgtcataaggaccagggggat

SEQ ID NO: 96 - DNA, 100 bp, Artificial Sequence
  Target sequence of MKNK1
tggagaactgaagattgccacctacgcacaaaggccattgagacacttcgtgtagctggaagacaccaacttcctgacaggagctttatttcatttggga

SEQ ID NO: 97 - DNA, 100 bp, Artificial Sequence
  Target sequence of NOD2
gggccagaatttcaaacggcctcactaggcttctggttgatgcctgtgaactgaactctgacaacagacttctgaaatagacccacaagaggcagttcca

SEQ ID NO: 98 - DNA, 100 bp, Artificial Sequence
  Target sequence of NOS2
ttgcctggggtccattatgactcccaaaagtttgaccagaggacccagggacaagcctacccctccagatgagcttctacctcaagctatcgaatttgtc

SEQ ID NO: 99 - DNA, 100 bp, Artificial Sequence
  Target sequence of TLR6
tgtctggaggtgcctccattatcctcatgccttcaggaaagacttaacaaaaacaatgtttcatctggggaactgagctaggcggtgaggttagcctgcc

SEQ ID NO: 100 - DNA, 100 bp, Artificial Sequence
  Target sequence of CCL3
cagttctctgcatcacttgctgctgacacgccgaccgcctgctgcttcagctacacctcccggcagattccacagaatttcatagctgactactttgaga

SEQ ID NO: 101 - DNA, 100 bp, Artificial Sequence
  Target sequence of CXCL1
tatgttaatatttctgaggagcctgcaacatgccagccactgtgatagaggctggcggatccaagcaaatggccaatgagatcattgtgaaggcagggga

SEQ ID NO: 102 - DNA, 100 bp, Artificial Sequence
  Target sequence of GRB2
aactggtacaaggcagagcttaatggaaaagacggcttcattcccaagaactacatagaaatgaaaccacatccgtttggaaacgatgtgcagcacttca

SEQ ID NO: 103 - DNA, 100 bp, Artificial Sequence
  Target sequence of HLA-DRB1
tcggcaactgcagaaaatgtcctcccttgtggcttcctcagctcctgcccttggcctgaagtcccagcattgatgacagcgcctcatcttcaacttttgt

SEQ ID NO: 104 - DNA, 100 bp, Artificial Sequence
  Target sequence of IL1B
gggaccaaaggcggccaggatataactgacttcaccatgcaatttgtgtcttcctaaagagagctgtacccagagagtcctgtgctgaatgtggactcaa

SEQ ID NO: 105 - DNA, 100 bp, Artificial Sequence
  Target sequence of IL22RA2
cacttgcaaccatgatgcctaaacattgctttctaggcttcctcatcagtttcttccttactggtgtagcaggaactcagtcaacgcatgagtctctgaa

SEQ ID NO: 106 - DNA, 100 bp, Artificial Sequence
  Target sequence of LTB
aggaacaggcgtttctgacgagcgggacgcagttctcggacgccgaggggctggcgctcccgcaggacggcctctattacctctactgtctcgtcggcta

SEQ ID NO: 107 - DNA, 100 bp, Artificial Sequence
  Target sequence of MMP9
cactactgtgcctttgagtccggtggacgatgcctgcaacgtgaacatcttcgacgccatcgcggagattgggaaccagctgtatttgttcaaggatggg

SEQ ID NO: 108 - DNA, 100 bp, Artificial Sequence
  Target sequence of PTGS2
gctacaaaagctgggaagccttctctaacctctcctattatactagagcccttcctcctgtgcctgatgattgcccgactcccttgggtgtcaaaggtaa

SEQ ID NO: 109 - DNA, 100 bp, Artificial Sequence; identical to SEQ ID NO: 54
  Target sequence of TCF4

SEQ ID NO: 110 - DNA, 100 bp, Artificial Sequence
  Target sequence od TGFB2
aagccagagtgcctgaacaacggattgagctatatcagattctcaagtccaaagatttaacatctccaacccagcgctacatcgacagcaaagttgtgaa

SEQ ID NO: 111 - DNA, 100 bp, Artificial Sequence
  Target sequence of TNFSF14
attttcagaagcctctggaaagtcgtgcacagcccaggagtgttgagcaatttcggtttcctctgaggttgaaggacccaggcgtgtcagccctgctcca

SEQ ID NO: 112 - DNA, 100 bp, Artificial Sequence
  Target sequence of CD55
ccctactccacccgtcttgtttgtcccacccttggtgacgcagagccccagcccagaccccgcccaaagcactcatttaactggtattgcggagccacga

SEQ ID NO: 113 - DNA, 100 bp, Artificial Sequence
  Target sequence of CXCL3
tccctgcccttaccagagctgaaaatgaaaaagagaacagcagctttctagggacagctggaaaggacttaatgtgtttgactatttcttacgagggttc

SEQ ID NO: 114 - DNA, 100 bp, Artificial Sequence
  Target sequence of IL15
tagccagcccatacaagatcgtattgtattgtaggaggcattgtggatggatggctgctggaaaccccttgccatagccagctcttcttcaatacttaag

SEQ ID NO: 115 - DNA, 100 bp, Artificial Sequence
  Target sequence of IL1RN
tcttcctgttccattcagagacgatctgccgaccctctgggagaaaatccagcaagatgcaagccttcagaatctgggatgttaaccagaagaccttcta

SEQ ID NO: 116 - DNA, 100 bp, Artificial Sequence
  Target sequence of PDGFA
cgtttgtggctgagtgacaacttgttccccgcagtgcacacctagaatgctgtgttcccacgcggcacgtgagatgcattgccgcttctgtctgtgttgt

SEQ ID NO: 117 - DNA, 100 bp, Artificial Sequence
  Target sequence of PRKCA
ccggcagtgcttcccttcctttcatccactggcctcgtgtggtccatgcagggccactgtctgccctttctgatgccacgtattaggctttcttactcag

SEQ ID NO: 118 - DNA, 100 bp, Artificial Sequence
  Target sequence of GNAQ
aaggagtacaatctggtctaattgtgcctcctagacacccgccctgcccttccctggtgggctattgaagatacacaagagggactgtatttctgtggaa

SEQ ID NO: 119 - DNA, 100 bp, Artificial Sequence
  Target sequence of PLA2G4A
aactggatgcagctgtagcagatcctgatgaatttgagcgaatatatgagcctctggatgtcaaaagtaaaaagattcatgtagtggacagtgggctcac

SEQ ID NO: 120 - DNA, 100 bp, Artificial Sequence
  Target sequence of CFL1
tccggaaacatggcctccggtgtggctgtctctgatggtgtcatcaaggtgttcaacgacatgaaggtgcgtaagtcttcaacgccagaggaggtgaaga

SEQ ID NO: 121 - DNA, 100 bp, Artificial Sequence
  Target sequence of CDC42
accgactgtttttgacaactatgcagtcacagttatgattggtggagaaccatatactcttggactttttgatactgcagggcaagaggattatgacaga

SEQ ID NO: 122 - DNA, 100 bp, Artificial Sequence
  Target sequence of SHC1
gcgctctcttccagaagatgccctccaatcctttccaccctattccctaactctcgggacctcgtttgggagtgttctgtgggcttggccttgtgtcaga

SEQ ID NO: 123 - DNA, 100 bp, Artificial Sequence
  Target sequence of CCR1
catcatttgggccctggccatcttggcttccatgccaggcttatacttttccaagacccaatgggaattcactcaccacacctgcagccttcactttcct

SEQ ID NO: 124 - DNA, 100 bp, Artificial Sequence
  Target sequence of CXCL2
atcacatgtcagccactgtgatagaggctgaggaatccaagaaaatggccagtgagatcaatgtgacggcagggaaatgtatgtgtgtctattttgtaac

SEQ ID NO: 125 - DNA, 100 bp, Artificial Sequence
  Target sequence of KEAP1
ctcattgaattcgcctacacggcctccatctccatgggcgagaagtgtgtcctccacgtcatgaacggtgctgtcatgtaccagatcgacagcgttgtcc

SEQ ID NO: 126 - DNA, 100 bp, Artificial Sequence
  Target sequence of CCL5
agtgtgtgccaacccagagaagaaatgggttcgggagtacatcaactctttggagatgagctaggatggagagtccttgaacctgaacttacacaaattt

SEQ ID NO: 127 - DNA, 100 bp, Artificial Sequence
  Target sequence of C1QB
aactcactactgggcatggagggtgccaacagcatcttttccgggttcctgctctttccagatatggaggcctgacctgtgggctgcttcacatccaccc

(19) United States
(12) Patent Application Publication
(10) Pub. No.: US 2022/0098669 A1
(43) Pub. Date: Mar. 31, 2022

(54) GENE SIGNATURE FOR THE PROGNOSIS OF DRY EYE DISEASE

(71) Applicant: SANTEN SAS, Evry (FR)

(72) Inventors: Christophe BAUDOUIN, Paris (FR); Philippe DAULL, Soisy-sur-Seine (FR); Karima KESSAL, Paris (FR)

(21) Appl. No.: 17/644,348

(22) Filed: Dec. 15, 2021

Related U.S. Application Data
(62) Division of application No. 16/080,702, filed on Aug. 29, 2018, now abandoned.

(30) Foreign Application Priority Data
Feb. 29, 2016 (EP) 16157955.2

(51) Int. Cl. C12Q 1/6883; G16H 50/30; G16H 50/20; G16B 30/00; C12Q 1/6837

(52) U.S. Cl. CPC C12Q 1/6883; G16H 50/30; G16H 50/20; C12Q 2600/158; C12Q 1/6837; C12Q 2600/112; G16B 30/00

(57) ABSTRACT

Disclosed is a signature including at least 3 markers, as well as a method for the prognosis of dry eye disease in a subject, wherein the method includes assessing the expression of markers of a signature in a sample from the subject. Also disclosed is a kit for implementing this method.

### CROSS-REFERENCE TO RELATED APPLICATIONS

This patent application is a divisional of U.S. patent application Ser. No. 16/080,702 filed on Aug. 29, 2018, which is a National Stage Entry of International Patent Application No. PCT/EP2017/054653 filed on Feb. 28, 2017, which claims priority to European Patent Application No. 16157955.2 filed on Feb. 29, 2016. Each of these applications is incorporated by reference herein in its entirety.

### INCORPORATION BY REFERENCE

The text file named 7238-0201 SEQ LISTING, created on May 7, 2021, and sized 34,319 bytes, which contains sequence ID listings, is herein expressly incorporated by reference.

### FIELD OF INVENTION

The present invention relates to the field of dry eye disease prognosis. More specifically, the present invention relates to a signature based on differential gene expression in different conditions of dry eye disease, for the prognosis of the disease in a subject.

### BACKGROUND OF INVENTION

Dry eye disease (DED) is a complex multifactorial disease in which inflammation is often considered as the core mechanism of the pathology. DED is characterized by the presence of signs and symptoms that are concomitant or not. Corneal epithelium alterations (measured by corneal fluorescein staining) are the paragon of DED signs, while stinging, burning or scratchy sensation in the eye, eye redness, sensitivity to light, a sensation of having something in the eyes, watery eyes, blurred vision and eye fatigue represent the most common symptoms associated with DED. The choice of treatment options for DED is based on the severity of the disease: for example, severe DED patients will not be treated the same way than mild patients.

An inadequate characterization of DED severity leads to an inappropriate treatment strategy, with the risk for the patient of a poor vision quality, and ultimately the risk of vision loss. It is therefore very important for the clinicians to be able to adequately characterize the severity of the disease that is affecting the patient, thereby bringing him/her the most effective treatment for his/her condition.

However, determining the severity of DED is actually based on signs and symptoms analysis. This determination is difficult and not straightforward since signs and symptoms do not generally correlate. Moreover, symptoms are highly subjective and are dependent on patients' sensibility. It is very common to have patients with severe symptoms but almost no signs of DED, or patients with severe signs with few or no symptoms.

Consequently, there exists a need to correctly identify the severity of the disease in DED patients, from mild to severe, for providing the appropriate treatment to patients.

The inventors herein showed that each dry eye stage, from mild to severe, has the potential to lead to common alterations in the expression of some transcripts. Therefore, the present invention relates to a signature of dry eye disease of particular clinical relevance for the prognosis of the severity of the disease in a subject.

### SUMMARY

The present invention relates to a method for the prognosis of DED in a subject, wherein said method comprises assessing the expression of markers of a signature comprising (i) at least one DED marker; (ii) at least one mild DED marker; and (iii) at least one severe DED marker in a sample from said subject.

In one embodiment, the at least one DED marker is a marker selected from the list of Table 2 or Table 9.

In one embodiment, the at least one DED marker is a marker whose expression is different between a DED patient and a “normal” patient.

In one embodiment, the at least one mild DED marker is a marker whose expression is different between a patient suffering from mild DED and a “normal” patient, or between a patient suffering from mild DED and a patient suffering from severe DED.

In one embodiment, the at least one severe DED marker is a marker whose expression is different between a patient suffering from severe DED and a “normal” patient, or between a patient suffering from severe DED and a patient suffering from mild DED.

In one embodiment, the signature comprises at least 3, preferably at least 4, more preferably at least 5 markers.

In one embodiment, the at least one DED marker is selected from the list of Table 3, preferably from the list of Table 4, more preferably from the list of Table 5, even more preferably from the list of Table 6, 7 or 8. In one embodiment, the at least one DED marker is selected from the list of Table 9 or Table 10.

In one embodiment, the at least one mild DED marker is selected from the list of Table 11, fragments, variants and equivalents thereof, preferably from the list of Table 12, more preferably from the list of Table 13, even more preferably from the list of Table 14; and/or from the list of Table 15, fragments, variants and equivalents thereof, preferably from the list of Table 16, more preferably from the list of Table 17, even more preferably from the list of Table 18.

In one embodiment, the at least one severe DED marker is selected from the list of Table 19, fragments, variants and equivalents thereof, preferably from the list of Table 20, more preferably from the list of Table 21, more preferably from the list of Table 22, even more preferably from the list of Table 23; and/or from the list of Table 24, fragments, variants and equivalents thereof, preferably from the list of Table 25, more preferably from the list of Table 26, even more preferably from the list of Table 27.

In one embodiment, the sample is conjunctival superficial cells of said subject.

In one embodiment, the subject is a human.

In one embodiment, the method of the invention further comprises comparing said expression with a reference expression profile.

In one embodiment, the method comprises the steps of:
extracting total RNA from the sample from the subject,
determining the expression profile of the markers of the signature, and
comparing said expression profile with a reference expression profile determined in a reference sample.

In one embodiment, the method is a non-invasive method.

Another object of the invention is a genechip specific for dry eye disease, comprising at least 3 of the genes selected from the group comprising markers of the lists of Table 1 or Table 9, Table 11, Table 15, Table 19 and Table 24, or homologs thereof.

The present invention further relates to kit for implementing the method according to the invention. In one embodiment, the kit of the invention comprises means for determining the expression of the markers of the signature.

Another object of the present invention is a method for the identification of patients with dry eye disease (DED) but not Sjögren's syndrome, wherein said method comprises assessing the expression of markers of at least one marker selected from Table 28, preferably Table 29, more preferably Table 30, even more preferably Table 31 or 32.

The present invention further relates to a method for the identification of the severity of dry eye disease (DED) in a subject that does not suffer from Sjögren's syndrome, wherein said method comprises assessing the expression of at least one marker selected from the group comprising CFD, GNAQ, PLA2G4A, CDC42, SHC1, CD4, IL7, CD55 and TGFBR1.

The present invention further also to a method for the prognosis of Sjögren's syndrome in a subject, wherein said method comprises assessing the expression at least one marker selected from Table 33, preferably Table 34, more preferably Table 35, even more preferably Table 36.

Another object of the present invention is a method for the identification of the severity of Sjögren's syndrome in a subject, wherein said method comprises assessing the expression of at least one marker selected from the group comprising IL6, CCR1, CCL4, MAFF, NOS2, ITGB2, HLA-DRB1, CXCL2, STAT1, IL1RN, IL15, GNGT1 and HSPB2.

### Definitions

In the present invention, the following terms have the following meanings:
“Prognosis” refers to the likelihood of dry eye disease progression during the natural history of the disease, or to the likelihood of a beneficial response to a specific treatment, wherein a beneficial response means an improvement in any measure of patient status including, but not limited to, osmolarity, burning eye, scratchy sensation in the eye, eye redness, sensitivity to light, watery eyes, blurred vision and eye fatigue. Accordingly, a “prognostic signature” refers to a signature that may be used for the prognosis of a subject. In one embodiment, the term “prognostic signature” also includes “predictive signature”, wherein said term refers to a signature that may be used for anticipating the response of a subject to a specific treatment.
“Signature” refers to a group of markers (i.e. at least 2, preferably at least 3, more preferably at least 5, and even more preferably at least 10 markers) whose combined expression profile is indicative of a biological condition, or of a particular prognosis or of a particular response of a subject to a treatment.
A “marker” corresponds to a nucleotide sequence isolated from the genome, preferably to a gene in the genome, i.e. each marker is identifiable as all or a portion of a gene. A marker may thus correspond to an entire gene, or to an EST (wherein EST stands for Expressed Sequence Tag) derived from this gene.
“Expression” refers interchangeably to expression of a marker, including the encoded polypeptide or protein. Expression of a marker may be determined, for example, by immunoassay using one or more antibody(ies) that bind(s) with the polypeptide. Alternatively, expression of a marker may be determined by measurement of mRNA levels, for example, by RT-PCR, RT-qPCR (wherein qPCR stands for quantitative PCR), or using a microarray, or using sequencing methods. In one embodiment, the term “expression” of a marker may also refer to modification of a protein or peptide, preferably to post-translational modification of a protein or peptide.
“Subject” refers to an animal, preferably a mammal, more preferably a human. In one embodiment, the subject is a patient, i.e. a recipient of health care services. In one embodiment, the subject is a DED patient, i.e. he/she was previously diagnosed with DED. In another embodiment, the subject is a patient suffering from Sjögren's syndrome (also referred as a SS patient). In another embodiment, the subject is a patient suffering from DED and Sjögren's syndrome. In another embodiment, the subject is a patient suffering from DED but not from Sjögren's syndrome (also referred as a DED-SS patient). In another embodiment, the subject is a healthy subject or a “normal” subject, i.e. a subject that does not suffer from DED or Sjögren's syndrome.
“About” preceding a figure means plus or less 10% of the value of said figure.

### DETAILED DESCRIPTION

The identification of biomarkers of inflammation involved in DED will help in the characterization of the pathways implicated in disease development and severity and in the diagnosis of DED. A better characterization of the disease background and a better understanding of the mechanism and pathways implicated in disease development will lead to a better management of its complication and of its treatment.

Using microarray analysis, the expression of 249 mRNA was analyzed in patients suffering from dry eye disease, from mild to severe, or in healthy people. Of the 249 genes analyzed, 61 were induced or down-regulated greater than 1.25-fold in DED patients as compared to healthy control; 22 other genes were induced or down-regulated greater than 1.25-fold in mild DED patients as compared to healthy control; and 12 still other genes were induced or down-regulated greater than 1.25-fold in severe DED patients as compared to healthy control.

The results of this analysis include a set of differentially expressed genes that can be used as a “signature” for the identification of DED and for the severity of DED.

The present invention relates to a signature for dry eye disease, wherein said signature comprises (i) DED markers, whose expression is different between a DED patient and a “normal” patient, i.e. a patient who does not have DED; (ii) mild markers, whose expression is different between a patient suffering from mild DED and a “normal” patient or between a patient suffering from mild DED and a severe patient; and (iii) severe DED markers, whose expression is different between a patient suffering from severe DED and a “normal” patient or between a patient suffering from severe DED and a mild patient.

In one embodiment, a DED patient is a patient suffering from DED. As used herein, the term “DED patient” encompasses all types of DED, whatever the severity, i.e. from mild to severe).

In one embodiment, the signature of the invention comprises at least 3 markers, preferably at least 4 markers, more preferably at least 5 markers, and even more preferably at least 6 markers. In one embodiment, the signature of the invention comprises at least 1 marker (i), preferably at least 2 markers; at least 1 marker (ii), preferably at least 2 markers; and at least 1 marker (iii), preferably at least 2 markers.

Methods for determining markers are well-known from the skilled artisan, and include, without limitation, comparing the transcriptome (in an embodiment wherein expression relates to transcription of a marker) or proteome (in an embodiment wherein expression relates to translation of a marker) in a DED patient, a mild DED patient or a severe DED patient, and, for example, a “normal” patient. An example of such a method, based on the comparison of transcriptomes, is presented in the Examples.

In one embodiment, the genes are identified as differentially expressed in DED, mild DED or severe DED, when there is at least about a 1.1 fold difference in expression from normal, preferably at least about a 1.2, 1.25, 1.3, 1.4 or 1.5 fold difference. In another embodiment, the genes are identified as differentially expressed in DED, mild DED or severe DED, when there is at least about a 2 fold difference in expression from normal. In a further embodiment, the genes are identified as differentially expressed in DED, mild DED or severe DED, when there is at least about a 2.3 fold difference in expression from normal. In a further embodiment, the genes are identified as differentially expressed in DED, mild DED or severe DED, when there is at least about a 2.5 fold difference in expression from normal, including at least about 2.6 fold, 2.7 fold, 2.8 fold, 2.9 fold, 3 fold, 3.5 fold, 4 fold, and 5 fold. However, some genes can show a higher difference in expression than others. These genes can be more involved or alternatively, equally involved in the manifestation of disease as a gene that is less differentially expressed.

In one embodiment, the genes are identified as differentially expressed in mild DED, when there is at least about a 1.25 fold difference in expression from severe DED, preferably at least about a 1.3, 1.4 or 1.5 fold difference. In another embodiment, the genes are identified as differentially expressed in mild DED, when there is at least about a 2 fold difference in expression from severe DED. In a further embodiment, the genes are identified as differentially expressed in mild DED, when there is at least about a 2.3 fold difference in expression from severe DED. In a further embodiment, the genes are identified as differentially expressed in mild DED, when there is at least about a 2.5 fold difference in expression from severe DED, including at least about 2.6 fold, 2.7 fold, 2.8 fold, 2.9 fold, 3 fold, 3.5 fold, 4 fold, and 5 fold.

In one embodiment, the genes are identified as differentially expressed in severe DED, when there is at least about a 1.25 fold difference in expression from mild DED, preferably at least about a 1.3, 1.4 or 1.5 fold difference. In another embodiment, the genes are identified as differentially expressed in severe DED, when there is at least about a 2 fold difference in expression from mild DED. In a further embodiment, the genes are identified as differentially expressed in severe DED, when there is at least about a 2.3 fold difference in expression from mild DED. In a further embodiment, the genes are identified as differentially expressed in severe DED, when there is at least about a 2.5 fold difference in expression from mild DED, including at least about 2.6 fold, 2.7 fold, 2.8 fold, 2.9 fold, 3 fold, 3.5 fold, 4 fold, and 5 fold.

In one embodiment, the difference in expression is positive or negative. In other words, in one embodiment, genes may be induced or down-regulated, respectively.

Markers whose expression is different between a DED patient and a “normal” patient are hereinafter referred as “DED markers”.

The present invention thus also relates to “DED markers”. In one embodiment, the signature for dry eye disease according to the invention comprises at least one DED marker.

In one embodiment, the at least one DED marker is selected from the list of the 61 DED markers of Table 1 below, as well as their variants, fragments or equivalents. Table 1 comprises DED markers, i.e. markers whose expression is different between a DED patient and a “normal” patient, identified in the conditions of the Example and presenting a fold-change, positive or negative, superior or equal to 1.25.

TABLE 1

| Name of | GeneBank | |
|---|---|---|
| the | Accession | |
| marker | Number | Target sequence (SEQ ID number) |
| ATF2 | NM_001880.2 | CCAGGCAATACAAGGACCTGTGGAATATGAGTGATGAC |
| | | AAACCCTTTCTATGTACTGCGCCTGGATGTGGCCAGCGT |
| | | TTTACCAACGAGGATCATTTGGC (SEQ ID NO: 1) |
| AGER | NM_001136.3 | GAAAGGAGACCAAGTCCAACTACCGAGTCCGTGTCTAC |
| | | CAGATTCCTGGGAAGCCAGAAATTGTAGATTCTGCCTCT |
| | | GAACTCACGGCTGGTGTTCCCAA (SEQ ID NO: 2) |
| AREG | NM_001657.2 | ACAAGACGGAAAGTGAAAATACTTCAGATAAACCCAAA |
| | | AGAAAGAAAAAGGGAGGCAAAAATGGAAAAAATAGAA |
| | | GAAACAGAAAGAAGAAAAATCCATG (SEQ ID NO: 3) |
| C1QA | NM_015991.2 | GGTGACCAGGTCTGGGTTGAAAAAGACCCCAAAAAGGG |
| | | TCACATTTACCAGGGCTCTGAGGCCGACAGCGTCTTCAG |
| | | CGGCTTCCTCATCTTCCCATCTG (SEQ ID NO: 4) |
| C1S | NM_001734.2 | ACTGCACTGATTGGGGAGATTGCAAGTCCCAATTATCCC |
| | | AAACCATATCCAGAGAACTCAAGGTGTGAATACCAGAT |
| | | CCGGTTGGAGAAAGGGTTCCAAG (SEQ ID NO: 5) |
| C2 | NM_000063.3 | ATCTCATGATGAACAACCAAATGCGACTCCTCGGCATG |
| | | GAAACGATGGCCTGGCAGGAAATCCGACATGCCATCAT |
| | | CCTTCTGACAGATGGAAAGTCCAA (SEQ ID NO: 6) |
| C3AR1 | NM_004054.2 | CAGTGTCTTCCTGCTTACTGCCATTAGCCTGGATCGCTG |
| | | TCTTGTGGTATTCAAGCCAATCTGGTGTCAGAATCATCG |
| | | CAATGTAGGGATGGCCTGCTCT (SEQ ID NO: 7) |
| CCL4 | NM_002984.2 | TTCTGCAGCCTCACCTCTGAGAAAACCTCTTTGCCACCA |
| | | ATACCATGAAGCTCTGCGTGACTGTCCTGTCTCTCCTCA |
| | | TGCTAGTAGCTGCCTTCTGCTC (SEQ ID NO: 8) |
| CCL17 | NM_002987.2 | GCCTGGAGTACTTCAAGGGAGCCATTCCCCTTAGAAAG |
| | | CTGAAGACGTGGTACCAGACATCTGAGGACTGCTCCAG |
| | | GGATGCCATCGTTTTTGTAACTGT (SEQ ID NO: 9) |
| CCL20 | NM_004591.1 | ATCTGTTCTTTGAGCTAAAAACCATGTGCTGTACCAAGA |
| | | GTTTGCTCCTGGCTGCTTTGATGTCAGTGCTGCTACTCC |
| | | ACCTCTGCGGCGAATCAGAAGC (SEQ ID NO: 10) |
| CCL24 | NM_002991.2 | ATAGTAACCAGCCTTCTGTTCCTTGGTGTCTGTGCCCAC |
| | | CACATCATCCCTACGGGCTCTGTGGTCATCCCCTCTCCC |
| | | TGCTGCATGTTCTTTGTTTCCA (SEQ ID NO: 11) |
| CCR2 | NM_001123041.2 | TCTGATCTGCTTTTTCTTATTACTCTCCCATTGTGGGCTC |
| | | ACTCTGCTGCAAATGAGTGGGTCTTTGGGAATGCAATGT |
| | | GCAAATTATTCACAGGGCTGT (SEQ ID NO: 12) |
| CD4 | NM_000616.3 | AGACATCGTGGTGCTAGCTTTCCAGAAGGCCTCCAGCAT |
| | | AGTCTATAAGAAAGAGGGGGAACAGGTGGAGTTCTCCT |
| | | TCCCACTCGCCTTTACAGTTGAA (SEQ ID NO: 13) |
| CD40 | NM_001250.4 | CCCAGGAAGCCATATACACAGATGCCCATTGCAGCATT |
| | | GTTTGTGATAGTGAACAACTGGAAGCTGCTTAACTGTCC |
| | | ATCAGCAGGAGACTGGCTAAATA (SEQ ID NO: 14) |
| CD163 | NM_004244.4 | CATCTGTGATTCGGACTTCTCTCTGGAAGCTGCCAGCGT |
| | | TCTATGCAGGGAATTACAGTGTGGCACAGTTGTCTCTAT |
| | | CCTGGGGGGAGCTCACTTTGGA (SEQ ID NO: 15) |
| CFD | NM_001928.2 | CTGGTTGGTCTTTATTGAGCACCTACTATATGCAGAAGG |
| | | GGAGGCCGAGGTGGGAGGATCATTGGATCTCAGGAGTT |
| | | CGAGATCAGCATGGGCCACGTAG (SEQ ID NO: 16) |
| CSF1 | NM_000757.4 | TTTCTATGAGACACCTCTCCAGTTGCTGGAGAAGGTCAA |
| | | GAATGTCTTTAATGAAACAAAGAATCTCCTTGACAAGG |
| | | ACTGGAATATTTTCAGCAAGAAC (SEQ ID NO: 17) |
| CXCL9 | NM_002416.1 | CACCATCTCCCATGAAGAAAGGGAACGGTGAAGTACTA |
| | | AGCGCTAGAGGAAGCAGCCAAGTCGGTTAGTGGAAGCA |
| | | TGATTGGTGCCCAGTTAGCCTCTG (SEQ ID NO: 18) |
| CXCR4 | NM_003467.2 | ATTGATGTGTGTCTAGGCAGGACCTGTGGCCAAGTTCTT |
| | | AGTTGCTGTATGTCTCGTGGTAGGACTGTAGAAAAGGG |
| | | AACTGAACATTCCAGAGCGTGTA (SEQ ID NO: 19) |
| FOS | NM_005252.2 | ACTCAAGTCCTTACCTCTTCCGGAGATGTAGCAAAACGC |
| | | ATGGAGTGTGTATTGTTCCCAGTGACACTTCAGAGAGCT |
| | | GGTAGTTAGTAGCATGTTGAGC (SEQ ID NO: 20) |
| GNGT1 | NM_021955.3 | AAGAAGTAAGAGATTACGTTGAAGAACGATCTGGCGAG |
| | | GATCCACTGGTAAAGGGCATCCCAGAGGACAAAAATCC |
| | | CTTCAAGGAGCTCAAAGGAGGCTG (SEQ ID NO: 21) |
| HDAC4 | NM_006037.3 | TCTGTCAGCTCACTCCAGCTTCACAAATGTGCTGAGAGC |
| | | ATTACTGTGTAGCCTTTTCTTTGAAGACACACTCGGCTC |
| | | TTCTCCACAGCAAGCGTCCAGG (SEQ ID NO: 22) |
| HMGB2 | NM_001130688.1 | CTGTCAACATGGGTAAAGGAGACCCCAACAAGCCGCGG |
| | | GGCAAAATGTCCTCGTACGCCTTCTTCGTGCAGACCTGC |
| | | CGGGAAGAGCACAAGAAGAAACA (SEQ ID NO: 23) |
| HSPB2 | NM_001541.3 | AGCTGCTCTCTCCCATGATGGCATCTTAAACCTGGAAGC |
| | | ACCTCGGGGTGGCCGACATTTGGACACAGAGGTCAATG |
| | | AGGTCTACATCTCCCTGCTCCCT (SEQ ID NO: 24) |
| IFIT2 | NM_001547.4 | TGCATCCCATAGAGGTTAGTCCTGCATAGCCAGTAATGT |
| | | GCTAAGTTCATCCAAAAGCTGGCGGACCAAAGTCTAAA |
| | | TAGGGCTCAGTATCCCCCATCGC (SEQ ID NO: 25) |
| IFNG | NM_000619.2 | ATACTATCCAGTTACTGCCGGTTTGAAAATATGCCTGCA |
| | | ATCTGAGCCAGTGCTTTAATGGCATGTCAGACAGAACTT |
| | | GAATGTGTCAGGTGACCCTGAT (SEQ ID NO: 26) |
| IL4 | NM_000589.2 | GACACTCGCTGCCTGGGTGCGACTGCACAGCAGTTCCA |
| | | CAGGCACAAGCAGCTGATCCGATTCCTGAAACGGCTCG |
| | | ACAGGAACCTCTGGGGCCTGGCGG (SEQ ID NO: 27) |
| IL6 | NM_000600.1 | TGACAAACAAATTCGGTACATCCTCGACGGCATCTCAG |
| | | CCCTGAGAAAGGAGACATGTAACAAGAGTAACATGTGT |
| | | GAAAGCAGCAAAGAGGCACTGGCA (SEQ ID NO: 28) |
| IL6R | NM_000565.2 | CTTTCTACATAGTGTCCATGTGCGTCGCCAGTAGTGTCG |
| | | GGAGCAAGTTCAGCAAAACTCAAACCTTTCAGGGTTGT |
| | | GGAATCTTGCAGCCTGATCCGCC (SEQ ID NO: 29) |
| IL8 | NM_000584.2 | ACAGCAGAGCACACAAGCTTCTAGGACAAGAGCCAGGA |
| | | AGAAACCACCGGAAGGAACCATCTCACTGTGTGTAAAC |
| | | ATGACTTCCAAGCTGGCCGTGGCT (SEQ ID NO: 30) |
| IL10RB | NM_000628.3 | TTCTACCAGATTATGGATGGACTGATCTGAAAATCGACC |
| | | TCAACTCAAGGGTGGTCAGCTCAATGCTACACAGAGCA |
| | | CGGACTTTTGGATTCTTTGCAGT (SEQ ID NO: 31) |
| IL18RAP | NM_003853.2 | GCTTGATGGACAATGGAGTGGGATTGAGACTGTGGTTT |
| | | AGAGCCTTTGATTTCCTGGACTGGACTGACGGCGAGTG |
| | | AATTCTCTAGACCTTGGGTACTTT (SEQ ID NO: 32) |
| IRF1 | NM_002198.1 | CTGTGCGAGTGTACCGGATGCTTCCACCTCTCACCAAGA |
| | | ACCAGAGAAAAGAAAGAAAGTCGAAGTCCAGCCGAGA |
| | | TGCTAAGAGCAAGGCCAAGAGGAA (SEQ ID NO: 33) |
| IRF7 | NM_001572.3 | CGCAGCGTGAGGGTGTGTCTTCCCTGGATAGCAGCAGC |
| | | CTCAGCCTCTGCCTGTCCAGCGCCAACAGCCTCTATGAC |
| | | GACATCGAGTGCTTCCTTATGGA (SEQ ID NO: 34) |
| JUN | NM_002228.3 | ACACAGCCAGCCAGCCAGGTCGGCAGTATAGTCCGAAC |
| | | TGCAAATCTTATTTTCTTTTCACCTTCTCTCTAACTGCCC |
| | | AGAGCTAGCGCCTGTGGCTCCC (SEQ ID NO: 35) |
| LTB4R | NM_181657.3 | GTAGTGCCCTGGAAAACGAACATGAGCCTGTGCTTCCC |
| | | GCGGTACCCCAGCGAAGGGCACCGGGCCTTCCATCTAA |
| | | TCTTCGAGGCTGTCACGGGCTTCC (SEQ ID NO: 36) |
| MAP2K6 | NM_002758.3 | AGCCAGGAACAGAAACGGCTACTGATGGATTTGGATAT |
| | | TTCCATGAGGACGGTGGACTGTCCATTCACTGTCACCTT |
| | | TTATGGCGCACTGTTTCGGGAGG (SEQ ID NO: 37) |
| MAP3K5 | NM_005923.3 | TTTAGGAAAAGGCACTTATGGGATAGTCTACGCAGGTC |
| | | GGGACTTGAGCAACCAAGTCAGAATTGCTATTAAGGAA |
| | | ATCCCAGAGAGAGACAGCAGATAC (SEQ ID NO: 38) |
| MEF2C | NM_002397.3 | ACATTTGCCAAAAGCGGCAGATCTTTACTGAAAGAGAG |
| | | AGCAGCTGTTATGCAACATATAGAAAAATGTATAGATG |
| | | CTTGGACAGACCCGGTAATGGGTG (SEQ ID NO: 39) |
| MRC1 | NM_002438.2 | CTATGGAACCACAGACAATCTGTGCTCCAGAGGTTATG |
| | | AAGCCATGTATACGCTACTAGGCAATGCCAATGGAGCA |
| | | ACCTGTGCATTCCCGTTCAAGTTT (SEQ ID NO: 40) |
| MX1 | NM_002462.2 | GCCTTTAATCAGGACATCACTGCTCTCATGCAAGGAGA |
| | | GGAAACTGTAGGGGAGGAAGACATTCGGCTGTTTACCA |
| | | GACTCCGACACGAGTTCCACAAAT (SEQ ID NO: 41) |
| MYC | NM_002467.3 | TCGGACACCGAGGAGAATGTCAAGAGGCGAACACACA |
| | | ACGTCTTGGAGCGCCAGAGGAGGAACGAGCTAAAACGG |
| | | AGCTTTTTTGCCCTGCGTGACCAGA (SEQ ID NO: 42) |
| MYD88 | NM_002468.3 | ACGTTTTTCTAGGTACAGCTCCCAGGAACAGCTAGGTGG |
| | | GAAAGTCCCATCACTGAGGGAGCCTAACCATGTCCCTG |
| | | AACAAAAATTGGGCACTCATCTA (SEQ ID NO: 43) |
| NLRP3 | NM_001079821.2 | AGTGGGGTTCAGATAATGCACGTGTTTCGAATCCCACTG |
| | | TGATATGCCAGGAAGACAGCATTGAAGAGGAGTGGATG |
| | | GGTTTACTGGAGTACCTTTCGAG (SEQ ID NO: 44) |
| NOD1 | NM_006092.1 | AGAGGATGCTTTCCTGTTCATGGGGTTTTTGCCCTGGAG |
| | | CCTCAGCAGCAAATGCCACTCTGGGCAGTCTTTTGTGTC |
| | | AGTGTCTTAAAGGGGCCTGCGC (SEQ ID NO: 45) |
| OASL | NM_198213.1 | GGCGTTTCTGAGCTGTTTCCACAGCTTCCAGGAGGCAGC |
| | | CAAGCATCACAAAGATGTTCTGAGGCTGATATGGAAAA |
| | | CCATGTGGCAAAGCCAGGACCTG (SEQ ID NO: 46) |
| PIK3C2G | NM_004570.4 | CTGGAATATCTCCCACAGCTAGTTCAGGCTGTCAAGTTT |
| | | GAATGGAACCTTGAGAGTCCTTTAGTGCAACTTCTACTC |
| | | CACCGCTCCTTGCAGAGCATCC (SEQ ID NO: 47) |
| PLCB1 | NM_182734.1 | TCAAAACAAGGAGACAGAGCTACTGGATCTCAGCCTTG |
| | | TCAAAGATGCCAGATGTGGGAGACACGCCAAAGCTCCC |
| | | AAGGACCCCAAATTACGTGAACTT (SEQ ID NO: 48) |
| PRKCB | NM_212535.1 | GCATTTGGAGTCCTGCTGTATGAAATGTTGGCTGGGCAG |
| | | GCACCCTTTGAAGGGGAGGATGAAGATGAACTCTTCCA |
| | | ATCCATCATGGAACACAACGTAG (SEQ ID NO: 49) |
| PTGER2 | NM_000956.2 | GTCAGAAGGAGCTACAAAACCTACCCTCAGTGAGCATG |
| | | GTACTTGGCCTTTGGAGGAACAATCGGCTGCATTGAAG |
| | | ATCCAGCTGCCTATTGATTTAAGC (SEQ ID NO: 50) |
| PTGFR | NM_000959.3 | AACAGCCTTGCCATCGCCATTCTCATGAAGGCATATCAG |
| | | AGATTTAGACAGAAGTCCAAGGCATCGTTTCTGCTTTTG |
| | | GCCAGTGGCCTGGTAATCACTG (SEQ ID NO: 51) |
| STAT1 | NM_007315.2 | TTTGCTGTATGCCATCCTCGAGAGCTGTCTAGGTTAACG |
| | | TTCGCACTCTGTGTATATAACCTCGACAGTCTTGGCACC |
| | | TAACGTGCTGTGCGTAGCTGCT (SEQ ID NO: 52) |
| STAT2 | NM_005419.2 | CCGTACACGAAGGAGGTGCTGCAGTCACTCCCGCTGAC |
| | | TGAAATCATCCGCCATTACCAGTTGCTCACTGAGGAGA |
| | | ATATACCTGAAAACCCACTGCGCT (SEQ ID NO: 53) |
| TCF4 | NM_003199.1 | TGGAATCATTGGACCTTCTCATAATGGAGCCATGGGTGG |
| | | TCTGGGCTCAGGGTATGGAACCGGCCTTCTTTCAGCCAA |
| | | CAGACATTCACTCATGGTGGGG (SEQ ID NO: 54) |
| TGFB1 | NM_000660.3 | TATATGTTCTTCAACACATCAGAGCTCCGAGAAGCGGTA |
| | | CCTGAACCCGTGTTGCTCTCCCGGGCAGAGCTGCGTCTG |
| | | CTGAGGCTCAAGTTAAAAGTGG (SEQ ID NO: 55) |
| TGFBR1 | NM_004612.2 | GGGGAAATACGACTTAGTGAGGCATAGACATCCCTGGT |
| | | CCATCCTTTCTGTCTCCAGCTGTTTCTTGGAACCTGCTCT |
| | | CCTGCTTGCTGGTCCCTGACGC (SEQ ID NO : 56) |
| TLR4 | NM_138554.2 | ACTCAGAAAAGCCCTGCTGGATGGTAAATCATGGAATC |
| | | CAGAAGGAACAGTGGGTACAGGATGCAATTGGCAGGA |
| | | AGCAACATCTATCTGAAGAGGAAAA (SEQ ID NO: 57) |
| TLR5 | NM_003268.3 | GTTCCTGGCACTACTACAAGATTCATACTCCTGATGCTA |
| | | CTGACAACGTGGCTTCTCCACAGTCACCAAACCAGGGA |
| | | TGCTATACTGGACTTCCCTACTC (SEQ ID NO: 58) |
| TLR8 | NM_016610.2 | TTTAACTGATAGCCTATCTGACTTTACATCTTCCCTTCGG |
| | | ACACTGCTGCTGAGTCATAACAGGATTTCCCACCTACCC |
| | | TCTGGCTTTCTTTCTGAAGTC (SEQ ID NO: 59) |
| TNF | NM_000594.2 | AGCAACAAGACCACCACTTCGAAACCTGGGATTCAGGA |
| | | ATGTGTGGCCTGCACAGTGAAGTGCTGGCAACCACTAA |
| | | GAATTCAAACTGGGGCCTCCAGAA (SEQ ID NO: 60) |
| TREM2 | NM_018965.3 | TTCCCACCCACTTCCATCCTTCTCCTCCTGGCCTGCATCT |
| | | TTCTCATCAAGATTCTAGCAGCCAGCGCCCTCTGGGCTG |
| | | CAGCCTGGCATGGACAGAAGC (SEQ ID NO: 61) |

In one embodiment, a variant of a nucleotide sequence SEQ ID NO: X is a nucleotide sequence comprising at least 25 contiguous nucleotides, preferably of at least 50, 100, 150, 200 or at least 500 contiguous nucleotides of said nucleotide sequence SEQ ID NO: X.

In another embodiment, a variant of a nucleotide sequence SEQ ID NO: X is a nucleotide sequence comprising the nucleotide sequence SEQ ID NO: X and additional nucleic acids in 3′ and/or 5′ of SEQ ID NO: X, wherein the number of additional nucleic acids ranges from 1 to 500, preferably from 1 to 200, more preferably from 1 to 100 nucleotides.

In another embodiment, a variant of a nucleotide sequence SEQ ID NO: X is a nucleotide sequence that typically differs from said nucleotide sequence SEQ ID NO: X in one or more substitutions, deletions, additions and/or insertions. In one embodiment, said substitutions, deletions, additions and/or insertions may affect 1, 2, 3, 4, 5, 6, 7, 8, 9, or 10 nucleic acids.

In another embodiment, a variant of a nucleotide sequence SEQ ID NO: X is a nucleotide sequence of at least 25, preferably of at least 50, 100, 150, 200, 300, 400, 500, 1000, 1500, 2000 or 3000 nucleotides having at least 75%, 80%, 90%, 95%, or at least 96%, 97%, 98%, 99% identity with the nucleotide sequence SEQ ID NO: X.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

In one embodiment of the invention, a fragment is a nucleotide sequence of at least 25 nucleotides, preferably of at least 50, 100, 150, 200 or at least 500 nucleotides. In one embodiment of the invention, a fragment of a sequence SEQ ID NO: X is a sequence of at least 25 contiguous nucleotides, preferably of at least 50, 100, 150, 200 or at least 500 contiguous nucleotides of SEQ ID NO: X.

In one embodiment, an equivalent of a nucleotide sequence SEQ ID NO: X, preferably of a gene having the sequence SEQ ID NO: X, is a nucleotide sequence, preferably a gene involved in the same pathway than the nucleotide sequence SEQ ID NO: X.

In another embodiment, the at least one DED marker is selected from the list of the 58 DED markers of Table 2 below, as well as their variants, fragments or equivalents. Table 2 comprises DED markers, i.e. markers whose expression is different between a DED patient and a “normal” patient, identified in the conditions of the Example and presenting a fold-change, positive or negative, superior or equal to 1.25.

TABLE 2

| Name of | GeneBank | |
|---|---|---|
| the | Accession | |
| marker | Number | Target sequence (SEQ ID number) |
| ATF2 | NM_001880.2 | CCAGGCAATACAAGGACCTGTGGAATATGAGTGATGAC |
| | | AAACCCTTTCTATGTACTGCGCCTGGATGTGGCCAGCGT |
| | | TTTACCAACGAGGATCATTTGGC (SEQ ID NO: 1) |
| AGER | NM_001136.3 | GAAAGGAGACCAAGTCCAACTACCGAGTCCGTGTCTAC |
| | | CAGATTCCTGGGAAGCCAGAAATTGTAGATTCTGCCTCT |
| | | GAACTCACGGCTGGTGTTCCCAA (SEQ ID NO: 2) |
| AREG | NM_001657.2 | ACAAGACGGAAAGTGAAAATACTTCAGATAAACCCAAA |
| | | AGAAAGAAAAAGGGAGGCAAAAATGGAAAAAATAGAA |
| | | GAAACAGAAAGAAGAAAAATCCATG (SEQ ID NO: 3) |
| C1QA | NM_015991.2 | GGTGACCAGGTCTGGGTTGAAAAAGACCCCAAAAAGGG |
| | | TCACATTTACCAGGGCTCTGAGGCCGACAGCGTCTTCAG |
| | | CGGCTTCCTCATCTTCCCATCTG (SEQ ID NO: 4) |
| C1S | NM_001734.2 | ACTGCACTGATTGGGGAGATTGCAAGTCCCAATTATCCC |
| | | AAACCATATCCAGAGAACTCAAGGTGTGAATACCAGAT |
| | | CCGGTTGGAGAAAGGGTTCCAAG (SEQ ID NO: 5) |
| C2 | NM_000063.3 | ATCTCATGATGAACAACCAAATGCGACTCCTCGGCATG |
| | | GAAACGATGGCCTGGCAGGAAATCCGACATGCCATCAT |
| | | CCTTCTGACAGATGGAAAGTCCAA (SEQ ID NO: 6) |
| C3AR1 | NM_004054.2 | CAGTGTCTTCCTGCTTACTGCCATTAGCCTGGATCGCTG |
| | | TCTTGTGGTATTCAAGCCAATCTGGTGTCAGAATCATCG |
| | | CAATGTAGGGATGGCCTGCTCT (SEQ ID NO: 7) |
| CCL4 | NM_002984.2 | TTCTGCAGCCTCACCTCTGAGAAAACCTCTTTGCCACCA |
| | | ATACCATGAAGCTCTGCGTGACTGTCCTGTCTCTCCTCA |
| | | TGCTAGTAGCTGCCTTCTGCTC (SEQ ID NO: 8) |
| CCL17 | NM_002987.2 | GCCTGGAGTACTTCAAGGGAGCCATTCCCCTTAGAAAG |
| | | CTGAAGACGTGGTACCAGACATCTGAGGACTGCTCCAG |
| | | GGATGCCATCGTTTTTGTAACTGT (SEQ ID NO: 9) |
| CCL20 | NM_004591.1 | ATCTGTTCTTTGAGCTAAAAACCATGTGCTGTACCAAGA |
| | | GTTTGCTCCTGGCTGCTTTGATGTCAGTGCTGCTACTCC |
| | | ACCTCTGCGGCGAATCAGAAGC (SEQ ID NO: 10) |
| CCL24 | NM_002991.2 | ATAGTAACCAGCCTTCTGTTCCTTGGTGTCTGTGCCCAC |
| | | CACATCATCCCTACGGGCTCTGTGGTCATCCCCTCTCCC |
| | | TGCTGCATGTTCTTTGTTTCCA (SEQ ID NO: 11) |
| CCR2 | NM_001123041.2 | TCTGATCTGCTTTTTCTTATTACTCTCCCATTGTGGGCTC |
| | | ACTCTGCTGCAAATGAGTGGGTCTTTGGGAATGCAATGT |
| | | GCAAATTATTCACAGGGCTGT (SEQ ID NO: 12) |
| CD4 | NM_000616.3 | AGACATCGTGGTGCTAGCTTTCCAGAAGGCCTCCAGCAT |
| | | AGTCTATAAGAAAGAGGGGGAACAGGTGGAGTTCTCCT |
| | | TCCCACTCGCCTTTACAGTTGAA (SEQ ID NO: 13) |
| CD40 | NM_001250.4 | CCCAGGAAGCCATATACACAGATGCCCATTGCAGCATT |
| | | GTTTGTGATAGTGAACAACTGGAAGCTGCTTAACTGTCC |
| | | ATCAGCAGGAGACTGGCTAAATA (SEQ ID NO: 14) |
| CD163 | NM_004244.4 | CATCTGTGATTCGGACTTCTCTCTGGAAGCTGCCAGCGT |
| | | TCTATGCAGGGAATTACAGTGTGGCACAGTTGTCTCTAT |
| | | CCTGGGGGGAGCTCACTTTGGA (SEQ ID NO: 15) |
| CFD | NM_001928.2 | CTGGTTGGTCTTTATTGAGCACCTACTATATGCAGAAGG |
| | | GGAGGCCGAGGTGGGAGGATCATTGGATCTCAGGAGTT |
| | | CGAGATCAGCATGGGCCACGTAG (SEQ ID NO: 16) |
| CSF1 | NM_000757.4 | TTTCTATGAGACACCTCTCCAGTTGCTGGAGAAGGTCAA |
| | | GAATGTCTTTAATGAAACAAAGAATCTCCTTGACAAGG |
| | | ACTGGAATATTTTCAGCAAGAAC (SEQ ID NO: 17) |
| CXCL9 | NM_002416.1 | CACCATCTCCCATGAAGAAAGGGAACGGTGAAGTACTA |
| | | AGCGCTAGAGGAAGCAGCCAAGTCGGTTAGTGGAAGCA |
| | | TGATTGGTGCCCAGTTAGCCTCTG (SEQ ID NO: 18) |
| CXCR4 | NM_003467.2 | ATTGATGTGTGTCTAGGCAGGACCTGTGGCCAAGTTCTT |
| | | AGTTGCTGTATGTCTCGTGGTAGGACTGTAGAAAAGGG |
| | | AACTGAACATTCCAGAGCGTGTA (SEQ ID NO: 19) |
| FOS | NM_005252.2 | ACTCAAGTCCTTACCTCTTCCGGAGATGTAGCAAAACGC |
| | | ATGGAGTGTGTATTGTTCCCAGTGACACTTCAGAGAGCT |
| | | GGTAGTTAGTAGCATGTTGAGC (SEQ ID NO: 20) |
| GNGT1 | NM_021955.3 | AAGAAGTAAGAGATTACGTTGAAGAACGATCTGGCGAG |
| | | GATCCACTGGTAAAGGGCATCCCAGAGGACAAAAATCC |
| | | CTTCAAGGAGCTCAAAGGAGGCTG (SEQ ID NO: 21) |
| HDAC4 | NM_006037.3 | TCTGTCAGCTCACTCCAGCTTCACAAATGTGCTGAGAGC |
| | | ATTACTGTGTAGCCTTTTCTTTGAAGACACACTCGGCTC |
| | | TTCTCCACAGCAAGCGTCCAGG (SEQ ID NO: 22) |
| HMGB2 | NM_001130688.1 | CTGTCAACATGGGTAAAGGAGACCCCAACAAGCCGCGG |
| | | GGCAAAATGTCCTCGTACGCCTTCTTCGTGCAGACCTGC |
| | | CGGGAAGAGCACAAGAAGAAACA (SEQ ID NO: 23) |
| HSPB2 | NM_001541.3 | AGCTGCTCTCTCCCATGATGGCATCTTAAACCTGGAAGC |
| | | ACCTCGGGGTGGCCGACATTTGGACACAGAGGTCAATG |
| | | AGGTCTACATCTCCCTGCTCCCT (SEQ ID NO: 24) |
| IFIT2 | NM_001547.4 | TGCATCCCATAGAGGTTAGTCCTGCATAGCCAGTAATGT |
| | | GCTAAGTTCATCCAAAAGCTGGCGGACCAAAGTCTAAA |
| | | TAGGGCTCAGTATCCCCCATCGC (SEQ ID NO: 25) |
| IFNG | NM_000619.2 | ATACTATCCAGTTACTGCCGGTTTGAAAATATGCCTGCA |
| | | ATCTGAGCCAGTGCTTTAATGGCATGTCAGACAGAACTT |
| | | GAATGTGTCAGGTGACCCTGAT (SEQ ID NO: 26) |
| IL4 | NM_000589.2 | GACACTCGCTGCCTGGGTGCGACTGCACAGCAGTTCCA |
| | | CAGGCACAAGCAGCTGATCCGATTCCTGAAACGGCTCG |
| | | ACAGGAACCTCTGGGGCCTGGCGG (SEQ ID NO: 27) |
| IL6 | NM_000600.1 | TGACAAACAAATTCGGTACATCCTCGACGGCATCTCAG |
| | | CCCTGAGAAAGGAGACATGTAACAAGAGTAACATGTGT |
| | | GAAAGCAGCAAAGAGGCACTGGCA (SEQ ID NO: 28) |
| IL6R | NM_000565.2 | CTTTCTACATAGTGTCCATGTGCGTCGCCAGTAGTGTCG |
| | | GGAGCAAGTTCAGCAAAACTCAAACCTTTCAGGGTTGT |
| | | GGAATCTTGCAGCCTGATCCGCC (SEQ ID NO: 29) |
| IL8 | NM_000584.2 | ACAGCAGAGCACACAAGCTTCTAGGACAAGAGCCAGGA |
| | | AGAAACCACCGGAAGGAACCATCTCACTGTGTGTAAAC |
| | | ATGACTTCCAAGCTGGCCGTGGCT (SEQ ID NO: 30) |
| IL10RB | NM_000628.3 | TTCTACCAGATTATGGATGGACTGATCTGAAAATCGACC |
| | | TCAACTCAAGGGTGGTCAGCTCAATGCTACACAGAGCA |
| | | CGGACTTTTGGATTCTTTGCAGT (SEQ ID NO: 31) |
| IL18RAP | NM_003853.2 | GCTTGATGGACAATGGAGTGGGATTGAGACTGTGGTTT |
| | | AGAGCCTTTGATTTCCTGGACTGGACTGACGGCGAGTG |
| | | AATTCTCTAGACCTTGGGTACTTT (SEQ ID NO: 32) |
| IRF1 | NM_002198.1 | CTGTGCGAGTGTACCGGATGCTTCCACCTCTCACCAAGA |
| | | ACCAGAGAAAAGAAAGAAAGTCGAAGTCCAGCCGAGA |
| | | TGCTAAGAGCAAGGCCAAGAGGAA (SEQ ID NO: 33) |
| IRF7 | NM_001572.3 | CGCAGCGTGAGGGTGTGTCTTCCCTGGATAGCAGCAGC |
| | | CTCAGCCTCTGCCTGTCCAGCGCCAACAGCCTCTATGAC |
| | | GACATCGAGTGCTTCCTTATGGA (SEQ ID NO: 34) |
| JUN | NM_002228.3 | ACACAGCCAGCCAGCCAGGTCGGCAGTATAGTCCGAAC |
| | | TGCAAATCTTATTTTCTTTTCACCTTCTCTCTAACTGCCC |
| | | AGAGCTAGCGCCTGTGGCTCCC (SEQ ID NO: 35) |
| LTB4R | NM_181657.3 | GTAGTGCCCTGGAAAACGAACATGAGCCTGTGCTTCCC |
| | | GCGGTACCCCAGCGAAGGGCACCGGGCCTTCCATCTAA |
| | | TCTTCGAGGCTGTCACGGGCTTCC (SEQ ID NO: 36) |
| MAP2K6 | NM_002758.3 | AGCCAGGAACAGAAACGGCTACTGATGGATTTGGATAT |
| | | TTCCATGAGGACGGTGGACTGTCCATTCACTGTCACCTT |
| | | TTATGGCGCACTGTTTCGGGAGG (SEQ ID NO: 37) |
| MAP3K5 | NM_005923.3 | TTTAGGAAAAGGCACTTATGGGATAGTCTACGCAGGTC |
| | | GGGACTTGAGCAACCAAGTCAGAATTGCTATTAAGGAA |
| | | ATCCCAGAGAGAGACAGCAGATAC (SEQ ID NO: 38) |
| MEF2C | NM_002397.3 | ACATTTGCCAAAAGCGGCAGATCTTTACTGAAAGAGAG |
| | | AGCAGCTGTTATGCAACATATAGAAAAATGTATAGATG |
| | | CTTGGACAGACCCGGTAATGGGTG (SEQ ID NO: 39) |
| MRC1 | NM_002438.2 | CTATGGAACCACAGACAATCTGTGCTCCAGAGGTTATG |
| | | AAGCCATGTATACGCTACTAGGCAATGCCAATGGAGCA |
| | | ACCTGTGCATTCCCGTTCAAGTTT (SEQ ID NO: 40) |
| MX1 | NM_002462.2 | GCCTTTAATCAGGACATCACTGCTCTCATGCAAGGAGA |
| | | GGAAACTGTAGGGGAGGAAGACATTCGGCTGTTTACCA |
| | | GACTCCGACACGAGTTCCACAAAT (SEQ ID NO: 41) |
| MYC | NM_002467.3 | TCGGACACCGAGGAGAATGTCAAGAGGCGAACACACA |
| | | ACGTCTTGGAGCGCCAGAGGAGGAACGAGCTAAAACGG |
| | | AGCTTTTTTGCCCTGCGTGACCAGA (SEQ ID NO: 42) |
| MYD88 | NM_002468.3 | ACGTTTTTCTAGGTACAGCTCCCAGGAACAGCTAGGTGG |
| | | GAAAGTCCCATCACTGAGGGAGCCTAACCATGTCCCTG |
| | | AACAAAAATTGGGCACTCATCTA (SEQ ID NO: 43) |
| NLRP3 | NM_001079821.2 | AGTGGGGTTCAGATAATGCACGTGTTTCGAATCCCACTG |
| | | TGATATGCCAGGAAGACAGCATTGAAGAGGAGTGGATG |
| | | GGTTTACTGGAGTACCTTTCGAG (SEQ ID NO: 44) |
| NOD1 | NM_006092.1 | AGAGGATGCTTTCCTGTTCATGGGGTTTTTGCCCTGGAG |
| | | CCTCAGCAGCAAATGCCACTCTGGGCAGTCTTTTGTGTC |
| | | AGTGTCTTAAAGGGGCCTGCGC (SEQ ID NO: 45) |
| OASL | NM_198213.1 | GGCGTTTCTGAGCTGTTTCCACAGCTTCCAGGAGGCAGC |
| | | CAAGCATCACAAAGATGTTCTGAGGCTGATATGGAAAA |
| | | CCATGTGGCAAAGCCAGGACCTG (SEQ ID NO: 46) |
| PIK3C2G | NM_004570.4 | CTGGAATATCTCCCACAGCTAGTTCAGGCTGTCAAGTTT |
| | | GAATGGAACCTTGAGAGTCCTTTAGTGCAACTTCTACTC |
| | | CACCGCTCCTTGCAGAGCATCC (SEQ ID NO: 47) |
| PLCB1 | NM_182734.1 | TCAAAACAAGGAGACAGAGCTACTGGATCTCAGCCTTG |
| | | TCAAAGATGCCAGATGTGGGAGACACGCCAAAGCTCCC |
| | | AAGGACCCCAAATTACGTGAACTT (SEQ ID NO: 48) |
| PRKCB | NM_212535.1 | GCATTTGGAGTCCTGCTGTATGAAATGTTGGCTGGGCAG |
| | | GCACCCTTTGAAGGGGAGGATGAAGATGAACTCTTCCA |
| | | ATCCATCATGGAACACAACGTAG (SEQ ID NO: 49) |
| PTGER2 | NM_000956.2 | GTCAGAAGGAGCTACAAAACCTACCCTCAGTGAGCATG |
| | | GTACTTGGCCTTTGGAGGAACAATCGGCTGCATTGAAG |
| | | ATCCAGCTGCCTATTGATTTAAGC (SEQ ID NO: 50) |
| PTGFR | NM_000959.3 | AACAGCCTTGCCATCGCCATTCTCATGAAGGCATATCAG |
| | | AGATTTAGACAGAAGTCCAAGGCATCGTTTCTGCTTTTG |
| | | GCCAGTGGCCTGGTAATCACTG (SEQ ID NO: 51) |
| STAT1 | NM_007315.2 | TTTGCTGTATGCCATCCTCGAGAGCTGTCTAGGTTAACG |
| | | TTCGCACTCTGTGTATATAACCTCGACAGTCTTGGCACC |
| | | TAACGTGCTGTGCGTAGCTGCT (SEQ ID NO: 52) |
| STAT2 | NM_005419.2 | CCGTACACGAAGGAGGTGCTGCAGTCACTCCCGCTGAC |
| | | TGAAATCATCCGCCATTACCAGTTGCTCACTGAGGAGA |
| | | ATATACCTGAAAACCCACTGCGCT (SEQ ID NO: 53) |
| TGFB1 | NM_000660.3 | TATATGTTCTTCAACACATCAGAGCTCCGAGAAGCGGTA |
| | | CCTGAACCCGTGTTGCTCTCCCGGGCAGAGCTGCGTCTG |
| | | CTGAGGCTCAAGTTAAAAGTGG (SEQ ID NO: 55) |
| TGFBR1 | NM_004612.2 | GGGGAAATACGACTTAGTGAGGCATAGACATCCCTGGT |
| | | CCATCCTTTCTGTCTCCAGCTGTTTCTTGGAACCTGCTCT |
| | | CCTGCTTGCTGGTCCCTGACGC (SEQ ID NO: 56) |
| TLR4 | NM_138554.2 | ACTCAGAAAAGCCCTGCTGGATGGTAAATCATGGAATC |
| | | CAGAAGGAACAGTGGGTACAGGATGCAATTGGCAGGA |
| | | AGCAACATCTATCTGAAGAGGAAAA (SEQ ID NO: 57) |
| TLR5 | NM_003268.3 | GTTCCTGGCACTACTACAAGATTCATACTCCTGATGCTA |
| | | CTGACAACGTGGCTTCTCCACAGTCACCAAACCAGGGA |
| | | TGCTATACTGGACTTCCCTACTC (SEQ ID NO: 58) |
| TLR8 | NM_016610.2 | TTTAACTGATAGCCTATCTGACTTTACATCTTCCCTTCGG |
| | | ACACTGCTGCTGAGTCATAACAGGATTTCCCACCTACCC |
| | | TCTGGCTTTCTTTCTGAAGTC (SEQ ID NO: 59) |
| TNF | NM_000594.2 | AGCAACAAGACCACCACTTCGAAACCTGGGATTCAGGA |
| | | ATGTGTGGCCTGCACAGTGAAGTGCTGGCAACCACTAA |
| | | GAATTCAAACTGGGGCCTCCAGAA (SEQ ID NO: 60) |
| TREM2 | NM_018965.3 | TTCCCACCCACTTCCATCCTTCTCCTCCTGGCCTGCATCT |
| | | TTCTCATCAAGATTCTAGCAGCCAGCGCCCTCTGGGCTG |
| | | CAGCCTGGCATGGACAGAAGC (SEQ ID NO: 61) |

In another embodiment, the at least one DED marker is selected from the list of the 51 DED markers of Table 3 below, as well as their variants, fragments or equivalents. Table 3 comprises DED markers, i.e. markers whose expression is different between a DED patient and a “normal” patient, identified in the conditions of the Example and presenting a fold-change, positive or negative, superior or equal to 1.25.

TABLE 3

| Name of | GeneBank | |
|---|---|---|
| the | Accession | |
| marker | Number | Target sequence (SEQ ID number) |
| ATF2 | NM_001880.2 | CCAGGCAATACAAGGACCTGTGGAATATGAGTGATGAC |
| | | AAACCCTTTCTATGTACTGCGCCTGGATGTGGCCAGCGT |
| | | TTTACCAACGAGGATCATTTGGC (SEQ ID NO: 1) |
| AGER | NM_001136.3 | GAAAGGAGACCAAGTCCAACTACCGAGTCCGTGTCTAC |
| | | CAGATTCCTGGGAAGCCAGAAATTGTAGATTCTGCCTCT |
| | | GAACTCACGGCTGGTGTTCCCAA (SEQ ID NO: 2) |
| AREG | NM_001657.2 | ACAAGACGGAAAGTGAAAATACTTCAGATAAACCCAAA |
| | | AGAAAGAAAAAGGGAGGCAAAAATGGAAAAAATAGAA |
| | | GAAACAGAAAGAAGAAAAATCCATG (SEQ ID NO: 3) |
| C1QA | NM_015991.2 | GGTGACCAGGTCTGGGTTGAAAAAGACCCCAAAAAGGG |
| | | TCACATTTACCAGGGCTCTGAGGCCGACAGCGTCTTCAG |
| | | CGGCTTCCTCATCTTCCCATCTG (SEQ ID NO: 4) |
| C1S | NM_001734.2 | ACTGCACTGATTGGGGAGATTGCAAGTCCCAATTATCCC |
| | | AAACCATATCCAGAGAACTCAAGGTGTGAATACCAGAT |
| | | CCGGTTGGAGAAAGGGTTCCAAG (SEQ ID NO: 5) |
| C2 | NM_000063.3 | ATCTCATGATGAACAACCAAATGCGACTCCTCGGCATG |
| | | GAAACGATGGCCTGGCAGGAAATCCGACATGCCATCAT |
| | | CCTTCTGACAGATGGAAAGTCCAA (SEQ ID NO: 6) |
| C3AR1 | NM_004054.2 | CAGTGTCTTCCTGCTTACTGCCATTAGCCTGGATCGCTG |
| | | TCTTGTGGTATTCAAGCCAATCTGGTGTCAGAATCATCG |
| | | CAATGTAGGGATGGCCTGCTCT (SEQ ID NO: 7) |
| CCL4 | NM_002984.2 | TTCTGCAGCCTCACCTCTGAGAAAACCTCTTTGCCACCA |
| | | ATACCATGAAGCTCTGCGTGACTGTCCTGTCTCTCCTCA |
| | | TGCTAGTAGCTGCCTTCTGCTC (SEQ ID NO: 8) |
| CCL17 | NM_002987.2 | GCCTGGAGTACTTCAAGGGAGCCATTCCCCTTAGAAAG |
| | | CTGAAGACGTGGTACCAGACATCTGAGGACTGCTCCAG |
| | | GGATGCCATCGTTTTTGTAACTGT (SEQ ID NO: 9) |
| CCL20 | NM_004591.1 | ATCTGTTCTTTGAGCTAAAAACCATGTGCTGTACCAAGA |
| | | GTTTGCTCCTGGCTGCTTTGATGTCAGTGCTGCTACTCC |
| | | ACCTCTGCGGCGAATCAGAAGC (SEQ ID NO: 10) |
| CCL24 | NM_002991.2 | ATAGTAACCAGCCTTCTGTTCCTTGGTGTCTGTGCCCAC |
| | | CACATCATCCCTACGGGCTCTGTGGTCATCCCCTCTCCC |
| | | TGCTGCATGTTCTTTGTTTCCA (SEQ ID NO: 11) |
| CCR2 | NM_001123041.2 | TCTGATCTGCTTTTTCTTATTACTCTCCCATTGTGGGCTC |
| | | ACTCTGCTGCAAATGAGTGGGTCTTTGGGAATGCAATGT |
| | | GCAAATTATTCACAGGGCTGT (SEQ ID NO: 12) |
| CD4 | NM_000616.3 | AGACATCGTGGTGCTAGCTTTCCAGAAGGCCTCCAGCAT |
| | | AGTCTATAAGAAAGAGGGGGAACAGGTGGAGTTCTCCT |
| | | TCCCACTCGCCTTTACAGTTGAA (SEQ ID NO: 13) |
| CD40 | NM_001250.4 | CCCAGGAAGCCATATACACAGATGCCCATTGCAGCATT |
| | | GTTTGTGATAGTGAACAACTGGAAGCTGCTTAACTGTCC |
| | | ATCAGCAGGAGACTGGCTAAATA (SEQ ID NO: 14) |
| CD163 | NM_004244.4 | CATCTGTGATTCGGACTTCTCTCTGGAAGCTGCCAGCGT |
| | | TCTATGCAGGGAATTACAGTGTGGCACAGTTGTCTCTAT |
| | | CCTGGGGGGAGCTCACTTTGGA (SEQ ID NO: 15) |
| CFD | NM_001928.2 | CTGGTTGGTCTTTATTGAGCACCTACTATATGCAGAAGG |
| | | GGAGGCCGAGGTGGGAGGATCATTGGATCTCAGGAGTT |
| | | CGAGATCAGCATGGGCCACGTAG (SEQ ID NO: 16) |
| CSF1 | NM_000757.4 | TTTCTATGAGACACCTCTCCAGTTGCTGGAGAAGGTCAA |
| | | GAATGTCTTTAATGAAACAAAGAATCTCCTTGACAAGG |
| | | ACTGGAATATTTTCAGCAAGAAC (SEQ ID NO: 17) |
| CXCR4 | NM_003467.2 | ATTGATGTGTGTCTAGGCAGGACCTGTGGCCAAGTTCTT |
| | | AGTTGCTGTATGTCTCGTGGTAGGACTGTAGAAAAGGG |
| | | AACTGAACATTCCAGAGCGTGTA (SEQ ID NO: 19) |
| FOS | NM_005252.2 | ACTCAAGTCCTTACCTCTTCCGGAGATGTAGCAAAACGC |
| | | ATGGAGTGTGTATTGTTCCCAGTGACACTTCAGAGAGCT |
| | | GGTAGTTAGTAGCATGTTGAGC (SEQ ID NO: 20) |
| GNGT1 | NM_021955.3 | AAGAAGTAAGAGATTACGTTGAAGAACGATCTGGCGAG |
| | | GATCCACTGGTAAAGGGCATCCCAGAGGACAAAAATCC |
| | | CTTCAAGGAGCTCAAAGGAGGCTG (SEQ ID NO: 21) |
| HDAC4 | NM_006037.3 | TCTGTCAGCTCACTCCAGCTTCACAAATGTGCTGAGAGC |
| | | ATTACTGTGTAGCCTTTTCTTTGAAGACACACTCGGCTC |
| | | TTCTCCACAGCAAGCGTCCAGG (SEQ ID NO: 22) |
| HMGB2 | NM_001130688.1 | CTGTCAACATGGGTAAAGGAGACCCCAACAAGCCGCGG |
| | | GGCAAAATGTCCTCGTACGCCTTCTTCGTGCAGACCTGC |
| | | CGGGAAGAGCACAAGAAGAAACA (SEQ ID NO: 23) |
| HSPB2 | NM_001541.3 | AGCTGCTCTCTCCCATGATGGCATCTTAAACCTGGAAGC |
| | | ACCTCGGGGTGGCCGACATTTGGACACAGAGGTCAATG |
| | | AGGTCTACATCTCCCTGCTCCCT (SEQ ID NO: 24) |
| IFIT2 | NM_001547.4 | TGCATCCCATAGAGGTTAGTCCTGCATAGCCAGTAATGT |
| | | GCTAAGTTCATCCAAAAGCTGGCGGACCAAAGTCTAAA |
| | | TAGGGCTCAGTATCCCCCATCGC (SEQ ID NO: 25) |
| IL6R | NM_000565.2 | CTTTCTACATAGTGTCCATGTGCGTCGCCAGTAGTGTCG |
| | | GGAGCAAGTTCAGCAAAACTCAAACCTTTCAGGGTTGT |
| | | GGAATCTTGCAGCCTGATCCGCC (SEQ ID NO: 29) |
| IL10RB | NM_000628.3 | TTCTACCAGATTATGGATGGACTGATCTGAAAATCGACC |
| | | TCAACTCAAGGGTGGTCAGCTCAATGCTACACAGAGCA |
| | | CGGACTTTTGGATTCTTTGCAGT (SEQ ID NO: 31) |
| IL18RAP | NM_003853.2 | GCTTGATGGACAATGGAGTGGGATTGAGACTGTGGTTT |
| | | AGAGCCTTTGATTTCCTGGACTGGACTGACGGCGAGTG |
| | | AATTCTCTAGACCTTGGGTACTTT (SEQ ID NO: 32) |
| IRF1 | NM_002198.1 | CTGTGCGAGTGTACCGGATGCTTCCACCTCTCACCAAGA |
| | | ACCAGAGAAAAGAAAGAAAGTCGAAGTCCAGCCGAGA |
| | | TGCTAAGAGCAAGGCCAAGAGGAA (SEQ ID NO: 33) |
| IRF7 | NM_001572.3 | CGCAGCGTGAGGGTGTGTCTTCCCTGGATAGCAGCAGC |
| | | CTCAGCCTCTGCCTGTCCAGCGCCAACAGCCTCTATGAC |
| | | GACATCGAGTGCTTCCTTATGGA (SEQ ID NO: 34) |
| JUN | NM_002228.3 | ACACAGCCAGCCAGCCAGGTCGGCAGTATAGTCCGAAC |
| | | TGCAAATCTTATTTTCTTTTCACCTTCTCTCTAACTGCCC |
| | | AGAGCTAGCGCCTGTGGCTCCC (SEQ ID NO: 35) |
| LTB4R | NM_181657.3 | GTAGTGCCCTGGAAAACGAACATGAGCCTGTGCTTCCC |
| | | GCGGTACCCCAGCGAAGGGCACCGGGCCTTCCATCTAA |
| | | TCTTCGAGGCTGTCACGGGCTTCC (SEQ ID NO: 36) |
| MAP2K6 | NM_002758.3 | AGCCAGGAACAGAAACGGCTACTGATGGATTTGGATAT |
| | | TTCCATGAGGACGGTGGACTGTCCATTCACTGTCACCTT |
| | | TTATGGCGCACTGTTTCGGGAGG (SEQ ID NO: 37) |
| MAP3K5 | NM_005923.3 | TTTAGGAAAAGGCACTTATGGGATAGTCTACGCAGGTC |
| | | GGGACTTGAGCAACCAAGTCAGAATTGCTATTAAGGAA |
| | | ATCCCAGAGAGAGACAGCAGATAC (SEQ ID NO: 38) |
| MEF2C | NM_002397.3 | ACATTTGCCAAAAGCGGCAGATCTTTACTGAAAGAGAG |
| | | AGCAGCTGTTATGCAACATATAGAAAAATGTATAGATG |
| | | CTTGGACAGACCCGGTAATGGGTG (SEQ ID NO: 39) |
| MRC1 | NM_002438.2 | CTATGGAACCACAGACAATCTGTGCTCCAGAGGTTATG |
| | | AAGCCATGTATACGCTACTAGGCAATGCCAATGGAGCA |
| | | ACCTGTGCATTCCCGTTCAAGTTT (SEQ ID NO: 40) |
| MX1 | NM_002462.2 | GCCTTTAATCAGGACATCACTGCTCTCATGCAAGGAGA |
| | | GGAAACTGTAGGGGAGGAAGACATTCGGCTGTTTACCA |
| | | GACTCCGACACGAGTTCCACAAAT (SEQ ID NO: 41) |
| MYC | NM_002467.3 | TCGGACACCGAGGAGAATGTCAAGAGGCGAACACACA |
| | | ACGTCTTGGAGCGCCAGAGGAGGAACGAGCTAAAACGG |
| | | AGCTTTTTTGCCCTGCGTGACCAGA (SEQ ID NO: 42) |
| MYD88 | NM_002468.3 | ACGTTTTTCTAGGTACAGCTCCCAGGAACAGCTAGGTGG |
| | | GAAAGTCCCATCACTGAGGGAGCCTAACCATGTCCCTG |
| | | AACAAAAATTGGGCACTCATCTA (SEQ ID NO: 43) |
| NLRP3 | NM_001079821.2 | AGTGGGGTTCAGATAATGCACGTGTTTCGAATCCCACTG |
| | | TGATATGCCAGGAAGACAGCATTGAAGAGGAGTGGATG |
| | | GGTTTACTGGAGTACCTTTCGAG (SEQ ID NO: 44) |
| NOD1 | NM_006092.1 | AGAGGATGCTTTCCTGTTCATGGGGTTTTTGCCCTGGAG |
| | | CCTCAGCAGCAAATGCCACTCTGGGCAGTCTTTTGTGTC |
| | | AGTGTCTTAAAGGGGCCTGCGC (SEQ ID NO: 45) |
| PIK3C2G | NM_004570.4 | CTGGAATATCTCCCACAGCTAGTTCAGGCTGTCAAGTTT |
| | | GAATGGAACCTTGAGAGTCCTTTAGTGCAACTTCTACTC |
| | | CACCGCTCCTTGCAGAGCATCC (SEQ ID NO: 47) |
| PLCB1 | NM_182734.1 | TCAAAACAAGGAGACAGAGCTACTGGATCTCAGCCTTG |
| | | TCAAAGATGCCAGATGTGGGAGACACGCCAAAGCTCCC |
| | | AAGGACCCCAAATTACGTGAACTT (SEQ ID NO: 48) |
| PRKCB | NM_212535.1 | GCATTTGGAGTCCTGCTGTATGAAATGTTGGCTGGGCAG |
| | | GCACCCTTTGAAGGGGAGGATGAAGATGAACTCTTCCA |
| | | ATCCATCATGGAACACAACGTAG (SEQ ID NO: 49) |
| PTGER2 | NM_000956.2 | GTCAGAAGGAGCTACAAAACCTACCCTCAGTGAGCATG |
| | | GTACTTGGCCTTTGGAGGAACAATCGGCTGCATTGAAG |
| | | ATCCAGCTGCCTATTGATTTAAGC (SEQ ID NO: 50) |
| PTGFR | NM_000959.3 | AACAGCCTTGCCATCGCCATTCTCATGAAGGCATATCAG |
| | | AGATTTAGACAGAAGTCCAAGGCATCGTTTCTGCTTTTG |
| | | GCCAGTGGCCTGGTAATCACTG (SEQ ID NO: 51) |
| STAT1 | NM_007315.2 | TTTGCTGTATGCCATCCTCGAGAGCTGTCTAGGTTAACG |
| | | TTCGCACTCTGTGTATATAACCTCGACAGTCTTGGCACC |
| | | TAACGTGCTGTGCGTAGCTGCT (SEQ ID NO: 52) |
| STAT2 | NM_005419.2 | CCGTACACGAAGGAGGTGCTGCAGTCACTCCCGCTGAC |
| | | TGAAATCATCCGCCATTACCAGTTGCTCACTGAGGAGA |
| | | ATATACCTGAAAACCCACTGCGCT (SEQ ID NO: 53) |
| TGFBR1 | NM_004612.2 | GGGGAAATACGACTTAGTGAGGCATAGACATCCCTGGT |
| | | CCATCCTTTCTGTCTCCAGCTGTTTCTTGGAACCTGCTCT |
| | | CCTGCTTGCTGGTCCCTGACGC (SEQ ID NO: 56) |
| TLR4 | NM_138554.2 | ACTCAGAAAAGCCCTGCTGGATGGTAAATCATGGAATC |
| | | CAGAAGGAACAGTGGGTACAGGATGCAATTGGCAGGA |
| | | AGCAACATCTATCTGAAGAGGAAAA (SEQ ID NO: 57) |
| TLR5 | NM_003268.3 | GTTCCTGGCACTACTACAAGATTCATACTCCTGATGCTA |
| | | CTGACAACGTGGCTTCTCCACAGTCACCAAACCAGGGA |
| | | TGCTATACTGGACTTCCCTACTC (SEQ ID NO: 58) |
| TLR8 | NM_016610.2 | TTTAACTGATAGCCTATCTGACTTTACATCTTCCCTTCGG |
| | | ACACTGCTGCTGAGTCATAACAGGATTTCCCACCTACCC |
| | | TCTGGCTTTCTTTCTGAAGTC (SEQ ID NO: 59) |
| TREM2 | NM_018965.3 | TTCCCACCCACTTCCATCCTTCTCCTCCTGGCCTGCATCT |
| | | TTCTCATCAAGATTCTAGCAGCCAGCGCCCTCTGGGCTG |
| | | CAGCCTGGCATGGACAGAAGC (SEQ ID NO: 61) |

In another embodiment, the at least one DED marker is selected from the list of the 42 DED markers of Table 4 below, as well as their variants, fragments or equivalents. Table 4 comprises DED markers, i.e. markers whose expression is different between a DED patient and a “normal” patient, identified in the conditions of the Example and presenting a fold-change, positive or negative, superior or equal to 1.5.

TABLE 4

| Name of | GeneBank | |
|---|---|---|
| the | Accession | |
| marker | Number | Target sequence (SEQ ID number) |
| AGER | NM_001136.3 | GAAAGGAGACCAAGTCCAACTACCGAGTCCGTGTCTAC |
| | | CAGATTCCTGGGAAGCCAGAAATTGTAGATTCTGCCTCT |
| | | GAACTCACGGCTGGTGTTCCCAA (SEQ ID NO: 2) |
| AREG | NM_001657.2 | ACAAGACGGAAAGTGAAAATACTTCAGATAAACCCAAA |
| | | AGAAAGAAAAAGGGAGGCAAAAATGGAAAAAATAGAA |
| | | GAAACAGAAAGAAGAAAAATCCATG (SEQ ID NO: 3) |
| C1QA | NM_015991.2 | GGTGACCAGGTCTGGGTTGAAAAAGACCCCAAAAAGGG |
| | | TCACATTTACCAGGGCTCTGAGGCCGACAGCGTCTTCAG |
| | | CGGCTTCCTCATCTTCCCATCTG (SEQ ID NO: 4) |
| C1S | NM_001734.2 | ACTGCACTGATTGGGGAGATTGCAAGTCCCAATTATCCC |
| | | AAACCATATCCAGAGAACTCAAGGTGTGAATACCAGAT |
| | | CCGGTTGGAGAAAGGGTTCCAAG (SEQ ID NO: 5) |
| C2 | NM_000063.3 | ATCTCATGATGAACAACCAAATGCGACTCCTCGGCATG |
| | | GAAACGATGGCCTGGCAGGAAATCCGACATGCCATCAT |
| | | CCTTCTGACAGATGGAAAGTCCAA (SEQ ID NO: 6) |
| C3AR1 | NM_004054.2 | CAGTGTCTTCCTGCTTACTGCCATTAGCCTGGATCGCTG |
| | | TCTTGTGGTATTCAAGCCAATCTGGTGTCAGAATCATCG |
| | | CAATGTAGGGATGGCCTGCTCT (SEQ ID NO: 7) |
| CCL4 | NM_002984.2 | TTCTGCAGCCTCACCTCTGAGAAAACCTCTTTGCCACCA |
| | | ATACCATGAAGCTCTGCGTGACTGTCCTGTCTCTCCTCA |
| | | TGCTAGTAGCTGCCTTCTGCTC (SEQ ID NO: 8) |
| CCL20 | NM_004591.1 | ATCTGTTCTTTGAGCTAAAAACCATGTGCTGTACCAAGA |
| | | GTTTGCTCCTGGCTGCTTTGATGTCAGTGCTGCTACTCC |
| | | ACCTCTGCGGCGAATCAGAAGC (SEQ ID NO: 10) |
| CCR2 | NM_001123041.2 | TCTGATCTGCTTTTTCTTATTACTCTCCCATTGTGGGCTC |
| | | ACTCTGCTGCAAATGAGTGGGTCTTTGGGAATGCAATGT |
| | | GCAAATTATTCACAGGGCTGT (SEQ ID NO: 12) |
| CD4 | NM_000616.3 | AGACATCGTGGTGCTAGCTTTCCAGAAGGCCTCCAGCAT |
| | | AGTCTATAAGAAAGAGGGGGAACAGGTGGAGTTCTCCT |
| | | TCCCACTCGCCTTTACAGTTGAA (SEQ ID NO: 13) |
| CD163 | NM_004244.4 | CATCTGTGATTCGGACTTCTCTCTGGAAGCTGCCAGCGT |
| | | TCTATGCAGGGAATTACAGTGTGGCACAGTTGTCTCTAT |
| | | CCTGGGGGGAGCTCACTTTGGA (SEQ ID NO: 15) |
| CFD | NM_001928.2 | CTGGTTGGTCTTTATTGAGCACCTACTATATGCAGAAGG |
| | | GGAGGCCGAGGTGGGAGGATCATTGGATCTCAGGAGTT |
| | | CGAGATCAGCATGGGCCACGTAG (SEQ ID NO: 16) |
| CSF1 | NM_000757.4 | TTTCTATGAGACACCTCTCCAGTTGCTGGAGAAGGTCAA |
| | | GAATGTCTTTAATGAAACAAAGAATCTCCTTGACAAGG |
| | | ACTGGAATATTTTCAGCAAGAAC (SEQ ID NO: 17) |
| CXCL9 | NM_002416.1 | CACCATCTCCCATGAAGAAAGGGAACGGTGAAGTACTA |
| | | AGCGCTAGAGGAAGCAGCCAAGTCGGTTAGTGGAAGCA |
| | | TGATTGGTGCCCAGTTAGCCTCTG (SEQ ID NO: 18) |
| CXCR4 | NM_003467.2 | ATTGATGTGTGTCTAGGCAGGACCTGTGGCCAAGTTCTT |
| | | AGTTGCTGTATGTCTCGTGGTAGGACTGTAGAAAAGGG |
| | | AACTGAACATTCCAGAGCGTGTA (SEQ ID NO: 19) |
| FOS | NM_005252.2 | ACTCAAGTCCTTACCTCTTCCGGAGATGTAGCAAAACGC |
| | | ATGGAGTGTGTATTGTTCCCAGTGACACTTCAGAGAGCT |
| | | GGTAGTTAGTAGCATGTTGAGC (SEQ ID NO: 20) |
| HSPB2 | NM_001541.3 | AGCTGCTCTCTCCCATGATGGCATCTTAAACCTGGAAGC |
| | | ACCTCGGGGTGGCCGACATTTGGACACAGAGGTCAATG |
| | | AGGTCTACATCTCCCTGCTCCCT (SEQ ID NO: 24) |
| IFIT2 | NM_001547.4 | TGCATCCCATAGAGGTTAGTCCTGCATAGCCAGTAATGT |
| | | GCTAAGTTCATCCAAAAGCTGGCGGACCAAAGTCTAAA |
| | | TAGGGCTCAGTATCCCCCATCGC (SEQ ID NO: 25) |
| IFNG | NM_000619.2 | ATACTATCCAGTTACTGCCGGTTTGAAAATATGCCTGCA |
| | | ATCTGAGCCAGTGCTTTAATGGCATGTCAGACAGAACTT |
| | | GAATGTGTCAGGTGACCCTGAT (SEQ ID NO: 26) |
| IL6R | NM_000565.2 | CTTTCTACATAGTGTCCATGTGCGTCGCCAGTAGTGTCG |
| | | GGAGCAAGTTCAGCAAAACTCAAACCTTTCAGGGTTGT |
| | | GGAATCTTGCAGCCTGATCCGCC (SEQ ID NO: 29) |
| IL8 | NM_000584.2 | ACAGCAGAGCACACAAGCTTCTAGGACAAGAGCCAGGA |
| | | AGAAACCACCGGAAGGAACCATCTCACTGTGTGTAAAC |
| | | ATGACTTCCAAGCTGGCCGTGGCT (SEQ ID NO: 30) |
| IL18RAP | NM_003853.2 | GCTTGATGGACAATGGAGTGGGATTGAGACTGTGGTTT |
| | | AGAGCCTTTGATTTCCTGGACTGGACTGACGGCGAGTG |
| | | AATTCTCTAGACCTTGGGTACTTT (SEQ ID NO: 32) |
| IRF1 | NM_002198.1 | CTGTGCGAGTGTACCGGATGCTTCCACCTCTCACCAAGA |
| | | ACCAGAGAAAAGAAAGAAAGTCGAAGTCCAGCCGAGA |
| | | TGCTAAGAGCAAGGCCAAGAGGAA (SEQ ID NO: 33) |
| IRF7 | NM_001572.3 | CGCAGCGTGAGGGTGTGTCTTCCCTGGATAGCAGCAGC |
| | | CTCAGCCTCTGCCTGTCCAGCGCCAACAGCCTCTATGAC |
| | | GACATCGAGTGCTTCCTTATGGA (SEQ ID NO: 34) |
| JUN | NM_002228.3 | ACACAGCCAGCCAGCCAGGTCGGCAGTATAGTCCGAAC |
| | | TGCAAATCTTATTTTCTTTTCACCTTCTCTCTAACTGCCC |
| | | AGAGCTAGCGCCTGTGGCTCCC (SEQ ID NO: 35) |
| LTB4R | NM_181657.3 | GTAGTGCCCTGGAAAACGAACATGAGCCTGTGCTTCCC |
| | | GCGGTACCCCAGCGAAGGGCACCGGGCCTTCCATCTAA |
| | | TCTTCGAGGCTGTCACGGGCTTCC (SEQ ID NO: 36) |
| MAP2K6 | NM_002758.3 | AGCCAGGAACAGAAACGGCTACTGATGGATTTGGATAT |
| | | TTCCATGAGGACGGTGGACTGTCCATTCACTGTCACCTT |
| | | TTATGGCGCACTGTTTCGGGAGG (SEQ ID NO: 37) |
| MEF2C | NM_002397.3 | ACATTTGCCAAAAGCGGCAGATCTTTACTGAAAGAGAG |
| | | AGCAGCTGTTATGCAACATATAGAAAAATGTATAGATG |
| | | CTTGGACAGACCCGGTAATGGGTG (SEQ ID NO: 39) |
| MRC1 | NM_002438.2 | CTATGGAACCACAGACAATCTGTGCTCCAGAGGTTATG |
| | | AAGCCATGTATACGCTACTAGGCAATGCCAATGGAGCA |
| | | ACCTGTGCATTCCCGTTCAAGTTT (SEQ ID NO: 40) |
| MX1 | NM_002462.2 | GCCTTTAATCAGGACATCACTGCTCTCATGCAAGGAGA |
| | | GGAAACTGTAGGGGAGGAAGACATTCGGCTGTTTACCA |
| | | GACTCCGACACGAGTTCCACAAAT (SEQ ID NO: 41) |
| MYC | NM_002467.3 | TCGGACACCGAGGAGAATGTCAAGAGGCGAACACACA |
| | | ACGTCTTGGAGCGCCAGAGGAGGAACGAGCTAAAACGG |
| | | AGCTTTTTTGCCCTGCGTGACCAGA (SEQ ID NO: 42) |
| NLRP3 | NM_001079821.2 | AGTGGGGTTCAGATAATGCACGTGTTTCGAATCCCACTG |
| | | TGATATGCCAGGAAGACAGCATTGAAGAGGAGTGGATG |
| | | GGTTTACTGGAGTACCTTTCGAG (SEQ ID NO: 44) |
| PIK3C2G | NM_004570.4 | CTGGAATATCTCCCACAGCTAGTTCAGGCTGTCAAGTTT |
| | | GAATGGAACCTTGAGAGTCCTTTAGTGCAACTTCTACTC |
| | | CACCGCTCCTTGCAGAGCATCC (SEQ ID NO: 47) |
| PLCB1 | NM_182734.1 | TCAAAACAAGGAGACAGAGCTACTGGATCTCAGCCTTG |
| | | TCAAAGATGCCAGATGTGGGAGACACGCCAAAGCTCCC |
| | | AAGGACCCCAAATTACGTGAACTT (SEQ ID NO: 48) |
| PTGFR | NM_000959.3 | AACAGCCTTGCCATCGCCATTCTCATGAAGGCATATCAG |
| | | AGATTTAGACAGAAGTCCAAGGCATCGTTTCTGCTTTTG |
| | | GCCAGTGGCCTGGTAATCACTG (SEQ ID NO: 51) |
| STAT1 | NM_007315.2 | TTTGCTGTATGCCATCCTCGAGAGCTGTCTAGGTTAACG |
| | | TTCGCACTCTGTGTATATAACCTCGACAGTCTTGGCACC |
| | | TAACGTGCTGTGCGTAGCTGCT (SEQ ID NO: 52) |
| TGFB1 | NM_000660.3 | TATATGTTCTTCAACACATCAGAGCTCCGAGAAGCGGTA |
| | | CCTGAACCCGTGTTGCTCTCCCGGGCAGAGCTGCGTCTG |
| | | CTGAGGCTCAAGTTAAAAGTGG (SEQ ID NO: 55) |
| TLR4 | NM_138554.2 | ACTCAGAAAAGCCCTGCTGGATGGTAAATCATGGAATC |
| | | CAGAAGGAACAGTGGGTACAGGATGCAATTGGCAGGA |
| | | AGCAACATCTATCTGAAGAGGAAAA (SEQ ID NO: 57) |
| TLR5 | NM_003268.3 | GTTCCTGGCACTACTACAAGATTCATACTCCTGATGCTA |
| | | CTGACAACGTGGCTTCTCCACAGTCACCAAACCAGGGA |
| | | TGCTATACTGGACTTCCCTACTC (SEQ ID NO: 58) |
| TLR8 | NM_016610.2 | TTTAACTGATAGCCTATCTGACTTTACATCTTCCCTTCGG |
| | | ACACTGCTGCTGAGTCATAACAGGATTTCCCACCTACCC |
| | | TCTGGCTTTCTTTCTGAAGTC (SEQ ID NO: 59) |
| TNF | NM_000594.2 | AGCAACAAGACCACCACTTCGAAACCTGGGATTCAGGA |
| | | ATGTGTGGCCTGCACAGTGAAGTGCTGGCAACCACTAA |
| | | GAATTCAAACTGGGGCCTCCAGAA (SEQ ID NO: 60) |
| TREM2 | NM_018965.3 | TTCCCACCCACTTCCATCCTTCTCCTCCTGGCCTGCATCT |
| | | TTCTCATCAAGATTCTAGCAGCCAGCGCCCTCTGGGCTG |
| | | CAGCCTGGCATGGACAGAAGC (SEQ ID NO: 61) |

In another embodiment, the at least one DED marker is selected from the list of the 37 DED markers of Table 5 below, as well as their variants, fragments or equivalents. Table 5 comprises DED markers, i.e. markers whose expression is different between a DED patient and a “normal” patient, identified in the conditions of the Example and presenting a fold-change, positive or negative, superior or equal to 1.5.

TABLE 5

| Name of | GeneBank | |
|---|---|---|
| the | Accession | |
| marker | Number | Target sequence (SEQ ID number) |
| AGER | NM_001136.3 | GAAAGGAGACCAAGTCCAACTACCGAGTCCGTGTCTAC |
| | | CAGATTCCTGGGAAGCCAGAAATTGTAGATTCTGCCTCT |
| | | GAACTCACGGCTGGTGTTCCCAA (SEQ ID NO: 2) |
| AREG | NM_001657.2 | ACAAGACGGAAAGTGAAAATACTTCAGATAAACCCAAA |
| | | AGAAAGAAAAAGGGAGGCAAAAATGGAAAAAATAGAA |
| | | GAAACAGAAAGAAGAAAAATCCATG (SEQ ID NO: 3) |
| C1QA | NM_015991.2 | GGTGACCAGGTCTGGGTTGAAAAAGACCCCAAAAAGGG |
| | | TCACATTTACCAGGGCTCTGAGGCCGACAGCGTCTTCAG |
| | | CGGCTTCCTCATCTTCCCATCTG (SEQ ID NO: 4) |
| C1S | NM_001734.2 | ACTGCACTGATTGGGGAGATTGCAAGTCCCAATTATCCC |
| | | AAACCATATCCAGAGAACTCAAGGTGTGAATACCAGAT |
| | | CCGGTTGGAGAAAGGGTTCCAAG (SEQ ID NO: 5) |
| C2 | NM_000063.3 | ATCTCATGATGAACAACCAAATGCGACTCCTCGGCATG |
| | | GAAACGATGGCCTGGCAGGAAATCCGACATGCCATCAT |
| | | CCTTCTGACAGATGGAAAGTCCAA (SEQ ID NO: 6) |
| C3AR1 | NM_004054.2 | CAGTGTCTTCCTGCTTACTGCCATTAGCCTGGATCGCTG |
| | | TCTTGTGGTATTCAAGCCAATCTGGTGTCAGAATCATCG |
| | | CAATGTAGGGATGGCCTGCTCT (SEQ ID NO: 7) |
| CCL4 | NM_002984.2 | TTCTGCAGCCTCACCTCTGAGAAAACCTCTTTGCCACCA |
| | | ATACCATGAAGCTCTGCGTGACTGTCCTGTCTCTCCTCA |
| | | TGCTAGTAGCTGCCTTCTGCTC (SEQ ID NO: 8) |
| CCR2 | NM_001123041.2 | TCTGATCTGCTTTTTCTTATTACTCTCCCATTGTGGGCTC |
| | | ACTCTGCTGCAAATGAGTGGGTCTTTGGGAATGCAATGT |
| | | GCAAATTATTCACAGGGCTGT (SEQ ID NO: 12) |
| CD4 | NM_000616.3 | AGACATCGTGGTGCTAGCTTTCCAGAAGGCCTCCAGCAT |
| | | AGTCTATAAGAAAGAGGGGGAACAGGTGGAGTTCTCCT |
| | | TCCCACTCGCCTTTACAGTTGAA (SEQ ID NO: 13) |
| CD163 | NM_004244.4 | CATCTGTGATTCGGACTTCTCTCTGGAAGCTGCCAGCGT |
| | | TCTATGCAGGGAATTACAGTGTGGCACAGTTGTCTCTAT |
| | | CCTGGGGGGAGCTCACTTTGGA (SEQ ID NO: 15) |
| CFD | NM_001928.2 | CTGGTTGGTCTTTATTGAGCACCTACTATATGCAGAAGG |
| | | GGAGGCCGAGGTGGGAGGATCATTGGATCTCAGGAGTT |
| | | CGAGATCAGCATGGGCCACGTAG (SEQ ID NO: 16) |
| CSF1 | NM_000757.4 | TTTCTATGAGACACCTCTCCAGTTGCTGGAGAAGGTCAA |
| | | GAATGTCTTTAATGAAACAAAGAATCTCCTTGACAAGG |
| | | ACTGGAATATTTTCAGCAAGAAC (SEQ ID NO: 17) |
| CXCL9 | NM_002416.1 | CACCATCTCCCATGAAGAAAGGGAACGGTGAAGTACTA |
| | | AGCGCTAGAGGAAGCAGCCAAGTCGGTTAGTGGAAGCA |
| | | TGATTGGTGCCCAGTTAGCCTCTG (SEQ ID NO: 18) |
| CXCR4 | NM_003467.2 | ATTGATGTGTGTCTAGGCAGGACCTGTGGCCAAGTTCTT |
| | | AGTTGCTGTATGTCTCGTGGTAGGACTGTAGAAAAGGG |
| | | AACTGAACATTCCAGAGCGTGTA (SEQ ID NO: 19) |
| FOS | NM_005252.2 | ACTCAAGTCCTTACCTCTTCCGGAGATGTAGCAAAACGC |
| | | ATGGAGTGTGTATTGTTCCCAGTGACACTTCAGAGAGCT |
| | | GGTAGTTAGTAGCATGTTGAGC (SEQ ID NO: 20) |
| HSPB2 | NM_001541.3 | AGCTGCTCTCTCCCATGATGGCATCTTAAACCTGGAAGC |
| | | ACCTCGGGGTGGCCGACATTTGGACACAGAGGTCAATG |
| | | AGGTCTACATCTCCCTGCTCCCT (SEQ ID NO: 24) |
| IFIT2 | NM_001547.4 | TGCATCCCATAGAGGTTAGTCCTGCATAGCCAGTAATGT |
| | | GCTAAGTTCATCCAAAAGCTGGCGGACCAAAGTCTAAA |
| | | TAGGGCTCAGTATCCCCCATCGC (SEQ ID NO: 25) |
| IL6R | NM_000565.2 | CTTTCTACATAGTGTCCATGTGCGTCGCCAGTAGTGTCG |
| | | GGAGCAAGTTCAGCAAAACTCAAACCTTTCAGGGTTGT |
| | | GGAATCTTGCAGCCTGATCCGCC (SEQ ID NO: 29) |
| IL18RAP | NM_003853.2 | GCTTGATGGACAATGGAGTGGGATTGAGACTGTGGTTT |
| | | AGAGCCTTTGATTTCCTGGACTGGACTGACGGCGAGTG |
| | | AATTCTCTAGACCTTGGGTACTTT (SEQ ID NO: 32) |
| IRF1 | NM_002198.1 | CTGTGCGAGTGTACCGGATGCTTCCACCTCTCACCAAGA |
| | | ACCAGAGAAAAGAAAGAAAGTCGAAGTCCAGCCGAGA |
| | | TGCTAAGAGCAAGGCCAAGAGGAA (SEQ ID NO: 33) |
| IRF7 | NM_001572.3 | CGCAGCGTGAGGGTGTGTCTTCCCTGGATAGCAGCAGC |
| | | CTCAGCCTCTGCCTGTCCAGCGCCAACAGCCTCTATGAC |
| | | GACATCGAGTGCTTCCTTATGGA (SEQ ID NO: 34) |
| JUN | NM_002228.3 | ACACAGCCAGCCAGCCAGGTCGGCAGTATAGTCCGAAC |
| | | TGCAAATCTTATTTTCTTTTCACCTTCTCTCTAACTGCCC |
| | | AGAGCTAGCGCCTGTGGCTCCC (SEQ ID NO: 35) |
| LTB4R | NM_181657.3 | GTAGTGCCCTGGAAAACGAACATGAGCCTGTGCTTCCC |
| | | GCGGTACCCCAGCGAAGGGCACCGGGCCTTCCATCTAA |
| | | TCTTCGAGGCTGTCACGGGCTTCC (SEQ ID NO: 36) |
| MAP2K6 | NM_002758.3 | AGCCAGGAACAGAAACGGCTACTGATGGATTTGGATAT |
| | | TTCCATGAGGACGGTGGACTGTCCATTCACTGTCACCTT |
| | | TTATGGCGCACTGTTTCGGGAGG (SEQ ID NO: 37) |
| MEF2C | NM_002397.3 | ACATTTGCCAAAAGCGGCAGATCTTTACTGAAAGAGAG |
| | | AGCAGCTGTTATGCAACATATAGAAAAATGTATAGATG |
| | | CTTGGACAGACCCGGTAATGGGTG (SEQ ID NO: 39) |
| MRC1 | NM_002438.2 | CTATGGAACCACAGACAATCTGTGCTCCAGAGGTTATG |
| | | AAGCCATGTATACGCTACTAGGCAATGCCAATGGAGCA |
| | | ACCTGTGCATTCCCGTTCAAGTTT (SEQ ID NO: 40) |
| MX1 | NM_002462.2 | GCCTTTAATCAGGACATCACTGCTCTCATGCAAGGAGA |
| | | GGAAACTGTAGGGGAGGAAGACATTCGGCTGTTTACCA |
| | | GACTCCGACACGAGTTCCACAAAT (SEQ ID NO: 41) |
| MYC | NM_002467.3 | TCGGACACCGAGGAGAATGTCAAGAGGCGAACACACA |
| | | ACGTCTTGGAGCGCCAGAGGAGGAACGAGCTAAAACGG |
| | | AGCTTTTTTGCCCTGCGTGACCAGA (SEQ ID NO: 42) |
| NLRP3 | NM_001079821.2 | AGTGGGGTTCAGATAATGCACGTGTTTCGAATCCCACTG |
| | | TGATATGCCAGGAAGACAGCATTGAAGAGGAGTGGATG |
| | | GGTTTACTGGAGTACCTTTCGAG (SEQ ID NO: 44) |
| PIK3C2G | NM_004570.4 | CTGGAATATCTCCCACAGCTAGTTCAGGCTGTCAAGTTT |
| | | GAATGGAACCTTGAGAGTCCTTTAGTGCAACTTCTACTC |
| | | CACCGCTCCTTGCAGAGCATCC (SEQ ID NO: 47) |
| PLCB1 | NM_182734.1 | TCAAAACAAGGAGACAGAGCTACTGGATCTCAGCCTTG |
| | | TCAAAGATGCCAGATGTGGGAGACACGCCAAAGCTCCC |
| | | AAGGACCCCAAATTACGTGAACTT (SEQ ID NO: 48) |
| PTGFR | NM_000959.3 | AACAGCCTTGCCATCGCCATTCTCATGAAGGCATATCAG |
| | | AGATTTAGACAGAAGTCCAAGGCATCGTTTCTGCTTTTG |
| | | GCCAGTGGCCTGGTAATCACTG (SEQ ID NO: 51) |
| STAT1 | NM_007315.2 | TTTGCTGTATGCCATCCTCGAGAGCTGTCTAGGTTAACG |
| | | TTCGCACTCTGTGTATATAACCTCGACAGTCTTGGCACC |
| | | TAACGTGCTGTGCGTAGCTGCT (SEQ ID NO: 52) |
| TLR5 | NM_003268.3 | GTTCCTGGCACTACTACAAGATTCATACTCCTGATGCTA |
| | | CTGACAACGTGGCTTCTCCACAGTCACCAAACCAGGGA |
| | | TGCTATACTGGACTTCCCTACTC (SEQ ID NO: 58) |
| TLR8 | NM_016610.2 | TTTAACTGATAGCCTATCTGACTTTACATCTTCCCTTCGG |
| | | ACACTGCTGCTGAGTCATAACAGGATTTCCCACCTACCC |
| | | TCTGGCTTTCTTTCTGAAGTC (SEQ ID NO: 59) |
| TREM2 | NM_018965.3 | TTCCCACCCACTTCCATCCTTCTCCTCCTGGCCTGCATCT |
| | | TTCTCATCAAGATTCTAGCAGCCAGCGCCCTCTGGGCTG |
| | | CAGCCTGGCATGGACAGAAGC (SEQ ID NO: 61) |

In another embodiment, the at least one DED marker is selected from the list of the 21 DED markers of Table 6 below, as well as their variants, fragments or equivalents. Table 6 comprises DED markers, i.e. markers whose expression is different between a DED patient and a “normal” patient, identified in the conditions of the Example and presenting a fold-change, positive or negative, superior or equal to 2.

TABLE 6

| | GeneBank | |
|---|---|---|
| Name of the | Accession | |
| marker | Number | Target sequence (SEQ ID number) |
| AGER | NM_001136.3 | GAAAGGAGACCAAGTCCAACTACCGAGTCCGTGTCTAC |
| | | CAGATTCCTGGGAAGCCAGAAATTGTAGATTCTGCCTCT |
| | | GAACTCACGGCTGGTGTTCCCAA (SEQ ID NO: 2) |
| AREG | NM_001657.2 | ACAAGACGGAAAGTGAAAATACTTCAGATAAACCCAAA |
| | | AGAAAGAAAAAGGGAGGCAAAAATGGAAAAAATAGAA |
| | | GAAACAGAAAGAAGAAAAATCCATG (SEQ ID NO: 3) |
| C1QA | NMO15991.2 | GGTGACCAGGTCTGGGTTGAAAAAGACCCCAAAAAGGG |
| | | TCACATTTACCAGGGCTCTGAGGCCGACAGCGTCTTCAG |
| | | CGGCTTCCTCATCTTCCCATCTG (SEQ ID NO: 4) |
| C1S | NM_001734.2 | ACTGCACTGATTGGGGAGATTGCAAGTCCCAATTATCCC |
| | | AAACCATATCCAGAGAACTCAAGGTGTGAATACCAGAT |
| | | CCGGTTGGAGAAAGGGTTCCAAG (SEQ ID NO: 5) |
| CCL20 | NM_004591.1 | ATCTGTTCTTTGAGCTAAAAACCATGTGCTGTACCAAGA |
| | | GTTTGCTCCTGGCTGCTTTGATGTCAGTGCTGCTACTCC |
| | | ACCTCTGCGGCGAATCAGAAGC (SEQ ID NO: 10) |
| CCR2 | NM_001123041.2 | TCTGATCTGCTTTTTCTTATTACTCTCCCATTGTGGGCTC |
| | | ACTCTGCTGCAAATGAGTGGGTCTTTGGGAATGCAATGT |
| | | GCAAATTATTCACAGGGCTGT (SEQ ID NO: 12) |
| CD4 | NM_000616.3 | AGACATCGTGGTGCTAGCTTTCCAGAAGGCCTCCAGCAT |
| | | AGTCTATAAGAAAGAGGGGGAACAGGTGGAGTTCTCCT |
| | | TCCCACTCGCCTTTACAGTTGAA (SEQ ID NO: 13) |
| CFD | NM_001928.2 | CTGGTTGGTCTTTATTGAGCACCTACTATATGCAGAAGG |
| | | GGAGGCCGAGGTGGGAGGATCATTGGATCTCAGGAGTT |
| | | CGAGATCAGCATGGGCCACGTAG (SEQ ID NO: 16) |
| CSF1 | NM_000757.4 | TTTCTATGAGACACCTCTCCAGTTGCTGGAGAAGGTCAA |
| | | GAATGTCTTTAATGAAACAAAGAATCTCCTTGACAAGG |
| | | ACTGGAATATTTTCAGCAAGAAC (SEQ ID NO: 17) |
| FOS | NM_005252.2 | ACTCAAGTCCTTACCTCTTCCGGAGATGTAGCAAAACGC |
| | | ATGGAGTGTGTATTGTTCCCAGTGACACTTCAGAGAGCT |
| | | GGTAGTTAGTAGCATGTTGAGC (SEQ ID NO: 20) |
| HSPB2 | NM_001541.3 | AGCTGCTCTCTCCCATGATGGCATCTTAAACCTGGAAGC |
| | | ACCTCGGGGTGGCCGACATTTGGACACAGAGGTCAATG |
| | | AGGTCTACATCTCCCTGCTCCCT (SEQ ID NO: 24) |
| IL6R | NM_000565.2 | CTTTCTACATAGTGTCCATGTGCGTCGCCAGTAGTGTCG |
| | | GGAGCAAGTTCAGCAAAACTCAAACCTTTCAGGGTTGT |
| | | GGAATCTTGCAGCCTGATCCGCC (SEQ ID NO: 29) |
| LTB4R | NM_181657.3 | GTAGTGCCCTGGAAAACGAACATGAGCCTGTGCTTCCC |
| | | GCGGTACCCCAGCGAAGGGCACCGGGCCTTCCATCTAA |
| | | TCTTCGAGGCTGTCACGGGCTTCC (SEQ ID NO: 36) |
| MRC1 | NM_002438.2 | CTATGGAACCACAGACAATCTGTGCTCCAGAGGTTATG |
| | | AAGCCATGTATACGCTACTAGGCAATGCCAATGGAGCA |
| | | ACCTGTGCATTCCCGTTCAAGTTT (SEQ ID NO: 40) |
| MYC | NM_002467.3 | TCGGACACCGAGGAGAATGTCAAGAGGCGAACACACA |
| | | ACGTCTTGGAGCGCCAGAGGAGGAACGAGCTAAAACGG |
| | | AGCTTTTTTGCCCTGCGTGACCAGA (SEQ ID NO: 42) |
| NLRP3 | NM_001079821.2 | AGTGGGGTTCAGATAATGCACGTGTTTCGAATCCCACTG |
| | | TGATATGCCAGGAAGACAGCATTGAAGAGGAGTGGATG |
| | | GGTTTACTGGAGTACCTTTCGAG (SEQ ID NO: 44) |
| PIK3C2G | NM_004570.4 | CTGGAATATCTCCCACAGCTAGTTCAGGCTGTCAAGTTT |
| | | GAATGGAACCTTGAGAGTCCTTTAGTGCAACTTCTACTC |
| | | CACCGCTCCTTGCAGAGCATCC (SEQ ID NO: 47) |
| PLCB1 | NM_182734.1 | TCAAAACAAGGAGACAGAGCTACTGGATCTCAGCCTTG |
| | | TCAAAGATGCCAGATGTGGGAGACACGCCAAAGCTCCC |
| | | AAGGACCCCAAATTACGTGAACTT (SEQ ID NO: 48) |
| PTGFR | NM_000959.3 | AACAGCCTTGCCATCGCCATTCTCATGAAGGCATATCAG |
| | | AGATTTAGACAGAAGTCCAAGGCATCGTTTCTGCTTTTG |
| | | GCCAGTGGCCTGGTAATCACTG (SEQ ID NO: 51) |
| TLR4 | NM_138554.2 | ACTCAGAAAAGCCCTGCTGGATGGTAAATCATGGAATC |
| | | CAGAAGGAACAGTGGGTACAGGATGCAATTGGCAGGA |
| | | AGCAACATCTATCTGAAGAGGAAAA (SEQ ID NO: 57) |
| TNF | NM_000594.2 | AGCAACAAGACCACCACTTCGAAACCTGGGATTCAGGA |
| | | ATGTGTGGCCTGCACAGTGAAGTGCTGGCAACCACTAA |
| | | GAATTCAAACTGGGGCCTCCAGAA (SEQ ID NO: 60) |

In another embodiment, the at least one DED marker is selected from the list of the 19 DED markers of Table 7 below, as well as their variants, fragments or equivalents. Table 7 comprises DED markers, i.e. markers whose expression is different between a DED patient and a “normal” patient, identified in the conditions of the Example and presenting a fold-change, positive or negative, superior or equal to 2.

TABLE 7

| | GeneBank | |
|---|---|---|
| Name of the | Accession | |
| marker | Number | Target sequence (SEQ ID number) |
| AGER | NM_001136.3 | GAAAGGAGACCAAGTCCAACTACCGAGTCCGTGTCTAC |
| | | CAGATTCCTGGGAAGCCAGAAATTGTAGATTCTGCCTCT |
| | | GAACTCACGGCTGGTGTTCCCAA (SEQ ID NO: 2) |
| AREG | NM_001657.2 | ACAAGACGGAAAGTGAAAATACTTCAGATAAACCCAAA |
| | | AGAAAGAAAAAGGGAGGCAAAAATGGAAAAAATAGAA |
| | | GAAACAGAAAGAAGAAAAATCCATG (SEQ ID NO: 3) |
| C1QA | NM_015991.2 | GGTGACCAGGTCTGGGTTGAAAAAGACCCCAAAAAGGG |
| | | TCACATTTACCAGGGCTCTGAGGCCGACAGCGTCTTCAG |
| | | CGGCTTCCTCATCTTCCCATCTG (SEQ ID NO: 4) |
| C1S | NM_001734.2 | ACTGCACTGATTGGGGAGATTGCAAGTCCCAATTATCCC |
| | | AAACCATATCCAGAGAACTCAAGGTGTGAATACCAGAT |
| | | CCGGTTGGAGAAAGGGTTCCAAG (SEQ ID NO: 5) |
| CCR2 | NM_001123041.2 | TCTGATCTGCTTTTTCTTATTACTCTCCCATTGTGGGCTC |
| | | ACTCTGCTGCAAATGAGTGGGTCTTTGGGAATGCAATGT |
| | | GCAAATTATTCACAGGGCTGT (SEQ ID NO: 12) |
| CD4 | NM_000616.3 | AGACATCGTGGTGCTAGCTTTCCAGAAGGCCTCCAGCAT |
| | | AGTCTATAAGAAAGAGGGGGAACAGGTGGAGTTCTCCT |
| | | TCCCACTCGCCTTTACAGTTGAA (SEQ ID NO: 13) |
| CFD | NM_001928.2 | CTGGTTGGTCTTTATTGAGCACCTACTATATGCAGAAGG |
| | | GGAGGCCGAGGTGGGAGGATCATTGGATCTCAGGAGTT |
| | | CGAGATCAGCATGGGCCACGTAG (SEQ ID NO: 16) |
| CSF1 | NM_000757.4 | TTTCTATGAGACACCTCTCCAGTTGCTGGAGAAGGTCAA |
| | | GAATGTCTTTAATGAAACAAAGAATCTCCTTGACAAGG |
| | | ACTGGAATATTTTCAGCAAGAAC (SEQ ID NO: 17) |
| FOS | NM_005252.2 | ACTCAAGTCCTTACCTCTTCCGGAGATGTAGCAAAACGC |
| | | ATGGAGTGTGTATTGTTCCCAGTGACACTTCAGAGAGCT |
| | | GGTAGTTAGTAGCATGTTGAGC (SEQ ID NO: 20) |
| HSPB2 | NM_001541.3 | AGCTGCTCTCTCCCATGATGGCATCTTAAACCTGGAAGC |
| | | ACCTCGGGGTGGCCGACATTTGGACACAGAGGTCAATG |
| | | AGGTCTACATCTCCCTGCTCCCT (SEQ ID NO: 24) |
| IL6R | NM_000565.2 | CTTTCTACATAGTGTCCATGTGCGTCGCCAGTAGTGTCG |
| | | GGAGCAAGTTCAGCAAAACTCAAACCTTTCAGGGTTGT |
| | | GGAATCTTGCAGCCTGATCCGCC (SEQ ID NO: 29) |
| LTB4R | NM_181657.3 | GTAGTGCCCTGGAAAACGAACATGAGCCTGTGCTTCCC |
| | | GCGGTACCCCAGCGAAGGGCACCGGGCCTTCCATCTAA |
| | | TCTTCGAGGCTGTCACGGGCTTCC (SEQ ID NO: 36) |
| MRC1 | NM_002438.2 | CTATGGAACCACAGACAATCTGTGCTCCAGAGGTTATG |
| | | AAGCCATGTATACGCTACTAGGCAATGCCAATGGAGCA |
| | | ACCTGTGCATTCCCGTTCAAGTTT (SEQ ID NO: 40) |
| MYC | NM_002467.3 | TCGGACACCGAGGAGAATGTCAAGAGGCGAACACACA |
| | | ACGTCTTGGAGCGCCAGAGGAGGAACGAGCTAAAACGG |
| | | AGCTTTTTTGCCCTGCGTGACCAGA (SEQ ID NO: 42) |
| NLRP3 | NM_001079821.2 | AGTGGGGTTCAGATAATGCACGTGTTTCGAATCCCACTG |
| | | TGATATGCCAGGAAGACAGCATTGAAGAGGAGTGGATG |
| | | GGTTTACTGGAGTACCTTTCGAG (SEQ ID NO: 44) |
| PIK3C2G | NM_004570.4 | CTGGAATATCTCCCACAGCTAGTTCAGGCTGTCAAGTTT |
| | | GAATGGAACCTTGAGAGTCCTTTAGTGCAACTTCTACTC |
| | | CACCGCTCCTTGCAGAGCATCC (SEQ ID NO: 47) |
| PLCB1 | NM_182734.1 | TCAAAACAAGGAGACAGAGCTACTGGATCTCAGCCTTG |
| | | TCAAAGATGCCAGATGTGGGAGACACGCCAAAGCTCCC |
| | | AAGGACCCCAAATTACGTGAACTT (SEQ ID NO: 48) |
| PTGFR | NM_000959.3 | AACAGCCTTGCCATCGCCATTCTCATGAAGGCATATCAG |
| | | AGATTTAGACAGAAGTCCAAGGCATCGTTTCTGCTTTTG |
| | | GCCAGTGGCCTGGTAATCACTG (SEQ ID NO: 51) |
| TLR4 | NM_138554.2 | ACTCAGAAAAGCCCTGCTGGATGGTAAATCATGGAATC |
| | | CAGAAGGAACAGTGGGTACAGGATGCAATTGGCAGGA |
| | | AGCAACATCTATCTGAAGAGGAAAA (SEQ ID NO: 57) |

In another embodiment, the at least one DED marker is selected from the list of the 6 DED markers of Table 8 below, as well as their variants, fragments or equivalents. Table 8 comprises DED markers, i.e. markers whose expression is different between a DED patient and a “normal” patient, identified in the conditions of the Example and presenting a fold-change, positive or negative, superior or equal to 5.

TABLE 8

| | GeneBank | |
|---|---|---|
| Name of the | Accession | |
| marker | Number | Target sequence (SEQ ID number) |
| AREG | NM_001657.2 | ACAAGACGGAAAGTGAAAATACTTCAGATAAACCCAAA |
| | | AGAAAGAAAAAGGGAGGCAAAAATGGAAAAAATAGAA |
| | | GAAACAGAAAGAAGAAAAATCCATG (SEQ ID NO: 3) |
| CCL20 | NM_004591.1 | ATCTGTTCTTTGAGCTAAAAACCATGTGCTGTACCAAGA |
| | | GTTTGCTCCTGGCTGCTTTGATGTCAGTGCTGCTACTCC |
| | | ACCTCTGCGGCGAATCAGAAGC (SEQ ID NO: 10) |
| HSPB2 | NM_001541.3 | AGCTGCTCTCTCCCATGATGGCATCTTAAACCTGGAAGC |
| | | ACCTCGGGGTGGCCGACATTTGGACACAGAGGTCAATG |
| | | AGGTCTACATCTCCCTGCTCCCT (SEQ ID NO: 24) |
| PIK3C2G | NM_004570.4 | CTGGAATATCTCCCACAGCTAGTTCAGGCTGTCAAGTTT |
| | | GAATGGAACCTTGAGAGTCCTTTAGTGCAACTTCTACTC |
| | | CACCGCTCCTTGCAGAGCATCC (SEQ ID NO: 47) |
| PLCB1 | NM_182734.1 | TCAAAACAAGGAGACAGAGCTACTGGATCTCAGCCTTG |
| | | TCAAAGATGCCAGATGTGGGAGACACGCCAAAGCTCCC |
| | | AAGGACCCCAAATTACGTGAACTT (SEQ ID NO: 48) |
| PTGFR | NM_000959.3 | AACAGCCTTGCCATCGCCATTCTCATGAAGGCATATCAG |
| | | AGATTTAGACAGAAGTCCAAGGCATCGTTTCTGCTTTTG |
| | | GCCAGTGGCCTGGTAATCACTG (SEQ ID NO: 51) |

In another embodiment, the at least one DED marker is selected from the list of the 9 DED markers of Table 9 below, as well as their variants, fragments or equivalents. Table 9 comprises DED markers, i.e. markers whose expression is different between a DED patient and a “normal” patient, identified in the conditions of the Example and presenting a fold-change, positive or negative, superior or equal to 5.

TABLE 9

| | GeneBank | |
|---|---|---|
| Name of the | Accession | |
| marker | Number | Target sequence (SEQ ID number) |
| CCR1 | NM_001295.2 | CATCATTTGGGCCCTGGCCATCTTGGCTTCCATGCCAG |
| | | GCTTATACTTTTCCAAGACCCAATGGGAATTCACTCAC |
| | | CACACCTGCAGCCTTCACTTTCCT (SEQ ID NO: 123) |
| CXCL2 | NM_002089.3 | ATCACATGTCAGCCACTGTGATAGAGGCTGAGGAATC |
| | | CAAGAAAATGGCCAGTGAGATCAATGTGACGGCAGGG |
| | | AAATGTATGTGTGTCTATTTTGTAAC (SEQ ID NO: 124) |
| KEAP1 | NM_012289.3 | CTCATTGAATTCGCCTACACGGCCTCCATCTCCATGGG |
| | | CGAGAAGTGTGTCCTCCACGTCATGAACGGTGCTGTC |
| | | ATGTACCAGATCGACAGCGTTGTCC (SEQ ID NO: 125) |
| GNAQ | NM_002072.2 | AAGGAGTACAATCTGGTCTAATTGTGCCTCCTAGACAC |
| | | CCGCCCTGCCCTTCCCTGGTGGGCTATTGAAGATACAC |
| | | AAGAGGGACTGTATTTCTGTGGAA (SEQ ID NO: 118) |
| PLA2G4A | NM_024420.2 | AACTGGATGCAGCTGTAGCAGATCCTGATGAATTTGA |
| | | GCGAATATATGAGCCTCTGGATGTCAAAAGTAAAAAG |
| | | ATTCATGTAGTGGACAGTGGGCTCAC (SEQ ID NO: 119) |
| CFL1 | NM_005507.2 | TCCGGAAACATGGCCTCCGGTGTGGCTGTCTCTGATGG |
| | | TGTCATCAAGGTGTTCAACGACATGAAGGTGCGTAAG |
| | | TCTTCAACGCCAGAGGAGGTGAAGA (SEQ ID NO: 120) |
| CDC42 | NM_001039802.1 | ACCGACTGTTTTTGACAACTATGCAGTCACAGTTATGA |
| | | TTGGTGGAGAACCATATACTCTTGGACTTTTTGATACT |
| | | GCAGGGCAAGAGGATTATGACAGA (SEQ ID NO: 121) |
| CCL5 | NM_002985.2 | AGTGTGTGCCAACCCAGAGAAGAAATGGGTTCGGGAG |
| | | TACATCAACTCTTTGGAGATGAGCTAGGATGGAGAGT |
| | | CCTTGAACCTGAACTTACACAAATTT (SEQ ID NO: 126) |
| ClQB | NM_000491.3 | AACTCACTACTGGGCATGGAGGGTGCCAACAGCATCT |
| | | TTTCCGGGTTCCTGCTCTTTCCAGATATGGAGGCCTGA |
| | | CCTGTGGGCTGCTTCACATCCACCC (SEQ ID NO: 127) |

Moreover, it is also important to consider the level of expression of the genes and their coefficient of variation. Indeed, genes with higher mean expression levels are more easily detected than those with lower levels of expression. Thus, among the selected genes, the ones with higher expression levels (for instance, those with mean expression levels of more than 100 transcripts per assay) are preferred to increase the sensitivity of the assay.

In one embodiment, the at least one DED marker has at least 100 transcripts per assay, preferably at least 150, more preferably at least 200, even more preferably at least 300.

The coefficient of variation (the ratio of the standard deviation to the mean) is a measure of the dispersion within a group. The CV (%) is an indication of the homogeneity within a group (of patients for example). Therefore, genes with lower CVs are preferred.

In one embodiment, the at least one DED marker has a coefficient of variation of at most 100, preferably at most 90, more preferably at most 80, more preferably at most 70.

In one embodiment, the at least one DED marker has at least 100 transcripts per assay, preferably at least 150, more preferably at least 200, even more preferably at least 300; or has a coefficient of variation of at most 100, preferably at most 90, more preferably at most 80, more preferably at most 70. In one embodiment, the at least one DED marker has at least 100 transcripts per assay, preferably at least 150, more preferably at least 200, even more preferably at least 300; and has a coefficient of variation of at most 100, preferably at most 90, more preferably at most 80, more preferably at most 70.

Accordingly, in one embodiment, the at least one DED marker is selected from the list of the 6 DED markers of Table 10 below, as well as their variants, fragments or equivalents. Table 10 comprises DED markers, i.e. markers whose expression is different between a DED patient and a “normal” patient, identified in the conditions of the Example and presenting a fold-change, positive or negative, superior or equal to 5.

TABLE 10

| | GeneBank | |
|---|---|---|
| Name of the | Accession | |
| marker | Number | Target sequence (SEQ ID number) |
| CXCL2 | NM_002089.3 | ATCACATGTCAGCCACTGTGATAGAGGCTGAGGAATC |
| | | CAAGAAAATGGCCAGTGAGATCAATGTGACGGCAGGG |
| | | AAATGTATGTGTGTCTATTTTGTAAC (SEQ ID NO: 124) |
| KEAP1 | NM_012289.3 | CTCATTGAATTCGCCTACACGGCCTCCATCTCCATGGG |
| | | CGAGAAGTGTGTCCTCCACGTCATGAACGGTGCTGTC |
| | | ATGTACCAGATCGACAGCGTTGTCC (SEQ ID NO: 125) |
| GNAQ | NM_002072.2 | AAGGAGTACAATCTGGTCTAATTGTGCCTCCTAGACAC |
| | | CCGCCCTGCCCTTCCCTGGTGGGCTATTGAAGATACAC |
| | | AAGAGGGACTGTATTTCTGTGGAA (SEQ ID NO: 118) |
| PLA2G4A | NM_024420.2 | AACTGGATGCAGCTGTAGCAGATCCTGATGAATTTGA |
| | | GCGAATATATGAGCCTCTGGATGTCAAAAGTAAAAAG |
| | | ATTCATGTAGTGGACAGTGGGCTCAC (SEQ ID NO: 119) |
| CFL1 | NM_005507.2 | TCCGGAAACATGGCCTCCGGTGTGGCTGTCTCTGATGG |
| | | TGTCATCAAGGTGTTCAACGACATGAAGGTGCGTAAG |
| | | TCTTCAACGCCAGAGGAGGTGAAGA (SEQ ID NO: 120) |
| CDC42 | NM_001039802.1 | ACCGACTGTTTTTGACAACTATGCAGTCACAGTTATGA |
| | | TTGGTGGAGAACCATATACTCTTGGACTTTTTGATACT |
| | | GCAGGGCAAGAGGATTATGACAGA (SEQ ID NO: 121) |

In one embodiment of the invention, the signature of the invention comprises 1, 2, 3, 4, 5, 6, 7, 8, 9 or 10 DED markers.

In one embodiment of the invention, the signature of the invention comprises at least 1 DED marker selected from the list of Table 1, preferably from the list of Table 2, more preferably from the list of Table 3, even more preferably from the list of Table 4, 5, 6, 7, 8, 9 or 10. In one embodiment, the signature of the invention comprises at least one DED marker selected from the list of Table 2 or Table 9, preferably at least one DED marker selected from the list of Table 2 or Table 10. In one embodiment, the signature of the invention comprises at least one DED marker selected from the list of Table 2 and at least one marker selected from the list of Table 9, preferably of Table 10.

In one embodiment of the invention, the signature of the invention comprises at least 2 DED markers selected from the list of Table 1, preferably from the list of Table 2, more preferably from the list of Table 3, even more preferably from the list of Table 4, 5, 6, 7, 8, 9 or 10. In one embodiment, the signature of the invention comprises at least 2 DED markers selected from the list of Table 2 or Table 9, preferably at least 2 DED markers selected from the list of Table 2 or Table 10. In one embodiment, the signature of the invention comprises at least 2 DED markers selected from the list of Table 2 and at least one marker selected from the list of Table 9, preferably of Table 10.

In one embodiment of the invention, the signature of the invention comprises 2 or 3 DED markers selected from the list of Table 1, preferably from the list of Table 2, more preferably from the list of Table 3, even more preferably from the list of Table 4, 5, 6, 7 or 8. In one embodiment, the signature of the invention comprises 2 or 3 DED markers selected from the list of Table 2 or Table 9, preferably 2 or 3 DED markers selected from the list of Table 2 or Table 10.

In one embodiment of the invention, the signature of the invention comprises at least 3 DED markers selected from the list of Table 1. In another embodiment of the invention, the signature of the invention comprises at least 3 DED markers selected from the list of Table 2 or Table 9, preferably at least 3 DED markers selected from the list of Table 2 or Table 10.

In one embodiment of the invention, the signature of the invention comprises one, two or three of AREG, CCL20 and PTGFR. In one embodiment of the invention, the signature of the invention comprises at least the three markers AREG, CCL20 and PTGFR.

In one embodiment, the signature of the invention comprises 1, 2 or 3 DED markers selected from the list of Table 8, preferably AREG, CCL20 and/or PTGFR, and 1, 2 or 3 other DED markers selected from the list of Table 1, preferably from the list of Table 2, more preferably from the list of Table 3, 4, 5, 6, 7, 9 or 10.

In one embodiment of the invention, the signature of the invention comprises at least one, two or three DED markers of the group comprising or consisting of CXCL2, KEAP1, STAT1, GNGT1, HMGB2, GNAQ, PLA2G4A, CFL1, CDC42, SHC1, CD4, TGFBR1 and CXCR4. In another embodiment of the invention, the signature of the invention comprises at least one, two or three DED markers of the group comprising or consisting of KEAP1, STAT1, GNAQ, PLA2G4A, CFL1, CDC42, SHC1, CD4 and TGFBR1. In another embodiment of the invention, the signature of the invention comprises at least one, two or three DED markers of the group comprising or consisting of KEAP1, STAT1, GNAQ, PLA2G4A, CFL1, CDC42 and SHC. In a particular embodiment of the invention, the signature of the invention comprises at least one, two or three of the DED markers CXCL2, GNAQ and PLA2G4A. In another particular embodiment of the invention, the signature of the invention comprises at least one, two or three of the DED markers STAT1, GNAQ and PLA2G4A. In another particular embodiment of the invention, the signature of the invention comprises at least one, two or three of the DED markers STAT1, GNAQ and PLA2G4A.

In one embodiment, the signature of the invention comprises the DED markers CXCL2 and KEAP1. In another embodiment of the invention, the signature of the invention comprises the DED markers CXCL2 and STAT1. In another embodiment of the invention, the signature of the invention comprises the DED markers CXCL2 and GNGT1. In another embodiment of the invention, the signature of the invention comprises the DED markers CXCL2 and HMGB2. In another embodiment of the invention, the signature of the invention comprises the DED markers CXCL2 and GNAQ. In another embodiment of the invention, the signature of the invention comprises the DED markers CXCL2 and PLA2G4A. In another embodiment of the invention, the signature of the invention comprises the DED markers CXCL2 and CFL1. In another embodiment of the invention, the signature of the invention comprises the DED markers CXCL2 and CDC42. In another embodiment of the invention, the signature of the invention comprises the DED markers CXCL2 and SHC1. In another embodiment of the invention, the signature of the invention comprises the DED markers CXCL2 and CD4. In another embodiment of the invention, the signature of the invention comprises the DED markers CXCL2 and TGFBR1. In another embodiment of the invention, the signature of the invention comprises the DED markers CXCL2 and CXCR4.

In one embodiment, the signature of the invention comprises the DED markers KEAP1 and STAT1. In another embodiment, the signature of the invention comprises the DED markers KEAP1 and GNGT1. In another embodiment, the signature of the invention comprises the DED markers KEAP1 and HMGB2. In another embodiment, the signature of the invention comprises the DED markers KEAP1 and GNAQ. In another embodiment, the signature of the invention comprises the DED markers KEAP1 and PLA2G4A. In another embodiment, the signature of the invention comprises the DED markers KEAP1 and CFL1. In another embodiment, the signature of the invention comprises the DED markers KEAP1 and CDC42. In another embodiment, the signature of the invention comprises the DED markers KEAP1 and SHC1. In another embodiment, the signature of the invention comprises the DED markers KEAP1 and CD4. In another embodiment, the signature of the invention comprises the DED markers KEAP1 and TGFBR1. In another embodiment, the signature of the invention comprises the DED markers KEAP1 and CXCR4.

In one embodiment, the signature of the invention comprises the DED markers STAT1 and GNGT1. In another embodiment, the signature of the invention comprises the DED markers STAT1 and HMGB2. In another embodiment, the signature of the invention comprises the DED markers STAT1 and GNAQ. In another embodiment, the signature of the invention comprises the DED markers STAT1 and PLA2G4A. In another embodiment, the signature of the invention comprises the DED markers STAT1 and CFL1. In another embodiment, the signature of the invention comprises the DED markers STAT1 and CDC42. In another embodiment, the signature of the invention comprises the DED markers STAT1 and SHC1. In another embodiment, the signature of the invention comprises the DED markers STAT1 and CD4. In another embodiment, the signature of the invention comprises the DED markers STAT1 and TGFBR1. In another embodiment, the signature of the invention comprises the DED markers STAT1 and CXCR4.

In one embodiment, the signature of the invention comprises the DED markers GNGT1 and HMGB2. In another embodiment, the signature of the invention comprises the DED markers GNGT1 and PLA2G4A. In another embodiment, the signature of the invention comprises the DED markers GNGT1 and CFL1. In another embodiment, the signature of the invention comprises the DED markers GNGT1 and CDC42. In another embodiment, the signature of the invention comprises the DED markers GNGT1 and SHC1. In another embodiment, the signature of the invention comprises the DED markers GNGT1 and CD4. In another embodiment, the signature of the invention comprises the DED markers GNGT1 and TGFBR1. In another embodiment, the signature of the invention comprises the DED markers GNGT1 and CXCR4.

In one embodiment, the signature of the invention comprises the DED markers HMGB2 and PLA2G4A. In another embodiment, the signature of the invention comprises the DED markers HMGB2 and CFL1. In another embodiment, the signature of the invention comprises the DED markers HMGB2 and CDC42. In another embodiment, the signature of the invention comprises the DED markers HMGB2 and SHC1. In another embodiment, the signature of the invention comprises the DED markers HMGB2 and CD4. In another embodiment, the signature of the invention comprises the DED markers HMGB2 and TGFBR1. In another embodiment, the signature of the invention comprises the DED markers HMGB2 and CXCR4.

In one embodiment, the signature of the invention comprises the DED markers PLA2G4A and CFL1. In another embodiment, the signature of the invention comprises the DED markers PLA2G4A and CDC42. In another embodiment, the signature of the invention comprises the DED markers PLA2G4A and SHC1. In another embodiment, the signature of the invention comprises the DED markers PLA2G4A and CD4. In another embodiment, the signature of the invention comprises the DED markers PLA2G4A and TGFBR1. In another embodiment, the signature of the invention comprises the DED markers PLA2G4A and CXCR4.

In one embodiment, the signature of the invention comprises the DED markers CFL1 and CDC42. In another embodiment, the signature of the invention comprises the DED markers CFL1 and SHC1. In another embodiment, the signature of the invention comprises the DED markers CFL1 and CD4. In another embodiment, the signature of the invention comprises the DED markers CFL1 and TGFBR1. In another embodiment, the signature of the invention comprises the DED markers CFL1 and CXCR4.

In one embodiment, the signature of the invention comprises the DED markers CDC42 and SHC1. In another embodiment, the signature of the invention comprises the DED markers CDC42 and CD4. In another embodiment, the signature of the invention comprises the DED markers CDC42 and TGFBR1. In another embodiment, the signature of the invention comprises the DED markers CDC42 and CXCR4.

In one embodiment, the signature of the invention comprises the DED markers SHC1 and CD4. In another embodiment, the signature of the invention comprises the DED markers SHC1 and TGFBR1. In another embodiment, the signature of the invention comprises the DED markers SHC1 and CXCR4.

In one embodiment, the signature of the invention comprises the DED markers CD4 and TGFBR1. In another embodiment, the signature of the invention comprises the DED markers CD4 and CXCR4.

In one embodiment, the signature of the invention comprises the DED markers TGFBR1 and CXCR4.

In one embodiment, the signature of the invention comprises the DED markers CXCL2, KEAP1 and STAT1. In another embodiment, the signature of the invention comprises the DED markers CXCL2, KEAP1 and GNGT1. In another embodiment, the signature of the invention comprises the DED markers CXCL2, KEAP1 and HMGB2. In another embodiment, the signature of the invention comprises the DED markers CXCL2, KEAP1 and GNAQ. In another embodiment, the signature of the invention comprises the DED markers CXCL2, KEAP1 and PLA2G4A. In another embodiment, the signature of the invention comprises the DED markers CXCL2, KEAP1 and CFL1. In another embodiment, the signature of the invention comprises the DED markers CXCL2, KEAP1 and SHC1. In another embodiment, the signature of the invention comprises the DED markers CXCL2, KEAP1 and CD4. In another embodiment, the signature of the invention comprises the DED markers CXCL2, KEAP1 and TGFBR1. In another embodiment, the signature of the invention comprises the DED markers CXCL2, KEAP1 and CXCR4.

In one embodiment, the signature of the invention comprises the DED markers CXCL2, STAT1 and GNGT1. In another embodiment, the signature of the invention comprises the DED markers CXCL2, STAT1 and HMGB2. In another embodiment, the signature of the invention comprises the DED markers CXCL2, STAT1 and GNAQ. In another embodiment, the signature of the invention comprises the DED markers CXCL2, STAT1 and PLA2G4A. In another embodiment, the signature of the invention comprises the DED markers CXCL2, STAT1 and CFL1. In another embodiment, the signature of the invention comprises the DED markers CXCL2, STAT1 and CDC42. In another embodiment, the signature of the invention comprises the DED markers CXCL2, STAT1 and SHC1. In another embodiment, the signature of the invention comprises the DED markers CXCL2, STAT1 and CD4. In another embodiment, the signature of the invention comprises the DED markers CXCL2, STAT1 and TGFBR1. In another embodiment, the signature of the invention comprises the DED markers CXCL2, STAT1 and CXCR4.

In one embodiment, the signature of the invention comprises the DED markers CXCL2, GNGT1 and HMGB2. In another embodiment, the signature of the invention comprises the DED markers CXCL2, GNGT1 and GNAQ. In another embodiment, the signature of the invention comprises the DED markers CXCL2, GNGT1 and PLA2G4A. In another embodiment, the signature of the invention comprises the DED markers CXCL2, GNGT1 and CFL1. In another embodiment, the signature of the invention comprises the DED markers CXCL2, GNGT1 and CDC42. In another embodiment, the signature of the invention comprises the DED markers CXCL2, GNGT1 and SHC1. In another embodiment, the signature of the invention comprises the DED markers CXCL2, GNGT1 and CD4. In another embodiment, the signature of the invention comprises the DED markers CXCL2, GNGT1 and TGFBR1. In another embodiment, the signature of the invention comprises the DED markers CXCL2, GNGT1 and CXCR4.

In one embodiment, the signature of the invention comprises the DED markers CXCL2, HMGB2 and GNAQ. In another embodiment, the signature of the invention comprises the DED markers CXCL2, HMGB2 and PLA2G4A. In another embodiment, the signature of the invention comprises the DED markers CXCL2, HMGB2 and CFL1. In another embodiment, the signature of the invention comprises the DED markers CXCL2, HMGB2 and CDC42. In another embodiment, the signature of the invention comprises the DED markers CXCL2, HMGB2 and SHC1. In another embodiment, the signature of the invention comprises the DED markers CXCL2, HMGB2 and CD4. In another embodiment, the signature of the invention comprises the DED markers CXCL2, HMGB2 and TGFBR1. In another embodiment, the signature of the invention comprises the DED markers CXCL2, HMGB2 and CXCR4.

In one embodiment, the signature of the invention comprises the DED markers CXCL2, GNAQ and PLA2G4A. In another embodiment, the signature of the invention comprises the DED markers CXCL2, GNAQ and CFL1. In another embodiment, the signature of the invention comprises the DED markers CXCL2, GNAQ and CDC42. In another embodiment, the signature of the invention comprises the DED markers CXCL2, GNAQ and SHC1. In another embodiment, the signature of the invention comprises the DED markers CXCL2, GNAQ and CD4. In another embodiment, the signature of the invention comprises the DED markers CXCL2, GNAQ and TGFBR1. In another embodiment, the signature of the invention comprises the DED markers CXCL2, GNAQ and CXCR4.

In one embodiment, the signature of the invention comprises the DED markers CXCL2, PLA2G4A and CFL1. In another embodiment, the signature of the invention comprises the DED markers CXCL2, PLA2G4A and CDC42. In another embodiment, the signature of the invention comprises the DED markers CXCL2, PLA2G4A and SHC1. In another embodiment, the signature of the invention comprises the DED markers CXCL2, PLA2G4A and CD4. In another embodiment, the signature of the invention comprises the DED markers CXCL2, PLA2G4A and TGFBR1. In another embodiment, the signature of the invention comprises the DED markers CXCL2, PLA2G4A and CXCR4.

In one embodiment, the signature of the invention comprises the DED markers CXCL2, CFL1 and CDC42. In another embodiment, the signature of the invention comprises the DED markers CXCL2, CFL1 and SHC1. In another embodiment, the signature of the invention comprises the DED markers CXCL2, CFL1 and CD4. In another embodiment, the signature of the invention comprises the DED markers CXCL2, CFL1 and TGFBR1. In another embodiment, the signature of the invention comprises the DED markers CXCL2, CFL1 and CXCR4.

In one embodiment, the signature of the invention comprises the DED markers CXCL2, CDC42 and SHC1. In another embodiment, the signature of the invention comprises the DED markers CXCL2, CDC42 and CD4. In another embodiment, the signature of the invention comprises the DED markers CXCL2, CDC42 and TGFBR1. In another embodiment, the signature of the invention comprises the DED markers CXCL2, CDC42 and CXCR4.

In one embodiment, the signature of the invention comprises the DED markers CXCL2, SHC1 and CD4. In another embodiment, the signature of the invention comprises the DED markers CXCL2, SHC1 and TGFBR1. In another embodiment, the signature of the invention comprises the DED markers CXCL2, SHC1 and CXCR4.

In one embodiment, the signature of the invention comprises the DED markers CXCL2, CD4 and TGFBR1. In another embodiment, the signature of the invention comprises the DED markers CXCL2, CD4 and CXCR4.

In one embodiment, the signature of the invention comprises the DED markers CXCL2, TGFBR1 and CXCR4.

In one embodiment, the signature of the invention comprises the DED markers KEAP1, STAT1 and GNGT1. In another embodiment, the signature of the invention comprises the DED markers KEAP1, STAT1 and HMGB2. In another embodiment, the signature of the invention comprises the DED markers KEAP1, STAT1 and GNAQ. In another embodiment, the signature of the invention comprises the DED markers KEAP1, STAT1 and PLA2G4A. In another embodiment, the signature of the invention comprises the DED markers KEAP1, STAT1 and CFL1. In another embodiment, the signature of the invention comprises the DED markers KEAP1, STAT1 and CDC42. In another embodiment, the signature of the invention comprises the DED markers KEAP1, STAT1 and SHC1. In another embodiment, the signature of the invention comprises the DED markers KEAP1, STAT1 and CD4. In another embodiment, the signature of the invention comprises the DED markers KEAP1, STAT1 and TGFBR1. In another embodiment, the signature of the invention comprises the DED markers KEAP1, STAT1 and CXCR4.

In one embodiment, the signature of the invention comprises the DED markers KEAP1, GNGT1 and HMGB2. In another embodiment, the signature of the invention comprises the DED markers KEAP1, GNGT1 and GNAQ. In another embodiment, the signature of the invention comprises the DED markers KEAP1, GNGT1 and PLA2G4A. In another embodiment, the signature of the invention comprises the DED markers KEAP1, GNGT1 and CFL1. In another embodiment, the signature of the invention comprises the DED markers KEAP1, GNGT1 and CDC42. In another embodiment, the signature of the invention comprises the DED markers KEAP1, GNGT1 and SHC1. In another embodiment, the signature of the invention comprises the DED markers KEAP1, GNGT1 and CD4. In another embodiment, the signature of the invention comprises the DED markers KEAP1, GNGT1 and TGFBR1. In another embodiment, the signature of the invention comprises the DED markers KEAP1, GNGT1 and CXCR4.

In one embodiment, the signature of the invention comprises the DED markers KEAP1, HMGB2 and GNAQ. In another embodiment, the signature of the invention comprises the DED markers KEAP1, HMGB2 and PLA2G4A. In another embodiment, the signature of the invention comprises the DED markers KEAP1, HMGB2 and CFL1. In another embodiment, the signature of the invention comprises the DED markers KEAP1, HMGB2 and CDC42. In another embodiment, the signature of the invention comprises the DED markers KEAP1, HMGB2 and SHC1. In another embodiment, the signature of the invention comprises the DED markers KEAP1, HMGB2 and CD4. In another embodiment, the signature of the invention comprises the DED markers KEAP1, HMGB2 and TGFBR1. In another embodiment, the signature of the invention comprises the DED markers KEAP1, HMGB2 and CXCR4.

In one embodiment, the signature of the invention comprises the DED markers KEAP1, GNAQ and PLA2G4A. In another embodiment, the signature of the invention comprises the DED markers KEAP1, GNAQ and CFL1. In another embodiment, the signature of the invention comprises the DED markers KEAP1, GNAQ and CDC42. In another embodiment, the signature of the invention comprises the DED markers KEAP1, GNAQ and SHC1. In another embodiment, the signature of the invention comprises the DED markers KEAP1, GNAQ and CD4. In another embodiment, the signature of the invention comprises the DED markers KEAP1, GNAQ and TGFBR1. In another embodiment, the signature of the invention comprises the DED markers KEAP1, GNAQ and CXCR4.

In one embodiment, the signature of the invention comprises the DED markers KEAP1, PLA2G4A and CFL1. In another embodiment, the signature of the invention comprises the DED markers KEAP1, PLA2G4A and CDC42. In another embodiment, the signature of the invention comprises the DED markers KEAP1, PLA2G4A and SHC1. In another embodiment, the signature of the invention comprises the DED markers KEAP1, PLA2G4A and CD4. In another embodiment, the signature of the invention comprises the DED markers KEAP1, PLA2G4A and TGFBR1. In another embodiment, the signature of the invention comprises the DED markers KEAP1, PLA2G4A and CXCR4.

In one embodiment, the signature of the invention comprises the DED markers KEAP1, CFL1 and CDC42. In another embodiment, the signature of the invention comprises the DED markers KEAP1, CFL1 and SHC1. In another embodiment, the signature of the invention comprises the DED markers KEAP1, CFL1 and CD4. In another embodiment, the signature of the invention comprises the DED markers KEAP1, CFL1 and TGFBR1. In another embodiment, the signature of the invention comprises the DED markers KEAP1, CFL1 and CXCR4.

In one embodiment, the signature of the invention comprises the DED markers KEAP1, CDC42 and SHC1. In another embodiment, the signature of the invention comprises the DED markers KEAP1, CDC42 and CD4. In another embodiment, the signature of the invention comprises the DED markers KEAP1, CDC42 and TGFBR1. In another embodiment, the signature of the invention comprises the DED markers KEAP1, CDC42 and CXCR4.

In one embodiment, the signature of the invention comprises the DED markers KEAP1, SHC1 and CD4. In another embodiment, the signature of the invention comprises the DED markers KEAP1, SHC1 and TGFBR1. In another embodiment, the signature of the invention comprises the DED markers KEAP1, SHC1 and CXCR4.

In one embodiment, the signature of the invention comprises the DED markers KEAP1, CD4 and TGFBR1. In another embodiment, the signature of the invention comprises the DED markers KEAP1, CD4 and CXCR4.

In one embodiment, the signature of the invention comprises the DED markers KEAP1, TGFBR1 and CXCR4.

In one embodiment, the signature of the invention comprises the DED markers STAT1, GNGT1 and HMGB2. In another embodiment, the signature of the invention comprises the DED markers STAT1, GNGT1 and GNAQ. In another embodiment, the signature of the invention comprises the DED markers STAT1, GNGT1 and PLA2G4A. In another embodiment, the signature of the invention comprises the DED markers STAT1, GNGT1 and CFL1. In another embodiment, the signature of the invention comprises the DED markers STAT1, GNGT1 and CDC42. In another embodiment, the signature of the invention comprises the DED markers STAT1, GNGT1 and SHC1. In another embodiment, the signature of the invention comprises the DED markers STAT1, GNGT1 and CD4. In another embodiment, the signature of the invention comprises the DED markers STAT1, GNGT1 and TGFBR1. In another embodiment, the signature of the invention comprises the DED markers STAT1, GNGT1 and CXCR4.

In one embodiment, the signature of the invention comprises the DED markers STAT1, HMGB2 and GNAQ. In another embodiment, the signature of the invention comprises the DED markers STAT1, HMGB2 and PLA2G4A. In another embodiment, the signature of the invention comprises the DED markers STAT1, HMGB2 and CFL1. In another embodiment, the signature of the invention comprises the DED markers STAT1, HMGB2 and CDC42. In another embodiment, the signature of the invention comprises the DED markers STAT1, HMGB2 and SHC1. In another embodiment, the signature of the invention comprises the DED markers STAT1, HMGB2 and CD4. In another embodiment, the signature of the invention comprises the DED markers STAT1, HMGB2 and TGFBR1. In another embodiment, the signature of the invention comprises the DED markers STAT1, HMGB2 and CXCR4.

In one embodiment, the signature of the invention comprises the DED markers STAT1, GNAQ and PLA2G4A. In another embodiment, the signature of the invention comprises the DED markers STAT1, GNAQ and CFL1. In another embodiment, the signature of the invention comprises the DED markers STAT1, GNAQ and CDC42. In another embodiment, the signature of the invention comprises the DED markers STAT1, GNAQ and SHC1. In another embodiment, the signature of the invention comprises the DED markers STAT1, GNAQ and CD4. In another embodiment, the signature of the invention comprises the DED markers STAT1, GNAQ and TGFBR1. In another embodiment, the signature of the invention comprises the DED markers STAT1, GNAQ and CXCR4.

In one embodiment, the signature of the invention comprises the DED markers STAT1, PLA2G4A and CFL1. In another embodiment, the signature of the invention comprises the DED markers STAT1, PLA2G4A and CDC42. In another embodiment, the signature of the invention comprises the DED markers STAT1, PLA2G4A and SHC1. In another embodiment, the signature of the invention comprises the DED markers STAT1, PLA2G4A and CD4. In another embodiment, the signature of the invention comprises the DED markers STAT1, PLA2G4A and TGFBR1. In another embodiment, the signature of the invention comprises the DED markers STAT1, PLA2G4A and CXCR4.

In one embodiment, the signature of the invention comprises the DED markers STAT1, CFL1 and CDC42. In another embodiment, the signature of the invention comprises the DED markers STAT1, CFL1 and SHC1. In another embodiment, the signature of the invention comprises the DED markers STAT1, CFL1 and CD4. In another embodiment, the signature of the invention comprises the DED markers STAT1, CFL1 and TGFBR1. In another embodiment, the signature of the invention comprises the DED markers STAT1, CFL1 and CXCR4.

In one embodiment, the signature of the invention comprises the DED markers STAT1, CDC42 and SHC1. In another embodiment, the signature of the invention comprises the DED markers STAT1, CDC42 and CD4. In another embodiment, the signature of the invention comprises the DED markers STAT1, CDC42 and TGFBR1. In another embodiment, the signature of the invention comprises the DED markers STAT1, CDC42 and CXCR4.

In one embodiment, the signature of the invention comprises the DED markers STAT1, SHC1 and CD4. In another embodiment, the signature of the invention comprises the DED markers STAT1, SHC1 and TGFBR1. In another embodiment, the signature of the invention comprises the DED markers STAT1, SHC1 and CXCR4.

In one embodiment, the signature of the invention comprises the DED markers STAT1, CD4 and TGFBR1. In another embodiment, the signature of the invention comprises the DED markers STAT1, CD4 and CXCR4.

In one embodiment, the signature of the invention comprises the DED markers STAT1, TGFBR1 and CXCR4.

In one embodiment, the signature of the invention comprises the DED markers GNGT1, HMGB2 and GNAQ. In another embodiment, the signature of the invention comprises the DED markers GNGT1, HMGB2 and PLA2G4A. In another embodiment, the signature of the invention comprises the DED markers GNGT1, HMGB2 and CFL1. In another embodiment, the signature of the invention comprises the DED markers GNGT1, HMGB2 and CDC42. In another embodiment, the signature of the invention comprises the DED markers GNGT1, HMGB2 and SHC1. In another embodiment, the signature of the invention comprises the DED markers GNGT1, HMGB2 and CD4. In another embodiment, the signature of the invention comprises the DED markers GNGT1, HMGB2 and TGFBR1. In another embodiment, the signature of the invention comprises the DED markers GNGT1, HMGB2 and CXCR4.

In one embodiment, the signature of the invention comprises the DED markers GNGT1, GNAQ and PLA2G4A. In another embodiment, the signature of the invention comprises the DED markers GNGT1, GNAQ and CFL1. In another embodiment, the signature of the invention comprises the DED markers GNGT1, GNAQ and CDC42. In another embodiment, the signature of the invention comprises the DED markers GNGT1, GNAQ and SHC1. In another embodiment, the signature of the invention comprises the DED markers GNGT1, GNAQ and CD4. In another embodiment, the signature of the invention comprises the DED markers GNGT1, GNAQ and TGFBR1. In another embodiment, the signature of the invention comprises the DED markers GNGT1, GNAQ and CXCR4.

In one embodiment, the signature of the invention comprises the DED markers GNGT1, PLA2G4A and CFL1. In another embodiment, the signature of the invention comprises the DED markers GNGT1, PLA2G4A and CDC42. In another embodiment, the signature of the invention comprises the DED markers GNGT1, PLA2G4A and SHC1. In another embodiment, the signature of the invention comprises the DED markers GNGT1, PLA2G4A and CD4. In another embodiment, the signature of the invention comprises the DED markers GNGT1, PLA2G4A and TGFBR1. In another embodiment, the signature of the invention comprises the DED markers GNGT1, PLA2G4A and CXCR4.

In one embodiment, the signature of the invention comprises the DED markers GNGT1, CFL1 and CDC42. In another embodiment, the signature of the invention comprises the DED markers GNGT1, CFL1 and SHC1. In another embodiment, the signature of the invention comprises the DED markers GNGT1, CFL1 and CD4. In another embodiment, the signature of the invention comprises the DED markers GNGT1, CFL1 and TGFBR1. In another embodiment, the signature of the invention comprises the DED markers GNGT1, CFL1 and CXCR4.

In one embodiment, the signature of the invention comprises the DED markers GNGT1, CDC42 and SHC1. In another embodiment, the signature of the invention comprises the DED markers GNGT1, CDC42 and CD4. In another embodiment, the signature of the invention comprises the DED markers GNGT1, CDC42 and TGFBR1. In another embodiment, the signature of the invention comprises the DED markers GNGT1, CDC42 and CXCR4.

In one embodiment, the signature of the invention comprises the DED markers GNGT1, SHC1 and CD4. In another embodiment, the signature of the invention comprises the DED markers GNGT1, SHC1 and TGFBR1. In another embodiment, the signature of the invention comprises the DED markers GNGT1, SHC1 and CXCR4.

In one embodiment, the signature of the invention comprises the DED markers GNGT1, CD4 and TGFBR1. In another embodiment, the signature of the invention comprises the DED markers GNGT1, CD4 and CXCR4.

In one embodiment, the signature of the invention comprises the DED markers GNGT1, TGFBR1 and CXCR4.

In one embodiment, the signature of the invention comprises the DED markers HMGB2, GNAQ and PLA2G4A. In another embodiment, the signature of the invention comprises the DED markers HMGB2, GNAQ and CFL1. In another embodiment, the signature of the invention comprises the DED markers HMGB2, GNAQ and CDC42. In another embodiment, the signature of the invention comprises the DED markers HMGB2, GNAQ and SHC1. In another embodiment, the signature of the invention comprises the DED markers HMGB2, GNAQ and CD4. In another embodiment, the signature of the invention comprises the DED markers HMGB2, GNAQ and TGFBR1. In another embodiment, the signature of the invention comprises the DED markers HMGB2, GNAQ and CXCR4.

In one embodiment, the signature of the invention comprises the DED markers HMGB2, PLA2G4A and CFL1. In another embodiment, the signature of the invention comprises the DED markers HMGB2, PLA2G4A and CDC42. In another embodiment, the signature of the invention comprises the DED markers HMGB2, PLA2G4A and SHC1. In another embodiment, the signature of the invention comprises the DED markers HMGB2, PLA2G4A and CD4. In another embodiment, the signature of the invention comprises the DED markers HMGB2, PLA2G4A and TGFBR1. In another embodiment, the signature of the invention comprises the DED markers HMGB2, PLA2G4A and CXCR4.

In one embodiment, the signature of the invention comprises the DED markers HMGB2, CFL1 and CDC42. In another embodiment, the signature of the invention comprises the DED markers HMGB2, CFL1 and SHC1. In another embodiment, the signature of the invention comprises the DED markers HMGB2, CFL1 and CD4. In another embodiment, the signature of the invention comprises the DED markers HMGB2, CFL1 and TGFBR1. In another embodiment, the signature of the invention comprises the DED markers HMGB2, CFL1 and CXCR4.

In one embodiment, the signature of the invention comprises the DED markers HMGB2, CDC42 and SHC1. In another embodiment, the signature of the invention comprises the DED markers HMGB2, CDC42 and CD4. In another embodiment, the signature of the invention comprises the DED markers HMGB2, CDC42 and TGFBR1. In another embodiment, the signature of the invention comprises the DED markers HMGB2, CDC42 and CXCR4.

In one embodiment, the signature of the invention comprises the DED markers HMGB2, SHC1 and CD4. In another embodiment, the signature of the invention comprises the DED markers HMGB2, SHC1 and TGFBR1. In another embodiment, the signature of the invention comprises the DED markers HMGB2, SHC1 and CXCR4.

In one embodiment, the signature of the invention comprises the DED markers HMGB2, CD4 and TGFBR1. In another embodiment, the signature of the invention comprises the DED markers HMGB2, CD4 and CXCR4.

In one embodiment, the signature of the invention comprises the DED markers HMGB2, TGFBR1 and CXCR4.

In one embodiment, the signature of the invention comprises the DED markers GNAQ, PLA2G4A and CFL1. In another embodiment, the signature of the invention comprises the DED markers GNAQ, PLA2G4A and CDC42. In another embodiment, the signature of the invention comprises the DED markers GNAQ, PLA2G4A and SHC1. In another embodiment, the signature of the invention comprises the DED markers GNAQ, PLA2G4A and CD4. In another embodiment, the signature of the invention comprises the DED markers GNAQ, PLA2G4A and TGFBR1. In another embodiment, the signature of the invention comprises the DED markers GNAQ, PLA2G4A and CXCR4.

In one embodiment, the signature of the invention comprises the DED markers GNAQ, CFL1 and CDC42. In another embodiment, the signature of the invention comprises the DED markers GNAQ, CFL1 and SHC1. In another embodiment, the signature of the invention comprises the DED markers GNAQ, CFL1 and CD4. In another embodiment, the signature of the invention comprises the DED markers GNAQ, CFL1 and TGFBR1. In another embodiment, the signature of the invention comprises the DED markers GNAQ, CFL1 and CXCR4.

In one embodiment, the signature of the invention comprises the DED markers GNAQ, CDC42 and SHC1. In another embodiment, the signature of the invention comprises the DED markers GNAQ, CDC42 and CD4. In another embodiment, the signature of the invention comprises the DED markers GNAQ, CDC42 and TGFBR1. In another embodiment, the signature of the invention comprises the DED markers GNAQ, CDC42 and CXCR4.

In one embodiment, the signature of the invention comprises the DED markers GNAQ, SHC1 and CD4. In another embodiment, the signature of the invention comprises the DED markers GNAQ, SHC1 and TGFBR1. In another embodiment, the signature of the invention comprises the DED markers GNAQ, SHC1 and CXCR4.

In one embodiment, the signature of the invention comprises the DED markers GNAQ, CD4 and TGFBR1. In another embodiment, the signature of the invention comprises the DED markers GNAQ, CD4 and CXCR4.

In one embodiment, the signature of the invention comprises the DED markers GNAQ, TGFBR1 and CXCR4.

In one embodiment, the signature of the invention comprises the DED markers PLA2G4A, CFL1 and CDC42. In another embodiment, the signature of the invention comprises the DED markers PLA2G4A, CFL1 and SHC1. In another embodiment, the signature of the invention comprises the DED markers PLA2G4A, CFL1 and CD4. In another embodiment, the signature of the invention comprises the DED markers PLA2G4A, CFL1 and TGFBR1. In another embodiment, the signature of the invention comprises the DED markers PLA2G4A, CFL1 and CXCR4.

In one embodiment, the signature of the invention comprises the DED markers PLA2G4A, CDC42 and SHC1. In another embodiment, the signature of the invention comprises the DED markers PLA2G4A, CDC42 and CD4. In another embodiment, the signature of the invention comprises the DED markers PLA2G4A, CDC42 and TGFBR1. In another embodiment, the signature of the invention comprises the DED markers PLA2G4A, CDC42 and CXCR4.

In one embodiment, the signature of the invention comprises the DED markers PLA2G4A, SHC1 and CD4. In another embodiment, the signature of the invention comprises the DED markers PLA2G4A, SHC1 and TGFBR1. In another embodiment, the signature of the invention comprises the DED markers PLA2G4A, SHC1 and CXCR4.

In one embodiment, the signature of the invention comprises the DED markers PLA2G4A, CD4 and TGFBR1. In another embodiment, the signature of the invention comprises the DED markers PLA2G4A, CD4 and CXCR4.

In one embodiment, the signature of the invention comprises the DED markers PLA2G4A, TGFBR1 and CXCR4.

In one embodiment, the signature of the invention comprises the DED markers CFL1, CDC42 and SHC1. In another embodiment, the signature of the invention comprises the DED markers CFL1, CDC42 and CD4. In another embodiment, the signature of the invention comprises the DED markers CFL1, CDC42 and TGFBR1. In another embodiment, the signature of the invention comprises the DED markers CFL1, CDC42 and CXCR4.

In one embodiment, the signature of the invention comprises the DED markers CFL1, SHC1 and CD4. In another embodiment, the signature of the invention comprises the DED markers CFL1, SHC1 and TGFBR1. In another embodiment, the signature of the invention comprises the DED markers CFL1, SHC1 and CXCR4.

In one embodiment, the signature of the invention comprises the DED markers CFL1, CD4 and TGFBR1. In another embodiment, the signature of the invention comprises the DED markers CFL1, CD4 and CXCR4.

In one embodiment, the signature of the invention comprises the DED markers CFL1, TGFBR1 and CXCR4.

In one embodiment, the signature of the invention comprises the DED markers CDC42, SHC1 and CD4. In another embodiment, the signature of the invention comprises the DED markers CDC42, SHC1 and TGFBR1. In another embodiment, the signature of the invention comprises the DED markers CDC42, SHC1 and CXCR4.

In one embodiment, the signature of the invention comprises the DED markers CDC42, CD4 and TGFBR1. In one another, the signature of the invention comprises the DED markers CDC42, CD4 and CXCR4.

In one embodiment, the signature of the invention comprises the DED markers CDC42, TGFBR1 and CXCR4.

In one embodiment, the signature of the invention comprises the DED markers SHC1, CD4 and TGFBR1. In another embodiment, the signature of the invention comprises the DED markers SHC1, CD4 and CXCR4.

In one embodiment, the signature of the invention comprises the DED markers SHC1, TGFBR1 and CXCR4.

In one embodiment, the signature of the invention comprises the DED markers CD4, TGFBR1 and CXCR4.

In one embodiment of the invention, the signature of the invention comprises at least the two DED markers GNAQ and PLA2G4A. In another embodiment of the invention, the signature of the invention comprises at least the two DED markers GNAQ and STAT1. In another embodiment of the invention, the signature of the invention comprises at least the two DED markers GNAQ and SHC1.

In one embodiment, the at least one DED marker is CXCL2. In one embodiment, the at least one DED marker is KEAP1. In one embodiment, the at least one DED marker is STAT1. In one embodiment, the at least one DED marker is GNGT1. In one embodiment, the at least one DED marker is HMGB2. In one embodiment, the at least one DED marker is GNAQ. In one embodiment, the at least one DED marker is PLA2G4A. In one embodiment, the at least one DED marker is CFL1. In one embodiment, the at least one DED marker is CDC42. In one embodiment, the at least one DED marker is SHC1. In another embodiment, the at least one DED marker is CD4. In one embodiment, the at least one DED marker is TGFBR1. In one embodiment, the at least one DED marker is CXCR4.

In one embodiment, the signature of the invention comprises 1 DED marker selected from the list of Table 8, and 1, 2, 3, 4, or 5 other DED markers selected from the list of Table 1, preferably from the list of Table 2, more preferably from the list of Table 3, 4, 5, 6 or 7. In another embodiment, the signature of the invention comprises 2 DED markers selected from the list of Table 8, and 1, 2, 3, or 4 other DED markers selected from the list of Table 1, preferably from the list of Table 2, more preferably from the list of Table 3, 4, 5, 6 or 7. In another embodiment, the signature of the invention comprises 3 DED markers selected from the list of Table 8, and 1, 2, or 3 other DED markers selected from the list of Table 1, preferably from the list of Table 2, more preferably from the list of Table 3, 4, 5, 6 or 7.

In one embodiment, the signature of the invention comprises 1 DED marker selected from the list of Table 9, preferably of Table 10, and 1, 2, 3, 4, or 5 other DED markers selected from the list of Table 1, preferably from the list of Table 2, more preferably from the list of Table 3, 4, 5, 6, 7 or 8. In another embodiment, the signature of the invention comprises 2 DED markers selected from the list of Table 9, preferably of Table 10, and 1, 2, 3, or 4 other DED markers selected from the list of Table 1, preferably from the list of Table 2, more preferably from the list of Table 3, 4, 5, 6, 7 or 8. In another embodiment, the signature of the invention comprises 3 DED markers selected from the list of Table 9, preferably of Table 10, and 1, 2, or 3 other DED markers selected from the list of Table 1, preferably from the list of Table 2, more preferably from the list of Table 3, 4, 5, 6, 7 or 8.

In one embodiment, the signature of the invention comprises 1 DED marker selected from the list of Table 8 or 9, preferably of Table 8 or 10, and 1, 2, 3, 4, or 5 other DED markers selected from the list of Table 1, preferably from the list of Table 2, more preferably from the list of Table 3, 4, 5, 6 or 7. In another embodiment, the signature of the invention comprises 2 DED markers selected from the list of Table 8 or 9, preferably of Table 8 or 10, and 1, 2, 3, or 4 other DED markers selected from the list of Table 1, preferably from the list of Table 2, more preferably from the list of Table 3, 4, 5, 6 or 7. In another embodiment, the signature of the invention comprises 3 DED markers selected from the list of Table 8 or 9, preferably of Table 8 or 10, and 1, 2, or 3 other DED markers selected from the list of Table 1, preferably from the list of Table 2, more preferably from the list of Table 3, 4, 5, 6 or 7.

In one embodiment, the at least one DED marker is not OASL. In one embodiment, the at least one DED marker is not CXCL9. In one embodiment, the at least one DED marker is not TCF4. In one embodiment, the at least one DED marker is not CXCL10. In one embodiment, the at least one DED marker is not CCL20. In one embodiment, the at least one DED marker is not DEFB2. In one embodiment, the at least one DED marker is not IFNG. In one embodiment, the at least one DED marker is not IL1B. In one embodiment, the at least one DED marker is not IL4. In one embodiment, the at least one DED marker is not IL6. In one embodiment, the at least one DED marker is not IL7. In one embodiment, the at least one DED marker is not IL8. In one embodiment, the at least one DED marker is not IL15. In one embodiment, the at least one DED marker is not IL23 or IL23A. In one embodiment, the at least one DED marker is not MMP9. In one embodiment, the at least one DED marker is not MUC4. In one embodiment, the at least one DED marker is not TGFB1. In one embodiment, the at least one DED marker is not TNF.

In one embodiment, the signature of the invention comprises mild DED markers.

As used herein, the term “mild DED markers” encompasses markers whose expression is different between a patient suffering from mild DED (i.e. a mild DED patient) and a “normal” patient, and markers whose expression is different between a patient suffering from mild DED (i.e. a mild DED patient) and a patient suffering from severe DED (i.e. a severe DED patient).

According to the invention, the term “mild DED” encompasses mild and moderate DED.

The present invention thus also relates to “mild DED markers”. In one embodiment, the signature according to the invention comprises at least one mild DED marker.

In one embodiment of the invention, the signature of the invention comprises 1, 2, 3, 4, 5, 6, 7, 8, 9 or 10 mild DED markers.

In one embodiment, mild DED markers are selected from markers whose expression is different between a mild DED patient and a “normal” patient.

In one embodiment, the at least one mild DED marker is selected from the list of the 22 mild DED markers of Table 11 below, as well as their variants, fragments or equivalents. Table 11 comprises mild DED markers whose expression is different between a mild DED patient and a “normal” patient, identified in the conditions of the Example and presenting a fold-change, positive or negative, superior or equal to 1.25.

TABLE 11

| Name of the | GeneBank | |
|---|---|---|
| marker | Accession Number | Target sequence (SEQ ID number) |
| CCL13 | NM_005408.2 | CCAGAATTATATGAAACACCTGGGCCGGAAAGCTCAC |
| | | ACCCTGAAGACTTGAACTCTGCTACCCCTACTGAAATC |
| | | AAGCTGGAGTACGTGAAATGACTTT (SEQ ID NO: 62) |
| C4A | NM_007293.2 | GGTCCGTGAGATGTCAGGCTCCCCAGCTTCTGGCATTC |
| | | CTGTCAAAGTTTCTGCCACGGTGTCTTCTCCTGGGTCT |
| | | GTTCCTGAAGTCCAGGACATTCAG (SEQ ID NO: 63) |
| CFB | NM_001710.5 | TAAGCTGAAATATGGCCAGACTATCAGGCCCATTTGTC |
| | | TCCCCTGCACCGAGGGAACAACTCGAGCTTTGAGGCT |
| | | TCCTCCAACTACCACTTGCCAGCAA (SEQ ID NO: 64) |
| CXCL5 | NM_002994.3 | AGAGAGCTGCGTTGCGTTTGTTTACAGACCACGCAAG |
| | | GAGTTCATCCCAAAATGATCAGTAATCTGCAAGTGTTC |
| | | GCCATAGGCCCACAGTGCTCCAAGG (SEQ ID NO: 65) |
| CXCL6 | NM_002993.3 | AGTAACAAAAAAGACCATGCATCATAAAATTGCCCAG |
| | | TCTTCAGCGGAGCAGTTTTCTGGAGATCCCTGGACCCA |
| | | GTAAGAATAAGAAGGAAGGGTTGGT (SEQ ID NO: 66) |
| CXCL9 | NM_002416.1 | CACCATCTCCCATGAAGAAAGGGAACGGTGAAGTACT |
| | | AAGCGCTAGAGGAAGCAGCCAAGTCGGTTAGTGGAAG |
| | | CATGATTGGTGCCCAGTTAGCCTCTG (SEQ ID NO: 67) |
| CXCR2 | NM_001557.2 | AGGAGAAACTGGAACTCTCGAGCGTTGCTGGGGGGGA |
| | | TTGTAAAATGGTGTGACCACTGCAGAAGACAGTATGG |
| | | CAGCTTTCCTCAAAACTTCAGACATA (SEQ ID NO: 68) |
| HLA-DRA | NM_019111.3 | GGCCAACATAGCTGTGGACAAAGCCAACCTGGAAATC |
| | | ATGACAAAGCGCTCCAACTATACTCCGATCACCAATG |
| | | TACCTCCAGAGGTAACTGTGCTCACG (SEQ ID NO: 69) |
| HSH2D | NM_032855.2 | GTGTCAGCACCAGGATTTGAAGCCAGGTGAATCCGAG |
| | | CCCTTTTCCCATATCATCTGTTTGTTCTGTTGTCTAAAA |
| | | GCACACTGCAAGCCGGGCTCAGTG (SEQ ID NO: 70) |
| IFI44 | NM_006417.4 | TTTCCAAGGGCATGTAACGCATCAGGCTTTGGTGGGC |
| | | ACTAATACAACTGGGATATCTGAGAAGTATAGGACAT |
| | | ACTCTATTAGAGACGGGAAAGATGGC (SEQ ID NO: 71) |
| IL7 | NM_000880.2 | AATAACCCAGCTTGCGTCCTGCACACTTGTGGCTTCCG |
| | | TGCACACATTAACAACTCATGGTTCTAGCTCCCAGTCG |
| | | CCAAGCGTTGCCAAGGCGTTGAGA (SEQ ID NO: 72) |
| IRF3 | NM_001571.5 | TCATGGCCCCAGGACCAGCCGTGGACCAAGAGGCTCG |
| | | TGATGGTCAAGGTTGTGCCCACGTGCCTCAGGGCCTTG |
| | | GTAGAAATGGCCCGGGTAGGGGGTG (SEQ ID NO: 73) |
| ITGB2 | NM_000211.2 | CATCGACCTGTACTATCTGATGGACCTCTCCTACTCCA |
| | | TGCTTGATGACCTCAGGAATGTCAAGAAGCTAGGTGG |
| | | CGACCTGCTCCGGGCCCTCAACGAG (SEQ ID NO: 74) |
| LTB4R2 | NM_019839.4 | CAGAGGTCAGTGTTCTGGGACATTTGGGGACCCTTCTT |
| | | TGACTAGAGTTTGGATCTGGCTGGGTAGGATTACTATA |
| | | CACTTGGGGCAGGCCCAGGCTCCT (SEQ ID NO: 75) |
| MAFF | NM_001161572.1 | AGGCCTGAGGTCACATTTCACTTAGTGGTTGTGTTGGG |
| | | ACCAAAACCTGGGTGTCCTCACTGCTGCCCTGAGTCCA |
| | | GCCATGGTTTTCAGGGGGACAGTG (SEQ ID NO: 76) |
| MX2 | NM_002463.1 | AATTTACCGGCTCACTCAGGCGCGACACGCACTCTGTC |
| | | AATTCTCCAGCAAAGAGATCCACTGAAGGGCGGCGAT |
| | | GCCTGTGGTTGTTTTCTTGTGCGTA (SEQ ID NO: 77) |
| NOX1 | NM_007052.4 | TCATTTTGCAGCCGCACACTGAGAAAGCAATTGGATC |
| | | ACAACCTCACCTTCCACAAGCTGGTGGCCTATATGATC |
| | | TGCCTACATACAGCTATTCACATCA (SEQ ID NO: 78) |
| OAS2 | NM_016817.2 | TGAAAAACAATTTCGAGATCCAGAAGTCCCTTGATGG |
| | | GTTCACCATCCAGGTGTTCACAAAAAATCAGAGAATC |
| | | TCTTTCGAGGTGCTGGCCGCCTTCAA (SEQ ID NO: 79) |
| OASL | NM_198213.1 | GGCGTTTCTGAGCTGTTTCCACAGCTTCCAGGAGGCAG |
| | | CCAAGCATCACAAAGATGTTCTGAGGCTGATATGGAA |
| | | AACCATGTGGCAAAGCCAGGACCTG (SEQ ID NO: 80) |
| PTGER4 | NM_000958.2 | CGCGGGCGCCGAGATCCAGATGGTCATCTTACTCATTG |
| | | CCACCTCCCTGGTGGTGCTCATCTGCTCCATCCCGCTC |
| | | GTGGTGCGAGTATTCGTCAACCAG (SEQ ID NO: 81) |
| RELB | NM_006509.2 | CACTCTCGCTCGCCGTTTCCAGGAGCACAGATGAATTG |
| | | GAGATCATCGACGAGTACATCAAGGAGAACGGCTTCG |
| | | GCCTGGACGGGGGACAGCCGGGCCC (SEQ ID NO: 82) |
| TNFAIP3 | NM_006290.2 | CAAAGCCCTCATCGACAGAAACATCCAGGCCACCCTG |
| | | GAAAGCCAGAAGAAACTCAACTGGTGTCGAGAAGTCC |
| | | GGAAGCTTGTGGCGCTGAAAACGAAC (SEQ ID NO: 83) |

In one embodiment, the at least one mild DED marker is selected from the list of the 21 mild DED markers of Table 12 below, as well as their variants, fragments or equivalents. Table 12 comprises mild DED markers whose expression is different between a mild DED patient and a “normal” patient, identified in the conditions of the Example and presenting a fold-change, positive or negative, superior or equal to 1.25.

TABLE 12

| Name of the | GeneBank | |
|---|---|---|
| marker | Accession Number | Target sequence (SEQ ID number) |
| CCL13 | NM_005408.2 | CCAGAATTATATGAAACACCTGGGCCGGAAAGCTCAC |
| | | ACCCTGAAGACTTGAACTCTGCTACCCCTACTGAAATC |
| | | AAGCTGGAGTACGTGAAATGACTTT (SEQ ID NO: 62) |
| C4A | NM_007293.2 | GGTCCGTGAGATGTCAGGCTCCCCAGCTTCTGGCATTC |
| | | CTGTCAAAGTTTCTGCCACGGTGTCTTCTCCTGGGTCT |
| | | GTTCCTGAAGTCCAGGACATTCAG (SEQ ID NO: 63) |
| CFB | NM_001710.5 | TAAGCTGAAATATGGCCAGACTATCAGGCCCATTTGTC |
| | | TCCCCTGCACCGAGGGAACAACTCGAGCTTTGAGGCT |
| | | TCCTCCAACTACCACTTGCCAGCAA (SEQ ID NO: 64) |
| CXCL5 | NM_002994.3 | AGAGAGCTGCGTTGCGTTTGTTTACAGACCACGCAAG |
| | | GAGTTCATCCCAAAATGATCAGTAATCTGCAAGTGTTC |
| | | GCCATAGGCCCACAGTGCTCCAAGG (SEQ ID NO: 65) |
| CXCL6 | NM_002993.3 | AGTAACAAAAAAGACCATGCATCATAAAATTGCCCAG |
| | | TCTTCAGCGGAGCAGTTTTCTGGAGATCCCTGGACCCA |
| | | GTAAGAATAAGAAGGAAGGGTTGGT (SEQ ID NO: 66) |
| CXCL9 | NM_002416.1 | CACCATCTCCCATGAAGAAAGGGAACGGTGAAGTACT |
| | | AAGCGCTAGAGGAAGCAGCCAAGTCGGTTAGTGGAAG |
| | | CATGATTGGTGCCCAGTTAGCCTCTG (SEQ ID NO: 67) |
| CXCR2 | NM_001557.2 | AGGAGAAACTGGAACTCTCGAGCGTTGCTGGGGGGGA |
| | | TTGTAAAATGGTGTGACCACTGCAGAAGACAGTATGG |
| | | CAGCTTTCCTCAAAACTTCAGACATA (SEQ ID NO: 68) |
| HLA-DRA | NM_019111.3 | GGCCAACATAGCTGTGGACAAAGCCAACCTGGAAATC |
| | | ATGACAAAGCGCTCCAACTATACTCCGATCACCAATG |
| | | TACCTCCAGAGGTAACTGTGCTCACG (SEQ ID NO: 69) |
| HSH2D | NM_032855.2 | GTGTCAGCACCAGGATTTGAAGCCAGGTGAATCCGAG |
| | | CCCTTTTCCCATATCATCTGTTTGTTCTGTTGTCTAAAA |
| | | GCACACTGCAAGCCGGGCTCAGTG (SEQ ID NO: 70) |
| IFI44 | NM_006417.4 | TTTCCAAGGGCATGTAACGCATCAGGCTTTGGTGGGC |
| | | ACTAATACAACTGGGATATCTGAGAAGTATAGGACAT |
| | | ACTCTATTAGAGACGGGAAAGATGGC (SEQ ID NO: 71) |
| IRF3 | NM_001571.5 | TCATGGCCCCAGGACCAGCCGTGGACCAAGAGGCTCG |
| | | TGATGGTCAAGGTTGTGCCCACGTGCCTCAGGGCCTTG |
| | | GTAGAAATGGCCCGGGTAGGGGGTG (SEQ ID NO: 73) |
| ITGB2 | NM_000211.2 | CATCGACCTGTACTATCTGATGGACCTCTCCTACTCCA |
| | | TGCTTGATGACCTCAGGAATGTCAAGAAGCTAGGTGG |
| | | CGACCTGCTCCGGGCCCTCAACGAG (SEQ ID NO: 74) |
| LTB4R2 | NM_019839.4 | CAGAGGTCAGTGTTCTGGGACATTTGGGGACCCTTCTT |
| | | TGACTAGAGTTTGGATCTGGCTGGGTAGGATTACTATA |
| | | CACTTGGGGCAGGCCCAGGCTCCT (SEQ ID NO: 75) |
| MAFF | NM_001161572.1 | AGGCCTGAGGTCACATTTCACTTAGTGGTTGTGTTGGG |
| | | ACCAAAACCTGGGTGTCCTCACTGCTGCCCTGAGTCCA |
| | | GCCATGGTTTTCAGGGGGACAGTG (SEQ ID NO: 76) |
| MX2 | NM_002463.1 | AATTTACCGGCTCACTCAGGCGCGACACGCACTCTGTC |
| | | AATTCTCCAGCAAAGAGATCCACTGAAGGGCGGCGAT |
| | | GCCTGTGGTTGTTTTCTTGTGCGTA (SEQ ID NO: 77) |
| NOX1 | NM_007052.4 | TCATTTTGCAGCCGCACACTGAGAAAGCAATTGGATC |
| | | ACAACCTCACCTTCCACAAGCTGGTGGCCTATATGATC |
| | | TGCCTACATACAGCTATTCACATCA (SEQ ID NO: 78) |
| OAS2 | NM_016817.2 | TGAAAAACAATTTCGAGATCCAGAAGTCCCTTGATGG |
| | | GTTCACCATCCAGGTGTTCACAAAAAATCAGAGAATC |
| | | TCTTTCGAGGTGCTGGCCGCCTTCAA (SEQ ID NO: 79) |
| OASL | NM_198213.1 | GGCGTTTCTGAGCTGTTTCCACAGCTTCCAGGAGGCAG |
| | | CCAAGCATCACAAAGATGTTCTGAGGCTGATATGGAA |
| | | AACCATGTGGCAAAGCCAGGACCTG (SEQ ID NO: 80) |
| PTGER4 | NM_000958.2 | CGCGGGCGCCGAGATCCAGATGGTCATCTTACTCATTG |
| | | CCACCTCCCTGGTGGTGCTCATCTGCTCCATCCCGCTC |
| | | GTGGTGCGAGTATTCGTCAACCAG (SEQ ID NO: 81) |
| RELB | NM_006509.2 | CACTCTCGCTCGCCGTTTCCAGGAGCACAGATGAATTG |
| | | GAGATCATCGACGAGTACATCAAGGAGAACGGCTTCG |
| | | GCCTGGACGGGGGACAGCCGGGCCC (SEQ ID NO: 82) |
| TNFAIP3 | NM_006290.2 | CAAAGCCCTCATCGACAGAAACATCCAGGCCACCCTG |
| | | GAAAGCCAGAAGAAACTCAACTGGTGTCGAGAAGTCC |
| | | GGAAGCTTGTGGCGCTGAAAACGAAC (SEQ ID NO: 83) |

In another embodiment, the at least one mild DED marker is selected from the list of the 12 mild DED markers of Table 13 below, as well as their variants, fragments or equivalents. Table 13 comprises mild DED markers whose expression is different between a mild DED patient and a “normal” patient, identified in the conditions of the Example and presenting a fold-change, positive or negative, superior or equal to 2.

TABLE 13

| Name of | GeneBank | |
|---|---|---|
| the | Accession | |
| marker | Number | Target sequence (SEQ ID number) |
| CXCL9 | NM_002416.1 | CACCATCTCCCATGAAGAAAGGGAACGGTGAAGTACT |
| | | AAGCGCTAGAGGAAGCAGCCAAGTCGGTTAGTGGAAG |
| | | CATGATTGGTGCCCAGTTAGCCTCTG (SEQ ID NO: 67) |
| CXCR2 | NM_001557.2 | AGGAGAAACTGGAACTCTCGAGCGTTGCTGGGGGGGA |
| | | TTGTAAAATGGTGTGACCACTGCAGAAGACAGTATGG |
| | | CAGCTTTCCTCAAAACTTCAGACATA (SEQ ID NO: 68) |
| HLA-DRA | NM_019111.3 | GGCCAACATAGCTGTGGACAAAGCCAACCTGGAAATC |
| | | ATGACAAAGCGCTCCAACTATACTCCGATCACCAATG |
| | | TACCTCCAGAGGTAACTGTGCTCACG (SEQ ID NO: 69) |
| IF144 | NM_006417.4 | TTTCCAAGGGCATGTAACGCATCAGGCTTTGGTGGGC |
| | | ACTAATACAACTGGGATATCTGAGAAGTATAGGACAT |
| | | ACTCTATTAGAGACGGGAAAGATGGC (SEQ ID NO: 71) |
| ITGB2 | NM_000211.2 | CATCGACCTGTACTATCTGATGGACCTCTCCTACTCCA |
| | | TGCTTGATGACCTCAGGAATGTCAAGAAGCTAGGTGG |
| | | CGACCTGCTCCGGGCCCTCAACGAG (SEQ ID NO: 74) |
| LTB4R2 | NM_019839.4 | CAGAGGTCAGTGTTCTGGGACATTTGGGGACCCTTCTT |
| | | TGACTAGAGTTTGGATCTGGCTGGGTAGGATTACTATA |
| | | CACTTGGGGCAGGCCCAGGCTCCT (SEQ ID NO: 75) |
| MAFF | NM_001161572.1 | AGGCCTGAGGTCACATTTCACTTAGTGGTTGTGTTGGG |
| | | ACCAAAACCTGGGTGTCCTCACTGCTGCCCTGAGTCCA |
| | | GCCATGGTTTTCAGGGGGACAGTG (SEQ ID NO: 76) |
| MX2 | NM_002463.1 | AATTTACCGGCTCACTCAGGCGCGACACGCACTCTGTC |
| | | AATTCTCCAGCAAAGAGATCCACTGAAGGGCGGCGAT |
| | | GCCTGTGGTTGTTTTCTTGTGCGTA (SEQ ID NO: 77) |
| NOX1 | NM_007052.4 | TCATTTTGCAGCCGCACACTGAGAAAGCAATTGGATC |
| | | ACAACCTCACCTTCCACAAGCTGGTGGCCTATATGATC |
| | | TGCCTACATACAGCTATTCACATCA (SEQ ID NO: 78) |
| OAS2 | NM_016817.2 | TGAAAAACAATTTCGAGATCCAGAAGTCCCTTGATGG |
| | | GTTCACCATCCAGGTGTTCACAAAAAATCAGAGAATC |
| | | TCTTTCGAGGTGCTGGCCGCCTTCAA (SEQ ID NO: 79) |
| OASL | NM_198213.1 | GGCGTTTCTGAGCTGTTTCCACAGCTTCCAGGAGGCAG |
| | | CCAAGCATCACAAAGATGTTCTGAGGCTGATATGGAA |
| | | AACCATGTGGCAAAGCCAGGACCTG (SEQ ID NO: 80) |
| TNFAIP3 | NM_006290.2 | CAAAGCCCTCATCGACAGAAACATCCAGGCCACCCTG |
| | | GAAAGCCAGAAGAAACTCAACTGGTGTCGAGAAGTCC |
| | | GGAAGCTTGTGGCGCTGAAAACGAAC (SEQ ID NO: 83) |

In another embodiment, the at least one mild DED marker is selected from the list of the 6 mild DED markers of Table 14 below, as well as their variants, fragments or equivalents. Table 14 comprises mild DED markers whose expression is different between a mild DED patient and a “normal” patient, identified in the conditions of the Example and presenting a fold-change, positive or negative, superior or equal to 3.

TABLE 14

| Name of | GeneBank | |
|---|---|---|
| the | Accession | |
| marker | Number | Target sequence (SEQ ID number) |
| CXCL9 | NM_002416.1 | CACCATCTCCCATGAAGAAAGGGAACGGTGAAGTACT |
| | | AAGCGCTAGAGGAAGCAGCCAAGTCGGTTAGTGGAAG |
| | | CATGATTGGTGCCCAGTTAGCCTCTG (SEQ ID NO: 67) |
| LTB4R2 | NM_019839.4 | CAGAGGTCAGTGTTCTGGGACATTTGGGGACCCTTCTT |
| | | TGACTAGAGTTTGGATCTGGCTGGGTAGGATTACTATA |
| | | CACTTGGGGCAGGCCCAGGCTCCT (SEQ ID NO: 75) |
| MAFF | NM_001161572.1 | AGGCCTGAGGTCACATTTCACTTAGTGGTTGTGTTGGG |
| | | ACCAAAACCTGGGTGTCCTCACTGCTGCCCTGAGTCCA |
| | | GCCATGGTTTTCAGGGGGACAGTG (SEQ ID NO: 76) |
| MX2 | NM_002463.1 | AATTTACCGGCTCACTCAGGCGCGACACGCACTCTGTC |
| | | AATTCTCCAGCAAAGAGATCCACTGAAGGGCGGCGAT |
| | | GCCTGTGGTTGTTTTCTTGTGCGTA (SEQ ID NO: 77) |
| NOX1 | NM_007052.4 | TCATTTTGCAGCCGCACACTGAGAAAGCAATTGGATC |
| | | ACAACCTCACCTTCCACAAGCTGGTGGCCTATATGATC |
| | | TGCCTACATACAGCTATTCACATCA (SEQ ID NO: 78) |
| OASL | NM_198213.1 | GGCGTTTCTGAGCTGTTTCCACAGCTTCCAGGAGGCAG |
| | | CCAAGCATCACAAAGATGTTCTGAGGCTGATATGGAA |
| | | AACCATGTGGCAAAGCCAGGACCTG (SEQ ID NO: 80) |

In one embodiment of the invention, the signature of the invention comprises at least 1 mild DED marker, preferably at least 2 mild DED markers selected from the list of Table 11, preferably from the list of Table 12, more preferably from the list of Table 13, even more preferably from Table 14.

In one embodiment, the at least one mild DED marker is selected from the list consisting of CXCL9, CXCR2, HLA-DRA, IFI44, ITGB2, LTB4R2, MAFF, MX2, NOX1, OAS2, OASL and TNFAIP3.

In one embodiment, the at least one mild DED marker is selected from the list consisting of CXCL9, LTB4R2, MAFF, MX2, NOX1 and OASL.

In one embodiment, the at least one mild DED marker of the invention is CXCL9. In one embodiment, the at least one mild DED marker of the invention is CXCR2. In one embodiment, the at least one mild DED marker of the invention is LTB4R2. In another embodiment, the at least one mild DED marker of the invention is MAFF. In one embodiment, the at least one mild DED marker of the invention is MX2. In another embodiment, the at least one mild DED marker of the invention is NOX1. In one embodiment, the at least one mild DED marker of the invention is OASL. In one embodiment, the at least one mild DED marker of the invention is TNFAIP3. In one embodiment, the at least one mild DED marker of the invention is OAS2. In one embodiment, the at least one mild DED marker of the invention is ITGB2. In one embodiment, the at least one mild DED marker of the invention is HLA-DRA. In one embodiment, the at least one mild DED marker of the invention is IFI44.

In one embodiment, the at least one mild DED marker of the invention is selected from the group comprising or consisting of CXCL9, LTB4R2, MAFF, MX2, NOX1 and OASL. In one embodiment, the at least one mild DED marker of the invention is selected from the group comprising or consisting of CXCL9, LTB4R2, MAFF, MX2 and NOX1.

In one embodiment, the at least one mild DED marker of the invention is selected from the group comprising or consisting of CXCL9, MAFF, MX2 and NOX1.

In one embodiment, the signature of the invention comprises 1, 2, 3 or 4 mild DED markers selected from the list of Table 14, preferably CXCL9, MAFF, MX2 and/or NOX1, and 1, 2 or 3 other mild DED markers selected from the list of Table 11, preferably from the list of Table 12, even more preferably from the list of Table 13.

In one embodiment of the invention, the signature of the invention comprises one, or two of the mild DED markers OASL and NOX1. In one embodiment of the invention, the signature of the invention comprises at least the two mild DED markers OASL and NOX1.

In one embodiment, the signature of the invention comprises the mild DED markers CXCL9 and LTB4R2. In another embodiment, the signature of the invention comprises the mild DED markers CXCL9 and MAFF. In another embodiment, the signature of the invention comprises the mild DED markers CXCL9 and MX2. In another embodiment, the signature of the invention comprises the mild DED markers CXCL9 and NOX1. In another embodiment, the signature of the invention comprises the mild DED markers CXCL9 and OASL.

In one embodiment, the signature of the invention comprises the mild DED markers LTB4R2 and MAFF. In another embodiment, the signature of the invention comprises the mild DED markers LTB4R2 and MX2. In another embodiment, the signature of the invention comprises the mild DED markers LTB4R2 and NOX1. In another embodiment, the signature of the invention comprises the mild DED markers LTB4R2 and OASL.

In one embodiment, the signature of the invention comprises the mild DED markers MAFF and MX2. In another embodiment, the signature of the invention comprises the mild DED markers MAFF and NOX1. In another embodiment, the signature of the invention comprises the mild DED markers MAFF and OASL.

In one embodiment, the signature of the invention comprises the mild DED markers MX2 and NOX1. In another embodiment, the signature of the invention comprises the mild DED markers MX2 and OASL.

In one embodiment, the signature of the invention comprises the mild DED markers NOX1 and OASL.

In one embodiment, the signature of the invention comprises the mild DED markers CXCL9, LTB4R2 and MAFF. In another embodiment, the signature of the invention comprises the mild DED markers CXCL9, LTB4R2 and MX2. In another embodiment, the signature of the invention comprises the mild DED markers CXCL9, LTB4R2 and NOX1. In another embodiment, the signature of the invention comprises the mild DED markers CXCL9, LTB4R2 and OASL.

In one embodiment, the signature of the invention comprises the mild DED markers CXCL9, MAFF and MX2. In another embodiment, the signature of the invention comprises the mild DED markers CXCL9, MAFF and NOX1. In another embodiment, the signature of the invention comprises the mild DED markers CXCL9, MAFF and OASL.

In one embodiment, the signature of the invention comprises the mild DED markers CXCL9, MX2 and NOX1. In another embodiment, the signature of the invention comprises the mild DED markers CXCL9, MX2 and OASL.

In one embodiment, the signature of the invention comprises the mild DED markers CXCL9, NOX1 and OASL.

In one embodiment, the signature of the invention comprises the mild DED markers LTB4R2, MAFF and MX2. In another embodiment, the signature of the invention comprises the mild DED markers LTB4R2, MAFF and NOX1. In another embodiment, the signature of the invention comprises the mild DED markers LTB4R2, MAFF and OASL.

In one embodiment, the signature of the invention comprises the mild DED markers LTB4R2, MX2 and NOX1. In another embodiment, the signature of the invention comprises the mild DED markers LTB4R2, MX2 and OASL.

In one embodiment, the signature of the invention comprises the mild DED markers LTB4R2, NOX1 and OASL.

In one embodiment, the signature of the invention comprises the mild DED markers MAFF, MX2 and NOX1. In another embodiment, the signature of the invention comprises the mild DED markers MAFF, MX2 and OASL.

In one embodiment, the signature of the invention comprises the mild DED markers MAFF, NOX1 and OASL.

In one embodiment, the signature of the invention comprises the mild DED markers MX2, NOX1 and OASL.

In one embodiment, the signature of the invention comprises 1, 2, 3 or 4 mild DED markers selected from the list of Table 14, preferably OASL and/or NOX1, and 1, 2 or 3 mild DED markers selected from the list of Table 11, preferably from the list of Table 12, even more preferably from the list of Table 13.

In one embodiment, the signature of the invention comprises 1 mild DED marker selected from the list of Table 14, and 1, 2, 3, 4, or 5 other mild DED markers selected from the list of Table 11, preferably from the list of Table 12, even more preferably from the list of Table 13. In another embodiment, the signature of the invention comprises 2 mild DED markers selected from the list of Table 14, and 1, 2, 3, or 4 other mild DED markers selected from the list of Table 11, preferably from the list of Table 12, even more preferably from the list of Table 13. In another embodiment, the signature of the invention comprises 3 mild DED markers selected from the list of Table 14, and 1, 2, or 3 other mild DED markers selected from the list of Table 11, preferably from the list of Table 12, even more preferably from the list of Table 13.

In one embodiment, mild DED markers are further selected from markers whose expression is different between a mild DED patient and a severe DED patient.

In one embodiment, markers whose expression is different between a mild DED patient and a severe DED patient are markers whose expression is substantially more different between a mild DED patient and a “normal” patient than between a severe DED patient and a “normal” patient. As a non-limitative illustration, a marker whose difference in expression between a mild DED patient and a “normal” patient is 4 fold while difference in expression between a severe DED patient and a “normal” patient is 2 fold is a mild DED marker according to the invention.

In one embodiment, the at least one mild DED marker is selected from the list of the 19 mild DED markers of Table 15 below, as well as their variants, fragments or equivalents. Table 15 comprises mild DED markers whose expression is different between a mild DED patient and a severe DED patient, identified in the conditions of the Example and presenting a fold-change, positive or negative, superior or equal to 1.25.

TABLE 15

| Name of | GeneBank | |
|---|---|---|
| the | Number | |
| marker | Accession | Target sequence (SEQ ID number) |
| CCL22 | NM_002990.3 | CTCGCCCAAGCAGCTGGTAATTCCATTTCATGTATTAG |
| | | ATGTCCCCTGGCCCTCTGTCCCCTCTTAATAACCCTAG |
| | | TCACAGTCTCCGCAGATTCTTGGG (SEQ ID NO: 84) |
| CD40LG | NM_000074.2 | GCATTTGATTTATCAGTGAAGATGCAGAAGGGAAATG |
| | | GGGAGCCTCAGCTCACATTCAGTTATGGTTGACTCTGG |
| | | GTTCCTATGGCCTTGTTGGAGGGGG (SEQ ID NO: 85) |
| CFB | NM_001710.5 | TAAGCTGAAATATGGCCAGACTATCAGGCCCATTTGTC |
| | | TCCCCTGCACCGAGGGAACAACTCGAGCTTTGAGGCT |
| | | TCCTCCAACTACCACTTGCCAGCAA (SEQ ID NO: 86) |
| CXCL10 | NM_001565.1 | GCAGAGGAACCTCCAGTCTCAGCACCATGAATCAAAC |
| | | TGCGATTCTGATTTGCTGCCTTATCTTTCTGACTCTAAG |
| | | TGGCATTCAAGGAGTACCTCTCTC (SEQ ID NO: 87) |
| CXCR2 | NM_001557.2 | AGGAGAAACTGGAACTCTCGAGCGTTGCTGGGGGGGA |
| | | TTGTAAAATGGTGTGACCACTGCAGAAGACAGTATGG |
| | | CAGCTTTCCTCAAAACTTCAGACATA (SEQ ID NO: 68) |
| ELK1 | NM_005229.3 | TTTTCAATAGGGGAGAGGGAGTCATCTCTTCCTATATT |
| | | TGGTGGGGTGGGTGGGAAGGAAGGGATTTGGGGGGG |
| | | AATCTTCCTGCCGCCTCCCCCACTCC (SEQ ID NO: 88) |
| IFIT1 | NM_001548.3 | GAGAAAGGCATTAGATCTGGAAAGCTTGAGCCTCCTT |
| | | GGGTTCGTCTACAAATTGGAAGGAAATATGAATGAAG |
| | | CCCTGGAGTACTATGAGCGGGCCCTG (SEQ ID NO: 89) |
| IFIT3 | NM_001031683.2 | CGCCTGCTAAGGGATGCCCCTTCAGGCATAGGCAGTA |
| | | TTTTCCTGTCAGCATCTGAGCTTGAGGATGGTAGTGAG |
| | | GAAATGGGCCAGGGCGCAGTCAGCT (SEQ ID NO: 90) |
| IL1R1 | NM_000877.2 | CCAGAGAGTGGGGGTGATGATGACCAAGAATTACAAG |
| | | TAGAATGGCAGCTGGAATTTAAGGAGGGACAAGAATC |
| | | AATGGATAAGCGTGGGTGGAGGAAGA (SEQ ID NO: 91) |
| IL22 | NM_020525.4 | CTATCTGATGAAGCAGGTGCTGAACTTCACCCTTGAAG |
| | | AAGTGCTGTTCCCTCAATCTGATAGGTTCCAGCCTTAT |
| | | ATGCAGGAGGTGGTGCCCTTCCTG (SEQ ID NO: 92) |
| IL23A | NM_016584.2 | CAGGGACAACAGTCAGTTCTGCTTGCAAAGGATCCAC |
| | | CAGGGTCTGATTTTTTATGAGAAGCTGCTAGGATCGGA |
| | | TATTTTCACAGGGGAGCCTTCTCTG (SEQ ID NO: 93) |
| LTB4R2 | NM_019839.4 | CAGAGGTCAGTGTTCTGGGACATTTGGGGACCCTTCTT |
| | | TGACTAGAGTTTGGATCTGGCTGGGTAGGATTACTATA |
| | | CACTTGGGGCAGGCCCAGGCTCCT (SEQ ID NO: 75) |
| LY96 | NM_015364.2 | AGAAGTTATTTGCCGAGGATCTGATGACGATTACTCTT |
| | | TTTGCAGAGCTCTGAAGGGAGAGACTGTGAATACAAC |
| | | AATATCATTCTCCTTCAAGGGAATA (SEQ ID NO: 94) |
| MAFK | NM_002360.3 | TGGGTCCCTACAGAGCAAAGGCCTGCGTGTTCCCAAA |
| | | CAGCCGTTGCCCCCGTGGCCGTGGTAGGTAATCCATAT |
| | | TGGATGTCATAAGGACCAGGGGGAT (SEQ ID NO: 95) |
| MKNK1 | NM_003684.3 | TGGAGAACTGAAGATTGCCACCTACGCACAAAGGCCA |
| | | TTGAGACACTTCGTGTAGCTGGAAGACACCAACTTCCT |
| | | GACAGGAGCTTTATTTCATTTGGGA (SEQ ID NO: 96) |
| NOD2 | NM_022162.1 | GGGCCAGAATTTCAAACGGCCTCACTAGGCTTCTGGTT |
| | | GATGCCTGTGAACTGAACTCTGACAACAGACTTCTGA |
| | | AATAGACCCACAAGAGGCAGTTCCA (SEQ ID NO: 97) |
| NOS2 | NM_000625.4 | TTGCCTGGGGTCCATTATGACTCCCAAAAGTTTGACCA |
| | | GAGGACCCAGGGACAAGCCTACCCCTCCAGATGAGCT |
| | | TCTACCTCAAGCTATCGAATTTGTC (SEQ ID NO: 98) |
| TLR6 | NM_006068.2 | TGTCTGGAGGTGCCTCCATTATCCTCATGCCTTCAGGA |
| | | AAGACTTAACAAAAACAATGTTTCATCTGGGGAACTG |
| | | AGCTAGGCGGTGAGGTTAGCCTGCC (SEQ ID NO: 99) |
| TNFAIP3 | NM_006290.2 | CAAAGCCCTCATCGACAGAAACATCCAGGCCACCCTG |
| | | GAAAGCCAGAAGAAACTCAACTGGTGTCGAGAAGTCC |
| | | GGAAGCTTGTGGCGCTGAAAACGAAC (SEQ ID NO: 83) |

In one embodiment, the at least one mild DED marker is selected from the list of the 17 mild DED markers of Table 16 below, as well as their variants, fragments or equivalents. Table 16 comprises mild DED markers whose expression is different between a mild DED patient and a severe DED patient, identified in the conditions of the Example and presenting a fold-change, positive or negative, superior or equal to 1.25.

TABLE 16

| Name of | GeneBank | |
|---|---|---|
| the | Accession | |
| marker | Number | Target sequence (SEQ ID number) |
| CCL22 | NM_002990.3 | CTCGCCCAAGCAGCTGGTAATTCCATTTCATGTATTAG |
| | | ATGTCCCCTGGCCCTCTGTCCCCTCTTAATAACCCTAG |
| | | TCACAGTCTCCGCAGATTCTTGGG (SEQ ID NO: 84) |
| CD40LG | NM_000074.2 | GCATTTGATTTATCAGTGAAGATGCAGAAGGGAAATG |
| | | GGGAGCCTCAGCTCACATTCAGTTATGGTTGACTCTGG |
| | | GTTCCTATGGCCTTGTTGGAGGGGG (SEQ ID NO: 85) |
| CFB | NM_001710.5 | TAAGCTGAAATATGGCCAGACTATCAGGCCCATTTGTC |
| | | TCCCCTGCACCGAGGGAACAACTCGAGCTTTGAGGCT |
| | | TCCTCCAACTACCACTTGCCAGCAA (SEQ ID NO: 86) |
| CXCR2 | NM_001557.2 | AGGAGAAACTGGAACTCTCGAGCGTTGCTGGGGGGGA |
| | | TTGTAAAATGGTGTGACCACTGCAGAAGACAGTATGG |
| | | CAGCTTTCCTCAAAACTTCAGACATA (SEQ ID NO: 68) |
| ELK1 | NM_005229.3 | TTTTCAATAGGGGAGAGGGAGTCATCTCTTCCTATATT |
| | | TGGTGGGGTGGGTGGGAAGGAAGGGATTTGGGGGGG |
| | | AATCTTCCTGCCGCCTCCCCCACTCC (SEQ ID NO: 88) |
| IFIT1 | NM_001548.3 | GAGAAAGGCATTAGATCTGGAAAGCTTGAGCCTCCTT |
| | | GGGTTCGTCTACAAATTGGAAGGAAATATGAATGAAG |
| | | CCCTGGAGTACTATGAGCGGGCCCTG (SEQ ID NO: 89) |
| IFIT3 | NM_001031683.2 | CGCCTGCTAAGGGATGCCCCTTCAGGCATAGGCAGTA |
| | | TTTTCCTGTCAGCATCTGAGCTTGAGGATGGTAGTGAG |
| | | GAAATGGGCCAGGGCGCAGTCAGCT (SEQ ID NO: 90) |
| IL1R1 | NM_000877.2 | CCAGAGAGTGGGGGTGATGATGACCAAGAATTACAAG |
| | | TAGAATGGCAGCTGGAATTTAAGGAGGGACAAGAATC |
| | | AATGGATAAGCGTGGGTGGAGGAAGA (SEQ ID NO: 91) |
| IL22 | NM_020525.4 | CTATCTGATGAAGCAGGTGCTGAACTTCACCCTTGAAG |
| | | AAGTGCTGTTCCCTCAATCTGATAGGTTCCAGCCTTAT |
| | | ATGCAGGAGGTGGTGCCCTTCCTG (SEQ ID NO: 92) |
| LTB4R2 | NM_019839.4 | CAGAGGTCAGTGTTCTGGGACATTTGGGGACCCTTCTT |
| | | TGACTAGAGTTTGGATCTGGCTGGGTAGGATTACTATA |
| | | CACTTGGGGCAGGCCCAGGCTCCT (SEQ ID NO: 75) |
| LY96 | NM_015364.2 | AGAAGTTATTTGCCGAGGATCTGATGACGATTACTCTT |
| | | TTTGCAGAGCTCTGAAGGGAGAGACTGTGAATACAAC |
| | | AATATCATTCTCCTTCAAGGGAATA (SEQ ID NO: 94) |
| MAFK | NM_002360.3 | TGGGTCCCTACAGAGCAAAGGCCTGCGTGTTCCCAAA |
| | | CAGCCGTTGCCCCCGTGGCCGTGGTAGGTAATCCATAT |
| | | TGGATGTCATAAGGACCAGGGGGAT (SEQ ID NO: 95) |
| MKNK1 | NM_003684.3 | TGGAGAACTGAAGATTGCCACCTACGCACAAAGGCCA |
| | | TTGAGACACTTCGTGTAGCTGGAAGACACCAACTTCCT |
| | | GACAGGAGCTTTATTTCATTTGGGA (SEQ ID NO: 96) |
| NOD2 | NM_022162.1 | GGGCCAGAATTTCAAACGGCCTCACTAGGCTTCTGGTT |
| | | GATGCCTGTGAACTGAACTCTGACAACAGACTTCTGA |
| | | AATAGACCCACAAGAGGCAGTTCCA (SEQ ID NO: 97) |
| NOS2 | NM_000625.4 | TTGCCTGGGGTCCATTATGACTCCCAAAAGTTTGACCA |
| | | GAGGACCCAGGGACAAGCCTACCCCTCCAGATGAGCT |
| | | TCTACCTCAAGCTATCGAATTTGTC (SEQ ID NO: 98) |
| TLR6 | NM_006068.2 | TGTCTGGAGGTGCCTCCATTATCCTCATGCCTTCAGGA |
| | | AAGACTTAACAAAAACAATGTTTCATCTGGGGAACTG |
| | | AGCTAGGCGGTGAGGTTAGCCTGCC (SEQ ID NO: 99) |
| TNFAIP3 | NM_006290.2 | CAAAGCCCTCATCGACAGAAACATCCAGGCCACCCTG |
| | | GAAAGCCAGAAGAAACTCAACTGGTGTCGAGAAGTCC |
| | | GGAAGCTTGTGGCGCTGAAAACGAAC (SEQ ID NO: 83) |

In one embodiment, the at least one mild DED marker is selected from the list of the 8 mild DED markers of Table 17 below, as well as their variants, fragments or equivalents. Table 17 comprises mild DED markers whose expression is different between a mild DED patient and a severe DED patient, identified in the conditions of the Example and presenting a fold-change, positive or negative, superior or equal to 2.

TABLE 17

| | GeneBank | |
|---|---|---|
| Name of the | Accession | |
| marker | Number | Target sequence (SEQ ID number) |
| CCL22 | NM_002990.3 | CTCGCCCAAGCAGCTGGTAATTCCATTTCATGTATTAG |
| | | ATGTCCCCTGGCCCTCTGTCCCCTCTTAATAACCCTAG |
| | | TCACAGTCTCCGCAGATTCTTGGG (SEQ ID NO: 84) |
| CXCL10 | NM_001565.1 | GCAGAGGAACCTCCAGTCTCAGCACCATGAATCAAAC |
| | | TGCGATTCTGATTTGCTGCCTTATCTTTCTGACTCTAAG |
| | | TGGCATTCAAGGAGTACCTCTCTC (SEQ ID NO: 87) |
| IFIT1 | NM_001548.3 | GAGAAAGGCATTAGATCTGGAAAGCTTGAGCCTCCTT |
| | | GGGTTCGTCTACAAATTGGAAGGAAATATGAATGAAG |
| | | CCCTGGAGTACTATGAGCGGGCCCTG (SEQ ID NO: 89) |
| IL23A | NM_016584.2 | CAGGGACAACAGTCAGTTCTGCTTGCAAAGGATCCAC |
| | | CAGGGTCTGATTTTTTATGAGAAGCTGCTAGGATCGGA |
| | | TATTTTCACAGGGGAGCCTTCTCTG (SEQ ID NO: 93) |
| LY96 | NM_15364.2 | AGAAGTTATTTGCCGAGGATCTGATGACGATTACTCTT |
| | | TTTGCAGAGCTCTGAAGGGAGAGACTGTGAATACAAC |
| | | AATATCATTCTCCTTCAAGGGAATA (SEQ ID NO: 94) |
| MAFK | NM_002360.3 | TGGGTCCCTACAGAGCAAAGGCCTGCGTGTTCCCAAA |
| | | CAGCCGTTGCCCCCGTGGCCGTGGTAGGTAATCCATAT |
| | | TGGATGTCATAAGGACCAGGGGGAT (SEQ ID NO: 95) |
| NOD2 | NM_022162.1 | GGGCCAGAATTTCAAACGGCCTCACTAGGCTTCTGGTT |
| | | GATGCCTGTGAACTGAACTCTGACAACAGACTTCTGA |
| | | AATAGACCCACAAGAGGCAGTTCCA (SEQ ID NO: 97) |
| NOS2 | NM_000625.4 | TTGCCTGGGGTCCATTATGACTCCCAAAAGTTTGACCA |
| | | GAGGACCCAGGGACAAGCCTACCCCTCCAGATGAGCT |
| | | TCTACCTCAAGCTATCGAATTTGTC (SEQ ID NO: 98) |

In one embodiment, the at least one mild DED marker is selected from the list of the 6 mild DED markers of Table 18 below, as well as their variants, fragments or equivalents. Table 18 comprises mild DED markers whose expression is different between a mild DED patient and a severe DED patient, identified in the conditions of the Example and presenting a fold-change, positive or negative, superior or equal to 2.

TABLE 18

| | GeneBank | |
|---|---|---|
| Name of the | Accession | |
| marker | Number | Target sequence (SEQ ID number) |
| CCL22 | NM_002990.3 | CTCGCCCAAGCAGCTGGTAATTCCATTTCATGTATTAG |
| | | ATGTCCCCTGGCCCTCTGTCCCCTCTTAATAACCCTAG |
| | | TCACAGTCTCCGCAGATTCTTGGG (SEQ ID NO: 84) |
| IFIT1 | NM_001548.3 | GAGAAAGGCATTAGATCTGGAAAGCTTGAGCCTCCTT |
| | | GGGTTCGTCTACAAATTGGAAGGAAATATGAATGAAG |
| | | CCCTGGAGTACTATGAGCGGGCCCTG (SEQ ID NO: 89) |
| LY96 | NM_015364.2 | AGAAGTTATTTGCCGAGGATCTGATGACGATTACTCTT |
| | | TTTGCAGAGCTCTGAAGGGAGAGACTGTGAATACAAC |
| | | AATATCATTCTCCTTCAAGGGAATA (SEQ ID NO: 94) |
| MAFK | NM_002360.3 | TGGGTCCCTACAGAGCAAAGGCCTGCGTGTTCCCAAA |
| | | CAGCCGTTGCCCCCGTGGCCGTGGTAGGTAATCCATAT |
| | | TGGATGTCATAAGGACCAGGGGGAT (SEQ ID NO: 95) |
| NOD2 | NM_022162.1 | GGGCCAGAATTTCAAACGGCCTCACTAGGCTTCTGGTT |
| | | GATGCCTGTGAACTGAACTCTGACAACAGACTTCTGA |
| | | AATAGACCCACAAGAGGCAGTTCCA (SEQ ID NO: 97) |
| NOS2 | NM_000625.4 | TTGCCTGGGGTCCATTATGACTCCCAAAAGTTTGACCA |
| | | GAGGACCCAGGGACAAGCCTACCCCTCCAGATGAGCT |
| | | TCTACCTCAAGCTATCGAATTTGTC (SEQ ID NO: 98) |

In one embodiment, the at least 1 mild DED marker is selected from the list consisting of CCL22, CXCL10, IFIT1, IL23A, LY96, MAFK, NOD2 and NOS2.

In one embodiment, the at least 1 mild DED marker is selected from the list consisting of CCL22, IFIT1, LY96, MAFK, NOD2 and NOS2.

In one embodiment of the invention, the signature of the invention comprises at least 1 mild DED marker, preferably at least 2 markers, selected from the list of Table 15, preferably from the list of Table 16, more preferably from the list of Table 17, even more preferably from the list of Table 18.

In one embodiment, the at least one mild DED marker is selected from the group comprising or consisting of CCL22, CXCL10, IFIT1, IL23A, LY96, MAFK, NOD2 and NOS2. In one embodiment, the at least one mild DED marker is selected from the group comprising or consisting of CCL22, CXCL10, IFIT1, IL23A, MAFK, NOD2 and NOS2. In one embodiment, the at least one mild DED marker is selected from the group comprising or consisting of CCL22, CXCL10, IFIT1, IL23A, LY96, NOD2 and NOS2. In one embodiment, the at least one mild DED marker is selected from the group comprising or consisting of CCL22, CXCL10, IFIT1, IL23A, LY96, MAFK and NOD2. In one embodiment, the at least one mild DED marker is selected from the group comprising or consisting of CCL22, CXCL10, IFIT1, IL23A and NOD2. In one embodiment, the at least one mild DED marker is selected from the group comprising or consisting of CXCL10, IFIT1, IL23A and NOD2. In one embodiment, the at least one mild DED marker is selected from the group comprising or consisting of CXCL10, IFIT1 and NOD2. In one embodiment, the at least one mild DED marker is CCL22. In one embodiment, the at least one mild DED marker is CXCL10. In another embodiment, the at least one mild DED marker is IFIT1. In another embodiment, the at least one mild DED marker is IL23A. In another embodiment, the at least one mild DED marker is LY96. In another embodiment, the at least one mild DED marker is MAFK. In another embodiment, the at least one mild DED marker is NOD2. In another embodiment, the at least one mild DED marker is NOS2.

In one embodiment, the signature of the invention comprises the mild DED markers CCL22 and IFIT1. In another embodiment, the signature of the invention comprises the mild DED markers CCL22 and LY96. In another embodiment, the signature of the invention comprises the mild DED markers CCL22 and MAFK. In another embodiment, the signature of the invention comprises the mild DED markers CCL22 and NOD2. In another embodiment, the signature of the invention comprises the mild DED markers CCL22 and NOS2.

In one embodiment, the signature of the invention comprises the mild DED markers IFIT1 and LY96. In another embodiment, the signature of the invention comprises the mild DED markers IFIT1 and MAFK. In another embodiment, the signature of the invention comprises the mild DED markers IFIT1 and NOD2. In another embodiment, the signature of the invention comprises the mild DED markers IFIT1 and NOS2.

In one embodiment, the signature of the invention comprises the mild DED markers LY96 and MAFK. In another embodiment, the signature of the invention comprises the mild DED markers LY96 and NOD2. In another embodiment, the signature of the invention comprises the mild DED markers LY96 and NOS2.

In one embodiment, the signature of the invention comprises the mild DED markers MAFK and NOD2. In another embodiment, the signature of the invention comprises the mild DED markers MAFK and NOS2.

In one embodiment, the signature of the invention comprises the mild DED markers NOD2 and NOS2.

In one embodiment, the signature of the invention comprises the mild DED markers CCL22, IFIT1 and LY96. In another embodiment, the signature of the invention comprises the mild DED markers CCL22, IFIT1 and MAFK. In another embodiment, the signature of the invention comprises the mild DED markers CCL22, IFIT1 and NOD2. In another embodiment, the signature of the invention comprises the mild DED markers CCL22, IFIT1 and NOS2.

In one embodiment, the signature of the invention comprises the mild DED markers CCL22, LY96 and MAFK. In another embodiment, the signature of the invention comprises the mild DED markers CCL22, LY96 and NOD2. In another embodiment, the signature of the invention comprises the mild DED markers CCL22, LY96 and NOS2.

In one embodiment, the signature of the invention comprises the mild DED markers CCL22, MAFK and NOD2. In another embodiment, the signature of the invention comprises the mild DED markers CCL22, MAFK and NOS2.

In one embodiment, the signature of the invention comprises the mild DED markers CCL22, NOD2 and NOS2.

In one embodiment, the signature of the invention comprises the mild DED markers IFIT1, LY96 and MAFK. In another embodiment, the signature of the invention comprises the mild DED markers IFIT1, LY96 and NOD2. In another embodiment, the signature of the invention comprises the mild DED markers IFIT1, LY96 and NOS2.

In one embodiment, the signature of the invention comprises the mild DED markers IFIT1, MAFK and NOD2. In another embodiment, the signature of the invention comprises the mild DED markers IFIT1, MAFK and NOS2.

In one embodiment, the signature of the invention comprises the mild DED markers IFIT1, NOD2 and NOS2.

In one embodiment, the signature of the invention comprises the mild DED markers LY96, MAFK and NOD2. In another embodiment, the signature of the invention comprises the mild DED markers LY96, MAFK and NOS2.

In one embodiment, the signature of the invention comprises the mild DED markers LY96, NOD2 and NOS2.

In one embodiment, the signature of the invention comprises the mild DED markers MAFK, NOD2 and NOS2.

In one embodiment, the signature of the invention comprises 1, 2, 3 or 4 mild DED markers selected from the list of Table 18, preferably MAFK2, NOD2, CCL22 and/or LY96, and 1, 2 or 3 other mild DED markers selected from the list of Table 15.

In one embodiment of the invention, the signature of the invention comprises one, two, or three of the mild DED markers IL23A, CCL22 and LY96. In one embodiment of the invention, the signature of the invention comprises at least the three mild DED markers IL23A, CCL22 and LY96.

In one embodiment, the signature of the invention comprises 1, 2, 3 or 4 mild DED markers selected from the list of Table 17, preferably IL23A, CCL22 and/or LY96, and 1, 2 or 3 other mild DED markers selected from the list of Table 15.

In one embodiment, the signature of the invention comprises at least one mild DED marker selected from the list of Table 16, and 1, 2, 3, 4, or 5 other mild DED markers selected from the list of Table 15. In another embodiment, the signature of the invention comprises at least 2 mild DED markers selected from the list of Table 16, and 1, 2, 3, or 4 other mild DED markers selected from the list of Table 15. In another embodiment, the signature of the invention comprises at least 3 mild DED markers selected from the list of Table 16, and 1, 2, or 3 other mild DED markers selected from the list of Table 15.

In one embodiment, the signature of the invention comprises at least 1 mild DED marker selected from the list of Table 18, and 1, 2, 3, 4, or 5 other mild DED markers selected from the list of Table 15, 16 or 17. In another embodiment, the signature of the invention comprises at least 2 mild DED markers selected from the list of Table 18, and 1, 2, 3, or 4 other mild DED markers selected from the list of Table 15, 16 or 17. In another embodiment, the signature of the invention comprises at least 3 mild DED markers selected from the list of Table 18, and 1, 2, or 3 other mild DED markers selected from the list of Table 15, 16 or 17.

In one embodiment, the at least one mild DED marker is not OASL. In one embodiment, the at least one mild DED marker is not CXCL9. In one embodiment, the at least one mild DED marker is not CXCL10. In one embodiment, the at least one mild DED marker is not TCF4. In one embodiment, the at least one mild DED marker is not CCL20. In one embodiment, the at least one mild DED marker is not DEFB2. In one embodiment, the at least one mild DED marker is not IFNG. In one embodiment, the at least one mild DED marker is not IL1B. In one embodiment, the at least one mild DED marker is not IL4. In one embodiment, the at least one mild DED marker is not IL6. In one embodiment, the at least one mild DED marker is not IL7. In one embodiment, the at least one mild DED marker is not IL8. In one embodiment, the at least one mild DED marker is not IL15. In one embodiment, the at least one mild DED marker is not IL23 or IL23A. In one embodiment, the at least one mild DED marker is not MMP9. In one embodiment, the at least one mild DED marker is not MUC4. In one embodiment, the at least one mild DED marker is not TGFB1. In one embodiment, the at least one mild DED marker is not TNF.

In one embodiment, the at least one mild DED marker of the invention is selected from the group comprising or consisting of IL23A, TNFAIP3, MAFF, NOS2, ITGB2 and IL7.

In one embodiment, the at least one mild DED marker has at least 100 transcripts per assay, preferably at least 150, more preferably at least 200, even more preferably at least 300.

In one embodiment, the at least one mild DED marker has a coefficient of variation of at most 100, preferably at most 90, more preferably at most 80, more preferably at most 70.

In one embodiment, the at least one mild DED marker has at least 100 transcripts per assay, preferably at least 150, more preferably at least 200, even more preferably at least 300; or has a coefficient of variation of at most 100, preferably at most 90, more preferably at most 80, more preferably at most 70. In one embodiment, the at least one mild DED marker has at least 100 transcripts per assay, preferably at least 150, more preferably at least 200, even more preferably at least 300; and has a coefficient of variation of at most 100, preferably at most 90, more preferably at most 80, more preferably at most 70.

Accordingly, in one embodiment, the at least one mild DED marker of the invention is selected from the group comprising or consisting of TNFAIP3, MAFF and NOS2. In one embodiment, the at least one mild DED marker of the invention is TNFAIP3. In another embodiment, the at least one mild DED marker of the invention is MAFF. In another embodiment, the at least one mild DED marker of the invention is NOS2.

In one embodiment, the signature of the invention comprises severe DED markers.

As used herein, the term “severe DED markers” encompasses markers whose expression is different between a patient suffering from severe DED (i.e. a severe DED patient) and a “normal” patient, and markers whose expression is different between a patient suffering from severe DED (i.e. a severe DED patient) and a patient suffering from mild DED (i.e. a mild DED patient).

The present invention thus also relates to “severe DED markers”. In one embodiment, the signature according to the invention comprises at least one severe DED marker.

In one embodiment of the invention, the signature of the invention comprises 1, 2, 3, 4, 5, 6, 7, 8, 9 or 10 severe DED markers.

In one embodiment, severe DED markers are selected from markers whose expression is different between a severe DED patient and a “normal” patient.

In one embodiment, the at least one severe DED marker is selected from the list of the 12 severe DED markers of Table 19 below, as well as their variants, fragments or equivalents. Table 19 comprises severe DED markers whose expression is different between a severe DED patient and a “normal” patient, identified in the conditions of the Example and presenting a fold-change, positive or negative, superior or equal to 1.25.

TABLE 19

| | GeneBank | |
|---|---|---|
| Name of the | Accession | |
| marker | Number | Target sequence (SEQ ID number) |
| CCL3 | NM_002983.2 | CAGTTCTCTGCATCACTTGCTGCTGACACGCCGACCGC |
| | | CTGCTGCTTCAGCTACACCTCCCGGCAGATTCCACAGA |
| | | ATTTCATAGCTGACTACTTTGAGA (SEQ ID NO: 100) |
| CXCL1 | NM_001511.1 | TATGTTAATATTTCTGAGGAGCCTGCAACATGCCAGCC |
| | | ACTGTGATAGAGGCTGGCGGATCCAAGCAAATGGCCA |
| | | ATGAGATCATTGTGAAGGCAGGGGA (SEQ ID NO: 101) |
| GRB2 | NM_203506.2 | AACTGGTACAAGGCAGAGCTTAATGGAAAAGACGGCT |
| | | TCATTCCCAAGAACTACATAGAAATGAAACCACATCC |
| | | GTTTGGAAACGATGTGCAGCACTTCA (SEQ ID NO: 102) |
| HLA-DRB1 | NM_002124.1 | TCGGCAACTGCAGAAAATGTCCTCCCTTGTGGCTTCCT |
| | | CAGCTCCTGCCCTTGGCCTGAAGTCCCAGCATTGATGA |
| | | CAGCGCCTCATCTTCAACTTTTGT (SEQ ID NO: 103) |
| IL1B | NM_000576.2 | GGGACCAAAGGCGGCCAGGATATAACTGACTTCACCA |
| | | TGCAATTTGTGTCTTCCTAAAGAGAGCTGTACCCAGAG |
| | | AGTCCTGTGCTGAATGTGGACTCAA (SEQ ID NO: 104) |
| IL22RA2 | NM_181309.1 | CACTTGCAACCATGATGCCTAAACATTGCTTTCTAGGC |
| | | TTCCTCATCAGTTTCTTCCTTACTGGTGTAGCAGGAAC |
| | | TCAGTCAACGCATGAGTCTCTGAA (SEQ ID NO: 105) |
| LTB | NM_002341.1 | AGGAACAGGCGTTTCTGACGAGCGGGACGCAGTTCTC |
| | | GGACGCCGAGGGGCTGGCGCTCCCGCAGGACGGCCTC |
| | | TATTACCTCTACTGTCTCGTCGGCTA (SEQ ID NO: 106) |
| MMP9 | NM_004994.2 | CACTACTGTGCCTTTGAGTCCGGTGGACGATGCCTGCA |
| | | ACGTGAACATCTTCGACGCCATCGCGGAGATTGGGAA |
| | | CCAGCTGTATTTGTTCAAGGATGGG (SEQ ID NO: 107) |
| PTGS2 | NM_000963.1 | GCTACAAAAGCTGGGAAGCCTTCTCTAACCTCTCCTAT |
| | | TATACTAGAGCCCTTCCTCCTGTGCCTGATGATTGCCC |
| | | GACTCCCTTGGGTGTCAAAGGTAA (SEQ ID NO: 108) |
| TCF4 | NM_003199.1 | TGGAATCATTGGACCTTCTCATAATGGAGCCATGGGTG |
| | | GTCTGGGCTCAGGGTATGGAACCGGCCTTCTTTCAGCC |
| | | AACAGACATTCACTCATGGTGGGG (SEQ ID NO: 109) |
| TGFB2 | NM_003238.2 | AAGCCAGAGTGCCTGAACAACGGATTGAGCTATATCA |
| | | GATTCTCAAGTCCAAAGATTTAACATCTCCAACCCAGC |
| | | GCTACATCGACAGCAAAGTTGTGAA (SEQ ID NO: 110) |
| TNFSF14 | NM_003807.2 | ATTTTCAGAAGCCTCTGGAAAGTCGTGCACAGCCCAG |
| | | GAGTGTTGAGCAATTTCGGTTTCCTCTGAGGTTGAAGG |
| | | ACCCAGGCGTGTCAGCCCTGCTCCA (SEQ ID NO: 111) |

In another embodiment, the at least one severe DED marker is selected from the list of the 10 severe DED markers of Table 20 below, as well as their variants, fragments or equivalents. Table 20 comprises severe DED markers whose expression is different between a severe DED patient and a “normal” patient, identified in the conditions of the Example and presenting a fold-change, positive or negative, superior or equal to 1.25.

TABLE 20

| | GeneBank | |
|---|---|---|
| Name of the | Accession | |
| marker | Number | Target sequence (SEQ ID number) |
| CCL3 | NM_002983.2 | CAGTTCTCTGCATCACTTGCTGCTGACACGCCGACCGC |
| | | CTGCTGCTTCAGCTACACCTCCCGGCAGATTCCACAGA |
| | | ATTTCATAGCTGACTACTTTGAGA (SEQ ID NO: 100) |
| CXCL1 | NM_001511.1 | TATGTTAATATTTCTGAGGAGCCTGCAACATGCCAGCC |
| | | ACTGTGATAGAGGCTGGCGGATCCAAGCAAATGGCCA |
| | | ATGAGATCATTGTGAAGGCAGGGGA (SEQ ID NO: 101) |
| GRB2 | NM_203506.2 | AACTGGTACAAGGCAGAGCTTAATGGAAAAGACGGCT |
| | | TCATTCCCAAGAACTACATAGAAATGAAACCACATCC |
| | | GTTTGGAAACGATGTGCAGCACTTCA (SEQ ID NO: 102) |
| HLA-DRB1 | NM_002124.1 | TCGGCAACTGCAGAAAATGTCCTCCCTTGTGGCTTCCT |
| | | CAGCTCCTGCCCTTGGCCTGAAGTCCCAGCATTGATGA |
| | | CAGCGCCTCATCTTCAACTTTTGT (SEQ ID NO: 103) |
| IL22RA2 | NM_181309.1 | CACTTGCAACCATGATGCCTAAACATTGCTTTCTAGGC |
| | | TTCCTCATCAGTTTCTTCCTTACTGGTGTAGCAGGAAC |
| | | TCAGTCAACGCATGAGTCTCTGAA (SEQ ID NO: 105) |
| LTB | NM_002341.1 | AGGAACAGGCGTTTCTGACGAGCGGGACGCAGTTCTC |
| | | GGACGCCGAGGGGCTGGCGCTCCCGCAGGACGGCCTC |
| | | TATTACCTCTACTGTCTCGTCGGCTA (SEQ ID NO: 106) |
| PTGS2 | NM_000963.1 | GCTACAAAAGCTGGGAAGCCTTCTCTAACCTCTCCTAT |
| | | TATACTAGAGCCCTTCCTCCTGTGCCTGATGATTGCCC |
| | | GACTCCCTTGGGTGTCAAAGGTAA (SEQ ID NO: 108) |
| TCF4 | NM_003199.1 | TGGAATCATTGGACCTTCTCATAATGGAGCCATGGGTG |
| | | GTCTGGGCTCAGGGTATGGAACCGGCCTTCTTTCAGCC |
| | | AACAGACATTCACTCATGGTGGGG (SEQ ID NO: 109) |
| TGFB2 | NM_003238.2 | AAGCCAGAGTGCCTGAACAACGGATTGAGCTATATCA |
| | | GATTCTCAAGTCCAAAGATTTAACATCTCCAACCCAGC |
| | | GCTACATCGACAGCAAAGTTGTGAA (SEQ ID NO: 110) |
| TNFSF14 | NM_003807.2 | ATTTTCAGAAGCCTCTGGAAAGTCGTGCACAGCCCAG |
| | | GAGTGTTGAGCAATTTCGGTTTCCTCTGAGGTTGAAGG |
| | | ACCCAGGCGTGTCAGCCCTGCTCCA (SEQ ID NO: 111) |

In another embodiment, the at least one severe DED marker is selected from the list of the 6 severe DED markers of Table 21 below, as well as their variants, fragments or equivalents. Table 21 comprises severe DED markers whose expression is different between a severe DED patient and a “normal” patient, identified in the conditions of the Example and presenting a fold-change, positive or negative, superior or equal to 2.

TABLE 21

| | GeneBank | |
|---|---|---|
| Name of the | Accession | |
| marker | Number | Target sequence (SEQ ID number) |
| CCL3 | NM_002983.2 | CAGTTCTCTGCATCACTTGCTGCTGACACGCCGACCGC |
| | | CTGCTGCTTCAGCTACACCTCCCGGCAGATTCCACAGA |
| | | ATTTCATAGCTGACTACTTTGAGA (SEQ ID NO: 100) |
| GRB2 | NM_203506.2 | AACTGGTACAAGGCAGAGCTTAATGGAAAAGACGGCT |
| | | TCATTCCCAAGAACTACATAGAAATGAAACCACATCC |
| | | GTTTGGAAACGATGTGCAGCACTTCA (SEQ ID NO: 102) |
| IL1B | NM_000576.2 | GGGACCAAAGGCGGCCAGGATATAACTGACTTCACCA |
| | | TGCAATTTGTGTCTTCCTAAAGAGAGCTGTACCCAGAG |
| | | AGTCCTGTGCTGAATGTGGACTCAA (SEQ ID NO: 104) |
| TCF4 | NM_003199.1 | TGGAATCATTGGACCTTCTCATAATGGAGCCATGGGTG |
| | | GTCTGGGCTCAGGGTATGGAACCGGCCTTCTTTCAGCC |
| | | AACAGACATTCACTCATGGTGGGG (SEQ ID NO: 109) |
| TGFB2 | NM_003238.2 | AAGCCAGAGTGCCTGAACAACGGATTGAGCTATATCA |
| | | GATTCTCAAGTCCAAAGATTTAACATCTCCAACCCAGC |
| | | GCTACATCGACAGCAAAGTTGTGAA (SEQ ID NO: 110) |
| TNFSF14 | NM_003807.2 | ATTTTCAGAAGCCTCTGGAAAGTCGTGCACAGCCCAG |
| | | GAGTGTTGAGCAATTTCGGTTTCCTCTGAGGTTGAAGG |
| | | ACCCAGGCGTGTCAGCCCTGCTCCA (SEQ ID NO: 111) |

In another embodiment, the at least one severe DED marker is selected from the list of the 5 severe DED markers of Table 22 below, as well as their variants, fragments or equivalents. Table 22 comprises severe DED markers whose expression is different between a severe DED patient and a “normal” patient, identified in the conditions of the Example and presenting a fold-change, positive or negative, superior or equal to 2.

TABLE 22

| | GeneBank | |
|---|---|---|
| Name of the | Accession | |
| marker | Number | Target sequence (SEQ ID number) |
| CCL3 | NM_002983.2 | CAGTTCTCTGCATCACTTGCTGCTGACACGCCGACCGC |
| | | CTGCTGCTTCAGCTACACCTCCCGGCAGATTCCACAGA |
| | | ATTTCATAGCTGACTACTTTGAGA (SEQ ID NO: 100) |
| GRB2 | NM_203506.2 | AACTGGTACAAGGCAGAGCTTAATGGAAAAGACGGCT |
| | | TCATTCCCAAGAACTACATAGAAATGAAACCACATCC |
| | | GTTTGGAAACGATGTGCAGCACTTCA (SEQ ID NO: 102) |
| TCF4 | NM_003199.1 | TGGAATCATTGGACCTTCTCATAATGGAGCCATGGGTG |
| | | GTCTGGGCTCAGGGTATGGAACCGGCCTTCTTTCAGCC |
| | | AACAGACATTCACTCATGGTGGGG (SEQ ID NO: 109) |
| TGFB2 | NM_003238.2 | AAGCCAGAGTGCCTGAACAACGGATTGAGCTATATCA |
| | | GATTCTCAAGTCCAAAGATTTAACATCTCCAACCCAGC |
| | | GCTACATCGACAGCAAAGTTGTGAA (SEQ ID NO: 110) |
| TNFSF14 | NM_003807.2 | ATTTTCAGAAGCCTCTGGAAAGTCGTGCACAGCCCAG |
| | | GAGTGTTGAGCAATTTCGGTTTCCTCTGAGGTTGAAGG |
| | | ACCCAGGCGTGTCAGCCCTGCTCCA (SEQ ID NO: 111) |

In another embodiment, the at least one severe DED marker is selected from the list of the 3 severe DED markers of Table 23 below, as well as their variants, fragments or equivalents. Table 23 comprises severe DED markers whose expression is different between a severe DED patient and a “normal” patient, identified in the conditions of the Example and presenting a fold-change, positive or negative, superior or equal to 2.5.

TABLE 23

| | GeneBank | |
|---|---|---|
| Name of the | Accession | |
| marker | Number | Target sequence (SEQ ID number) |
| GRB2 | NM_203506.2 | AACTGGTACAAGGCAGAGCTTAATGGAAAAGACGGCT |
| | | TCATTCCCAAGAACTACATAGAAATGAAACCACATCC |
| | | GTTTGGAAACGATGTGCAGCACTTCA (SEQ ID NO: 102) |
| TGF32 | NM_003238.2 | AAGCCAGAGTGCCTGAACAACGGATTGAGCTATATCA |
| | | GATTCTCAAGTCCAAAGATTTAACATCTCCAACCCAGC |
| | | GCTACATCGACAGCAAAGTTGTGAA (SEQ ID NO: 110) |
| TNFSF14 | NM_003807.2 | ATTTTCAGAAGCCTCTGGAAAGTCGTGCACAGCCCAG |
| | | GAGTGTTGAGCAATTTCGGTTTCCTCTGAGGTTGAAGG |
| | | ACCCAGGCGTGTCAGCCCTGCTCCA (SEQ ID NO: 111) |

In one embodiment of the invention, the signature of the invention comprises at least 1 severe DED marker, preferably at least 2 markers, selected from the list of Table 19, preferably from the list of Table 20, more preferably from the list of Table 21, even more preferably from the list of Table 22, even more preferably from the list of Table 23.

In one embodiment, the at least one severe DED marker of the invention is CCL22. In another embodiment, the at least one severe DED marker of the invention is CXCL1. In another embodiment, the at least one severe DED marker of the invention is GRB2. In another embodiment, the at least one severe DED marker of the invention is HLA-DRB1. In another embodiment, the at least one severe DED marker of the invention is IL1B. In another embodiment, the at least one severe DED marker of the invention is IL22RA2. In another embodiment, the at least one severe DED marker of the invention is LTB. In another embodiment, the at least one severe DED marker of the invention is PTGS2. In another embodiment, the at least one severe DED marker of the invention is TCF4. In another embodiment, the at least one severe DED marker of the invention is YGFB2. In another embodiment, the at least one severe DED marker of the invention is TNFSF14.

In one embodiment of the invention, the signature of the invention comprises one or two of GRB2 and/or TNFS14. In one embodiment of the invention, the signature of the invention comprises at least the two markers GRB2 and TNFS14. In one embodiment of the invention, the signature of the invention comprises at least GRB2. In another embodiment of the invention, the signature of the invention comprises at least TNFS14.

In one embodiment, the signature of the invention comprises 1, 2, or 3 severe DED markers selected from the list of Table 19, and 1, 2 or 3 other severe DED markers selected from the list of Table 20, 21, 22 or 23.

In one embodiment, the signature of the invention comprises at least 1 severe DED marker selected from the list of Table 19, and 1, 2, 3, 4, or 5 other severe DED markers selected from the list of Table 20, 21, 22 or 23. In another embodiment, the signature of the invention comprises at least 2 severe DED markers selected from the list of Table 19, and 1, 2, 3, or 4 other severe DED markers selected from the list of Table 20, 21, 22 or 23. In another embodiment, the signature of the invention comprises at least 3 severe DED markers of Table 19, and 1, 2, or 3 other severe DED markers selected from the list of Table 20, 21, 22 or 23.

In one embodiment, the signature of the invention comprises 1, 2, or 3 severe DED markers selected from the list of Table 20, and 1, 2 or 3 other severe DED markers selected from the list of Table 19, 21, 22 or 23.

In one embodiment, the signature of the invention comprises at least 1 severe DED marker selected from the list of Table 20, and 1, 2, 3, 4, or 5 other severe DED markers selected from the list of Table 19, 21, 22 or 23. In another embodiment, the signature of the invention comprises at least 2 severe DED markers selected from the list of Table 20, and 1, 2, 3, or 4 other severe DED markers selected from the list of Table 19, 21, 22 or 23. In another embodiment, the signature of the invention comprises at least 3 severe DED markers of Table 20, and 1, 2, or 3 other severe DED markers selected from the list of Table 19, 21, 22 or 23.

In one embodiment, the signature of the invention comprises 1, 2, or 3 severe DED markers selected from the list of Table 22, and 1, 2 or 3 other severe DED markers selected from the list of Table 19, 20, 21 or 23.

In one embodiment, the signature of the invention comprises 1 severe DED marker selected from the list of Table 22, and 1, 2, 3, 4, or 5 other severe DED markers selected from the list of Table 19, 20, 21 or 23. In another embodiment, the signature of the invention comprises 2 severe DED markers selected from the list of Table 22, and 1, 2, 3, or 4 other severe DED markers selected from the list of Table 19, 20, 21 or 23. In another embodiment, the signature of the invention comprises the 3 severe DED markers of Table 22, and 1, 2, or 3 other severe DED markers selected from the list of Table 19, 20, 21 or 23.

In one embodiment, the signature of the invention comprises 1, 2, or 3 severe DED markers selected from the list of Table 23, and 1, 2 or 3 other severe DED markers selected from the list of Table 19, 20, 21 or 22.

In one embodiment, the signature of the invention comprises 1 severe DED marker selected from the list of Table 23, and 1, 2, 3, 4, or 5 other markers selected from the list of Table 19, 20, 21 or 22. In another embodiment, the signature of the invention comprises 2 severe DED markers selected from the list of Table 23, and 1, 2, 3, or 4 other severe DED markers selected from the list of Table 19, 20, 21 or 22. In another embodiment, the signature of the invention comprises the 3 severe DED markers of Table 23, and 1, 2, or 3 other severe DED markers selected from the list of Table 19, 20, 21 or 22.

In one embodiment, the at least one severe DED marker of the invention is CCL3. In one embodiment, the at least one severe DED marker of the invention is CXCL1. In one embodiment, the at least one severe DED marker of the invention is GRB2. In one embodiment, the at least one severe DED marker of the invention is HLA-DRB1. In one embodiment, the at least one severe DED marker of the invention is IL1B. In one embodiment, the at least one severe DED marker of the invention is IL22RA2. In one embodiment, the at least one severe DED marker of the invention is LTB. In one embodiment, the at least one severe DED marker of the invention is PTGS2. In one embodiment, the at least one severe DED marker of the invention is TCF4. In one embodiment, the at least one severe DED marker of the invention is TGFB2. In one embodiment, the at least one severe DED marker of the invention is TNFSF14.

In one embodiment, the signature of the invention comprises the severe DED markers CCL3 and CXCL1. In another embodiment, the signature of the invention comprises the severe DED markers CCL3 and GRB2. In another embodiment, the signature of the invention comprises the severe DED markers CCL3 and HLA-DRB1. In another embodiment, the signature of the invention comprises the severe DED markers CCL3 and IL1B. In another embodiment, the signature of the invention comprises the severe DED markers CCL3 and IL22RA2. In another embodiment, the signature of the invention comprises the severe DED markers CCL3 and LTB. In another embodiment, the signature of the invention comprises the severe DED markers CCL3 and PTGS2. In another embodiment, the signature of the invention comprises the severe DED markers CCL3 and TCF4. In another embodiment, the signature of the invention comprises the severe DED markers CCL3 and TGFB2. In another embodiment, the signature of the invention comprises the severe DED markers CCL3 and TNFSF14.

In one embodiment, the signature of the invention comprises the severe DED markers CXCL1 and GRB2. In another embodiment, the signature of the invention comprises the severe DED markers CXCL1 and HLA-DRB1. In another embodiment, the signature of the invention comprises the severe DED markers CXCL1 and IL1B. In another embodiment, the signature of the invention comprises the severe DED markers CXCL1 and IL22RA2. In another embodiment, the signature of the invention comprises the severe DED markers CXCL1 and LTB. In another embodiment, the signature of the invention comprises the severe DED markers CXCL1 and PTGS2. In another embodiment, the signature of the invention comprises the severe DED markers CXCL1 and TCF4. In another embodiment, the signature of the invention comprises the severe DED markers CXCL1 and TGFB2. In another embodiment, the signature of the invention comprises the severe DED markers CXCL1 and TNFSF14.

In one embodiment, the signature of the invention comprises the severe DED markers GRB2 and HLA-DRB1. In another embodiment, the signature of the invention comprises the severe DED markers GRB2 and IL1B. In another embodiment, the signature of the invention comprises the severe DED markers GRB2 and IL22RA2. In another embodiment, the signature of the invention comprises the severe DED markers GRB2 and LTB. In another embodiment, the signature of the invention comprises the severe DED markers GRB2 and PTGS2. In another embodiment, the signature of the invention comprises the severe DED markers GRB2 and TCF4. In another embodiment, the signature of the invention comprises the severe DED markers GRB2 and TGFB2. In another embodiment, the signature of the invention comprises the severe DED markers GRB2 and TNFSF14.

In one embodiment, the signature of the invention comprises the severe DED markers HLA-DRB1 and IL1B. In another embodiment, the signature of the invention comprises the severe DED markers HLA-DRB1 and IL22RA2. In another embodiment, the signature of the invention comprises the severe DED markers HLA-DRB1 and LTB. In another embodiment, the signature of the invention comprises the severe DED markers HLA-DRB1 and PTGS2. In another embodiment, the signature of the invention comprises the severe DED markers HLA-DRB1 and TCF4. In another embodiment, the signature of the invention comprises the severe DED markers HLA-DRB1 and TGFB2. In another embodiment, the signature of the invention comprises the severe DED markers HLA-DRB1 and TNFSF14.

In one embodiment, the signature of the invention comprises the severe DED markers IL1B and IL22RA2. In another embodiment, the signature of the invention comprises the severe DED markers IL1B and LTB. In another embodiment, the signature of the invention comprises the severe DED markers IL1B and PTGS2. In another embodiment, the signature of the invention comprises the severe DED markers IL1B and TCF4. In another embodiment, the signature of the invention comprises the severe DED markers IL1B and TGFB2. In another embodiment, the signature of the invention comprises the severe DED markers IL and TNFSF14.

In one embodiment, the signature of the invention comprises the severe DED markers IL22RA2 and LTB. In another embodiment, the signature of the invention comprises the severe DED markers IL22RA2 and PTGS2. In another embodiment, the signature of the invention comprises the severe DED markers IL22RA2 and TCF4. In another embodiment, the signature of the invention comprises the severe DED markers IL22RA2 and TGFB2. In another embodiment, the signature of the invention comprises the severe DED markers IL22RA2 and TNFSF14.

In one embodiment, the signature of the invention comprises the severe DED markers LTB and PTGS2. In another embodiment, the signature of the invention comprises the severe DED markers LTB and TCF4. In another embodiment, the signature of the invention comprises the severe DED markers LTB and TGFB2. In another embodiment, the signature of the invention comprises the severe DED markers LTB and TNFSF14.

In one embodiment, the signature of the invention comprises the severe DED markers PTGS2 and TCF4. In another embodiment, the signature of the invention comprises the severe DED markers PTGS2 and TGFB2. In another embodiment, the signature of the invention comprises the severe DED markers PTGS2 and TNFSF14.

In one embodiment, the signature of the invention comprises the severe DED markers TCF4 and TGFB2. In another embodiment, the signature of the invention comprises the severe DED markers TCF4 and TNFSF14.

In one embodiment, the signature of the invention comprises the severe DED markers TGFB2 and TNFSF14.

In one embodiment, the signature of the invention comprises the severe DED markers CCL3, CXCL1 and GRB2. In another embodiment, the signature of the invention comprises the severe DED markers CCL3, CXCL1 and HLA-DRB1. In another embodiment, the signature of the invention comprises the severe DED markers CCL3, CXCL1 and IL1B. In another embodiment, the signature of the invention comprises the severe DED markers CCL3, CXCL1 and IL22RA2. In another embodiment, the signature of the invention comprises the severe DED markers CCL3, CXCL1 and LTB. In another embodiment, the signature of the invention comprises the severe DED markers CCL3, CXCL1 and PTGS2. In another embodiment, the signature of the invention comprises the severe DED markers CCL3, CXCL1 and TCF4. In another embodiment, the signature of the invention comprises the severe DED markers CCL3, CXCL1 and TGFB2. In another embodiment, the signature of the invention comprises the severe DED markers CCL3, CXCL1 and TNFSF14.

In one embodiment, the signature of the invention comprises the severe DED markers CCL3, GRB2 and HLA-DRB1. In another embodiment, the signature of the invention comprises the severe DED markers CCL3, GRB2 and IL1B. In another embodiment, the signature of the invention comprises the severe DED markers CCL3, GRB2 and IL22RA2. In another embodiment, the signature of the invention comprises the severe DED markers CCL3, GRB2 and LTB. In another embodiment, the signature of the invention comprises the severe DED markers CCL3, GRB2 and PTGS2. In another embodiment, the signature of the invention comprises the severe DED markers CCL3, GRB2 and TCF4. In another embodiment, the signature of the invention comprises the severe DED markers CCL3, GRB2 and TGFB2. In another embodiment, the signature of the invention comprises the severe DED markers CCL3, GRB2 and TNFSF14.

In one embodiment, the signature of the invention comprises the severe DED markers CCL3, HLA-DRB1 and IL1B. In another embodiment, the signature of the invention comprises the severe DED markers CCL3, HLA-DRB1 and IL22RA2. In another embodiment, the signature of the invention comprises the severe DED markers CCL3, HLA-DRB1 and LTB. In another embodiment, the signature of the invention comprises the severe DED markers CCL3, HLA-DRB1 and PTGS2. In another embodiment, the signature of the invention comprises the severe DED markers CCL3, HLA-DRB1 and TCF4. In another embodiment, the signature of the invention comprises the severe DED markers CCL3, HLA-DRB1 and TGFB2. In another embodiment, the signature of the invention comprises the severe DED markers CCL3, HLA-DRB1 and TNFSF14.

In one embodiment, the signature of the invention comprises the severe DED markers CCL3, IL1B and IL22RA2. In another embodiment, the signature of the invention comprises the severe DED markers CCL3, IL1B and LTB. In another embodiment, the signature of the invention comprises the severe DED markers CCL3, IL1B and PTGS2. In another embodiment, the signature of the invention comprises the severe DED markers CCL3, IL1B and TCF4. In another embodiment, the signature of the invention comprises the severe DED markers CCL3, IL1B and TGFB2. In another embodiment, the signature of the invention comprises the severe DED markers CCL3, IL1B and TNFSF14.

In one embodiment, the signature of the invention comprises the severe DED markers CCL3, IL22RA2 and LTB. In another embodiment, the signature of the invention comprises the severe DED markers CCL3, IL22RA2 and PTGS2. In another embodiment, the signature of the invention comprises the severe DED markers CCL3, IL22RA2 and TCF4. In another embodiment, the signature of the invention comprises the severe DED markers CCL3, IL22RA2 and TGFB2. In another embodiment, the signature of the invention comprises the severe DED markers CCL3, IL22RA2 and TNFSF14.

In one embodiment, the signature of the invention comprises the severe DED markers CCL3, LTB and PTGS2. In another embodiment, the signature of the invention comprises the severe DED markers CCL3, LTB and TCF4. In another embodiment, the signature of the invention comprises the severe DED markers CCL3, LTB and TGFB2. In another embodiment, the signature of the invention comprises the severe DED markers CCL3, LTB and TNFSF14.

In one embodiment, the signature of the invention comprises the severe DED markers CCL3, PTGS2 and TCF4. In another embodiment, the signature of the invention comprises the severe DED markers CCL3, PTGS2 and TGFB2. In another embodiment, the signature of the invention comprises the severe DED markers CCL3, PTGS2 and TNFSF14.

In one embodiment, the signature of the invention comprises the severe DED markers CCL3, TCF4 and TGFB2. In another embodiment, the signature of the invention comprises the severe DED markers CCL3, TCF4 and TNFSF14.

In one embodiment, the signature of the invention comprises the severe DED markers CCL3, TGFB2 and TNFSF14.

In one embodiment, the signature of the invention comprises the severe DED markers CXCL1, GRB2 and HLA-DRB1. In another embodiment, the signature of the invention comprises the severe DED markers CXCL1, GRB2 and IL1B. In another embodiment, the signature of the invention comprises the severe DED markers CXCL1, GRB2 and IL22RA2. In another embodiment, the signature of the invention comprises the severe DED markers CXCL1, GRB2 and LTB. In another embodiment, the signature of the invention comprises the severe DED markers CXCL1, GRB2 and PTGS2. In another embodiment, the signature of the invention comprises the severe DED markers CXCL1, GRB2 and TCF4. In another embodiment, the signature of the invention comprises the severe DED markers CXCL1, GRB2 and TGFB2. In another embodiment, the signature of the invention comprises the severe DED markers CXCL1, GRB2 and TNFSF14.

In one embodiment, the signature of the invention comprises the severe DED markers CXCL1, HLA-DRB1 and IL1B. In another embodiment, the signature of the invention comprises the severe DED markers CXCL1, HLA-DRB1 and IL22RA2. In another embodiment, the signature of the invention comprises the severe DED markers CXCL1, HLA-DRB1 and LTB. In another embodiment, the signature of the invention comprises the severe DED markers CXCL1, HLA-DRB1 and PTGS2. In another embodiment, the signature of the invention comprises the severe DED markers CXCL1, HLA-DRB1 and TCF4. In another embodiment, the signature of the invention comprises the severe DED markers CXCL1, HLA-DRB1 and TGFB2. In another embodiment, the signature of the invention comprises the severe DED markers CXCL1, HLA-DRB1 and TNFSF14.

In one embodiment, the signature of the invention comprises the severe DED markers CXCL1, IL1B and IL22RA2. In another embodiment, the signature of the invention comprises the severe DED markers CXCL1, IL1B and LTB. In another embodiment, the signature of the invention comprises the severe DED markers CXCL1, IL1B and PTGS2. In another embodiment, the signature of the invention comprises the severe DED markers CXCL1, IL1B and TCF4. In another embodiment, the signature of the invention comprises the severe DED markers CXCL1, IL1B and TGFB2. In another embodiment, the signature of the invention comprises the severe DED markers CXCL1, IL1B and TNFSF14.

In one embodiment, the signature of the invention comprises the severe DED markers CXCL1, IL22RA2 and LTB. In another embodiment, the signature of the invention comprises the severe DED markers CXCL1, IL22RA2 and PTGS2. In another embodiment, the signature of the invention comprises the severe DED markers CXCL1, IL22RA2 and TCF4. In another embodiment, the signature of the invention comprises the severe DED markers CXCL1, IL22RA2 and TGFB2. In another embodiment, the signature of the invention comprises the severe DED markers CXCL1, IL22RA2 and TNFSF14.

In one embodiment, the signature of the invention comprises the severe DED markers CXCL1, LTB and PTGS2. In another embodiment, the signature of the invention comprises the severe DED markers CXCL1, LTB and TCF4. In another embodiment, the signature of the invention comprises the severe DED markers CXCL1, LTB and TGFB2. In another embodiment, the signature of the invention comprises the severe DED markers CXCL1, LTB and TNFSF14.

In one embodiment, the signature of the invention comprises the severe DED markers CXCL1, PTGS2 and TCF4. In another embodiment, the signature of the invention comprises the severe DED markers CXCL1, PTGS2 and TGFB2. In another embodiment, the signature of the invention comprises the severe DED markers CXCL1, PTGS2 and TNFSF14.

In one embodiment, the signature of the invention comprises the severe DED markers CXCL1, TCF4 and TGFB2. In another embodiment, the signature of the invention comprises the severe DED markers CXCL1, TCF4 and TNFSF14.

In one embodiment, the signature of the invention comprises the severe DED markers CXCL1, TGFB2 and TNFSF14.

In one embodiment, the signature of the invention comprises the severe DED markers GRB2, HLA-DRB1 and IL1B. In another embodiment, the signature of the invention comprises the severe DED markers GRB2, HLA-DRB1 and IL22RA2. In another embodiment, the signature of the invention comprises the severe DED markers GRB2, HLA-DRB1 and LTB. In another embodiment, the signature of the invention comprises the severe DED markers GRB2, HLA-DRB1 and PTGS2. In another embodiment, the signature of the invention comprises the severe DED markers GRB2, HLA-DRB1 and TCF4. In another embodiment, the signature of the invention comprises the severe DED markers GRB2, HLA-DRB1 and TGFB2. In another embodiment, the signature of the invention comprises the severe DED markers GRB2, HLA-DRB1 and TNFSF14.

In one embodiment, the signature of the invention comprises the severe DED markers GRB2, IL1B and IL22RA2. In another embodiment, the signature of the invention comprises the severe DED markers GRB2, IL1B and LTB. In another embodiment, the signature of the invention comprises the severe DED markers GRB2, IL1B and PTGS2. In another embodiment, the signature of the invention comprises the severe DED markers GRB2, IL1B and TCF4. In another embodiment, the signature of the invention comprises the severe DED markers GRB2, IL1B and TGFB2. In another embodiment, the signature of the invention comprises the severe DED markers GRB2, IL1B and TNFSF14.

In one embodiment, the signature of the invention comprises the severe DED markers GRB2, IL22RA2 and LTB. In another embodiment, the signature of the invention comprises the severe DED markers GRB2, IL22RA2 and PTGS2. In another embodiment, the signature of the invention comprises the severe DED markers GRB2, IL22RA2 and TCF4. In another embodiment, the signature of the invention comprises the severe DED markers GRB2, IL22RA2 and TGFB2. In another embodiment, the signature of the invention comprises the severe DED markers GRB2, IL22RA2 and TNFSF14.

In one embodiment, the signature of the invention comprises the severe DED markers GRB2, LTB and PTGS2. In another embodiment, the signature of the invention comprises the severe DED markers GRB2, LTB and TCF4. In another embodiment, the signature of the invention comprises the severe DED markers GRB2, LTB and TGFB2. In another embodiment, the signature of the invention comprises the severe DED markers GRB2, LTB and TNFSF14.

In one embodiment, the signature of the invention comprises the severe DED markers GRB2, PTGS2 and TCF4. In another embodiment, the signature of the invention comprises the severe DED markers GRB2, PTGS2 and TGFB2. In another embodiment, the signature of the invention comprises the severe DED markers GRB2, PTGS2 and TNFSF14.

In one embodiment, the signature of the invention comprises the severe DED markers GRB2, TCF4 and TGFB2. In another embodiment, the signature of the invention comprises the severe DED markers GRB2, TCF4 and TNFSF14.

In one embodiment, the signature of the invention comprises the severe DED markers GRB2, TGFB2 and TNFSF14.

In one embodiment, the signature of the invention comprises the severe DED markers HLA-DRB1, IL1B and IL22RA2. In another embodiment, the signature of the invention comprises the severe DED markers HLA-DRB1, IL1B and LTB. In another embodiment, the signature of the invention comprises the severe DED markers HLA-DRB1, IL1B and PTGS2. In another embodiment, the signature of the invention comprises the severe DED markers HLA-DRB1, IL1B and TCF4. In another embodiment, the signature of the invention comprises the severe DED markers HLA-DRB1, IL1B and TGFB2. In another embodiment, the signature of the invention comprises the severe DED markers HLA-DRB1, IL1B and TNFSF14.

In one embodiment, the signature of the invention comprises the severe DED markers HLA-DRB1, IL22RA2 and LTB. In another embodiment, the signature of the invention comprises the severe DED markers HLA-DRB1, IL22RA2 and PTGS2. In another embodiment, the signature of the invention comprises the severe DED markers HLA-DRB1, IL22RA2 and TCF4. In another embodiment, the signature of the invention comprises the severe DED markers HLA-DRB1, IL22RA2 and TGFB2. In another embodiment, the signature of the invention comprises the severe DED markers HLA-DRB1, IL22RA2 and TNFSF14.

In one embodiment, the signature of the invention comprises the severe DED markers HLA-DRB1, LTB and PTGS2. In another embodiment, the signature of the invention comprises the severe DED markers HLA-DRB1, LTB and TCF4. In another embodiment, the signature of the invention comprises the severe DED markers HLA-DRB1, LTB and TGFB2. In another embodiment, the signature of the invention comprises the severe DED markers HLA-DRB1, LTB and TNFSF14.

In one embodiment, the signature of the invention comprises the severe DED markers HLA-DRB1, PTGS2 and TCF4. In another embodiment, the signature of the invention comprises the severe DED markers HLA-DRB1, PTGS2 and TGFB2. In another embodiment, the signature of the invention comprises the severe DED markers HLA-DRB1, PTGS2 and TNFSF14.

In one embodiment, the signature of the invention comprises the severe DED markers HLA-DRB1, TCF4 and TGFB2. In another embodiment, the signature of the invention comprises the severe DED markers HLA-DRB1, TCF4 and TNFSF14.

In one embodiment, the signature of the invention comprises the severe DED markers HLA-DRB1, TGFB2 and TNFSF14.

In one embodiment, the signature of the invention comprises the severe DED markers IL1B, IL22RA2 and LTB. In another embodiment, the signature of the invention comprises the severe DED markers IL1B, IL22RA2 and PTGS2. In another embodiment, the signature of the invention comprises the severe DED markers IL1B, IL22RA2 and TCF4. In another embodiment, the signature of the invention comprises the severe DED markers IL1B, IL22RA2 and TGFB2. In another embodiment, the signature of the invention comprises the severe DED markers IL1B, IL22RA2 and TNFSF14.

In one embodiment, the signature of the invention comprises the severe DED markers IL1B, LTB and PTGS2. In another embodiment, the signature of the invention comprises the severe DED markers IL1B, LTB and TCF4. In another embodiment, the signature of the invention comprises the severe DED markers IL1B, LTB and TGFB2. In another embodiment, the signature of the invention comprises the severe DED markers IL1B, LTB and TNFSF14.

In one embodiment, the signature of the invention comprises the severe DED markers IL1B, PTGS2 and TCF4. In another embodiment, the signature of the invention comprises the severe DED markers IL1B, PTGS2 and TGFB2. In another embodiment, the signature of the invention comprises the severe DED markers IL1B, PTGS2 and TNFSF14.

In one embodiment, the signature of the invention comprises the severe DED markers IL1B, TCF4 and TGFB2. In another embodiment, the signature of the invention comprises the severe DED markers IL1B, TCF4 and TNFSF14.

In one embodiment, the signature of the invention comprises the severe DED markers IL1B, TGFB2 and TNFSF14.

In one embodiment, the signature of the invention comprises the severe DED markers IL22RA2, LTB and PTGS2. In another embodiment, the signature of the invention comprises the severe DED markers IL22RA2, LTB and TCF4. In another embodiment, the signature of the invention comprises the severe DED markers IL22RA2, LTB and TGFB2. In another embodiment, the signature of the invention comprises the severe DED markers IL22RA2, LTB and TNFSF14.

In one embodiment, the signature of the invention comprises the severe DED markers IL22RA2, PTGS2 and TCF4. In another embodiment, the signature of the invention comprises the severe DED markers IL22RA2, PTGS2 and TGFB2. In another embodiment, the signature of the invention comprises the severe DED markers IL22RA2, PTGS2 and TNFSF14.

In one embodiment, the signature of the invention comprises the severe DED markers IL22RA2, TCF4 and TGFB2. In another embodiment, the signature of the invention comprises the severe DED markers IL22RA2, TCF4 and TNFSF14.

In one embodiment, the signature of the invention comprises the severe DED markers IL22RA2, TGFB2 and TNFSF14.

In one embodiment, the signature of the invention comprises the severe DED markers LTB, PTGS2 and TCF4. In another embodiment, the signature of the invention comprises the severe DED markers LTB, PTGS2 and TGFB2. In another embodiment, the signature of the invention comprises the severe DED markers LTB, PTGS2 and TNFSF14.

In one embodiment, the signature of the invention comprises the severe DED markers LTB, TCF4 and TGFB2. In another embodiment, the signature of the invention comprises the severe DED markers LTB, TCF4 and TNFSF14.

In one embodiment, the signature of the invention comprises the severe DED markers LTB, TGFB2 and TNFSF14.

In one embodiment, the signature of the invention comprises the severe DED markers PTGS2, TCF4 and TGFB2. In another embodiment, the signature of the invention comprises the severe DED markers PTGS2, TCF4 and TNFSF14.

In one embodiment, the signature of the invention comprises the severe DED markers PTGS2, TGFB2 and TNFSF14.

In one embodiment, the signature of the invention comprises the severe DED markers TCF4, TGFB2 and TNFSF14.

In one embodiment, severe DED markers are further selected from markers whose expression is different between a severe DED patient and a mild DED patient.

In one embodiment, markers whose expression is different between a severe DED patient and a mild DED patient are markers whose expression is substantially more different between a severe DED patient and a “normal” patient than between a mild DED patient and a “normal” patient. As a non-limitative illustration, a marker whose difference in expression between a severe DED patient and a “normal” patient is 4 fold while difference in expression between a mild DED patient and a “normal” patient is 2 fold is a severe DED marker according to the invention.

In one embodiment, the at least one severe DED marker is selected from the list of the 6 severe DED markers of Table 24 below, as well as their variants, fragments or equivalents. Table 24 comprises severe DED markers whose expression is different between a severe DED patient and a mild DED patient, identified in the conditions of the Example and presenting a fold-change, positive or negative, superior or equal to 1.25.

TABLE 24

| | GeneBank | |
|---|---|---|
| Name of the | Accession | |
| marker | Number | Target sequence (SEQ ID number) |
| CD55 | NM_000574.3 | CCCTACTCCACCCGTCTTGTTTGTCCCACCCTTGGTGA |
| | | CGCAGAGCCCCAGCCCAGACCCCGCCCAAAGCACTCA |
| | | TTTAACTGGTATTGCGGAGCCACGA (SEQ ID NO: 112) |
| CXCL3 | NM_002090.2 | TCCCTGCCCTTACCAGAGCTGAAAATGAAAAAGAGAA |
| | | CAGCAGCTTTCTAGGGACAGCTGGAAAGGACTTAATG |
| | | TGTTTGACTATTTCTTACGAGGGTTC (SEQ ID NO: 113) |
| IL15 | NM_000585.3 | TAGCCAGCCCATACAAGATCGTATTGTATTGTAGGAG |
| | | GCATTGTGGATGGATGGCTGCTGGAAACCCCTTGCCAT |
| | | AGCCAGCTCTTCTTCAATACTTAAG (SEQ ID NO: 114) |
| IL1RN | NM_173842.1 | TCTTCCTGTTCCATTCAGAGACGATCTGCCGACCCTCT |
| | | GGGAGAAAATCCAGCAAGATGCAAGCCTTCAGAATCT |
| | | GGGATGTTAACCAGAAGACCTTCTA (SEQ ID NO: 115) |
| PDGFA | NM_002607.5 | CGTTTGTGGCTGAGTGACAACTTGTTCCCCGCAGTGCA |
| | | CACCTAGAATGCTGTGTTCCCACGCGGCACGTGAGAT |
| | | GCATTGCCGCTTCTGTCTGTGTTGT (SEQ ID NO: 116) |
| PRKCA | NM_002737.2 | CCGGCAGTGCTTCCCTTCCTTTCATCCACTGGCCTCGT |
| | | GTGGTCCATGCAGGGCCACTGTCTGCCCTTTCTGATGC |
| | | CACGTATTAGGCTTTCTTACTCAG (SEQ ID NO: 117) |

In one embodiment, the at least one severe DED marker is selected from the list of the 5 severe DED markers of Table 25 below, as well as their variants, fragments or equivalents. Table 25 comprises severe DED markers whose expression is different between a severe DED patient and a mild DED patient, identified in the conditions of the Example and presenting a fold-change, positive or negative, superior or equal to 1.25.

TABLE 25

| | GeneBank | |
|---|---|---|
| Name of the | Accession | |
| marker | Number | Target sequence (SEQ ID number) |
| CD55 | NM_000574.3 | CCCTACTCCACCCGTCTTGTTTGTCCCACCCTTGGTGA |
| | | CGCAGAGCCCCAGCCCAGACCCCGCCCAAAGCACTCA |
| | | TTTAACTGGTATTGCGGAGCCACGA (SEQ ID NO: 112) |
| CXCL3 | NM_002090.2 | TCCCTGCCCTTACCAGAGCTGAAAATGAAAAAGAGAA |
| | | CAGCAGCTTTCTAGGGACAGCTGGAAAGGACTTAATG |
| | | TGTTTGACTATTTCTTACGAGGGTTC (SEQ ID NO: 113) |
| IL1RN | NM_173842.1 | TCTTCCTGTTCCATTCAGAGACGATCTGCCGACCCTCT |
| | | GGGAGAAAATCCAGCAAGATGCAAGCCTTCAGAATCT |
| | | GGGATGTTAACCAGAAGACCTTCTA (SEQ ID NO: 115) |
| PDGFA | NM_002607.5 | CGTTTGTGGCTGAGTGACAACTTGTTCCCCGCAGTGCA |
| | | CACCTAGAATGCTGTGTTCCCACGCGGCACGTGAGAT |
| | | GCATTGCCGCTTCTGTCTGTGTTGT (SEQ ID NO: 116) |
| PRKCA | NM_002737.2 | CCGGCAGTGCTTCCCTTCCTTTCATCCACTGGCCTCGT |
| | | GTGGTCCATGCAGGGCCACTGTCTGCCCTTTCTGATGC |
| | | CACGTATTAGGCTTTCTTACTCAG (SEQ ID NO: 117) |

In another embodiment, the at least one severe DED marker is selected from the list of the 4 severe DED markers of Table 26 below, as well as their variants, fragments or equivalents. Table 26 comprises severe DED markers whose expression is different between a severe DED patient and a mild DED patient, identified in the conditions of the Example and presenting a fold-change, positive or negative, superior or equal to 3.

TABLE 26

| | GeneBank | |
|---|---|---|
| Name of the | Accession | |
| marker | Number | Target sequence (SEQ ID number) |
| CD55 | NM_000574.3 | CCCTACTCCACCCGTCTTGTTTGTCCCACCCTTGGTGA |
| | | CGCAGAGCCCCAGCCCAGACCCCGCCCAAAGCACTCA |
| | | TTTAACTGGTATTGCGGAGCCACGA (SEQ ID NO: 112) |
| CXCL3 | NM_002090.2 | TCCCTGCCCTTACCAGAGCTGAAAATGAAAAAGAGAA |
| | | CAGCAGCTTTCTAGGGACAGCTGGAAAGGACTTAATG |
| | | TGTTTGACTATTTCTTACGAGGGTTC (SEQ ID NO: 113) |
| IL15 | NM_000585.3 | TAGCCAGCCCATACAAGATCGTATTGTATTGTAGGAG |
| | | GCATTGTGGATGGATGGCTGCTGGAAACCCCTTGCCAT |
| | | AGCCAGCTCTTCTTCAATACTTAAG (SEQ ID NO: 114) |
| PRKCA | NM_002737.2 | CCGGCAGTGCTTCCCTTCCTTTCATCCACTGGCCTCGT |
| | | GTGGTCCATGCAGGGCCACTGTCTGCCCTTTCTGATGC |
| | | CACGTATTAGGCTTTCTTACTCAG (SEQ ID NO: 117) |

In another embodiment, the at least one severe DED marker is selected from the list of the 3 severe DED markers of Table 27 below, as well as their variants, fragments or equivalents. Table 27 comprises severe DED markers whose expression is different between a severe DED patient and a mild DED patient, identified in the conditions of the Example and presenting a fold-change, positive or negative, superior or equal to 3.

TABLE 27

| | GeneBank | |
|---|---|---|
| Name of the | Accession | |
| marker | Number | Target sequence (SEQ ID number) |
| CD55 | NM_000574.3 | CCCTACTCCACCCGTCTTGTTTGTCCCACCCTTGGTGA |
| | | CGCAGAGCCCCAGCCCAGACCCCGCCCAAAGCACTCA |
| | | TTTAACTGGTATTGCGGAGCCACGA (SEQ ID NO: 112) |
| CXCL3 | NM_002090.2 | TCCCTGCCCTTACCAGAGCTGAAAATGAAAAAGAGAA |
| | | CAGCAGCTTTCTAGGGACAGCTGGAAAGGACTTAATG |
| | | TGTTTGACTATTTCTTACGAGGGTTC (SEQ ID NO: 113) |
| PRKCA | NM_002737.2 | CCGGCAGTGCTTCCCTTCCTTTCATCCACTGGCCTCGT |
| | | GTGGTCCATGCAGGGCCACTGTCTGCCCTTTCTGATGC |
| | | CACGTATTAGGCTTTCTTACTCAG (SEQ ID NO: 117) |

In one embodiment of the invention, the signature of the invention comprises at least 1 severe DED marker, preferably at least 2 severe DED markers, selected from the list of Table 24, preferably from the list of Table 25, more preferably from the list of Table 26, even more preferably from the list of Table 27.

In a particular embodiment, the at least one severe DED marker is selected from the list comprising or consisting of CD55, CXCL3 and PRKCA.

In one embodiment, the signature of the invention comprises the severe DED markers CD55 and CXCL3. In another embodiment, the signature of the invention comprises the severe DED markers CD55 and IL15. In another embodiment, the signature of the invention comprises the severe DED markers CD55 and IL1RN. In another embodiment, the signature of the invention comprises the severe DED markers CD55 and PDGFA. In another embodiment, the signature of the invention comprises the severe DED markers CD55 and PRKCA.

In one embodiment, the signature of the invention comprises the severe DED markers CXCL3 and IL15. In another embodiment, the signature of the invention comprises the severe DED markers CXCL3 and IL1RN. In another embodiment, the signature of the invention comprises the severe DED markers CXCL3 and PDGFA. In another embodiment, the signature of the invention comprises the severe DED markers CXCL3 and PRKCA.

In one embodiment, the signature of the invention comprises the severe DED markers IL15 and IL1RN. In another embodiment, the signature of the invention comprises the severe DED markers IL15 and PDGFA. In another embodiment, the signature of the invention comprises the severe DED markers IL15 and PRKCA.

In one embodiment, the signature of the invention comprises the severe DED markers IL1RN and PDGFA. In another embodiment, the signature of the invention comprises the severe DED markers IL1RN and PRKCA.

In one embodiment, the signature of the invention comprises the severe DED markers PDGFA and PRKCA.

In one embodiment, the signature of the invention comprises the severe DED markers CD55, CXCL3 and IL15. In another embodiment, the signature of the invention comprises the severe DED markers CD55, CXCL3 and IL1RN. In another embodiment, the signature of the invention comprises the severe DED markers CD55, CXCL3 and PDGFA. In another embodiment, the signature of the invention comprises the severe DED markers CD55, CXCL3 and PRKCA.

In one embodiment, the signature of the invention comprises the severe DED markers CD55, IL15 and IL1RN. In another embodiment, the signature of the invention comprises the severe DED markers CD55, IL15 and PDGFA. In another embodiment, the signature of the invention comprises the severe DED markers CD55, IL15 and PRKCA.

In one embodiment, the signature of the invention comprises the severe DED markers CD55, IL1RN and PDGFA. In another embodiment, the signature of the invention comprises the severe DED markers CD55, IL1RN and PRKCA.

In one embodiment, the signature of the invention comprises the severe DED markers CD55, PDGFA and PRKCA.

In one embodiment, the signature of the invention comprises the severe DED markers CXCL3, IL15 and IL1RN. In another embodiment, the signature of the invention comprises the severe DED markers CXCL3, IL15 and PDGFA. In another embodiment, the signature of the invention comprises the severe DED markers CXCL3, IL15 and PRKCA.

In one embodiment, the signature of the invention comprises the severe DED markers CXCL3, IL1RN and PDGFA. In another embodiment, the signature of the invention comprises the severe DED markers CXCL3, IL1RN and PRKCA.

In one embodiment, the signature of the invention comprises the severe DED markers CXCL3, PDGFA and PRKCA.

In one embodiment, the signature of the invention comprises the severe DED markers IL15, IL1RN and PDGFA. In another embodiment, the signature of the invention comprises the severe DED markers IL15, IL1RN and PRKCA.

In one embodiment, the signature of the invention comprises the severe DED markers IL15, PDGFA and PRKCA.

In one embodiment, the signature of the invention comprises the severe DED markers IL1RN, PDGFA and PRKCA.

In one embodiment of the invention, the signature of the invention comprises one or two or three of the severe DED markers CD55, CXCL3 and/or PRKCA. In one embodiment of the invention, the signature of the invention comprises at least the severe DED marker CD55. In another embodiment of the invention, the signature of the invention comprises at least the severe DED marker CXCL3. In another embodiment of the invention, the signature of the invention comprises at least the severe DED marker IL15. In another embodiment of the invention, the signature of the invention comprises at least the severe DED marker IL1RN. In another embodiment of the invention, the signature of the invention comprises at least the severe DED marker PDGFA. In another embodiment of the invention, the signature of the invention comprises at least the severe DED marker PRKCA.

In a particular embodiment, the at least one severe DED marker is selected from the list comprising or consisting of CD55, CXCL3, IL15, IL1RN, PDGFA and PRKCA. In a particular embodiment, the at least one severe DED marker is selected from the list comprising or consisting of CD55, CXCL3, IL1RN, PDGFA and PRKCA. In a particular embodiment, the at least one severe DED marker is selected from the list comprising or consisting of CD55, HLA-DRB1, IL1RN and IL15. In a particular embodiment, the at least one severe DED marker is selected from the list comprising or consisting of CD55, CXCL3, PDGFA and IL15. In a particular embodiment, the at least one severe DED marker is selected from the list comprising or consisting of CD55, CXCL3 and PDGFA.

In one embodiment, the at least one severe DED marker has at least 100 transcripts per assay, preferably at least 150, more preferably at least 200, even more preferably at least 300.

In one embodiment, the at least one severe DED marker has a coefficient of variation of at most 100, preferably at most 90, more preferably at most 80, more preferably at most 70.

In one embodiment, the at least one severe DED marker has at least 100 transcripts per assay, preferably at least 150, more preferably at least 200, even more preferably at least 300; or has a coefficient of variation of at most 100, preferably at most 90, more preferably at most 80, more preferably at most 70. In one embodiment, the at least one severe DED marker has at least 100 transcripts per assay, preferably at least 150, more preferably at least 200, even more preferably at least 300; and has a coefficient of variation of at most 100, preferably at most 90, more preferably at most 80, more preferably at most 70.

Accordingly, in one embodiment, the at least one severe DED marker is selected from the list comprising or consisting of HLA-DRB1, IL1RN and IL15. In a preferred embodiment, the at least one severe DED marker is HLA-DRB1, IL1RN or IL15. In one embodiment, the at least one severe DED marker is HLA-DRB1. In one embodiment, the at least one severe DED marker is IL1RN. In one embodiment, the at least one severe DED marker is IL15.

In one embodiment, the signature of the invention comprises 1, 2, or 3 severe DED markers selected from the list of Table 26, and 1, 2 or 3 other severe DED markers selected from the list of Table 24 or 25.

In one embodiment, the signature of the invention comprises 1 severe DED marker selected from the list of Table 27, and 1, 2, 3, 4, or 5 other severe DED markers selected from the list of Table 24, 25 or 26. In another embodiment, the signature of the invention comprises 2 severe DED markers selected from the list of Table 27, and 1, 2, 3, or 4 other severe DED markers selected from the list of Table 24, 25 or 26. In another embodiment, the signature of the invention comprises the 3 severe DED markers of Table 27, and 1, 2, or 3 other severe DED markers selected from the list of Table 24, 25 or 26.

In one embodiment, the at least one severe DED marker is not TCF4. In one embodiment, the at least one severe DED marker is not CXCL10. In one embodiment, the at least one severe DED marker is not CCL20. In one embodiment, the at least one severe DED marker is not DEFB2. In one embodiment, the at least one severe DED marker is not IFNG. In one embodiment, the at least one severe DED marker is not IL1B. In one embodiment, the at least one severe DED marker is not IL4. In one embodiment, the at least one severe DED marker is not IL6. In one embodiment, the at least one severe DED marker is not IL7. In one embodiment, the at least one severe DED marker is not IL8. In one embodiment, the at least one severe DED marker is not IL15. In one embodiment, the at least one severe DED marker is not IL23 or IL23A. In one embodiment, the at least one severe DED marker is not MMP9. In one embodiment, the at least one severe DED marker is not MUC4. In one embodiment, the at least one severe DED marker is not TGFB1. In one embodiment, the at least one severe DED marker is not TNF.

In one embodiment, the signature of the invention is an array or a genechip that includes the genes that are identified as differentially expressed in one or all manifestations of DED, i.e. from mild DED to severe DED, which can be referred to as a “human dry eye disease genechip”. In one embodiment, the array or the genechip of the invention is specific for dry eye disease.

A variety of genechips can be produced that are specific to different aspects of the disease. In one embodiment, a genechip can be produced that includes only those genes that are expressed in mild disease or in severe disease. In a further embodiment, a genechip can be produced with only those genes that are identified as possessing key roles in each aspect of the disease.

In one embodiment, the array or the genechip of the invention comprises at least 3 of the genes selected from the group comprising markers of Table 1 or Table 9, Table 11, Table 15, Table 19 and Table 24 or homologs thereof.

In a particular embodiment, the array or the genechip of the invention comprises at least one marker of Table 1, preferably of Table 2, more preferably of Table 3, even more preferably of Table 4, even more preferably of Table 5, 6, 7 or 8; at least one marker of Table 11, preferably of Table 12, more preferably of Table 13, even more preferably of Table 14; and at least one marker of Table 19, preferably of Table 20, more preferably of Table 21, even more preferably of Table 22, even more preferably of Table 23, or homologs thereof.

In another particular embodiment, the array or the genechip of the invention comprises at least one marker of Table 1, preferably of Table 2, more preferably of Table 3, even more preferably of Table 4, even more preferably of Table 5, 6, 7 or 8; at least one marker of Table 15, preferably of Table 16, even more preferably of Table 17, even more preferably of Table 18; and at least one marker of Table 24, preferably of Table 25, even more preferably of Table 26, even more preferably of Table 27, or homologs thereof.

In another particular embodiment, the array or the genechip of the invention comprises at least one marker of Table 1, preferably of Table 2, more preferably of Table 3, even more preferably of Table 4, even more preferably of Table 5, 6, 7 or 8; at least one marker of Table 11, preferably of Table 12, more preferably of Table 13, even more preferably of Table 14; and at least one marker of Table 24, preferably of Table 25, even more preferably of Table 26, even more preferably of Table 27, or homologs thereof.

In another particular embodiment, the array or the genechip of the invention comprises at least one marker of Table 1, preferably of Table 2, more preferably of Table 3, even more preferably of Table 4, even more preferably of Table 5, 6, 7 or 8; at least one marker of Table 15, preferably of Table 16, even more preferably of Table 17, even more preferably of Table 18; and at least one marker of Table 19, preferably of Table 20, more preferably of Table 21, even more preferably of Table 22, even more preferably of Table 23, or homologs thereof.

In another particular embodiment, the array or the genechip of the invention comprises at least one marker of Table 1, preferably of Table 2, more preferably of Table 3, even more preferably of Table 4, even more preferably of Table 5, 6, 7 or 8; at least one marker of Table 11, preferably of Table 12, more preferably of Table 13, even more preferably of Table 14; at least one marker of Table 15, preferably of Table 16, even more preferably of Table 17, even more preferably of Table 18; at least one marker of Table 19, preferably of Table 20, more preferably of Table 21, even more preferably of Table 22, even more preferably of Table 23; and at least one marker of Table 24, preferably of Table 25, even more preferably of Table 26, even more preferably of Table 27, or homologs thereof.

In a particular embodiment, the array or the genechip of the invention comprises at least one marker of Table 9, preferably of Table 10; at least one marker of Table 11, preferably of Table 12, more preferably of Table 13, even more preferably of Table 14; and at least one marker of Table 19, preferably of Table 20, more preferably of Table 21, even more preferably of Table 22, even more preferably of Table 23, or homologs thereof.

In another particular embodiment, the array or the genechip of the invention comprises at least one marker of Table 9, preferably of Table 10; at least one marker of Table 15, preferably of Table 16, even more preferably of Table 17, even more preferably of Table 18; and at least one marker of Table 24, preferably of Table 25, even more preferably of Table 26, even more preferably of Table 27, or homologs thereof.

In another particular embodiment, the array or the genechip of the invention comprises at least one marker of Table 9, preferably of Table 10; at least one marker of Table 11, preferably of Table 12, more preferably of Table 13, even more preferably of Table 14; and at least one marker of Table 24, preferably of Table 25, even more preferably of Table 26, even more preferably of Table 27, or homologs thereof.

In another particular embodiment, the array or the genechip of the invention comprises at least one marker of Table 9, preferably of Table 10; at least one marker of Table 15, preferably of Table 16, even more preferably of Table 17, even more preferably of Table 18; and at least one marker of Table 19, preferably of Table 20, more preferably of Table 21, even more preferably of Table 22, even more preferably of Table 23, or homologs thereof.

In another particular embodiment, the array or the genechip of the invention comprises at least one marker of Table 9, preferably of Table 10; at least one marker of Table 11, preferably of Table 12, more preferably of Table 13, even more preferably of Table 14; at least one marker of Table 15, preferably of Table 16, even more preferably of Table 17, even more preferably of Table 18; at least one marker of Table 19, preferably of Table 20, more preferably of Table 21, even more preferably of Table 22, even more preferably of Table 23; and at least one marker of Table 24, preferably of Table 25, even more preferably of Table 26, even more preferably of Table 27, or homologs thereof.

The present invention also relates to a signature as hereinabove described, for the identification of DED in a subject.

As used herein, the term “identification of DED in a subject” means identification of a subject suffering from DED. In one embodiment, the term “identification” may be replaced by “determination”, “detection” or “diagnostic”.

The present invention further relates to a signature as hereinabove described, for the prognosis of DED in a subject.

The present invention further relates to a method for the prognosis of DED in a subject, wherein said method comprises assessing the expression of markers in a sample of said subject, whose expressions are different between a DED patient or a “normal” patient.

The present invention also relates to a method for the prognosis of the DED severity in a subject, i.e. for determining if a subject is suffering mild DED or severe DED. In one embodiment, the method of the invention comprises assessing the expression of markers in a sample of said subject, whose expressions are different between a mild DED patient and a “normal” patient or between a mild DED patient and a severe DED patient, and/or the expression of markers whose expressions are different between a severe DED patient and a “normal” patient or between a severe DED patient and a mild DED patient.

In one embodiment, the method of the invention is a non-invasive method.

In one embodiment of the invention, the method of the invention is for determining a personalized course of treatment of the subject. Indeed, according to the prognosis obtained, a personalized treatment may be administered to the subject.

In one embodiment of the invention, the expression of at least 2, preferably of at least 3, more preferably of at least 6 markers is assessed.

In one embodiment, the subject is susceptible or suspected of having DED. In one embodiment, the subject suffer from at least one of the following signs: corneal epithelium alterations stinging, burning or scratchy sensation in the eye, eye redness, sensitivity to light, sensation of having something in the eyes, watery eyes, blurred vision and eye fatigue.

In one embodiment, the subject is at risk of developing DED. In one embodiment, the risks of developing DED are the age; certain medical conditions, including diabetes, rheumatoid arthritis, lupus, scleroderma, Sjögren's syndrome, thyroid disorders and vitamin A deficiency; certain medications, including antihistamines, decongestants, hormone replacement therapy, antidepressants, and drugs for high blood pressure, acne, birth control and Parkinson's disease; laser eye surgery; or tear gland damage from inflammation or radiation.

In one embodiment, the subject is a DED patient. According to this embodiment, the severity of the disease is not yet determined.

Still according to this embodiment, the signature or the method may be for identifying if patients suffer from mild DED or severe DED and thereby identifying the appropriate treatment, such as, for example, artificial tears, anti-inflammatory treatment such as corticosteroids, cyclosporine, and the like.

In one embodiment, the signature or the method may be for identifying if patients suffer from mild DED or severe DED, thereby identifying the appropriate treatment.

In one embodiment, the signature or the method may be for assessing the likelihood of a beneficial response of the patient to a specific anti-DED treatment.

Sjögren's syndrome is an autoimmune disease that attacks and destroys glands responsible for keeping the eyes, mouth and other parts of the body moist and lubricated. For this reason, dry eyes are a common symptom of Sjögren's syndrome.

Because dry eyes are such a distinctive feature of Sjögren's syndrome, many cases of the disease go unreported. It's estimated that 1 in 10 dry eye patients also have Sjögren's syndrome; and it can take up to four years or longer from onset of the disease to get an accurate diagnosis, according to researchers.

Therefore, there is an urgent need to appropriately identify patients with Sjögren's syndrome from patients with dry eye only. As used herein, the term “dry eye only” may be replaced by dry eye without Sjögren's syndrome, or dry eye disease not associated with Sjögren's syndrome.

The present invention also relates to a method for prognosis of dry eye disease in a subject, wherein said dry eye disease is not associated with Sjögren's syndrome (SS). In one embodiment, the method comprises assessing the expression of at least one marker.

Accordingly, in one embodiment, the method according to the invention is for the identification of DED in a patient and for the identification that the patient does not suffer from Sjögren's syndrome.

In one embodiment, the method of the invention is for distinguishing patients with dry eye disease and Sjögren's syndrome from patients with dry eye but not Sjögren's syndrome. In one embodiment, the method of the invention is for the identification of patients with dry eye but not Sjögren's syndrome. In one embodiment, the method of the invention is for the identification of patients with dry eye but not Sjögren's syndrome in a population of DED patients.

In one embodiment, the at least one marker is differently expressed between a DED patient that does not suffer from Sjögren's syndrome (also named DED-SS patient) and a patient suffering from Sjögren's syndrome (also named SS patient).

As used herein, the term “DED-SS marker” refers to a marker whose expression is different between a DED patient that does not suffer from Sjögren's syndrome and a patient suffering from Sjögren's syndrome.

In one embodiment, the genes are identified as differentially expressed in DED-SS, when there is at least about a 1.25 fold difference in expression from SS, preferably at least about a 1.3, 1.4 or 1.5 fold difference. In another embodiment, the genes are identified as differentially expressed in DED-SS, when there is at least about a 2 fold, 2.5 fold, 3 fold, 3.5 fold, 4 fold, 5 fold or more difference in expression from SS.

In one embodiment, the at least one DED-SS marker is selected from the list of Table 28 below, as well as their variants, fragments or equivalents. Table 28 comprises 16 markers whose expression is different between a DED patient that does not suffer from Sjögren's syndrome and a patient suffering from Sjögren's syndrome, identified in the conditions of the Example and presenting a fold-change, positive or negative, superior or equal to 1.2.

TABLE 28

| | GeneBank | |
|---|---|---|
| Name of the | Accession | |
| marker | Number | Target sequence (SEQ ID number) |
| IL8 | NM_000584.2 | ACAGCAGAGCACACAAGCTTCTAGGACAAGAGCCAGG |
| | | AAGAAACCACCGGAAGGAACCATCTCACTGTGTGTAA |
| | | ACATGACTTCCAAGCTGGCCGTGGCT (SEQ ID NO: 30) |
| CCL20 | NM_004591.1 | ATCTGTTCTTTGAGCTAAAAACCATGTGCTGTACCAAG |
| | | AGTTTGCTCCTGGCTGCTTTGATGTCAGTGCTGCTACT |
| | | CCACCTCTGCGGCGAATCAGAAGC (SEQ ID NO: 10) |
| CFD | NM_001928.2 | CTGGTTGGTCTTTATTGAGCACCTACTATATGCAGAAG |
| | | GGGAGGCCGAGGTGGGAGGATCATTGGATCTCAGGAG |
| | | TTCGAGATCAGCATGGGCCACGTAG (SEQ ID NO: 16) |
| GNAQ | NM_002072.2 | AAGGAGTACAATCTGGTCTAATTGTGCCTCCTAGACAC |
| | | CCGCCCTGCCCTTCCCTGGTGGGCTATTGAAGATACAC |
| | | AAGAGGGACTGTATTTCTGTGGAA (SEQ ID NO: 118) |
| PLA2G4A | NM_024420.2 | AACTGGATGCAGCTGTAGCAGATCCTGATGAATTTGA |
| | | GCGAATATATGAGCCTCTGGATGTCAAAAGTAAAAAG |
| | | ATTCATGTAGTGGACAGTGGGCTCAC (SEQ ID NO: 119) |
| CFL1 | NM_005507.2 | TCCGGAAACATGGCCTCCGGTGTGGCTGTCTCTGATGG |
| | | TGTCATCAAGGTGTTCAACGACATGAAGGTGCGTAAG |
| | | TCTTCAACGCCAGAGGAGGTGAAGA (SEQ ID NO: 120) |
| MAP3K5 | NM_005923.3 | TTTAGGAAAAGGCACTTATGGGATAGTCTACGCAGGT |
| | | CGGGACTTGAGCAACCAAGTCAGAATTGCTATTAAGG |
| | | AAATCCCAGAGAGAGACAGCAGATAC (SEQ ID NO: 38) |
| CDC42 | NM_001039802.1 | ACCGACTGTTTTTGACAACTATGCAGTCACAGTTATGA |
| | | TTGGTGGAGAACCATATACTCTTGGACTTTTTGATACT |
| | | GCAGGGCAAGAGGATTATGACAGA (SEQ ID NO: 121) |
| IL10RB | NM_000628.3 | TTCTACCAGATTATGGATGGACTGATCTGAAAATCGAC |
| | | CTCAACTCAAGGGTGGTCAGCTCAATGCTACACAGAG |
| | | CACGGACTTTTGGATTCTTTGCAGT (SEQ ID NO: 31) |
| TLR5 | NM_003268.3 | GTTCCTGGCACTACTACAAGATTCATACTCCTGATGCT |
| | | ACTGACAACGTGGCTTCTCCACAGTCACCAAACCAGG |
| | | GATGCTATACTGGACTTCCCTACTC (SEQ ID NO: 58) |
| SHC1 | NM_001130040.1 | GCGCTCTCTTCCAGAAGATGCCCTCCAATCCTTTCCAC |
| | | CCTATTCCCTAACTCTCGGGACCTCGTTTGGGAGTGTT |
| | | CTGTGGGCTTGGCCTTGTGTCAGA (SEQ ID NO: 122) |
| NOD1 | NM_006092.1 | AGAGGATGCTTTCCTGTTCATGGGGTTTTTGCCCTGGA |
| | | GCCTCAGCAGCAAATGCCACTCTGGGCAGTCTTTTGTG |
| | | TCAGTGTCTTAAAGGGGCCTGCGC (SEQ ID NO: 45) |
| CD4 | NM_000616.3 | AGACATCGTGGTGCTAGCTTTCCAGAAGGCCTCCAGC |
| | | ATAGTCTATAAGAAAGAGGGGGAACAGGTGGAGTTCT |
| | | CCTTCCCACTCGCCTTTACAGTTGAA (SEQ ID NO: 13) |
| IL7 | NM_000880.2 | AATAACCCAGCTTGCGTCCTGCACACTTGTGGCTTCCG |
| | | TGCACACATTAACAACTCATGGTTCTAGCTCCCAGTCG |
| | | CCAAGCGTTGCCAAGGCGTTGAGA (SEQ ID NO: 72) |
| CD55 | NM_000574.3 | CCCTACTCCACCCGTCTTGTTTGTCCCACCCTTGGTGA |
| | | CGCAGAGCCCCAGCCCAGACCCCGCCCAAAGCACTCA |
| | | TTTAACTGGTATTGCGGAGCCACGA (SEQ ID NO: 112) |
| TGFBR1 | NM_004612.2 | GGGGAAATACGACTTAGTGAGGCATAGACATCCCTGG |
| | | TCCATCCTTTCTGTCTCCAGCTGTTTCTTGGAACCTGCT |
| | | CTCCTGCTTGCTGGTCCCTGACGC (SEQ ID NO: 56) |

In another embodiment, the at least one DED-SS marker is selected from the list of 13 markers of Table 29 below, as well as their variants, fragments or equivalents. Table 29 comprises 13 markers whose expression is different between a DED patient that does not suffer from Sjögren's syndrome and a patient suffering from Sjögren's syndrome, identified in the conditions of the Example and presenting a fold-change, positive or negative, superior or equal to 1.2.

TABLE 29

| Name of | GeneBank | |
|---|---|---|
| the | Accession | |
| marker | Number | Target sequence (SEQ ID number) |
| CCL20 | NM_004591.1 | ATCTGTTCTTTGAGCTAAAAACCATGTGCTGTACCAAG |
| | | AGTTTGCTCCTGGCTGCTTTGATGTCAGTGCTGCTACT |
| | | CCACCTCTGCGGCGAATCAGAAGC (SEQ ID NO: 10) |
| CFD | NM_001928.2 | CTGGTTGGTCTTTATTGAGCACCTACTATATGCAGAAG |
| | | GGGAGGCCGAGGTGGGAGGATCATTGGATCTCAGGAG |
| | | TTCGAGATCAGCATGGGCCACGTAG (SEQ ID NO: 16) |
| GNAQ | NM_002072.2 | AAGGAGTACAATCTGGTCTAATTGTGCCTCCTAGACAC |
| | | CCGCCCTGCCCTTCCCTGGTGGGCTATTGAAGATACAC |
| | | AAGAGGGACTGTATTTCTGTGGAA (SEQ ID NO: 118) |
| PLA2G4A | NM_024420.2 | AACTGGATGCAGCTGTAGCAGATCCTGATGAATTTGA |
| | | GCGAATATATGAGCCTCTGGATGTCAAAAGTAAAAAG |
| | | ATTCATGTAGTGGACAGTGGGCTCAC (SEQ ID NO: 119) |
| CFL1 | NM_005507.2 | TCCGGAAACATGGCCTCCGGTGTGGCTGTCTCTGATGG |
| | | TGTCATCAAGGTGTTCAACGACATGAAGGTGCGTAAG |
| | | TCTTCAACGCCAGAGGAGGTGAAGA (SEQ ID NO: 120) |
| MAP3K5 | NM_005923.3 | TTTAGGAAAAGGCACTTATGGGATAGTCTACGCAGGT |
| | | CGGGACTTGAGCAACCAAGTCAGAATTGCTATTAAGG |
| | | AAATCCCAGAGAGAGACAGCAGATAC (SEQ ID NO: 38) |
| CDC42 | NM_001039802.1 | ACCGACTGTTTTTGACAACTATGCAGTCACAGTTATGA |
| | | TTGGTGGAGAACCATATACTCTTGGACTTTTTGATACT |
| | | GCAGGGCAAGAGGATTATGACAGA (SEQ ID NO: 121) |
| IL10RB | NM_000628.3 | TTCTACCAGATTATGGATGGACTGATCTGAAAATCGAC |
| | | CTCAACTCAAGGGTGGTCAGCTCAATGCTACACAGAG |
| | | CACGGACTTTTGGATTCTTTGCAGT (SEQ ID NO: 31) |
| TLR5 | NM_003268.3 | GTTCCTGGCACTACTACAAGATTCATACTCCTGATGCT |
| | | ACTGACAACGTGGCTTCTCCACAGTCACCAAACCAGG |
| | | GATGCTATACTGGACTTCCCTACTC (SEQ ID NO: 58) |
| SHC1 | NM_001130040.1 | GCGCTCTCTTCCAGAAGATGCCCTCCAATCCTTTCCAC |
| | | CCTATTCCCTAACTCTCGGGACCTCGTTTGGGAGTGTT |
| | | CTGTGGGCTTGGCCTTGTGTCAGA (SEQ ID NO: 122) |
| NOD1 | NM_006092.1 | AGAGGATGCTTTCCTGTTCATGGGGTTTTTGCCCTGGA |
| | | GCCTCAGCAGCAAATGCCACTCTGGGCAGTCTTTTGTG |
| | | TCAGTGTCTTAAAGGGGCCTGCGC (SEQ ID NO: 45) |
| CD55 | NM_000574.3 | CCCTACTCCACCCGTCTTGTTTGTCCCACCCTTGGTGA |
| | | CGCAGAGCCCCAGCCCAGACCCCGCCCAAAGCACTCA |
| | | TTTAACTGGTATTGCGGAGCCACGA (SEQ ID NO: 112) |
| TGFBR1 | NM_004612.2 | GGGGAAATACGACTTAGTGAGGCATAGACATCCCTGG |
| | | TCCATCCTTTCTGTCTCCAGCTGTTTCTTGGAACCTGCT |
| | | CTCCTGCTTGCTGGTCCCTGACGC (SEQ ID NO: 56) |

In another embodiment, the at least one DED-SS marker is selected from the list of 10 markers of Table 30 below, as well as their variants, fragments or equivalents. Table 30 comprises 10 markers whose expression is different between a DED patient that does not suffer from Sjögren's syndrome and a patient suffering from Sjögren's syndrome, identified in the conditions of the Example and presenting a fold-change, positive or negative, superior or equal to 1.2.

TABLE 30

| | GeneBank | |
|---|---|---|
| Name of the | Accession | |
| marker | Number | Target sequence (SEQ ID number) |
| CFD | NM_001928.2 | CTGGTTGGTCTTTATTGAGCACCTACTATATGCAGAAG |
| | | GGGAGGCCGAGGTGGGAGGATCATTGGATCTCAGGAG |
| | | TTCGAGATCAGCATGGGCCACGTAG (SEQ ID NO: 16) |
| GNAQ | NM_002072.2 | AAGGAGTACAATCTGGTCTAATTGTGCCTCCTAGACAC |
| | | CCGCCCTGCCCTTCCCTGGTGGGCTATTGAAGATACAC |
| | | AAGAGGGACTGTATTTCTGTGGAA (SEQ ID NO: 118) |
| PLA2G4A | NM_024420.2 | AACTGGATGCAGCTGTAGCAGATCCTGATGAATTTGA |
| | | GCGAATATATGAGCCTCTGGATGTCAAAAGTAAAAAG |
| | | ATTCATGTAGTGGACAGTGGGCTCAC (SEQ ID NO: 119) |
| CFL1 | NM_005507.2 | TCCGGAAACATGGCCTCCGGTGTGGCTGTCTCTGATGG |
| | | TGTCATCAAGGTGTTCAACGACATGAAGGTGCGTAAG |
| | | TCTTCAACGCCAGAGGAGGTGAAGA (SEQ ID NO: 120) |
| CDC42 | NM_001039802.1 | ACCGACTGTTTTTGACAACTATGCAGTCACAGTTATGA |
| | | TTGGTGGAGAACCATATACTCTTGGACTTTTTGATACT |
| | | GCAGGGCAAGAGGATTATGACAGA (SEQ ID NO: 121) |
| SHC1 | NM_001130040.1 | GCGCTCTCTTCCAGAAGATGCCCTCCAATCCTTTCCAC |
| | | CCTATTCCCTAACTCTCGGGACCTCGTTTGGGAGTGTT |
| | | CTGTGGGCTTGGCCTTGTGTCAGA (SEQ ID NO: 122) |
| CD4 | NM_000616.3 | AGACATCGTGGTGCTAGCTTTCCAGAAGGCCTCCAGC |
| | | ATAGTCTATAAGAAAGAGGGGGAACAGGTGGAGTTCT |
| | | CCTTCCCACTCGCCTTTACAGTTGAA (SEQ ID NO: 13) |
| IL7 | NM_000880.2 | AATAACCCAGCTTGCGTCCTGCACACTTGTGGCTTCCG |
| | | TGCACACATTAACAACTCATGGTTCTAGCTCCCAGTCG |
| | | CCAAGCGTTGCCAAGGCGTTGAGA (SEQ ID NO: 72) |
| CD55 | NM_000574.3 | CCCTACTCCACCCGTCTTGTTTGTCCCACCCTTGGTGA |
| | | CGCAGAGCCCCAGCCCAGACCCCGCCCAAAGCACTCA |
| | | TTTAACTGGTATTGCGGAGCCACGA (SEQ ID NO: 112) |
| TGFBR1 | NM_004612.2 | GGGGAAATACGACTTAGTGAGGCATAGACATCCCTGG |
| | | TCCATCCTTTCTGTCTCCAGCTGTTTCTTGGAACCTGCT |
| | | CTCCTGCTTGCTGGTCCCTGACGC (SEQ ID NO: 56) |

In another embodiment, the at least one DED-SS marker is selected from the list of 7 markers of Table 31 below, as well as their variants, fragments or equivalents. Table 31 comprises 6 DED-SS markers having at least 100 transcripts per assay or having a coefficient of variation of at most 100.

TABLE 31

| | GeneBank | |
|---|---|---|
| Name of the | Accession | |
| marker | Number | Target sequence (SEQ ID number) |
| GNAQ | NM_002072.2 | AAGGAGTACAATCTGGTCTAATTGTGCCTCCTAGACAC |
| | | CCGCCCTGCCCTTCCCTGGTGGGCTATTGAAGATACAC |
| | | AAGAGGGACTGTATTTCTGTGGAA (SEQ ID NO: 118) |
| PLA2G4A | NM_024420.2 | AACTGGATGCAGCTGTAGCAGATCCTGATGAATTTGA |
| | | GCGAATATATGAGCCTCTGGATGTCAAAAGTAAAAAG |
| | | ATTCATGTAGTGGACAGTGGGCTCAC (SEQ ID NO: 119) |
| CFL1 | NM_005507.2 | TCCGGAAACATGGCCTCCGGTGTGGCTGTCTCTGATGG |
| | | TGTCATCAAGGTGTTCAACGACATGAAGGTGCGTAAG |
| | | TCTTCAACGCCAGAGGAGGTGAAGA (SEQ ID NO: 120) |
| CDC42 | NM_001039802.1 | ACCGACTGTTTTTGACAACTATGCAGTCACAGTTATGA |
| | | TTGGTGGAGAACCATATACTCTTGGACTTTTTGATACT |
| | | GCAGGGCAAGAGGATTATGACAGA (SEQ ID NO: 121) |
| SHC1 | NM_001130040.1 | GCGCTCTCTTCCAGAAGATGCCCTCCAATCCTTTCCAC |
| | | CCTATTCCCTAACTCTCGGGACCTCGTTTGGGAGTGTT |
| | | CTGTGGGCTTGGCCTTGTGTCAGA (SEQ ID NO: 122) |
| TGFBR1 | NM_004612.2 | GGGGAAATACGACTTAGTGAGGCATAGACATCCCTGG |
| | | TCCATCCTTTCTGTCTCCAGCTGTTTCTTGGAACCTGCT |
| | | CTCCTGCTTGCTGGTCCCTGACGC (SEQ ID NO: 56) |

In another embodiment, the at least one DED-SS marker is selected from the list of 5 markers of Table 32 below, as well as their variants, fragments or equivalents. Table 32 comprises 5 DED-SS markers having at least 100 transcripts per assay or having a coefficient of variation of at most 100.

TABLE 32

| | GeneBank | |
|---|---|---|
| Name of the | Accession | |
| marker | Number | Target sequence (SEQ ID number) |
| CCL20 | NM_004591.1 | ATCTGTTCTTTGAGCTAAAAACCATGTGCTGTACCAAG |
| | | AGTTTGCTCCTGGCTGCTTTGATGTCAGTGCTGCTACT |
| | | CCACCTCTGCGGCGAATCAGAAGC (SEQ ID NO: 10) |
| CFD | NM_001928.2 | CTGGTTGGTCTTTATTGAGCACCTACTATATGCAGAAG |
| | | GGGAGGCCGAGGTGGGAGGATCATTGGATCTCAGGAG |
| | | TTCGAGATCAGCATGGGCCACGTAG (SEQ ID NO: 16) |
| GNAQ | NM_002072.2 | AAGGAGTACAATCTGGTCTAATTGTGCCTCCTAGACAC |
| | | CCGCCCTGCCCTTCCCTGGTGGGCTATTGAAGATACAC |
| | | AAGAGGGACTGTATTTCTGTGGAA (SEQ ID NO: 118) |
| CD55 | NM_000574.3 | CCCTACTCCACCCGTCTTGTTTGTCCCACCCTTGGTGA |
| | | CGCAGAGCCCCAGCCCAGACCCCGCCCAAAGCACTCA |
| | | TTTAACTGGTATTGCGGAGCCACGA (SEQ ID NO: 112) |
| TGFBR1 | NM_004612.2 | GGGGAAATACGACTTAGTGAGGCATAGACATCCCTGG |
| | | TCCATCCTTTCTGTCTCCAGCTGTTTCTTGGAACCTGCT |
| | | CTCCTGCTTGCTGGTCCCTGACGC (SEQ ID NO: 56) |

In one embodiment, the method comprises assessing the expression of at least one marker, preferably at least two markers, more preferably at least three markers. In one embodiment, the method comprises assessing the expression of at least one DED-SS marker of Table 28, preferably of Table 29, more preferably of Table 30, even more preferably of Table 31 or 32. In another embodiment, the method comprises assessing the expression of at least two DED-SS markers of Table 28, preferably of Table 29, more preferably of Table 30, even more preferably of Table 31 or 32. In another embodiment, the method comprises assessing the expression of at least three DED-SS markers of Table 28, preferably of Table 29, more preferably of Table 30, even more preferably of Table 31 or 32.

In one embodiment, the at least one DED-SS marker is selected from the group comprising or consisting of CCL220, CD55, CFD, GNAQ, PLA2G4A, CFL1, CDC42, SHC1, and TGFBR1. In one embodiment, the at least one DED-SS marker is selected from the group comprising or consisting of GNAQ, PLA2G4A, CFL1, CDC42, SHC1, and TGFBR1. In one embodiment, the at least one DED-SS marker is selected from the group comprising or consisting of CCL20, CFD, GNAQ, CD55 and TGFBR1.

In one embodiment, the at least one DED-SS marker is CCL20. In one embodiment, the at least one DED-SS marker is CD55. In one embodiment, the at least one DED-SS marker is CFD. In one embodiment, the at least one DED-SS marker is GNAQ. In one embodiment, the at least one DED-SS marker is PLA2G4A. In one embodiment, the at least one DED-SS marker is CFL1. In one embodiment, the at least one DED-SS marker is CDC42. In one embodiment, the at least one DED-SS marker is SHC1. In one embodiment, the at least one DED-SS marker is TGFBR1. In one embodiment, the at least one DED-SS marker is NOD1. In one embodiment, the at least one DED-SS marker is CD4.

In one embodiment, the at least two DED-SS markers are CCL20 and CD4. In another embodiment, the at least two DED-SS markers are CCL20 and CD55. In another embodiment, the at least two DED-SS markers are CCL20 and CFD. In another embodiment, the at least two DED-SS markers are CCL20 and GNAQ. In another embodiment, the at least two DED-SS markers are CCL20 and PLA2G4A. In another embodiment, the at least two DED-SS markers are CCL20 and CFL1. In another embodiment, the at least two DED-SS markers are CCL20 and CDC42. In another embodiment, the at least two DED-SS markers are CCL20 and SHC1. In another embodiment, the at least two DED-SS markers are CCL20 and TGFBR1.

In one embodiment, the at least two DED-SS markers are CD4 and CD55. In another embodiment, the at least two DED-SS markers are CD4 and CFD. In another embodiment, the at least two DED-SS markers are CD4 and GNAQ. In another embodiment, the at least two DED-SS markers are CD4 and PLA2G4A. In another embodiment, the at least two DED-SS markers are CD4 and CFL1. In another embodiment, the at least two DED-SS markers are CD4 and CDC42. In another embodiment, the at least two DED-SS markers are CD4 and SHC1. In another embodiment, the at least two DED-SS markers are CD4 and TGFBR1.

In one embodiment, the at least two DED-SS markers are CD55 and CFD. In another embodiment, the at least two DED-SS markers are CD55 and GNAQ. In another embodiment, the at least two DED-SS markers are CD55 and PLA2G4A. In another embodiment, the at least two DED-SS markers are CD55 and CFL1. In another embodiment, the at least two DED-SS markers are CD55 and CDC42. In another embodiment, the at least two DED-SS markers are CD55 and SHC1. In another embodiment, the at least two DED-SS markers are CD55 and TGFBR1.

In one embodiment, the at least two DED-SS markers are CFD and GNAQ. In another embodiment, the at least two DED-SS markers are CFD and PLA2G4A. In another embodiment, the at least two DED-SS markers are CFD and CFL1. In another embodiment, the at least two DED-SS markers are CFD and CDC42. In another embodiment, the at least two DED-SS markers are CFD and SHC1. In another embodiment, the at least two DED-SS markers are CFD and TGFBR1.

In one embodiment, the at least two DED-SS markers are GNAQ and PLA2G4A. In another embodiment, the at least two DED-SS markers are GNAQ and CFL1. In another embodiment, the at least two DED-SS markers are GNAQ and CDC42. In another embodiment, the at least two DED-SS markers are GNAQ and SHC1. In another embodiment, the at least two DED-SS markers are GNAQ and TGFBR1.

In one embodiment, the at least two DED-SS markers are PLA2G4A and CFL1. In another embodiment, the at least two DED-SS markers are PLA2G4A and CDC42. In another embodiment, the at least two DED-SS markers are PLA2G4A and SHC1. In another embodiment, the at least two DED-SS markers are PLA2G4A and TGFBR1.

In one embodiment, the at least two DED-SS markers are CFL1 and CDC42. In another embodiment, the at least two DED-SS markers are CFL1 and SHC1. In another embodiment, the at least two DED-SS markers are CFL1 and TGFBR1.

In one embodiment, the at least two DED-SS markers are CDC42 and SHC1. In another embodiment, the at least two DED-SS markers are CDC42 and TGFBR1.

In one embodiment, the at least two DED-SS markers are SHC1 and TGFBR1.

Moreover, it is also important to consider the level of expression of the genes and their coefficient of variation. Indeed, genes with higher mean expression levels are more easily detected than those with lower levels of expression. Thus, among the selected genes, the ones with higher expression levels (for instance, those with mean expression levels of more than 100 transcripts per assay) are preferred to increase the sensitivity of the assay.

In one embodiment, the at least one DED-SS marker has at least 100 transcripts per assay, preferably at least 150, more preferably at least 200, even more preferably at least 300.

The coefficient of variation (the ratio of the standard deviation to the mean) is a measure of the dispersion within a group. The CV (%) is an indication of the homogeneity within a group (of patients for example). Therefore, genes with lower CVs are preferred.

In one embodiment, the at least one DED-SS marker has a coefficient of variation of at most 100, preferably at most 90, more preferably at most 80, more preferably at most 70.

In one embodiment, the at least one DED-SS marker has at least 100 transcripts per assay, preferably at least 150, more preferably at least 200, even more preferably at least 300; or has a coefficient of variation of at most 100, preferably at most 90, more preferably at most 80, more preferably at most 70. In one embodiment, the at least one DED-SS marker has at least 100 transcripts per assay, preferably at least 150, more preferably at least 200, even more preferably at least 300; and has a coefficient of variation of at most 100, preferably at most 90, more preferably at most 80, more preferably at most 70.

Accordingly, in one embodiment, the at least one DED-SS marker is selected from the group comprising or consisting of GNAQ, PLA2G4A, CFL1, CDC42, SHC1, CD4 and TGFBR1. In another embodiment, the at least one DED-SS marker is selected from the group comprising or consisting of GNAQ, PLA2G4A, SHC1, CD4 and TGFBR1. In another embodiment, the at least one DED-SS marker is selected from the group comprising or consisting of GNAQ, PLA2G4A, CD4 and TGFBR1.

The present invention also relates to a method for the identification of the severity of DED, wherein said dry eye disease is not associated with Sjögren's syndrome (SS).

In one embodiment, the method of the invention is for distinguishing patients with mild, moderate and/or severe dry eye disease in a population of patients that do not suffer from Sjögren's syndrome.

In one embodiment, the method of the invention is for the identification of the severity of DED in a patient that does not suffer from Sjögren's syndrome. In one embodiment, the method of the invention comprises assessing the expression of at least one marker.

In one embodiment, the at least one marker correlates with at least one other test for dry eye. Dry eye disease is currently assessed by several tests, such as for example questionnaire OSDI (Ocular Surface Disease Index), breakup time (BUT), Schirmer test, osmolarity, and corneal fluorescein staining (CFS).

In one embodiment, the severity of DED in patients that do not suffer from SS may be assessed by the correlation between at least one marker as described hereinabove and at least one dry eye test. In a preferred embodiment, the severity of DED in patients that do not suffer from SS may be assessed by the correlation between at least one marker as described hereinabove and at least one dry eye test selected from the group comprising or consisting of OSDI, BUT, Schirmer test, osmolarity and CFS, preferably selected from the group comprising or consisting of OSDI, BUT and CFS.

In one embodiment, the method of the invention comprises assessing the expression of at least one marker, preferably of at least two markers, more preferably of at least three markers.

In one embodiment, the at least one marker for the identification of severity of DED in patients that do not suffer from SS is selected from the group comprising or consisting of CFD, GNAQ, PLA2G4A, CDC42, SHC1, CD4, IL7, CD55 and TGFBR1. In a particular embodiment, the at least one marker for the identification of severity of DED in patients that do not suffer from SS is selected from the group comprising or consisting of CFD, GNAQ, PLA2G4A and CDC42. In a particular embodiment, the at least one marker for the identification of severity of DED in patients that do not suffer from SS is selected from the group comprising or consisting of SHC1, CD4, IL7, CD55 and TGFBR1.

In a preferred embodiment, the at least one marker for the identification of severity of DED in patients that do not suffer from SS is CFD. In a preferred embodiment, the at least one marker for the identification of severity of DED in patients that do not suffer from SS is SHC1. In a preferred embodiment, the at least one marker for the identification of severity of DED in patients that do not suffer from SS is CD55.

The present invention further relates to a method for prognosis of Sjögren's syndrome (SS). In one embodiment, the method comprises assessing the expression of at least one marker.

Accordingly, in one embodiment, the method according to the invention is for the identification of Sjögren's syndrome in a patient.

In one embodiment, the method of the invention is for distinguishing patients with Sjögren's syndrome from healthy subjects. In one embodiment, the at least one marker is differently expressed between a patient with Sjögren's syndrome (also named SS patient) and a healthy subject.

As used herein, the term “SS marker” refers to a marker whose expression is different between a SS patient and a healthy subject.

In one embodiment, the genes are identified as differentially expressed in SS, when there is at least about a 1.25 fold difference in expression from healthy subject, preferably at least about a 1.3, 1.4 or 1.5 fold difference. In another embodiment, the genes are identified as differentially expressed in SS, when there is at least about a 2 fold, 2.5 fold, 3 fold, 3.5 fold, 4 fold, 5 fold or more difference in expression from healthy subject.

In one embodiment, the at least one SS marker is selected from the list of Table 33 below, as well as their variants, fragments or equivalents. Table 33 comprises markers whose expression is different between a patient suffering from Sjögren's syndrome and a healthy subject, identified in the conditions of the Example and presenting a fold-change, positive or negative, superior or equal to 1.2, a p-value inferior to 0.05.

TABLE 33

| | GeneBank | |
|---|---|---|
| Name of the | Accession | Target sequence (SEQ ID number) |
| marker | Number | |
| IL6 | NM_000600.1 | TGACAAACAAATTCGGTACATCCTCGACGGCATCTCA |
| | | GCCCTGAGAAAGGAGACATGTAACAAGAGTAACATGT |
| | | GTGAAAGCAGCAAAGAGGCACTGGCA (SEQ ID NO: 28) |
| IL23A | NM_016584.2 | CAGGGACAACAGTCAGTTCTGCTTGCAAAGGATCCAC |
| | | CAGGGTCTGATTTTTTATGAGAAGCTGCTAGGATCGGA |
| | | TATTTTCACAGGGGAGCCTTCTCTG (SEQ ID NO: 93) |
| CCR1 | NM_001295.2 | CATCATTTGGGCCCTGGCCATCTTGGCTTCCATGCCAG |
| | | GCTTATACTTTTCCAAGACCCAATGGGAATTCACTCAC |
| | | CACACCTGCAGCCTTCACTTTCCT (SEQ ID NO: 123) |
| CCL4 | NM_002984.2 | TTCTGCAGCCTCACCTCTGAGAAAACCTCTTTGCCACC |
| | | AATACCATGAAGCTCTGCGTGACTGTCCTGTCTCTCCT |
| | | CATGCTAGTAGCTGCCTTCTGCTC (SEQ ID NO: 8) |
| TNFAIP3 | NM_006290.2 | CAAAGCCCTCATCGACAGAAACATCCAGGCCACCCTG |
| | | GAAAGCCAGAAGAAACTCAACTGGTGTCGAGAAGTCC |
| | | GGAAGCTTGTGGCGCTGAAAACGAAC (SEQ ID NO: 83) |
| MAFF | NM_001161572.1 | AGGCCTGAGGTCACATTTCACTTAGTGGTTGTGTTGGG |
| | | ACCAAAACCTGGGTGTCCTCACTGCTGCCCTGAGTCCA |
| | | GCCATGGTTTTCAGGGGGACAGTG (SEQ ID NO: 76) |
| NOS2 | NM_000625.4 | TTGCCTGGGGTCCATTATGACTCCCAAAAGTTTGACCA |
| | | GAGGACCCAGGGACAAGCCTACCCCTCCAGATGAGCT |
| | | TCTACCTCAAGCTATCGAATTTGTC (SEQ ID NO: 98) |
| ITGB2 | NM_000211.2 | CATCGACCTGTACTATCTGATGGACCTCTCCTACTCCA |
| | | TGCTTGATGACCTCAGGAATGTCAAGAAGCTAGGTGG |
| | | CGACCTGCTCCGGGCCCTCAACGAG (SEQ ID NO: 74) |
| HLA-DRB1 | NM_002124.1 | TCGGCAACTGCAGAAAATGTCCTCCCTTGTGGCTTCCT |
| | | CAGCTCCTGCCCTTGGCCTGAAGTCCCAGCATTGATGA |
| | | CAGCGCCTCATCTTCAACTTTTGT (SEQ ID NO: 103) |
| CXCL2 | NM_002089.3 | ATCACATGTCAGCCACTGTGATAGAGGCTGAGGAATC |
| | | CAAGAAAATGGCCAGTGAGATCAATGTGACGGCAGGG |
| | | AAATGTATGTGTGTCTATTTTGTAAC (SEQ ID NO: 124) |
| STAT1 | NM_007315.2 | TTTGCTGTATGCCATCCTCGAGAGCTGTCTAGGTTAAC |
| | | GTTCGCACTCTGTGTATATAACCTCGACAGTCTTGGCA |
| | | CCTAACGTGCTGTGCGTAGCTGCT (SEQ ID NO: 52) |
| IL1RN | NM_173842.1 | TCTTCCTGTTCCATTCAGAGACGATCTGCCGACCCTCT |
| | | GGGAGAAAATCCAGCAAGATGCAAGCCTTCAGAATCT |
| | | GGGATGTTAACCAGAAGACCTTCTA (SEQ ID NO: 115) |
| IL15 | NM_000585.3 | TAGCCAGCCCATACAAGATCGTATTGTATTGTAGGAG |
| | | GCATTGTGGATGGATGGCTGCTGGAAACCCCTTGCCAT |
| | | AGCCAGCTCTTCTTCAATACTTAAG (SEQ ID NO: 114) |
| LTB4R2 | NM_019839.4 | CAGAGGTCAGTGTTCTGGGACATTTGGGGACCCTTCTT |
| | | TGACTAGAGTTTGGATCTGGCTGGGTAGGATTACTATA |
| | | CACTTGGGGCAGGCCCAGGCTCCT (SEQ ID NO: 75) |
| GNGT1 | NM_021955.3 | AAGAAGTAAGAGATTACGTTGAAGAACGATCTGGCGA |
| | | GGATCCACTGGTAAAGGGCATCCCAGAGGACAAAAAT |
| | | CCCTTCAAGGAGCTCAAAGGAGGCTG (SEQ ID NO: 21) |
| HSPB2 | NM_001541.3 | AGCTGCTCTCTCCCATGATGGCATCTTAAACCTGGAAG |
| | | CACCTCGGGGTGGCCGACATTTGGACACAGAGGTCAA |
| | | TGAGGTCTACATCTCCCTGCTCCCT (SEQ ID NO: 24) |
| HMGB2 | NM_001130688.1 | CTGTCAACATGGGTAAAGGAGACCCCAACAAGCCGCG |
| | | GGGCAAAATGTCCTCGTACGCCTTCTTCGTGCAGACCT |
| | | GCCGGGAAGAGCACAAGAAGAAACA (SEQ ID NO: 23) |
| KEAP1 | NM_012289.3 | CTCATTGAATTCGCCTACACGGCCTCCATCTCCATGGG |
| | | CGAGAAGTGTGTCCTCCACGTCATGAACGGTGCTGTC |
| | | ATGTACCAGATCGACAGCGTTGTCC (SEQ ID NO: 125) |

In another embodiment, the at least one SS marker is selected from the list of markers of Table 34 below, as well as their variants, fragments or equivalents. Table 34 comprises markers whose expression is different between a patient suffering from Sjögren's syndrome and a healthy subject, identified in the conditions of the Example and presenting a fold-change, positive or negative, superior or equal to 1.2, a p-value inferior to 0.05.

TABLE 34

| | GeneBank | |
|---|---|---|
| Name of the | Accession | |
| marker | Number | Target sequence (SEQ ID number) |
| IL23A | NM_016584.2 | CAGGGACAACAGTCAGTTCTGCTTGCAAAGGATCCAC |
| | | CAGGGTCTGATTTTTTATGAGAAGCTGCTAGGATCGGA |
| | | TATTTTCACAGGGGAGCCTTCTCTG (SEQ ID NO: 93) |
| CCR1 | NM_001295.2 | CATCATTTGGGCCCTGGCCATCTTGGCTTCCATGCCAG |
| | | GCTTATACTTTTCCAAGACCCAATGGGAATTCACTCAC |
| | | CACACCTGCAGCCTTCACTTTCCT (SEQ ID NO: 123) |
| CCL4 | NM_002984.2 | TTCTGCAGCCTCACCTCTGAGAAAACCTCTTTGCCACC |
| | | AATACCATGAAGCTCTGCGTGACTGTCCTGTCTCTCCT |
| | | CATGCTAGTAGCTGCCTTCTGCTC (SEQ ID NO: 8) |
| MAFF | NM_001161572.1 | AGGCCTGAGGTCACATTTCACTTAGTGGTTGTGTTGGG |
| | | ACCAAAACCTGGGTGTCCTCACTGCTGCCCTGAGTCCA |
| | | GCCATGGTTTTCAGGGGGACAGTG (SEQ ID NO: 76) |
| NOS2 | NM_000625.4 | TTGCCTGGGGTCCATTATGACTCCCAAAAGTTTGACCA |
| | | GAGGACCCAGGGACAAGCCTACCCCTCCAGATGAGCT |
| | | TCTACCTCAAGCTATCGAATTTGTC (SEQ ID NO: 98) |
| ITGB2 | NM_000211.2 | CATCGACCTGTACTATCTGATGGACCTCTCCTACTCCA |
| | | TGCTTGATGACCTCAGGAATGTCAAGAAGCTAGGTGG |
| | | CGACCTGCTCCGGGCCCTCAACGAG (SEQ ID NO: 74) |
| CXCL2 | NM_002089.3 | ATCACATGTCAGCCACTGTGATAGAGGCTGAGGAATC |
| | | CAAGAAAATGGCCAGTGAGATCAATGTGACGGCAGGG |
| | | AAATGTATGTGTGTCTATTTTGTAAC (SEQ ID NO: 124) |
| STAT1 | NM_007315.2 | TTTGCTGTATGCCATCCTCGAGAGCTGTCTAGGTTAAC |
| | | GTTCGCACTCTGTGTATATAACCTCGACAGTCTTGGCA |
| | | CCTAACGTGCTGTGCGTAGCTGCT (SEQ ID NO: 52) |
| IL1RN | NM_173842.1 | TCTTCCTGTTCCATTCAGAGACGATCTGCCGACCCTCT |
| | | GGGAGAAAATCCAGCAAGATGCAAGCCTTCAGAATCT |
| | | GGGATGTTAACCAGAAGACCTTCTA (SEQ ID NO: 15) |
| LTB4R2 | NM_019839.4 | CAGAGGTCAGTGTTCTGGGACATTTGGGGACCCTTCTT |
| | | TGACTAGAGTTTGGATCTGGCTGGGTAGGATTACTATA |
| | | CACTTGGGGCAGGCCCAGGCTCCT (SEQ ID NO: 75) |
| GNGT1 | NM_021955.3 | AAGAAGTAAGAGATTACGTTGAAGAACGATCTGGCGA |
| | | GGATCCACTGGTAAAGGGCATCCCAGAGGACAAAAAT |
| | | CCCTTCAAGGAGCTCAAAGGAGGCTG (SEQ ID NO: 21) |
| HSPB2 | NM_001541.3 | AGCTGCTCTCTCCCATGATGGCATCTTAAACCTGGAAG |
| | | CACCTCGGGGTGGCCGACATTTGGACACAGAGGTCAA |
| | | TGAGGTCTACATCTCCCTGCTCCCT (SEQ ID NO: 24) |
| HMGB2 | NM_001130688.1 | CTGTCAACATGGGTAAAGGAGACCCCAACAAGCCGCG |
| | | GGGCAAAATGTCCTCGTACGCCTTCTTCGTGCAGACCT |
| | | GCCGGGAAGAGCACAAGAAGAAACA (SEQ ID NO: 23) |
| KEAP1 | NM_012289.3 | CTCATTGAATTCGCCTACACGGCCTCCATCTCCATGGG |
| | | CGAGAAGTGTGTCCTCCACGTCATGAACGGTGCTGTC |
| | | ATGTACCAGATCGACAGCGTTGTCC (SEQ ID NO: 125) |

In another embodiment, the at least one SS marker is selected from the list of markers of Table 35 below, as well as their variants, fragments or equivalents. Table 35 comprises markers whose expression is different between a patient suffering from Sjögren's syndrome and a healthy subject, identified in the conditions of the Example and presenting a fold-change, positive or negative, superior or equal to 1.5 and a p-value inferior to 0.05.

TABLE 35

| | GeneBank | |
|---|---|---|
| Name of the | Accession | |
| marker | Number | Target sequence (SEQ ID number) |
| IL23A | NM_016584.2 | CAGGGACAACAGTCAGTTCTGCTTGCAAAGGATCCAC |
| | | CAGGGTCTGATTTTTTATGAGAAGCTGCTAGGATCGGA |
| | | TATTTTCACAGGGGAGCCTTCTCTG (SEQ ID NO: 93) |
| CCR1 | NM_001295.2 | CATCATTTGGGCCCTGGCCATCTTGGCTTCCATGCCAG |
| | | GCTTATACTTTTCCAAGACCCAATGGGAATTCACTCAC |
| | | CACACCTGCAGCCTTCACTTTCCT (SEQ ID NO: 123) |
| CCL4 | NM_002984.2 | TTCTGCAGCCTCACCTCTGAGAAAACCTCTTTGCCACC |
| | | AATACCATGAAGCTCTGCGTGACTGTCCTGTCTCTCCT |
| | | CATGCTAGTAGCTGCCTTCTGCTC (SEQ ID NO: 8) |
| MAFF | NM_001161572.1 | AGGCCTGAGGTCACATTTCACTTAGTGGTTGTGTTGGG |
| | | ACCAAAACCTGGGTGTCCTCACTGCTGCCCTGAGTCCA |
| | | GCCATGGTTTTCAGGGGGACAGTG (SEQ ID NO: 76) |
| NOS2 | NM_000625.4 | TTGCCTGGGGTCCATTATGACTCCCAAAAGTTTGACCA |
| | | GAGGACCCAGGGACAAGCCTACCCCTCCAGATGAGCT |
| | | TCTACCTCAAGCTATCGAATTTGTC (SEQ ID NO: 98) |
| ITGB2 | NM_000211.2 | CATCGACCTGTACTATCTGATGGACCTCTCCTACTCCA |
| | | TGCTTGATGACCTCAGGAATGTCAAGAAGCTAGGTGG |
| | | CGACCTGCTCCGGGCCCTCAACGAG (SEQ ID NO: 74) |
| LTB4R2 | NM_019839.4 | CAGAGGTCAGTGTTCTGGGACATTTGGGGACCCTTCTT |
| | | TGACTAGAGTTTGGATCTGGCTGGGTAGGATTACTATA |
| | | CACTTGGGGCAGGCCCAGGCTCCT (SEQ ID NO: 75) |
| GNGT1 | NM_021955.3 | AAGAAGTAAGAGATTACGTTGAAGAACGATCTGGCGA |
| | | GGATCCACTGGTAAAGGGCATCCCAGAGGACAAAAAT |
| | | CCCTTCAAGGAGCTCAAAGGAGGCTG (SEQ ID NO: 21) |
| HSPB2 | NM_001541.3 | AGCTGCTCTCTCCCATGATGGCATCTTAAACCTGGAAG |
| | | CACCTCGGGGTGGCCGACATTTGGACACAGAGGTCAA |
| | | TGAGGTCTACATCTCCCTGCTCCCT (SEQ ID NO: 24) |

In another embodiment, the at least one SS marker is selected from the list of markers of Table 36 below, as well as their variants, fragments or equivalents. Table 36 comprises markers whose expression is different between a patient suffering from Sjögren's syndrome and a healthy subject, identified in the conditions of the Example and presenting a fold-change, positive or negative, superior or equal to 1.8 and a p-value inferior to 0.05.

TABLE 36

| | GeneBank | |
|---|---|---|
| Name of the | Accession | |
| marker | Number | Target sequence (SEQ ID number) |
| IL23A | NM_016584.2 | CAGGGACAACAGTCAGTTCTGCTTGCAAAGGATCCAC |
| | | CAGGGTCTGATTTTTTATGAGAAGCTGCTAGGATCGGA |
| | | TATTTTCACAGGGGAGCCTTCTCTG (SEQ ID NO: 93) |
| CCR1 | NM_001295.2 | CATCATTTGGGCCCTGGCCATCTTGGCTTCCATGCCAG |
| | | GCTTATACTTTTCCAAGACCCAATGGGAATTCACTCAC |
| | | CACACCTGCAGCCTTCACTTTCCT (SEQ ID NO: 123) |
| CCL4 | NM_002984.2 | TTCTGCAGCCTCACCTCTGAGAAAACCTCTTTGCCACC |
| | | AATACCATGAAGCTCTGCGTGACTGTCCTGTCTCTCCT |
| | | CATGCTAGTAGCTGCCTTCTGCTC (SEQ ID NO: 8) |
| LTB4R2 | NMO19839.4 | CAGAGGTCAGTGTTCTGGGACATTTGGGGACCCTTCTT |
| | | TGACTAGAGTTTGGATCTGGCTGGGTAGGATTACTATA |
| | | CACTTGGGGCAGGCCCAGGCTCCT (SEQ ID NO: 75) |
| GNGT1 | NM_021955.3 | AAGAAGTAAGAGATTACGTTGAAGAACGATCTGGCGA |
| | | GGATCCACTGGTAAAGGGCATCCCAGAGGACAAAAAT |
| | | CCCTTCAAGGAGCTCAAAGGAGGCTG (SEQ ID NO: 21) |

In one embodiment, the method comprises assessing the expression of at least one SS marker, preferably at least two SS markers, more preferably at least three SS markers.

In one embodiment, the method comprises assessing the expression of at least one SS marker of Table 33, preferably of Table 34, more preferably of Table 35, even more preferably of Table 36. In another embodiment, the method comprises assessing the expression of at least two SS markers of Table 33, preferably of Table 34, more preferably of Table 35, even more preferably of Table 36. In another embodiment, the method comprises assessing the expression of at least three SS markers of Table 33, preferably of Table 34, more preferably of Table 35, even more preferably of Table 36.

In one embodiment, the at least one SS marker is selected from the group comprising or consisting of IL23A, CCR1, CCL4, GNGT1, LTB4R2, HSPB2, MAFF, NOS2, ITGB2, CXCL2, STAT1, IL1RN, HMGB2 and KEAP1. In one embodiment, the at least one SS marker is selected from the group comprising or consisting of IL23A, CCR1, CCL4, GNGT1 and LTB4R2. In one embodiment, the at least one SS marker is selected from the group comprising or consisting of IL23A, CCR1, CCL4, GNGT1 and HSPB2. In another embodiment, the at least one SS marker is selected from the group comprising or consisting of IL23A, CCR1, and CCL4. In another embodiment, the at least one SS marker is selected from the group comprising or consisting of LTB4R2, GNGT1 and HSPB2. In another embodiment, the at least one SS marker is selected from the group comprising or consisting of IL23A, CCR1 and GNGT1.

In one embodiment, the at least one SS marker is IL23A. In another embodiment, the at least one SS marker is CCR1. In another embodiment, the at least one SS marker is CCL4. In another embodiment, the at least one SS marker is GNGT1. In another embodiment, the at least one SS marker is HSPB2. In another embodiment, the at least one SS marker is LTB4R2. In another embodiment, the at least one SS marker is MAFF. In another embodiment, the at least one SS marker is NOS2. In another embodiment, the at least one SS marker is ITGB2. In another embodiment, the at least one SS marker is CXCL2. In another embodiment, the at least one SS marker is STAT1. In another embodiment, the at least one SS marker is IL1RN. In another embodiment, the at least one SS marker is HMGB2. In another embodiment, the at least one SS marker is KEAP1.

In one embodiment, the at least two SS markers are IL23A and CCR1. In another embodiment, the at least two SS markers are IL23A and CCL4. In another embodiment, the at least two SS markers are IL23A and LTB4R2. In another embodiment, the at least two SS markers are IL23A and GNGT1. In another embodiment, the at least two SS markers are IL23A and HSPB2.

In one embodiment, the at least two SS markers are CCR1 and CCL4. In another embodiment, the at least two SS markers are CCR1 and LTB4R2. In another embodiment, the at least two SS markers are CCR1 and GNGT1. In another embodiment, the at least two SS markers are CCR1 and HSPB2.

In one embodiment, the at least two SS markers are CCL4 and LTB4R2. In another embodiment, the at least two SS markers are CCL4 and GNGT1. In another embodiment, the at least two SS markers are CCL4 and HSPB2.

In one embodiment, the at least two SS markers are LTB4R2 and GNGT1. In another embodiment, the at least two SS markers are LTB4R2 and HSPB2.

In one embodiment, the at least two SS markers are GNGT1 and HSPB2.

In one embodiment, the at least one SS marker is not IL6. In one embodiment, the at least one SS marker is not IL1B. In one embodiment, the at least one SS marker is not IL8. In one embodiment, the at least one SS marker is not MMP9. In one embodiment, the at least one SS marker is not TNF.

In one embodiment, the at least one SS marker has at least 100 transcripts per assay, preferably at least 150, more preferably at least 200, even more preferably at least 300; or has a coefficient of variation of at most 100, preferably at most 90, more preferably at most 80, more preferably at most 70. In one embodiment, the at least one SS marker has at least 100 transcripts per assay, preferably at least 150, more preferably at least 200, even more preferably at least 300; and has a coefficient of variation of at most 100, preferably at most 90, more preferably at most 80, more preferably at most 70.

Accordingly, in one embodiment, the at least one SS marker is selected from the group comprising or consisting of TNFAIP3, MAFF, NOS2, HLA-DRB1, CXCL2, STAT1, IL1RN, IL15, GNGT1, HMGB2 and KEAP1. In another embodiment, the at least one SS marker is selected from the group comprising or consisting of MAFF, NOS2, HLA-DRB1, CXCL2, STAT1, IL1RN, GNGT1, HMGB2 and KEAP1. In another embodiment, the at least one SS marker is selected from the group comprising or consisting of NOS2, CXCL2, STAT1, IL1RN, GNGT1 and KEAP1. In another embodiment, the at least one SS marker is selected from the group comprising or consisting of STAT1, IL1RN and KEAP1.

In one embodiment, the at least one SS marker is TNFAIP3. In another embodiment, the at least one SS marker is MAFF. In another embodiment, the at least one SS marker is NOS2. In another embodiment, the at least one SS marker is HLA-DRB1. In another embodiment, the at least one SS marker is CXCL2. In another embodiment, the at least one SS marker is STAT1. In another embodiment, the at least one SS marker is IL1RN. In another embodiment, the at least one SS marker is IL15. In another embodiment, the at least one SS marker is GNGT1. In another embodiment, the at least one SS marker is HMGB2. In another embodiment, the at least one SS marker is KEAP1.

The present invention also relates to a method for the identification of the severity of Sjögren's syndrome (SS) in a patient.

In one embodiment, the method of the invention is for distinguishing patients with mild, moderate and/or severe Sjögren's syndrome.

In one embodiment, the method of the invention comprises assessing the expression of at least one marker. In one embodiment, the at least one marker correlates with at least one other test for dry eye. Dry eye disease is currently assessed by several tests, such as for example questionnaire OSDI (Ocular Surface Disease Index), breakup time (BUT), Schirmer test, osmolarity, and corneal fluorescein staining (CFS).

In one embodiment, the severity of SS in patients may be assessed by the correlation between at least one marker as described hereinabove and at least one dry eye test. In a preferred embodiment, the severity of SS in patients may be assessed by the correlation between at least one marker as described hereinabove and at least one dry eye test selected from the group comprising or consisting of OSDI, BUT, Schirmer test, osmolarity and CFS, preferably selected from the group comprising or consisting of OSDI, BUT and CFS.

In one embodiment, the method of the invention comprises assessing the expression of at least one marker, preferably of at least two markers, more preferably of at least three markers.

In one embodiment, the at least one marker for the identification of severity of SS in a patient is selected from the group comprising or consisting of IL6, CCR1, CCL4, MAFF, NOS2, ITGB2, HLA-DRB1, CXCL2, STAT1, IL1RN, IL15, GNGT1 and HSPB2. In a particular embodiment, the at least one marker for the identification of severity of SS in a patient is selected from the group comprising or consisting of CCR1, CCL4, MAFF, NOS2, ITGB2, CXCL2, STAT1, GNGT1 and HSPB2. In another particular embodiment, the at least one marker for the identification of severity of SS in a patient is selected from the group comprising or consisting of CCR1, STAT1, GNGT1 and HSPB2.

In a preferred embodiment, the at least one marker for the identification of severity of SS in a patient is IL6. In another preferred embodiment, the at least one marker for the identification of severity of SS in a patient is CCR1. In another preferred embodiment, the at least one marker for the identification of severity of SS in a patient is STAT1. In another preferred embodiment, the at least one marker for the identification of severity of SS in a patient is GNGT1. In another preferred embodiment, the at least one marker for the identification of severity of SS in a patient is HSPB2.

In a preferred embodiment, the at least one marker for the identification of severity of SS in a patient is not IL6. In a preferred embodiment, the at least one marker for the identification of severity of SS in a patient is not IL1B. In a preferred embodiment, the at least one marker for the identification of severity of SS in a patient is not IL8. In a preferred embodiment, the at least one marker for the identification of severity of SS in a patient is not MMP9. In a preferred embodiment, the at least one marker for the identification of severity of SS in a patient is not TNF.

In one embodiment, the subject of the invention is elderly. As used herein, the term “elderly” means that the subject is at least 50 years old, at least 55, 60, 65, 70, 75, 80, 85 or 90 years old.

In one embodiment, the subject is a woman. In another embodiment, the subject is a man.

In one embodiment of the invention, the method of the invention for the prognosis of DED, the prognosis of the severity of DED, the prognosis of DED not associated with Sjögren's syndrome, the prognosis of the severity of DED not associated with Sjögren's syndrome, the prognosis of Sjögren's syndrome, or the prognosis of the severity of Sjögren's syndrome in a subject comprises determining the expression profile of at least one marker of the invention in a sample of said subject.

According to a preferred embodiment, the sample was previously taken from the subject, i.e. the method of the invention does not comprise a step of recovering a sample from the subject. Consequently, according to this embodiment, the method of the invention is a non-invasive method.

In one embodiment of the invention, the sample is conjunctival superficial cells or cells from tears. In a preferred embodiment, the sample is conjunctival superficial cells.

As used herein, the term “conjunctival superficial cells” means cells from superficial layers of the ocular surface epithelium. In one embodiment, the ocular surface epithelium englobes two, three or more layers of cells. In one embodiment, conjunctival superficial cells include conjunctival epithelial cells and inflammatory cells integrated in the tissue, i.e. the ocular surface epithelium.

In one embodiment of the invention, the method of the invention comprises a step of comparing the expression profile of the markers of the signature of the invention measured in the sample of the subject with a reference expression profile, measured in a reference sample.

A reference expression profile can be relative to an expression profile derived from population studies, including without limitation, such subjects having similar age range, subjects in the same or similar ethnic group, similar DED history and the like.

In one embodiment, the reference expression profile is constructed using algorithms and other methods of statistical and structural classification.

In one embodiment of the invention, the reference expression profile is derived from the measurement of the expression profile of markers of a signature of the invention in a control sample derived from one or more substantially healthy subjects, also called “normal” patients. As used herein, a “substantially healthy subject” has not been previously diagnosed or identified as having or suffering from DED.

In one embodiment of the invention, the reference expression profile is derived from the measurement of the expression profile of markers of a signature of the invention in a control sample derived from one or more substantially DED patients suffering from mild DED.

In another embodiment of the invention, the reference expression profile is derived from the measurement of the expression profile of markers of a signature of the invention in a control sample derived from one or more substantially DED patients suffering from severe DED.

In one embodiment of the invention, the reference expression profile is derived from the previous measurement of the expression profile of markers of a signature of the invention in a reference sample derived from the same subject, such as, for example, the expression profile measured one month before, preferably six months before, more preferably one year before or more.

In another embodiment of the invention, the reference expression profile is derived from the measurement of the expression profile of markers of a signature of the invention in a reference population. In one embodiment, the reference sample is thus derived from a reference population.

In one embodiment, the reference population comprises substantially healthy subjects, preferably at least 5, more preferably at least 10, more preferably at least 20 and even more preferably at least 50 substantially healthy subjects (or “normal” patients).

In another embodiment, the reference population comprises subjects diagnosed with DED, preferably at least 5, more preferably at least 10, more preferably at least 20 and even more preferably at least 50 subjects diagnosed with DED. In one embodiment, the reference population comprises or consists on mild DED patients. In another embodiment, the reference population comprises or consists on severe DED patients.

In one embodiment, the reference expression profile corresponds to the mean expression profile of the markers of the signature of the invention measured in the reference population.

In one embodiment of the invention, the reference expression profile corresponds to the median expression profile of the markers of the genetic signature of the invention measured in the reference population.

In one embodiment of the invention, the expression of the DED markers, mild DED markers, severe DED markers of the invention corresponds to the transcription level (i.e. expression of the RNA), or to the translation level (i.e. expression of the protein) of the marker.

In one embodiment of the invention, the expression of the DED markers, mild DED markers, severe DED markers is assessed at the protein level. Methods for determining a protein level in a sample are well-known in the art. Examples of such methods include, but are not limited to, immunohistochemistry, Multiplex methods (Luminex), western blot, enzyme-linked immunosorbent assay (ELISA), sandwich ELISA, fluorescent-linked immunosorbent assay (FLISA), enzyme immunoassay (EIA), radioimmunoassay (RIA) and the like.

In another embodiment of the invention, the expression of the DED markers, mild DED markers, severe DED markers is assessed at the RNA level. Methods for assessing the transcription level of a marker are well known in the prior art. Examples of such methods include, but are not limited to, RT-PCR, RT-qPCR, Northern Blot, hybridization techniques such as, for example, use of microarrays, and combination thereof including but not limited to, hybridization of amplicons obtained by RT-PCR, sequencing such as, for example, next-generation DNA sequencing (NGS) or RNA-seq (also known as “Whole Transcriptome Shotgun Sequencing”) and the like.

In one embodiment, the method comprises the steps of:
extracting total RNA from the sample from the subject,
determining the expression profile of the markers of the signature, and
comparing said expression profile with a reference expression profile determined in a reference sample.

According to this embodiment, total RNA from the sample are not retro-transcribing to obtain cDNA.

In another embodiment, the method comprises the steps of:
extracting total RNA from the sample from the subject,
retro-transcribing these total RNA, thereby obtaining total cDNA,
specifically amplifying by PCR, preferably by qPCR, the cDNA corresponding to the DED markers, mild DED markers, or severe DED markers of the signature of the invention, thereby determining the expression profile of the markers of the signature, and
comparing said expression profile with a reference expression profile determined in a reference sample.

In one embodiment, the expression profile of markers of the signature of the invention is measured using a polynucleotide microarray, so that the expression profiles of each of the markers of the signature of the invention are simultaneously measured.

In one embodiment, the method comprises the steps of:
extracting total RNA from the sample from the subject, retro-transcribing these total RNA, thereby obtaining total cDNA from the sample, and sequencing the total cDNA from the sample from the subject,
extracting total RNA from the reference sample, retro-transcribing these total RNA, thereby obtaining total cDNA from the reference sample, and sequencing the total cDNA from the reference sample, and
comparing the results of the cDNA sequencing and identifying markers which are differentially expressed between the sample from the subject and the reference sample.

In another embodiment, the method comprises the steps of:
extracting total RNA from the sample from the subject, and sequencing the total RNA, preferably the total mRNA, from the sample from the subject,
extracting total RNA from the reference sample, and sequencing the total RNA, preferably the total mRNA from the reference sample, and
comparing the results of the RNA, preferably mRNA, sequencing and identifying markers which are differentially expressed between the sample from the subject and the reference sample.

In one embodiment of the invention, a marker of the invention is considered as differentially expressed in the sample from the subject as compared to a reference sample if both expression levels differ by a factor of at least 1.1, preferably at least 1.5, more preferably at least 2 and even more preferably at least 5.

In one embodiment, the method of the invention further comprises a step of comparing the results of markers expression with signs and symptoms commonly tested in DED and/or in Sjögren's syndrome.

Accordingly, in one embodiment, the method of the invention comprises a step of comparing the results of markers expression with results of OSDI, BUT, fluorescein coloration, osmolarity and/or age.

The present invention also relates to a kit for measuring the expression profile of markers of the signature of the invention, and/or for implementing the method of the invention. In one embodiment, the kit comprises means for determining the expression of the markers of the signature of the invention.

In one embodiment of the invention, the expression profile is measured at the protein level, and the kit of the invention comprises means for total protein extraction, as well as antibodies for detecting the markers of the invention.

In another embodiment, the expression profile is measured at the RNA level, and the kit of the invention comprises means for total RNA extraction, means for reverse transcription of total RNA, and means for quantifying the expression of RNA corresponding to the markers of the invention.

In another embodiment, the expression profile is measured at the RNA level, and the kit of the invention comprises means for total RNA extraction and means for quantifying the expression of RNA corresponding to the markers of the invention, without reverse transcription of total RNA.

In one embodiment, the means for determining the expression of the markers are PCR primers, for example qPCR primers, specific for said markers. In one embodiment, said means for determining the expression of the markers are probes to detect qPCR amplicons obtained with qPCR primers as hereinabove described.

In one embodiment, said means for quantifying the expression of RNA corresponding to the markers of the invention is PCR, for example qPCR.

In one embodiment of the invention, the kit of the invention also comprises primers for amplifying reference genes. Reference genes are genes expressed at a constant level among different tissues and/or conditions. Examples of reference genes include, but are not limited to, β-actin, genes encoding ribosomal proteins and the like.

In one embodiment of the invention, the kit of the invention comprises means for total RNA extraction, means for reverse transcription of total RNA, and/or reagents for carrying out a quantitative PCR as hereinabove described (such as, for example, primers, buffers, enzyme, and the like).

In one embodiment of the invention, the kit of the invention comprises means for total RNA extraction, and means for quantification of the expression of RNA corresponding to the markers of the invention.

In one embodiment, the kit of the invention also comprises a reference sample.

In one embodiment, the means for determining the expression of the markers of the signature comprise probes specific for said markers. In one embodiment, probes are reporter and capture probes used generate a target-probes complex with RNA, and/or plates for counting of the reporter probes.

In another embodiment, the means for determining the expression of the markers of the signature is a microarray comprising probes specific for said markers.

In one embodiment, said means for quantifying the expression of RNA corresponding to the markers of the invention is a microarray. The present invention thus also relates to microarrays for measuring the RNA expression profile of markers of the signature of the invention, and/or for implementing the non-invasive method of the invention.

In one embodiment of the invention, the microarray of the invention comprises DNA probes, which may be hybridized to the retro-transcribed RNA corresponding to the markers of the invention.

### EXAMPLES

The present invention is further illustrated by the following examples.

### Example 1

### Materials and Methods

Conjunctival superficial cells were harvested by impression cytology from patients with dry eye disease (DED) of various etiologies, including patients with mild DED and patients with severe DED and from healthy individuals (corresponding to “normal” patients). Mild and severe DED patients were identified according to usual signs and symptoms for DED, such as OSDI (Ocular Surface Disease Index), BUT (break-up time), fluorescein coloration, Schrimer test and osmolarity test.

Total RNAs were extracted according to standard procedures with commercially available extraction and purification kits. The quality and quantity of mRNA was confirmed via the determination of the RNA integrity number (RIN) with an Agilent 2100 bioanalyzer, and UV spectroscopy was used to quantitate mRNA.

NanoString nCounter technology with inflammatory human Code Set was used to analyze expressed transcripts (nCounter GX Human Inflammation Kit). Briefly, total mRNA (100 ng) is hybridized with the reporter and capture probes to generate a target-probes complex. The probes in excess are washed away and the target-probes complexes are bind onto the plate for counting of the reporter (colored) probes.

The number of colored probes is directly linked to the number of target sequences and allows for a direct quantitation of the expressed target sequences. Expression levels are tabulated in Excel files and analyzed relatively to the controls to give “fold-change” values.

### Results

Lists of markers differentially expressed between DED patients and “normal” patients are presented in Table 37. The difference in expression (average “fold-change”) and p-values are also indicated.

TABLE 37
Lists of markers differentially expressed
between DED patients and “normal” patients.

| Name of | Fold-change DED | |
|---|---|---|
| the marker | vs. normal | p-value |
| AGER | −2.5 | 0.3857 |
| AREG | 46.51 | 0.0000 |
| ATF2 | −1.45 | 0.0063 |
| C1QA | −2.66 | 0.3613 |
| C1S | −2.65 | 0.2659 |
| C2 | 1.63 | 0.0389 |
| C3AR1 | −1.76 | 0.5885 |
| CCL4 | 1.89 | 0.5782 |
| CCL13 | 1.26 | 0.7008 |
| CCL17 | −1.33 | 0.7719 |
| CCL20 | 34.74 | 0.0089 |
| CCL24 | −1.27 | 0.8114 |
| CCR2 | −2.48 | 0.0896 |
| CD4 | −2.19 | 0.1123 |
| CD40 | 1.30 | 0.0658 |
| CD163 | 1.64 | 0.5097 |
| CFD | 2.86 | 0.0153 |
| CSF1 | 2.48 | 0.2933 |
| CXCL9 | 1.78 | 0.7255 |
| CXCR4 | 1.77 | 0.5664 |
| FOS | 2.84 | 0.1485 |
| GNGT1 | −1.38 | 0.7054 |
| HDAC4 | −1.32 | 0.0169 |
| HMGB2 | −1.36 | 0.1426 |
| HSPB2 | −5.55 | 0.1021 |
| IFIT2 | 1.63 | 0.2961 |
| IFNG | 1.58 | 0.4826 |
| IL4 | −1.29 | 0.0001 |
| IL6 | 1.40 | 0.7432 |
| IL6R | −4.91 | 0.1321 |
| IL8 | 1.54 | 0.6835 |
| IL10RB | 1.39 | 0.0183 |
| IL18RAP | −1.65 | 0.2060 |
| IRF1 | 1.60 | 0.0425 |
| IRF7 | 1.51 | 0.1990 |
| JUN | 1.81 | 0.0641 |
| LTB4R | −3.31 | 0.2577 |
| MAP2K6 | −1.54 | 0.0757 |
| MAP3K5 | 1.27 | 0.3810 |
| MEF2C | −1.60 | 0.6833 |
| MRC1 | −3.92 | 0.0905 |
| MX1 | 1.54 | 0.1513 |
| MYC | −2.65 | 0.3849 |
| MYD88 | 1.37 | 0.0304 |
| NLRP3 | −2.44 | 0.0308 |
| NOD1 | −1.49 | 0.2193 |
| OASL | 3.78 | 0.2952 |
| PIK3C2G | −5.10 | 0.1627 |
| PLCB1 | −5.04 | 0.2046 |
| PRKCB | 1.32 | 0.5583 |
| PTGER2 | 1.25 | 0.5968 |
| PTGFR | −59.41 | 0.0005 |
| STAT1 | 1.59 | 0.1213 |
| STAT2 | 1.36 | 0.0917 |
| TCF4 | 1.49 | 0.6615 |
| TGFB1 | −1.67 | 0.0981 |
| TGFBR1 | −1.31 | 0.1340 |
| TLR4 | −2.02 | 0.4948 |
| TLR5 | −1.50 | 0.0005 |
| TLR8 | 1.56 | 0.4879 |
| TNF | 3.16 | 0.2353 |
| TREM2 | 1.54 | 0.4676 |

p < 0.05 *, p < 0.01 **, p < 0.001 ***

Lists of markers differentially expressed between mild DED patients and “normal” patients and their fold-change and p-values are presented in Table 38.

TABLE 38
Lists of markers differentially expressed between
mild DED patients and “normal” patients.

| Name of | Fold-change | |
|---|---|---|
| the marker | mild vs. normal | p-value |
| CCL13 | 1.66 | 0.4359 |
| C4A | −1.86 | 0.5759 |
| CFB | 1.86 | 0.1425 |
| CXCL5 | 1.80 | 0.1439 |
| CXCL6 | 1.91 | 0.4566 |
| CXCL9 | 3.67 | 0.4587 |
| CXCR2 | −2.95 | 0.1321 |
| HLA-DRA | 2.03 | 0.0394 |
| HSH2D | 1.56 | 0.1005 |
| IFI44 | 2.19 | 0.0382 |
| IFIT1 | 2.34 | 0.0920 |
| IFIT3 | 2.62 | 0.0234 |
| IL7 | 1.42 | 0.7336 |
| IRF3 | 1.48 | 0.2468 |
| ITGB2 | 2.04 | 0.5573 |
| LTB4R2 | −3.32 | 0.0441 |
| MAFF | 3.15 | 0.0128 |
| MX2 | 3.57 | 0.0244 |
| NOX1 | 3.93 | 0.2378 |
| OAS2 | 2.11 | 0.0421 |
| OASL | 9.19 | 0.1051 |
| PTGER4 | −1.30 | 0.4212 |
| RELB | 1.73 | 0.1334 |
| TNFAIP3 | 2.32 | 0.0421 |

p < 0.05 *, p < 0.01 **, p < 0.001 ***

Lists of markers differentially expressed between mild DED patients and severe DED patients and their fold-change and p-values are presented in Table 39.

TABLE 39
Lists of markers differentially expressed between
mild DED patients and severe DED patients.

| Name of | Fold-change | |
|---|---|---|
| the marker | mild vs. severe | p-value |
| CCL22 | −3.66 | 0.1513 |
| CD40LG | −1.32 | 0.2498 |
| CFB | −1.59 | 0.1056 |
| CXCL10 | −2.04 | 0.2028 |
| CXCR2 | 1.71 | 0.2686 |
| ELK1 | −1.41 | 0.0708 |
| IFIT1 | −2.11 | 0.0312 |
| IFIT3 | −1.88 | 0.0280 |
| IL1R1 | −1.36 | 0.0766 |
| IL22 | −1.26 | 0.2498 |
| IL23A | −6.08 | 0.0148 |
| LTB4R2 | 1.70 | 0.1807 |
| LY96 | −3.18 | 0.0777 |
| MAFK | −1.26 | 0.0249 |
| NOD2 | −2.50 | 0.1809 |
| NOS2 | −2.17 | 0.0072 |
| TLR6 | −1.38 | 0.6964 |
| TNFAIP3 | −1.47 | 0.1620 |

p < 0.05 *, p < 0.01 **, p < 0.001 ***

Lists of markers differentially expressed between severe DED patients and “normal” patients and their fold-change and p-values are presented in Table 40.

TABLE 40
Lists of markers differentially expressed between
severe DED patients and “normal” patients.

| Name of | Fold-change | |
|---|---|---|
| the marker | severe vs. control | p-value |
| CCL3 | 2.15 | 0.2221 |
| CXCL1 | −1.68 | 0.4196 |
| GRB2 | −3.12 | 0.0666 |
| HLA-DRB1 | −1.52 | 0.3381 |
| IL1B | 2.12 | 0.2702 |
| IL22RA2 | 1.67 | 0.2702 |
| LTB | 1.72 | 0.3053 |
| MMP9 | 1.72 | 0.4040 |
| PTGS2 | 1.84 | 0.2702 |
| TCF4 | 2.09 | 0.4707 |
| TGH32 | 2.60 | 0.1460 |
| TNFSF14 | −4.97 | 0.1334 |

p < 0.05 *, p < 0.01 **, p < 0.001 ***

Lists of markers differentially expressed between severe DED patients and mild DED patients and their fold-change and p-values are presented in Table 41.

TABLE 41
Lists of markers differentially expressed between
severe DED patients and mild DED patients.

| Name of | Fold-change | |
|---|---|---|
| the marker | severe vs. mild | p-value |
| CD55 | −6.56 | 0.0638 |
| CXCL3 | −3.63 | 0.3548 |
| IL15 | −3.55 | 0.2141 |
| IL1RN | −2.21 | 0.0276 |
| PDGFA | −2.29 | 0.1359 |
| PRKCA | −7.00 | 0.0796 |

p < 0.05 *, p < 0.01 **, p < 0.001 ***

For all tables presented in this Example, markers showing a fold-change superior to 1.2, or inferior to −1.2, and a p-value inferior to 0.05 were considered as relevant makers.

### Example 2

### Materials and Methods

A study was conducted in 88 dry eye disease (DED) patients (among which 58 DED patients (with various severity and etiologies) and 30 Sjögren's syndrome (SS) patients) and 15 aged matched healthy controls. Ocular symptom scores, ocular staining grades in the cornea (corneal fluorescein staining (CFS) as graded according to the Oxford scale, referred herein as Fluo Oxford) and conjunctiva (fluorescein dye as graded according to the Van Bijsterveld scale, referred herein as Fluo VB), tear film breakup time (TBUT), and Schirmer test, and osmolarity (solute concentration in a fluid) determination were performed to characterize patients' clinical signs and symptoms. Conjunctival superficial cells were harvested by impression cytology (Eyeprim™) and total RNAs were extracted by standard extraction procedures (Qiagen RNeasy mini kit) and RNA integrity was assessed with Agilent Bioanalyzer. The expressed transcripts were quantitated using the nCounter human inflammation code set (NanoString®). Fold changes were calculated by comparing the mean expressions values of control (healthy controls) and DED or SS patients. A t-test statistical analysis was performed using Excel; the statistical significance was set at a p value of 0.05. Spearman's rank order correlation was used to explore the correlations between the various inflammatory genes (249 genes) and both clinical signs and symptoms in the DED and SS patients. Mean expression levels were also determined, by averaging RNA levels of a marker for each assay. Finally, the coefficient of variation of expression levels of a marker was measured through all assays to determine the homogeneity of the marker within the group. Coefficients of variation were calculated as the ratio of the standard deviation to the mean of expression levels.

### Results

Lists of markers differentially expressed between SS patients and “normal” patients are presented in Table 42. The difference in expression (average “fold-change”) and p-values are indicated. The correlation factor (R) with other dry eye tests is also indicated.

TABLE 42
List of markers differentially expressed between
SS patients and “normal” subjects.

| | Expression | | | | |
|---|---|---|---|---|---|
| Name | Fold-change | | Correlation R | | |
| of the | SS vs. | | | Fluo | Fluo |
| marker | normal | p-value | OSDI | VB | Oxford |
| AREG | 2.44 | 0.106 | N/A | N/A | N/A |
| AGER | −1.58 | 0.239 | N/A | N/A | N/A |
| ATF2 | −1.24 | 0.000 | −0.205 | −0.405 | −0.285 |
| C1QA | 1.72 | 0.267 | 0.315 | 0.278 | 0.233 |
| C1QB | 1.93 | 0.030 | 0.388 | 0.288 | 0.326 |
| C2 | 1.82 | 0.000 | 0.457 | 0.264 | 0.485 |
| C3AR1 | 1.98 | 0.052 | 0.329 | 0.202 | 0.276 |
| C4A | 2.25 | 0.091 | 0.504 | 0.167 | 0.240 |
| C5 | −1.24 | 0.664 | −0.319 | 0.023 | −0.080 |
| CCL17 | −1.63 | 0.158 | −0.272 | −0.176 | −0.122 |
| CCL20 | 5.25 | 0.101 | N/A | N/A | N/A |
| CCL22 | 3.82 | 0.000 | 0.311 | 0.236 | 0.408 |
| CCL24 | 16.62 | 0.032 | 0.361 | 0.316 | 0.441 |
| CCL3 | 2.85 | 0.240 | 0.321 | 0.199 | 0.383 |
| CCL4 | 4.24 | 0.017 | 0.283 | 0.323 | 0.436 |
| CCL5 | 1.70 | 0.002 | 0.335 | 0.475 | 0.531 |
| CCR1 | 4.90 | 0.033 | 0.384 | 0.197 | 0.306 |
| CFB | 1.89 | 0.000 | 0.376 | 0.263 | 0.400 |
| CFD | 1.56 | 0.118 | −0.071 | 0.392 | 0.164 |
| CSF1 | 2.30 | 0.055 | 0.292 | 0.262 | 0.392 |
| CXCL1 | 1.72 | 0.450 | 0.061 | −0.128 | −0.137 |
| CXCL10 | 2.14 | 0.193 | 0.325 | 0.183 | 0.271 |
| CXCL2 | 1.88 | 0.049 | 0.398 | 0.233 | 0.177 |
| CXCL3 | 2.56 | 0.116 | 0.322 | 0.068 | 0.112 |
| CXCL6 | 1.91 | 0.109 | 0.144 | −0.128 | −0.098 |
| CXCL9 | 7.12 | 0.012 | 0.354 | 0.201 | 0.294 |
| CXCR2 | −1.22 | 0.325 | N/A | N/A | N/A |
| CXCR4 | 3.93 | 0.039 | 0.471 | 0.539 | 0.552 |
| FASLG | 2.27 | 0.182 | 0.124 | 0.245 | 0.314 |
| FOS | 2.01 | 0.110 | 0.462 | 0.459 | 0.562 |
| GNGT1 | −1.95 | 0.032 | −0.696 | −0.215 | −0.413 |
| HLA-DRA | 2.40 | 0.001 | 0.482 | 0.388 | 0.491 |
| HLA-DRB1 | 1.99 | 0.005 | 0.285 | 0.202 | 0.381 |
| HMGB1 | −1.31 | 0.001 | −0.211 | −0.110 | −0.218 |
| HMGB2 | −1.46 | 0.021 | N/A | N/A | N/A |
| HSPB2 | −1.74 | 0.038 | −0.530 | −0.182 | −0.340 |
| IFNG | 2.38 | 0.250 | 0.187 | 0.256 | 0.420 |
| IL12A | −1.80 | 0.459 | −0.080 | −0.017 | 0.092 |
| IL15 | 1.52 | 0.013 | 0.250 | 0.127 | 0.323 |
| IL1RN | 1.57 | 0.004 | 0.284 | 0.010 | 0.178 |
| IL23A | 5.36 | 0.025 | 0.230 | −0.220 | −0.056 |
| IL6 | 16.73 | 0.014 | 0.445 | 0.223 | 0.348 |
| IL6R | −1.66 | 0.280 | −0.021 | 0.010 | 0.026 |
| IL8 | 7.76 | 0.127 | 0.391 | 0.137 | 0.166 |
| ITGB2 | 2.16 | 0.009 | 0.129 | 0.249 | 0.385 |
| JUN | 1.64 | 0.064 | 0.496 | 0.431 | 0.495 |
| KEAP1 | −1.29 | 0.044 | −0.095 | −0.152 | −0.194 |
| LTB | 2.81 | 0.207 | 0.370 | 0.218 | 0.311 |
| LTB4R | −1.68 | 0.013 | N/A | N/A | N/A |
| LTB4R2 | −1.81 | 0.007 | N/A | N/A | N/A |
| LY96 | 2.59 | 0.006 | 0.340 | 0.292 | 0.345 |
| MAFF | 1.83 | 0.021 | 0.392 | 0.071 | 0.253 |
| MAP2K6 | −1.26 | 0.045 | −0.387 | 0.035 | −0.116 |
| MMP9 | 11.71 | 0.139 | N/A | N/A | N/A |
| MRC1 | 1.52 | 0.598 | N/A | N/A | N/A |
| MX1 | 1.51 | 0.081 | N/A | N/A | N/A |
| MX2 | 1.57 | 0.353 | N/A | N/A | N/A |
| NFATC2 | −1.33 | 0.000 | −0.198 | −0.199 | −0.254 |
| NOD2 | 8.77 | 0.002 | 0.312 | −0.022 | 0.132 |
| NOS2 | 2.11 | 0.004 | 0.312 | −0.022 | 0.132 |
| NOX1 | 3.14 | 0.001 | 0.480 | 0.190 | 0.287 |
| OAS2 | 1.56 | 0.120 | N/A | N/A | N/A |
| PDGFA | −1.29 | 0.127 | −0.011 | −0.178 | −0.174 |
| PIK3C2G | −1.76 | 0.328 | −0.255 | −0.192 | −0.190 |
| PLCB1 | −2.17 | 0.040 | −0.264 | 0.082 | −0.157 |
| PRKCA | −1.43 | 0.015 | −0.128 | −0.082 | −0.261 |
| PTGFR | −3.75 | 0.040 | N/A | N/A | N/A |
| PTGS2 | 17.60 | 0.180 | N/A | N/A | N/A |
| STAT1 | 1.84 | 0.003 | 0.363 | 0.355 | 0.451 |
| TCF4 | −1.61 | 0.224 | N/A | N/A | N/A |
| TLR4 | 2.03 | 0.174 | 0.302 | 0.053 | 0.127 |
| TLR5 | −1.17 | 0.078 | −0.374 | 0.059 | −0.154 |
| TLR6 | −1.31 | 0.525 | −0.132 | 0.059 | 0.056 |
| TLR8 | 1.66 | 0.543 | N/A | N/A | N/A |
| TNF | 3.84 | 0.004 | 0.277 | 0.309 | 0.417 |
| TNFAIP3 | 1.78 | 0.023 | N/A | N/A | N/A |
| TNFSF14 | 1.80 | 0.065 | 0.388 | 0.028 | 0.249 |

p < 0.05 *, p < 0.01 **, p < 0.001 ***, R ≥ 0.5, high positive correlation; 0.3 ≤ R < 0.5, mild positive correlation; −0.5 < R ≤ −0.3, mild negative correlation; and −0.5 ≤ R, high negative correlation.
N/A, not analyzed.

Markers showing a fold-change superior to 1.2, or inferior to −1.2, and a p-value inferior to 0.05 were considered as relevant makers. For example, CCL17 shows a fold-change superior to 1.2 but a p-value superior to 0.05, and was thus not considered as relevant.

Moreover, the correlation between each marker and at least one dry eye test OSDI, VB CFS and Oxford CFS was analyzed to determine the severity of the disease. Markers showing a correlation superior to 0.3 or inferior to −0.3 were considered as relevant makers. For example, NFATC3 shows a fold-change inferior to −1.2 and a p-value inferior to 0.05, but no significant correlation with any other dry eye test, and was thus not considered as relevant.

Mean expression levels and coefficients of variation were also analyzed and optionally used for determining relevancy of the marker. Results are presented in Table 43.

TABLE 43
Mean expression levels and coefficients of variation of markers
differentially expressed between SS patients and “normal” subjects.

| Name of the | Mean expression | Coefficient of |
|---|---|---|
| marker | level | variation |
| CCL4 | 56-114 | 156 |
| CCR1 | 14-43 | 178 |
| CXCL2 | 1412-1951 | 111 |
| GNGT1 | 233-361 | 94 |
| HLA-DRB1 | 11030-15123 | 75 |
| HMGB2 | 261-307 | 43 |
| HSPB2 | 40-62 | 107 |
| IL15 | 157-190 | 53 |
| IL1RN | 49328-63068 | 48 |
| IL23A | 36-31 | 177 |
| IL6 | 45-140 | 194 |
| ITGB2 | 78-117 | 93 |
| KEAP1 | 425-503 | 45 |
| LTB4R2 | 169-235 | 60 |
| MAFF | 707-797 | 92 |
| NOS2 | 3679-5233 | 84 |
| STAT1 | 6523-9045 | 66 |
| TNFAIP3 | 1397-1414 | 86 |

In sample of small size (as it will be the case for the genechip assays) markers with higher expression levels will be more easily detected than those with lower levels of expression. Thus, among the selected genes, the ones with higher expression levels (for instance, those with mean expression levels of more than 100 transcripts per assay) are preferred to have an improved genechip assay sensitivity. The coefficient of variation (the ratio of the standard deviation to the mean) is a measure of the dispersion within a group. The CV (%) is an indication of the homogeneity within a group (of patients for example). Therefore, genes with lower CVs are preferred.

Furthermore, the list of markers differentially expressed between SS patients and DED patients that do not suffer from SS is presented in Table 44. The difference in expression (average “fold-change”) and p-values are indicated. The correlation factor (R) with other dry eye tests is also indicated.

TABLE 44
List of markers differentially expressed between SS
patients and DED patients that do not suffer from SS.

| | Expression | | | |
|---|---|---|---|---|
| Name | Fold-change | | Correlation R | |
| of the | SS vs. | | | Fluo |
| marker | DED-SS | p-value | Fluo VB | Oxford |
| AGER | −1.93 | 0.004 | 0.047 | 0.027 |
| ALOX15 | 1.33 | 0.014 | −0.139 | −0.254 |
| AREG | 1.98 | 0.133 | −0.111 | −0.131 |
| C1QA | 2.71 | 0.046 | −0.034 | 0.014 |
| C1QB | 1.21 | 0.374 | −0.129 | −0.155 |
| C1S | −2.90 | 0.059 | −0.187 | −0.148 |
| C3AR1 | 1.73 | 0.076 | N/A | N/A |
| C4A | 2.35 | 0.079 | N/A | N/A |
| C5 | −1.71 | 0.121 | 0.064 | 0.129 |
| CCL17 | −1.56 | 0.078 | −0.019 | 0.022 |
| CCL20 | 1.43 | 0.552 | −0.208 | −0.192 |
| CCL24 | 1.40 | 0.593 | 0.011 | −0.031 |
| CCL4 | 2.02 | 0.117 | N/A | N/A |
| CCR1 | 3.14 | 0.053 | 0.034 | 0.078 |
| CCR2 | −2.13 | 0.062 | N/A | N/A |
| CD4 | 1.67 | 0.001 | −0.022 | −0.007 |
| CD40 | 1.24 | 0.003 | −0.017 | −0.005 |
| CD55 | 1.45 | 0.056 | 0.121 | 0.050 |
| CD86 | 1.72 | 0.248 | −0.056 | 0.004 |
| CSF1 | 2.71 | 0.030 | 0.290 | 0.208 |
| CXCL1 | 1.62 | 0.478 | 0.136 | 0.211 |
| CXCL2 | 1.38 | 0.236 | −0.025 | −0.019 |
| CXCL3 | 1.20 | 0.697 | 0.011 | 0.072 |
| CXCL6 | 1.32 | 0.402 | N/A | N/A |
| CXCL9 | 1.33 | 0.527 | N/A | N/A |
| CXCR4 | 1.86 | 0.188 | 0.038 | −0.016 |
| DDIT3 | −1.26 | 0.409 | N/A | N/A |
| ELK1 | 1.20 | 0.008 | 0.097 | −0.018 |
| FOS | 2.04 | 0.009 | N/A | N/A |
| GNGT1 | −1.55 | 0.031 | N/A | N/A |
| HLA-DRA | 1.45 | 0.057 | N/A | N/A |
| HLA-DRB1 | 1.37 | 0.100 | −0.074 | −0.136 |
| HMGB2 | −1.18 | 0.091 | N/A | N/A |
| HSH2D | −1.21 | 0.374 | −0.138 | −0.372 |
| HSPB2 | −1.58 | 0.034 | −0.142 | −0.151 |
| IFIT1 | −1.31 | 0.525 | N/A | N/A |
| IFIT3 | −1.29 | 0.422 | 0.078 | −0.083 |
| IFNG | 1.62 | 0.377 | 0.177 | 0.019 |
| IL1B | 9.83 | 0.253 | N/A | N/A |
| IL12A | −1.22 | 0.713 | N/A | N/A |
| IL15 | 1.21 | 0.183 | −0.131 | −0.077 |
| IL1R1 | 1.35 | 0.012 | 0.188 | 0.154 |
| IL1RN | 1.28 | 0.078 | −0.142 | −0.124 |
| IL23A | −1.16 | 0.792 | −0.129 | −0.350 |
| IL6 | 3.11 | 0.085 | 0.105 | 0.288 |
| IL7 | 1.71 | 0.009 | N/A | N/A |
| IL8 | 2.49 | 0.294 | 0.132 | 0.235 |
| IRF3 | 1.23 | 0.015 | 0.202 | 0.058 |
| IRF7 | 1.54 | 0.097 | 0.112 | 0.192 |
| ITGB2 | 1.51 | 0.090 | N/A | N/A |
| JUN | 1.34 | 0.043 | −0.033 | 0.029 |
| KEAP1 | −1.18 | 0.074 | N/A | N/A |
| LTB4R | 1.27 | 0.161 | 0.068 | −0.054 |
| LTB4R2 | −1.39 | 0.024 | 0.005 | −0.001 |
| MAFK | 1.23 | 0.003 | N/A | N/A |
| MAP2K1 | −1.15 | 0.057 | −0.129 | −0.350 |
| MAPKAPK2 | 1.28 | 0.000 | N/A | N/A |
| MRC1 | 1.46 | 0.570 | −0.119 | −0.009 |
| MX2 | −1.17 | 0.743 | −0.327 | −0.324 |
| MYC | −1.19 | 0.322 | N/A | N/A |
| NOD1 | 1.40 | 0.012 | 0.068 | 0.041 |
| NOS2 | 1.42 | 0.107 | N/A | N/A |
| NOX1 | 1.71 | 0.039 | 0.091 | 0.120 |
| OASL | −1.45 | 0.619 | 0.059 | 0.082 |
| OXER1 | 2.28 | 0.212 | N/A | N/A |
| PIK3C2G | −1.44 | 0.360 | N/A | N/A |
| PLA2G4A | −1.21 | 0.021 | 0.204 | 0.096 |
| PRKCA | 1.58 | 0.001 | N/A | N/A |
| PTGER4 | 1.76 | 0.002 | 0.149 | 0.135 |
| PTGFR | 1.41 | 0.447 | 0.139 | 0.238 |
| SMAD7 | −1.19 | 0.003 | −0.032 | 0.030 |
| STAT1 | 1.39 | 0.068 | 0.080 | 0.059 |
| TGFB1 | 1.30 | 0.075 | 0.046 | 0.067 |
| TGFBR1 | 1.26 | 0.025 | 0.117 | 0.012 |
| TLR4 | 1.78 | 0.187 | N/A | N/A |
| TLR6 | 1.26 | 0.376 | 0.065 | 0.042 |
| TNF | 1.41 | 0.328 | 0.139 | 0.120 |
| TNFSF14 | 2.64 | 0.006 | N/A | N/A |
| TRADD | 1.31 | 0.002 | 0.164 | 0.021 |
| TREM2 | 2.04 | 0.296 | 0.135 | 0.261 |

p < 0.05 *, p < 0.01 **, p < 0.001 ***, R ≥ 0.5, high positive correlation; 0.3 ≤ R < 0.5, mild positive correlation; −0.5 < R ≤ −0.3, mild negative correlation; and −0.5 ≤ R, high negative correlation.
N/A, not analyzed.

Furthermore, the list of markers differentially expressed between DED patients that do not suffer from SS and healthy subjects is presented in Table 45. The difference in expression (average “fold-change”) and p-values are indicated. The correlation factor (R) with other dry eye tests is also indicated.

TABLE 45
List of markers differentially expressed between DED
patients that do not suffer from SS and healthy subjects.

| | Expression | | | | |
|---|---|---|---|---|---|
| Name | Fold-change | | Correlation R | | |
| of the | SS vs. | | | Fluo | Fluo |
| marker | normal | p-value | OSDI | VB | Oxford |
| AGER | 1.22 | 0.475 | N/A | N/A | N/A |
| ALOX15 | 0.74 | 0.085 | N/A | N/A | N/A |
| AREG | 1.23 | 0.661 | N/A | N/A | N/A |
| ATF2 | 0.75 | 0.000 | −0.271 | −0.224 | −0.200 |
| BCL6 | 0.84 | 0.073 | −0.435 | −0.145 | −0.343 |
| C1QA | 0.63 | 0.401 | −0.054 | 0.169 | 0.043 |
| C1QB | 1.59 | 0.046 | −0.002 | 0.248 | 0.011 |
| C1S | 3.03 | 0.058 | −0.167 | 0.208 | 0.043 |
| C2 | 1.57 | 0.001 | −0.026 | −0.075 | −0.142 |
| C5 | 1.38 | 0.423 | −0.078 | 0.286 | 0.111 |
| CCL20 | 3.67 | 0.017 | N/A | N/A | N/A |
| CCL22 | 3.28 | 0.006 | −0.089 | −0.177 | −0.214 |
| CCL24 | 11.88 | 0.055 | −0.014 | 0.081 | −0.034 |
| CCL4 | 2.10 | 0.096 | −0.152 | 0.050 | −0.077 |
| CCL5 | 1.61 | 0.009 | 0.115 | 0.274 | 0.136 |
| CCR1 | 1.56 | 0.495 | −0.088 | 0.100 | −0.001 |
| CD4 | 0.55 | 0.007 | −0.283 | −0.015 | −0.325 |
| CD55 | 0.64 | 0.030 | −0.311 | −0.295 | −0.228 |
| CD86 | 0.77 | 0.775 | N/A | N/A | N/A |
| CDC42 | 1.29 | 0.031 | 0.235 | 0.235 | −0.071 |
| CFB | 1.66 | 0.004 | 0.079 | −0.144 | −0.145 |
| CFD | 1.73 | 0.035 | 0.190 | 0.247 | 0.190 |
| CFL1 | 1.29 | 0.001 | 0.107 | 0.143 | −0.149 |
| CSF1 | 0.85 | 0.647 | −0.053 | −0.141 | −0.017 |
| CXCL10 | 2.33 | 0.112 | 0.028 | 0.023 | 0.000 |
| CXCL2 | 1.36 | 0.193 | 0.074 | 0.075 | 0.063 |
| CXCL3 | 2.13 | 0.088 | −0.056 | −0.149 | −0.110 |
| CXCL6 | 1.45 | 0.203 | −0.148 | 0.012 | −0.164 |
| CXCL9 | 5.34 | 0.014 | −0.079 | −0.081 | −0.067 |
| CXCR4 | 2.12 | 0.004 | 0.117 | 0.348 | 0.106 |
| DDIT3 | 1.43 | 0.241 | −0.036 | 0.117 | −0.153 |
| FASLG | 2.45 | 0.167 | −0.042 | 0.174 | 0.077 |
| GNAQ | 1.40 | 0.000 | 0.274 | 0.207 | −0.043 |
| GNGT1 | 0.79 | 0.342 | −0.019 | 0.018 | −0.107 |
| HLA-DRA | 1.65 | 0.009 | −0.057 | 0.104 | −0.079 |
| HLA-DRB1 | 1.45 | 0.079 | −0.104 | −0.042 | −0.092 |
| HMGB1 | 0.77 | 0.001 | −0.017 | −0.073 | −0.037 |
| HMGB2 | 0.81 | 0.121 | N/A | N/A | N/A |
| HSH2D | 1.49 | 0.124 | N/A | N/A | N/A |
| IFI44 | 1.26 | 0.485 | N/A | N/A | N/A |
| IFIT1 | 1.68 | 0.335 | N/A | N/A | N/A |
| IFIT2 | 1.26 | 0.489 | N/A | N/A | N/A |
| IFIT3 | 1.83 | 0.169 | N/A | N/A | N/A |
| IFNG | 1.47 | 0.629 | −0.003 | 0.123 | 0.098 |
| IL10RB | 1.25 | 0.007 | 0.113 | −0.041 | −0.175 |
| IL12A | 0.68 | 0.588 | −0.088 | −0.137 | −0.112 |
| IL15 | 1.26 | 0.174 | −0.256 | −0.123 | −0.315 |
| IL1B | 1.86 | 0.393 | −0.016 | 0.112 | 0.118 |
| IL1RN | 1.23 | 0.214 | −0.175 | −0.027 | −0.196 |
| IL23A | 6.21 | 0.065 | −0.047 | −0.160 | −0.263 |
| IL6 | 5.38 | 0.055 | −0.137 | −0.021 | −0.069 |
| IL6R | 0.61 | 0.268 | −0.174 | 0.142 | 0.014 |
| IL7 | 0.54 | 0.033 | −0.438 | −0.152 | −0.198 |
| IL8 | 3.11 | 0.005 | 0.047 | −0.050 | 0.037 |
| IRF1 | 1.33 | 0.061 | N/A | N/A | N/A |
| IRF7 | 0.64 | 0.219 | N/A | N/A | N/A |
| ITGB2 | 1.43 | 0.125 | −0.181 | 0.074 | 0.079 |
| JUN | 1.22 | 0.467 | 0.134 | 0.185 | 0.091 |
| LTB4R | 0.47 | 0.002 | N/A | N/A | N/A |
| LTB4R2 | 0.77 | 0.135 | N/A | N/A | N/A |
| LY96 | 2.69 | 0.017 | 0.075 | 0.075 | −0.026 |
| MAFF | 1.62 | 0.072 | −0.030 | −0.231 | 0.013 |
| MAP2K6 | 0.76 | 0.015 | −0.164 | 0.073 | −0.164 |
| MAP3K5 | 1.27 | 0.002 | −0.040 | 0.072 | −0.018 |
| MX1 | 1.31 | 0.347 | N/A | N/A | N/A |
| MX2 | 1.84 | 0.346 | N/A | N/A | N/A |
| MYD88 | 1.29 | 0.010 | 0.030 | −0.048 | −0.227 |
| NFATC3 | 0.79 | 0.000 | −0.004 | −0.029 | 0.085 |
| NOD1 | 0.62 | 0.008 | N/A | N/A | N/A |
| NOD2 | 9.33 | 0.021 | N/A | N/A | N/A |
| NOS2 | 1.48 | 0.062 | −0.013 | −0.160 | −0.201 |
| NOX1 | 1.84 | 0.013 | 0.050 | −0.029 | 0.000 |
| OAS2 | 1.43 | 0.287 | N/A | N/A | N/A |
| OASL | 1.86 | 0.488 | N/A | N/A | N/A |
| OXER1 | 0.43 | 0.496 | N/A | N/A | N/A |
| PDGFA | 0.81 | 0.196 | −0.196 | 0.140 | −0.119 |
| PIK3C2G | 0.82 | 0.683 | −0.116 | 0.089 | 0.106 |
| PLA2G4A | 1.36 | 0.001 | 0.250 | 0.091 | −0.082 |
| PLCB1 | 0.47 | 0.037 | −0.260 | 0.025 | −0.019 |
| PRKCA | 0.44 | 0.000 | −0.409 | −0.100 | −0.283 |
| PTGER4 | 0.77 | 0.151 | N/A | N/A | N/A |
| PTGFR | 0.19 | 0.025 | N/A | N/A | N/A |
| RELA | 0.83 | 0.058 | N/A | N/A | N/A |
| SHC1 | 0.77 | 0.005 | −0.405 | −0.446 | −0.248 |
| STAT1 | 1.32 | 0.137 | −0.086 | −0.095 | −0.223 |
| TCF4 | 0.69 | 0.313 | N/A | N/A | N/A |
| TGFB2 | 2.02 | 0.093 | −0.232 | 0.142 | 0.033 |
| TGFBR1 | 0.73 | 0.032 | −0.420 | −0.024 | −0.239 |
| TLR3 | 1.20 | 0.175 | −0.004 | −0.008 | −0.311 |
| TLR5 | 0.81 | 0.025 | −0.293 | 0.024 | −0.237 |
| TLR6 | 0.61 | 0.270 | −0.170 | 0.075 | −0.055 |
| TLR8 | 1.73 | 0.461 | N/A | N/A | N/A |
| TNF | 2.72 | 0.031 | −0.154 | 0.039 | 0.101 |
| TNFAIP3 | 1.75 | 0.086 | N/A | N/A | N/A |
| TNFSF14 | 0.68 | 0.162 | −0.173 | −0.131 | −0.242 |
| TREM2 | 0.67 | 0.656 | N/A | N/A | N/A |

p < 0.05 *, p < 0.01 **, p < 0.001 ***, R ≥ 0.5, high positive correlation; 0.3 ≤ R < 0.5, mild positive correlation; −0.5 < R ≤ −0.3, mild negative correlation; and −0.5 ≤ R, high negative correlation.
N/A, not analyzed.

Markers showing a fold-change superior to 1.2, or inferior to −1.2, and a p-value inferior to 0.05 were considered as relevant makers.

Moreover, the correlation between each marker and at least one dry eye test OSDI, VB CFS and Oxford CFS was analyzed to determine the severity of the disease. Markers showing a correlation superior to 0.3 or inferior to −0.3 were considered as relevant makers.

Mean expression levels and coefficients of variation were also determined. Results are presented in Table 46.

TABLE 46
Mean expression levels and coefficients of variation of markers differentially
expressed between DED-SS patients and “normal” subjects.

| Name of the | Mean expression | Coefficients of |
|---|---|---|
| marker | levels | variation |
| IL8 | 1374-3427 | 169 |
| CCL20 | 202-289 | 185 |
| CFD | 2963-2671 | 89 |
| GNAQ | 2957-2670 | 24 |
| PLA2G4A | 1901-1574 | 35 |
| CFL1 | 19139-18525 | 30 |
| MAP3K5 | 443-438 | 39 |
| CDC42 | 36614-33819 | 37 |
| IL10RB | 6699-6132 | 41 |
| TLR5 | 740-780 | 32 |
| SHC1 | 1563-1753 | 32 |
| NOD1 | 130-182 | 39 |
| CD4 | 122-203 | 44 |
| IL7 | 40-68 | 88 |
| CD55 | 47-68 | 80 |
| TGFBR1 | 952-1197 | 52 |

1. A method for the prognosis of dry eye disease (DED) in a subject, wherein said method comprises assessing the expression of markers of a signature comprising (i) at least one DED marker selected from the list of Table 2 or Table 9; (ii) at least one mild DED marker; and (iii) at least one severe DED marker in a sample from said subject.

2. The method according to claim 1, wherein said at least one DED marker is selected from the list of Table 3 or Table 9.

3. The method according to claim 1, wherein said at least one mild DED marker is a marker whose expression is different between a patient suffering from mild DED and a “normal” patient, or between a patient suffering from mild DED and a patient suffering from severe DED.

4. The method according to claim 1, wherein said at least one mild DED marker is selected from the list of Table 11, fragments, variants and equivalents thereof; and/or from the list of Table 15, fragments, variants and equivalents thereof.

5. The method according to claim 1, wherein said at least one severe DED marker is a marker whose expression is different between a patient suffering from severe DED and a “normal” patient, or between a patient suffering from severe DED and a patient suffering from mild DED.

6. The method according to claim 1, wherein said at least one severe DED marker is selected from the list of Table 19, fragments, variants and equivalents thereof; and/or from the list of Table 24, fragments, variants and equivalents thereof.

7. The method according to claim 1, wherein said sample is conjunctival superficial cells of said subject.

8. The method according to claim 1, wherein said subject is a human.

9. The method according to claim 1, further comprising comparing said expression with a reference expression profile.

10. The method according to claim 1, wherein said method comprises the steps of:
extracting total RNA from the sample from the subject,
determining the expression profile of the markers of the signature, and
comparing said expression profile with a reference expression profile determined in a reference sample.

11. The method according to claim 1, wherein said method is a non-invasive method.

12. A kit for implementing the method according to claim 1, wherein said kit comprises means for determining the expression of the markers of the signature.

13. The kit according to claim 12, wherein said kit comprises a genechip specific for dry eye disease, comprising at least 3 of the genes selected from the group comprising markers of the lists of Table 1 or Table 9, Table 11, Table 15, Table 19 and Table 24, or homologs thereof.

14. A method for the identification of patients with dry eye disease (DED) but not Sjögren's syndrome or for the identification of the severity of dry eye disease (DED) in a subject that does not suffer from Sjögren's syndrome, wherein said method comprises assessing the expression of the following markers:
for the identification of patients with dry eye disease (DED) but not Sjögren's syndrome, at least one marker selected from Table 28, and
for the identification of the severity of dry eye disease (DED) in a subject that does not suffer from Sjögren's syndrome, at least one marker selected from the group comprising CFD, GNAQ, PLA2G4A, CDC42, SHC1, CD4, IL7, CD55 and TGFBR1.

15. A method for the prognosis or for the identification of the severity of Sjögren's syndrome in a subject, wherein said method comprises assessing the expression of the following markers:
for the prognosis of Sjögren's syndrome, at least one marker selected from Table 33, and
for the identification of the severity of Sjögren's syndrome, at least one marker selected from the group comprising IL6, CCR1, CCL4, MAFF, NOS2, ITGB2, HLA-DRB1, CXCL2, STAT1, IL1RN, IL15, GNGT1 and HSPB2.
